(19) United States
(12) Patent Application Publication
(10) Pub. No.: US 2005/0019841 A1
(43) Pub. Date: Jan. 27, 2005

(54) Modulation of signaling pathways

(75) Inventors: Jonathan David Garman, San Jose, CA (US); Peter S. Lu, Mountain View, CA (US)

Correspondence Address: Jonathan David Garman, 772 Lucerne Drive, Sunnyvale, CA, 94085, US

(73) Assignee: Arbor Vita Corporation, Sunnyvale, CA (US)

(21) Appl. No.: 10/684,796

(22) Filed: Oct. 14, 2003

Related U.S. Application Data
(63) Continuation-in-part of application No. PCT/US02/24655, filed on Aug. 2, 2002.
(63) Continuation-in-part of application No. 10/080,273, filed on Feb. 19, 2002.
(63) Continuation-in-part of application No. 09/724,553, filed on Nov. 28, 2000.
(63) Continuation-in-part of application No. 09/570,118, filed on May 12, 2000, now abandoned.
(60) Provisional application No. 60/418,042, filed on Oct. 11, 2002.
(60) Provisional application No. 60/426,212, filed on Nov. 14, 2002.
(60) Provisional application No. 60/309,841, filed on Aug. 3, 2001.
(60) Provisional application No. 60/360,061, filed on Feb. 25, 2002.
(60) Provisional application No. 60/269,523, filed on Feb. 16, 2001.
(60) Provisional application No. 60/134,114, filed on May 14, 1999.

(51) Int. Cl. G01N 33/53

(52) U.S. Cl. 435/7.5; 530/350

(57) ABSTRACT

The invention provides reagents and methods for inhibiting or enhancing interactions between proteins in cells, particularly interactions between PDZ proteins and a PL protein. Methods and compositions provided herein are useful for treatment of a variety of diseases and conditions mediated signal transduction.

### CROSS-REFERENCE

This application: a) claims the benefit of U.S. Provisional Application No. 60/418,042, filed Oct. 11, 2002,and U.S. Provisional Application No. 60/426,212, filed Nov. 14, 2002, and b) is a CIP of of PCT Application No. US02/24655, filed Aug. 2, 2002, which application claims the benefit of U.S. Provisional Application No. 60/309841, filed Aug. 3, 2001, and U.S. Provisional Application No. 60/360061, filed Feb. 25, 2002, and c) is a CIP of U.S. Non-Provisional application Ser. No. 10/080,273, filed Feb. 19, 2002, which application claims the benefit of U.S. Provisional Application No. 60/269,523, filed Feb. 16, 2001, and d) is a CIP of U.S. Non-Provisional application Ser. No. 09/724,553,filed Nov. 28, 2000, and e) is a CIP of U.S. Non-Provisional application Ser. No. 09/570,118, filed May 12, 2000, which application claims the benefit of U.S. Provisional Application No. 60/134,114, filed May 14, 1999, all of which applications are incorporated herein by reference in their entirety.

### FIELD OF THE INVENTION

The present invention relates to small molecules, peptides, peptide analogues, proteins, and methods for using such compositions to regulate signalling pathways in cells. In one aspect, the invention provides methods of modulating localization or function of receptors that bind heterotrimeric G proteins by antagonizing or promoting binding between a PDZ domain containing protein and a protein that binds a PDZ domain.

### BACKGROUND

G-Protein-Coupled-Receptors (GPCRs) constitute the largest family of cell surface molecules involved in signal transmission. These receptors play key physiological roles and their dysfunction results in diseases or disorders such as immune and cardiovascular disorders including asthma and inflammation, neurological disorders including anxiety, memory, depression, and cognition, endocrine disorders and more. Their importance in physiological systems has made them one of the most-often targeted classes of proteins for drug discovery.

Estimates of the number of different GPCRs in the human genome range from 300 to over 1000. This makes drug discovery a complex process requiring significant trial and error in the identification of compounds that will inhibit a single GPCR. In an effort to reduce the complexity of this effort, many groups are attempting to develop drugs that inhibit interactions between hetero- or homo-dimerized GPCRs, regulatory features such as phosphorylation sites, or inhibition of specific G-protein binding to GPCRs. There still exists a great need to be able to effectively identify therapeutics that target this class of proteins.

One class of GPCRs in need of additional therapeutic inhibitors is the alpha adrenergic receptors. Six alpha adrenergic receptors have been identified at this time: alpha 1A, 1B and 1C and alpha 2A, 2B and 2C. Alpha 1 receptors have been shown to mediate actions in the sympathetic nervous system through binding of hormones such as catecholamines, epinephrine and norepinephrine. Alpha 2 receptors have been shown to play roles in regulting neurotransmitter release from sympathetic and adrenergic neurons in the central nervous-system. The tissue distributions differ between members of each group of receptors, arguing a need for type specific or sub-type specific therapeutics. Specific antagonists and agonists of certain alpha adrenergic receptors (aAR's) have been identified, but the pharmacokinetic profiles of certain alpha 1 adrenergeric receptors (a1AR's) demonstrate that they penetrate the blood brain barrier, potentially giving rise to adverse side effects (Pool J L. Int. Urol. Nephrol 2001, 33(3):407). However, several indications merit therapeutic targeting of brain functions, so the need for blood brain barrier penetrance will be receptor type and disease specific. Alpha 1 receptors have been experimentally implicated in depression, lower urinary tract storage, migraines, prostate apoptosis, and hypertrophy/proliferation/migration of vascular smooth muscle following carotid balloon injury. Alpha 2 receptors have been experimentally implicated in migraine, glucose metabolism, coronary flow reserve after stenting, Alzheimer's, Parkinson's, neuroprotection, glaucoma, and opioid withdrawl management. We have demonstrated binding between alpha adrenergic receptors and PDZ proteins, thus allowing a novel ,set of targets to treat the disorders listed above.

We have identified that PDZ proteins can organize and regulate the expression and function of a subset of GPCRs. PDZ domain-containing proteins have since been shown to regulate a myriad of cellular functions from vesicular trafficking,tumor suppression, protein sorting, establishment of membrane polarity, and apoptosis. A common function of this family is to facilitate the assembly of multi-protein complexes, often serving as a bridge between several proteins. By possessing multiple PDZ domains, many PDZ-containing proteins act as organizers within the cell by increasing the local concentration of one or more proteins, and by regulating the localization of the clusters through interactions with the cytoskeleton or other organelles. One such protein, EBP50 has been shown to be an essential link between the β2-adrenergic receptor and the actin cytoskeleton, regulating its proper endocytosis and recycling to the plasma membrane. Another protein containing multiple PDZ domains, PDZK1, is essential for regulating ion conductance and polarized membrane distribution of the cystic fibrosis chloride channel. Others contain intrinsic enzymatic activity, and use their PDZ domains to localize the enzyme with its appropriate substrates. Thus, PDZ domains represent an important means by which the cell regulates the organization, localization, and function of proteins. The function of PDZ domains in certain biological systems is described, for example, in published PCT applications that are commonly owned by the assignee of the instant application (see, e.g., WO 00/13161, WO 00/69898 and WO 00/69897), each of which is incorporated herein by reference in its entirety for all purposes). PDZ interactions with their ligands have been shown to be amenable to therpaeutic intervention (Aarts et al., Science 2002, 298:846), thus underscoring the therpaeutic potential for these interactions.

The following publications are of interest: Stone (2003) Neuropsychopharmacology 28(8):1387-99; Djavan (2003) Urology 62:6-14; Willems (2003) Cephalalgia 23(4):245-57;Anglin (2002) Prostate Cancer Prostatic Dis. 5(2):88-95; Pool (2001) Int Urol Nephrol 33(3):407-12; Roehrborn (2002) 59:3-6; Velliquette (2003) J Pharmacol Exp Ther 306(2):646-57; Stewart (2002) Circulation 106(23):2946-54; Gregorini (2002) Circulcation 106(23):2901-7; Debeir (2002) Neurosci 115(1):41-53; Teeters (2003) Am J Physiol Heart Circ Physiol 284(1):H385-92; Savola (2003) Mov Disord 18(8):872-83; Wheeler (2003) Surv Ophthalmol 48supl:S47-51; Tatton (2003) Surv Ophthalmol48sup1:S25-37; Gowing (2003) Cochrane Database Sst Rev (2):CD002024; Pupo (2002) BMC Pharmacology 2:17-33.

In addition, the following patents and patent applications are of interest: Soppet, U.S. Pat. No. 5,994,506; Pausch U.S. Pat. No. 6,406,871.

### SUMMARY

Methods and compositions for modulating biological function in a variety of cell types (e.g., hematopoietic, neuronal, brain, stem, epidermal and epithelial) are provided herein. These methods and compositions can be utilized to treat various maladies such as immune disorders, nervous system disorders and muscle disorders, for example. More specifically, these methods and compositions are for modulating binding between certain PDZ proteins and PL protein binding pairs as shown in Table 8.

Certain methods involve introducing into the cell an agent that alters binding between a PDZ protein and a PL protein in the cell, whereby the biological function is modulated in the cell, and wherein the PDZ protein and PL protein are a binding pair as specified in Table 2. In some of these methods, the agent is a polypeptide comprising at least the two or three carboxy-terminal residues of the PL protein.

Screening methods to identify compounds that modulate binding between PDZ proteins and PL peptides or proteins are also provided. Some screening methods involve contacting under suitable binding conditions (i) a PDZ—domain polypeptide having a sequence from a PDZ protein, and (ii) a PL peptide, wherein the PL peptide comprises a C-terminal sequence of the PL protein, the PDZ—domain polypeptide and the PL peptide are a binding pair as specified in Table 2; and contacting is performed in the presence of the test compound. Presence or absence of complex is then detected. The presence of the complex at a level that is statistically significantly higher in the presence of the test compound than in the absence of test compound is an indication that the test compound is an agonist, whereas, the presence of the complex at a level that is statistically significantly lower in the presence of the test compound than in the absence of test compound is an indication that the test compound is an antagonist.

Modulators of binding between a PDZ protein and a PL protein are also described herein. In certain instances, the modulator is (a) a peptide comprising at least 3 residues of a C-terminal sequence of a PL protein, and wherein the PDZ protein and the PL protein are a binding pair as specified in Table 2; or (b) a peptide mimetic of the peptide of section (a); or (c) a small molecule having similar functional activity with respect to the PDZ and PL protein binding pair as the peptide of section (a). The modulator can be either an agonist or antagonist. Such modulators can be formulated as a pharmaceutical composition.

Methods of treating a disease correlated with binding between a PDZ protein and a PL protein are also disclosed herein, the method comprising administering a therapeutically effective amount of a modulator as provided herein, wherein the PDZ protein and the PL protein are a binding pair as specified in Table 2. As indicated supra, such methods can be used to treat a variety of diseases such as neurological disease, an immune response disease, a muscular disease, or a cancer.

### BRIEF DESCRIPTION OF THE DRAWINGS

FIG. 1 shows the interaction between Interleukin8 receptor A (IL8RA) and the PDZ proteins MAGI1 (domain 2 of 6), TIP1 (domain 1 of 1) and MINT2 (domains 1 & 2) in the in vitro “G” Assay. For each of the three PDZ proteins, the OD (A450) of the interaction with IL8RA is shown in dark gray. The negative control for each of these three reactions is the interaction of GST with IL8RA peptide, the results of which are shown in light gray.

### DESCRIPTION

I. Definitions

A “fusion protein” or “fusion polypeptide” as used herein refers to a composite protein, i.e., a single contiguous amino acid sequence, made up of two (or more) distinct, heterologous polypeptides which are not normally fused together in a single amino acid sequence. Thus, a fusion protein can include a single amino acid sequence that contains two entirely distinct amino acid sequences or two similar or identical polypeptide sequences, provided that these sequences are not normally found together in the same configuration in a single amino acid sequence found in nature. Fusion proteins can generally be prepared using either recombinant nucleic acid methods, i.e., as a result of transcription and translation of a recombinant gene fusion product, which fusion comprises a segment encoding a polypeptide of the invention and a segment encoding a heterologous protein, or by chemical synthesis methods well known in the art.

A “fusion protein construct” as used herein is a polynucleotide encoding a fusion protein.

As used herein, the term “PDZ domain” refers to protein sequence (i.e., modular protein domain) of approximately 90 amino acids, characterized by homology to the brain synaptic protein PSD-95, the Drosophila septate junction protein Discs-Large (DLG), and the epithelial tight junction protein ZO1 (ZO1). PDZ domains are also known as Discs-Large homology repeats (“DHRs”) and GLGF repeats. PDZ domains generally appear to maintain a core consensus sequence (Doyle, D. A., 1996, Cell 85: 1067-76).

PDZ domains are found in diverse membrane-associated proteins including members of the MAGUK family of guanylate kinase homologs, several protein phosphatases and kinases, neuronal nitric oxide synthase, and several dystrophin-associated proteins, collectively known as syntrophins.

Exemplary PDZ domain-containing proteins and PDZ domain sequences are shown in TABLE 6. The term “PDZ domain” also encompasses variants (e.g., naturally occurring variants) of the sequences of TABLE 6 (e.g., polymorphic variants, variants with conservative substitutions, and the like). Typically, PDZ domains are substantially identical to those shown in TABLE 6, e.g., at least about 70%, at least about 80%, or at least about 90% amino acid residue identity when compared and aligned for maximum correspondence.

As used herein, .the term “PDZ protein” refers to a naturally occurring protein containing a PDZ domain. Exemplary PDZ proteins include CASK, MPP1, DLG1, PSD95, NeDLG, TIP33, SYN1a, TIP43, LDP, LIM, LIMK1, LIMK2, MPP2, NOS1, AF6, PTN-4, prIL16, 41.8 kD, KIAA0559,. RGS12, KIAA0316, DVL1, TIP40, TIAM1, MINT1, KIAA0303, CBP, MINT3, TIP2, KIAA0561, and those listed in TABLE 6.

As used herein, the terma“PDZ-domain polypeptide” or “PDZ polypeptide” refer to a polypeptide containing a PDZ domain, such as a fusion protein including a PDZ domain sequence, a naturally occurring PDZ protein, or an isolated PDZ domain peptide.

As used herein, the term “G-protein coupled receptor,” or “GPCR,” refers to a naturally occuring polypeptide receptor, or a polynucleotide encoding a receptor, known to interact with G-proteins or have homology to proteins known to interact with G proteins. In addition, this definition includes polypeptide receptors, or polynucleotides encoding receptors, that are similar to those known to interact with G-proteins. A partial list of known GPCR's is presented in TABLE 3.

As used herein, the term “PL protein” or “PDZ Ligand protein” refers to a naturally occurring protein that forms a molecular complex with a PDZ-domain, or to a protein whose carboxy-terminus, when expressed separately from the full length protein (e.g., as a peptide fragment of 4-25,residues, e.g., 8, 10, 12, 14 or 16 residues), forms such a molecular complex. The molecular complex can be observed in vitro using the “A assay” or “G assay” described infra, or in vivo. Exemplary PL proteins listed in TABLE 2 are demonstrated to bind specific PDZ proteins. This definition is not intended to include anti-PDZ antibodies and the like.

As used herein, GPCR-PL refers to a PDZ ligand sequence that occurs within a GPCR polypeptide sequence.

As used herein, a “PL sequence” refers to the amino acid sequence of the C-terminus of a PL protein (e.g., the C-terminal 2, 3, 4, 5, 6, 7, 8, 9, 10, 12, 14, 16, 20 or 25 residues) (“C-terminal PL sequence”) or to an internal sequence known to bind a PDZ domain (“internal PL sequence).

As used herein, a “PL peptide” is a peptide of having a sequence from, or based on, the sequence of the C-terminus of a PL protein. Exemplary PL peptides (biotinylated) are listed in TABLE 2.

As used herein, a “PL fusion protein” is a fusion protein that has a PL sequence as one domain, typically as the C-terminal domain of the fusion protein. An exemplary PL fusion protein is a tat-PL sequence fusion.

As used herein, the term “PL inhibitor peptide sequence” refers to PL peptide amino acid sequence that (in the form of a peptide or PL fusion protein) inhibits the interaction between a PDZ domain polypeptide and a PL peptide (e.g., in an A assay or a G assay).

As used herein, a “PDZ-domain encoding sequence” -means a segment of a polynucleotide encoding a PDZ domain. In various embodiments, the polynucleotide is DNA, RNA, single stranded or double stranded.

As used herein, the terms “antagonist” and “inhibitor,“when used in the context of modulating a binding interaction (such as the binding of a PDZ domain sequence to a PL sequence), are used interchangeably and refer to an agent that reduces the binding of the, e.g., PL sequence (e.g., PL peptide) and the, e.g., PDZ domain sequence (e.g., PDZ protein, PDZ domain peptide).

As used herein, the terms “agonist” and “enhancer,” when used in the context of modulating a binding interaction (such as the binding of a PDZ domain sequence to a PL sequence), are used interchangeably and refer to an agent that increases the binding of the, e.g., PL sequence (e.g., PL peptide) and the, e.g., PDZ domain sequence (e.g., PDZ protein, PDZ domain peptide).

As used herein, the terms “peptide mimetic, ” “peptidomimetic,” and “peptide analog” are used interchangeably and refer to a synthetic chemical compound which has substantially the same structural and/or functional characteristics of an PL inhibitory or PL binding peptide of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or inhibitory or binding activity. As with polypeptides of the invention which are conservative variants, routine experimentation will determine whether a mimetic is within the scope of the invention, i.e., that its structure and/or function is not substantially altered. Thus, a mimetic composition is within the scope of the invention if it is capable of binding to a P6Z domain and/or inhibiting a PL-PDZ interaction.

Polypeptide mimetic compositions can contain any combination of nonnatural structural components, which are typically from three structural groups: a), residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like.

A polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N=-dicyclohexylcarbodiimide (DCC) or N,N=-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, A Peptide Backbone Modifications, Marcell Dekker, NY).

A polypeptide can also be characterized as a mimetic by containing all or some non-natural residues in place of naturally occurring amino acid residues. Nonnatural residues are well described in the scientific and patent literature; a few exemplary nonnatural compositions useful as mimetics of natural amino acid residues and guidelines are described below.

Mimetics of aromatic amino acids can be generated by replacing by, e.g., D- or L-naphylalanine; D- or L-phenylglycine; D- or L-2 thieneylalanine; D- or L-1, -2, 3-, or 4-pyreneylalanine; D- or L-3 thieneylalanine; D- or L-(2-pyridinyl)-alanine; D- or L-(3-pyridinyl)-alanine, D- or L-(2-pyrazinyl)-alanine; D- or L-(4-isopropyl)-phenylglycine; D-(trifluoromethyl)-phenylglycine; D-(trifluoromethyl)-phenylalanine;D-p-fluorophenylalanine; D- or L-p-biphenylphenylalanine; K— or L-p-methoxybiphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a nonnatural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides(R═—N—C—N—R═) such as, e.g., 1-cyclohexyl-3(2-morpholinyl-(4-ethyl)carbodiimide or 1-ethyl-3(4-azonia-4,4-dimetholpentyl)carbodiimide. Aspartyl or glutamyl can also be converted to asparaginyl and glutaminyl residues by reaction with ammonium ions.

Mimetics of basic amino acids can be generated by substitution with, e.g., (in addition to lysine and arginine) the amino acids omithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues.

Arginine residue mimetics can be generated by reacting arginyl with, e.g., one or more conventional reagents, including, e.g., phenylglyoxal, -2,3-butanedione, 1,2-cyclohexanedione, or ninhydrin, preferably under alkaline conditions.

Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively.

Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl)propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol, or, chloro-7-nitrobenzo-oxa-1,3-diazole.

Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate.

Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide.

Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A component of a natural polypeptide (e.g., a PL polypeptide or PDZ polypeptide) can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, generally referred to as the D-amino acid, but which can additionally be referred to as the R— or S-form.

The mimetics of the invention can also include compositions that contain a structural mimetic residue, particularly a residue that induces or mimics secondary structures, such as a beta turn, beta sheet, alpha helix structures, gamma turns, and the like. For example, substitution of natural amino acid residues with D-amino acids; N-alpha-methyl amino acids; C-alpha-methyl amino acids; or dehydroamino acids within a peptide can induce or stabilize beta turns, gamma turns, beta sheets or alpha helix conformations. Beta turn mimetic structures have been described, e.g., by Nagai (1985) Tet. Lett. 26:647-650; Feigl (1986) J. Amer. Chem. Soc. 108:181-182; Kahn (1988) J. Amer. Chem. Soc. 110:1638-1639; Kemp (1988) Tet. Lett. 29:5057-5060;Kahn (1988) J. Molec. Recognition 1:75-79. Beta sheet mimetic structures have been described, e.g., by Smith (1992) J. Amer. Chem. Soc. 114:10672-10674. For example, a type VI beta turn induced by a cis amide surrogate,1,5-disubstituted tetrazol, is described by Beusen (1995) Biopolymers 36:181-200. Incorporation of achiral omega-amino acid residues to generate polymethylene units as a substitution for amide bonds is described by Banerjee (1996) Biopolymers 39:769-777. Secondary structures of polypeptides can be analyzed by, e.g., high-field 1H NMR or 2D NMR spectroscopy, see, e.g., Higgins (1997) J. Pept. Res. 50:421-435. See also, Hruby (1997) Biopolymers 43:219-266, Balaji, et al., U.S. Pat. No. 5,612,895.

As used herein, “peptide variants” and “conservative amino acid substitutions” refer to peptides that differ from a reference peptide (e.g., a peptide having the sequence of the carboxy-terminus of a specified PL protein) by substitution of an amino acid residue having similar properties (based on size, polarity, hydrophobicity, and the like). Thus, insofar as the compounds that are encompassed within the scope of the invention are partially defined in terms of amino acid residues of designated classes, the amino acids may be generally categorized into three main classes: hydrophilic amino acids, hydrophobic amino acids and cysteine-like amino acids, depending primarily on the characteristics of the amino acid side chain. These main classes may be further divided into subclasses. Hydrophilic amino acids include amino acids having acidic, basic or polar side chains and hydrophobic amino acids include amino acids having aromatic or a polar side chains. A polar amino acids may be further subdivided to include, among others, aliphatic amino acids. The definitions of the classes of amino acids as used herein are as follows:

“Hydrophobic Amino Acid” refers to an amino acid having a side chain that is uncharged at physiological pH and that is repelled by aqueous solution. Examples of genetically encoded hydrophobic amino acids include Ile, Leu and Val. Examples of non-genetically encoded hydrophobic amino acids include t-BuA.

“Aromatic Amino Acid” refers to a hydrophobic amino acid having a side chain containing at least one ring having a conjugated π-electron system (aromatic group). The aromatic group may be further substituted with groups such as alkyl, alkenyl, alkynyl, hydroxyl, sulfanyl, nitro and amino groups, as well as others. Examples of genetically encoded aromatic amino acids include Phe, Tyr and Trp. Commonly encountered non-genetically encoded aromatic amino acids include phenylglycine, 2-naphthylalanine, β-2-thienylalanine, 1,2,3,4-tetrahydroisoquinoline-3-carboxylic acid, 4-chloro-phenylalanine, 2-fluorophenyl-alanine, 3-fluorophenylalanine and 4-fluorophenylalanine.

“Apolar Amino Acid” refers to a hydrophobic amino acid having a side chain that is generally uncharged at physiological pH and that is not polar. Examples of genetically. encoded apolar amino acids include Gly, Pro and Met. Examples of non-encoded apolar amino acids include Cha.

“Aliphatic Amino Acid” refers to an apolar amino acid having a saturated or unsaturated straight chain, branched or cyclic hydrocarbon side chain. Examples of genetically encoded aliphatic amino acids include Ala, Leu, Val and Ile. Examples of non-encoded aliphatic amino acids include Nle.

“Hydrophilic Amino Acid” refers to an amino acid having a side chain that is attracted by aqueous solution. Examples of genetically encoded hydrophilic amino acids include Ser and, Lys. Examples of non-encoded hydrophilic amino acids include Cit and hCys.

“Acidic Amino Acid” refers to a hydrophilic amino acid having a side chain pK value of less than 7. Acidic, amino acids typically have negatively charged side chains at physiological pH due to loss of a hydrogen ion. Examples of genetically encoded acidic amino acids include Asp and Glu.

“Basic Amino Acid” refers to a hydrophilic amino acid having a side chain pK value of greater than 7. Basic amino acids typically have positively charged side chains at physiological pH due to association with hydronium ion. Examples of genetically encoded basic amino acids include Arg, Lys and His. Examples of non-genetically encoded basic amino acids include the non-cyclic amino acids omithine, 2,3-diaminopropionic acid, 2,4-diaminobutyric acid and homoarginine.

“Polar Amino Acid” refers to a hydrophilic amino acid having a side chain that is uncharged at physiological pH, but which has a bond in which the pair of electrons shared in common by two atoms is held more closely by one of the atoms. Examples of genetically encoded polar amino acids include Asx and Glx. Examples of non-genetically encoded polar amino acids include citrulline, N-acetyl lysine and methionine sulfoxide.

“Cysteine-Like Amino Acid” refers to an amino acid having a side chain capable of forming a covalent linkage with a side chain of another amino acid residue, such as a disulfide linkage. Typically, cysteine-like amino acids generally have a side chain containing at least one thiol (SH) group. Examples of genetically encoded cysteine-like amino acids include Cys. Examples of non-genetically encoded cysteine-like amino acids include homocysteine and penicillamine.

As will be appreciated by those having skill in the art, the above classification are not absolute—several amino acids exhibit more than one characteristic property, and can therefore be included in more than one category. For example, tyrosine has both an aromatic ring and a polar hydroxyl group. Thus, tyrosine has dual properties and can be included in both the aromatic and polar categories. Similarly, in addition to being able to form disulfide linkages, cysteine also has apolar character. Thus, while not strictly classified as a hydrophobic or apolar amino acid, in many instances cysteine can be used to confer hydrophobicity to a peptide.

Certain commonly encountered amino acids which are not genetically encoded of which the peptides and peptide analogues of the invention may be composed include, but are not limited to, β-alanine(b-Ala) and other omega-amino acids such as 3-aminopropionicacid (Dap), 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine or sarcosine (MeGly); ornithine (Orn); citrulline (Cit); t-butylalanine (t-BuA); t-butylglycine (t-BuG); N-methylisoleucine (Melle); phenylglycine (Phg); cyclohexylalanine (Cha); norleucine (Nle); 2-naphthylalanine (2-Nal); 4-chlorophenylalanine (Phe(4-Cl)); 2-fluorophenylalanine (Phe(2-F)); 3-fluorophenylalanine (Phe(3-F)); 4-fluorophenylalanine(Phe(4-F)); penicillamine (Pen); 1,2,3,4-tetrahydroisoquinoline-3-carboxylic acid(Tic); β-2-thienylalanine (Thi); methionine sulfoxide (MSO); homoarginine (hArg); N-acetyl lysine (AcLys); 2,3-diaminobutyricacid(Dab); 2,3-diaminobutyricacid (Dbu);p-aminophenylalanine(Phe(pNR₂)); N-methyl valine (MeVal); homocysteine (hCys) and homoserine (hSer). These amino acids also fall conveniently into the categories defined above.

The classifications of the above-described genetically encoded and non-encoded amino acids are summarized in TABLE 1, below. It is to be understood that TABLE 1 is for illustrative purposes only and does not purport to be an exhaustive list of amino acid residues which may comprise the peptides and peptide analogues described herein. Other amino acid residues which are useful for making the peptides and peptide analogues described herein can be found, e.g., in Fasman, 1989, CRC Practical Handbook of Biochemistry and Molecular Biology, CRC Press, Inc., and the references cited therein. Amino acids not specifically mentioned herein can be conveniently classified into the above-described categories on the basis of known behavior and/or their characteristic chemical and/or physical properties as compared with amino acids specifically identified.

TABLE I

Genetically

| | | |
|---|---|---|
| Classification | Encoded | Genetically Non-Encoded |

| | | |
|---|---|---|
| Hydrophobic | | |
| Aromatic | F, Y, W | Phg, Nal, Thi, Tic, Phe(4-Cl), Phe(2-F), |
| | | Phe(3-F), Phe(4-F), Pyridyl Ala, |
| | | Benzothienyl Ala |
| Apolar | M, G, P | |
| Aliphatic | A, V, L, I | t-BuA, t-BuG, Melle, Nle, MeVal, Cha, |
| | | bAla, MeGly, Aib |
| Hydrophilic | | |
| Acidic | D, E | |
| Basic | H, K, R | Dpr, Orn, hArg, Phe(p-NH₂), DBU, A₂BU |
| Polar | Q, N, S, T, Y | Cit, AcLys, MSO, hSer |
| Cysteine-Like | C | Pen, hCys, p-methyl Cys |

As used herein, a “detectable label” has the ordinary meaning in the art and refers to an atom (e.g., radionuclide), molecule (e.g., fluorescein), or complex, that is or can be used to detect (e.g., due to a physical or chemical property), indicate the presence of a molecule or to enable binding of another molecule to which it is, covalently bound or otherwise associated. The term “label” also refers to covalently bound or otherwise associated molecules (e.g., a biomolecule such as an enzyme) that act on a substrate to produce a detectable atom, molecule or complex. Detectable labels suitable for use in the present invention include any composition detectable by spectroscopic, photochemical, biochemical, immunochemical, electrical, optical or chemical means. Labels useful in the present invention include biotin for staining with labeled streptavidin conjugate, magnetic beads (e.g. Dynabeads™), fluorescent dyes (e.g., fluorescein, Texas red, rhodamine, green fluorescent protein, enhanced green fluorescent protein, and the like), radiolabels (e.g., ³H, ¹²⁵I, ³⁵S, ¹⁴C, or ³²P), enzymes (e.g., hydrolases, particularly phosphatases such as alkaline phosphatase, esterases and glycosidases, or oxidoreductases, particularly peroxidases such as horse radish peroxidase, and others commonly used in ELISAs), substrates, cofactors; inhibitors, chemiluminescent groups, chromogenic agents, and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241. Means of detecting such labels are well known to those of skill in the art. Thus, for example, radiolabels and chemiluminescent labels may be detected using photographic film or scintillation counters, fluorescent markers may be detected using a photodetector to detect emitted light (e.g., as in fluorescence-activated cell sorting). Enzymatic labels are typically detected by providing the enzyme with a substrate and detecting the reaction product produced by the action of the enzyme on the substrate, and colormetric labels are detected by simply visualizing the colored label. Thus, a label is any composition detectable by spectroscopic, photochemical, biochemical, immunochemical, electrical, optical or chemical means. The label may be coupled directly or indirectly to the desired component of the assay according to methods well known in the art. Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal generating system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with the labeled, naturally occurring anti-ligands. Alternatively, any haptenic or antigenic compound can be used in combination with an antibody. The molecules can also be conjugated directly to signal generating compounds, e.g., by conjugation with an enzyme or fluorophore. Means of detecting labels are well known to those of skill in the art. Thus, for example, where the label is a radioactive label, means for detection include a scintillation counter, photographic film as in autoradiography, or storage phosphor imaging. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Also, simple calorimetric labels may be detected by observing the color associated with the label. It will be appreciated that when pairs of fluorophores are used in an assay, it is often preferred that they have distinct emission patterns (wavelengths) so that they can be easily distinguished.

As used herein, the term “substantially identical” in the context of comparing amino acid sequences, means that the'sequences have at least about 70%, at least about 80%, or at least about 90% amino acid residue identity when compared and aligned for maximum correspondence. An algorithm that is suitable for determining percent sequence identity and sequence similarity is the FASTA algorithm, which is described in Pearson, W. R. & Lipman, D. J., 1988, Proc. Natl. Acad. Sci. U.S.A. 85: 2444. See also W. R. Pearson, 1996, Methods Enzymol. 266: 227-258. Preferred parameters used in a FASTA alignment of DNA sequences to calculate percent identity are optimized, BL50 Matrix 15: −5, k-tuple=2; joining penalty=40, optimization=28; gap penalty −12, gap length penalty=−2; and width=16.

As used herein, “hematopoietic cells” include leukocytes including lymphocytes (T cells, B cells and NK cells), monocytes, and granulocytes (i.e., neutrophils, basophils and eosinophils), macrophages, dendritic cells, megakaryocytes, reticulocytes, erythrocytes, and CD34⁺ stem cells.

As used herein, the terms “test compound” or “test agent” are used interchangeably and refer to a candidate agent that may have enhancer/agonist, or inhibitor/antagonist activity, e.g., inhibiting or enhancing an interaction such as PDZ-PL binding. The candidate agents or test compounds may be any of a large variety of compounds, both naturally occurring and synthetic, organic and inorganic, and including polymers (e.g., oligopeptides, polypeptides, oligonucleotides, and polynucleotides), small molecules,, antibodies (as broadly defined herein), sugars, fatty acids, nucleotides and nucleotide analogs, analogs of naturally occurring structures (e.g., peptide mimetics, nucleic acid analogs, and the like), and numerous other compounds. In certain embodiment, test agents are prepared from diversity libraries, such as random or combinatorial peptide or non-peptide libraries. Many libraries are known in the art that can be, used, e.g., chemically synthesized libraries, recombinant (e.g., phage display libraries), and in vitro translation-based libraries. Examples of chemically synthesized libraries are described in Fodor et al., 1991, Science 251:767-773; Houghten et al., 1991, Nature 354:84-86; Lam et al., 1991, Nature 354:82-84; Medynski, 1994, Bio/Technology 12:709-710; Gallop et al., 1994, J. Medicinal Chemistry 37(9):1233-1251; Ohlmeyer et al., 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; Salmon et al., 1993, Proc. Natl. Acad. Sci. USA 90:11708-11712; PCT Publication No. WO 93/20242; and Brenner and Lerner, 1992, Proc. Natl. Acad. Sci. USA 89:5381-5383. Examples of phage display libraries are described in Scott and Smith, 1990, Science 249:386-390; Devlin et al., 1990, Science, 249:404-406; Christian, R. B., et al., 1992, J. Mol. Biol. 227:711-718); Lenstra, 1992, J. Immunol. Meth. 152:149-157; Kay et al., 1993, Gene 128:59-65; and PCT Publication No. WO 94/18318 dated Aug. 18, 1994. In vitro translation-based libraries include but are not limited to those described in PCT Publication No. WO 91/05058 dated Apr. 18, 1991; and Mattheakis et al., 1994, Proc. Natl. Acad. Sci. USA 91:9022-9026. By way of examples of nonpeptide libraries, a benzodiazepine library (see e.g., Buninet al., 1994, Proc. Natl. Acad. Sci. USA 91:4708-4712) can be adapted for use. Peptoid libraries (Simon et al., 1992, Proc. Natl. Acad. Sci. USA 89:9367-9371) can also be used. Another example of a library that can-be used, in which the amide functionalities in peptides have been permethylated to generate a chemically transformed combinatorial library, is described by Ostresh et al. (1994, Proc. Natl. Acad. Sci. USA 91:11138-11142).

The term “specific binding” refers to binding between two molecules, for example, a ligand and a receptor, characterized by the ability of a molecule (ligand) to associate with another specific molecule (receptor) even in the presence of many other diverse molecules, i.e., to show preferential binding of one molecule for another in a heterogeneous mixture of molecules. Specific binding of a ligand to a receptor is also evidenced by reduced binding of a detectably labeled ligand to the receptor in the presence of excess unlabeled ligand (i.e., a binding competition assay).

As used herein, a “plurality” of PDZ proteins (or corresponding PDZ domains or PDZ fusion polypeptides) has its usual meaning. In some embodiments the plurality is at least 5, and often at least 25, at least 40, or at least 60 different PDZ proteins. In some embodiments, the plurality is selected from the list of PDZ polypeptides listed in TABLE 6. In some embodiments, the plurality of different PDZ proteins are from (i.e., expressed in) a particular specified tissue or a particular class or type of cell. In some embodiments, the plurality of different PDZ proteins represents a substantial fraction (e.g., typically at least 50%, more often at least 80%) of all of the PDZ proteins known to be, or suspected of being, expressed in the tissue or cell(s), e.g., all of the PDZ proteins known to be present in lymphocytes or hematopoetic cells. In some embodiments, the plurality is at least 50%, usually at least 80%, at least 90% or all of the PDZ proteins disclosed hereinas being expressed in a particular cell.

When referring to PL peptides (or the corresponding proteins, e.g., corresponding to those listed in TABLE 3, or elsewhere herein) a “plurality” may refer to, at least 5, at least 10, and often at least 25 PLs such as those specifcally listed herein, or to the classes and percentages set forth supra for PDZ domains.

II. Overview

The present inventors have identified a large number of interactions between PDZ proteins and GPCR proteins containing a PL motif. These interactions can play a significant role in the biological function of certain cells and in a variety of physiological systems. As used herein, the term “biological function” in the context of a cell, refers to a detectable biological activity normally carried out by the cell, e.g., a phenotypic change such as proliferation, cell activation (e.g., T cell activation, B cell activation, T-B cell conjugate formation), cytokine release, degranulation, tyrosine phosphorylation, ion (e.g., calcium) flux, metabolic activity, apoptosis, changes in gene expression, maintenance of cell structure, cell migration, adherence to a substrate, signal transduction, cell-cell interactions, and others described herein or known in the art.

Because the interactions involve proteins which are involved in diverse physiological systems (see Background section supra), the methods provided herein can be utilized to broadly or selectively modulate a number of different biological functions. Methods are also disclosed herein for determining whether a test compound acts as a modulator of binding between a particular PDZ protein and PL protein binding pair. Both agonists and antagonists of the binding pairs can be identified by such screening methods. Modulators so identified can subsequently be formulated as a pharmaceutical composition and used in the treatment of various diseases that are correlated with binding between a particular PDZ protein and PL protein or set of such proteins.

III. PDZ Protein and PL Protein Interactions

TABLE 2 lists PDZ proteins and GPCR-PL proteins which the current inventors have identified as binding to one another. Each page of TABLE 2 includes six columns. The columns are numbered from left to right such that the left-most column is column 1 and the right-most column is column 6. Thus, the first column is labeled “AVC PL ID” and lists AA numbers that serve as unique internal designations for each PL peptide. These ID numbers correspond to those listed in column 6 of TABLE 3. The second column is labeled “PL” and lists the various PL proteins/PDZ-Ligands that were examined. This column lists gene abbreviations, with full gene names listed in parentheses, for peptides corresponding to the carboxyl-terminal 20 amino acids of the protein listed. The third column, labeled “PL 20mer Sequence,” lists the carboxyl-terminal 20 amino acids of the protein. All ligands are biotinylated at the amino-terminus. Some have been modified to eliminate cysteine amino acids from the 20mer sequence. Modifications have been noted in column 2, and wildtype sequences are presented in TABLE 3.

The PDZ protein (or proteins) that interact(s) with a PL peptide are listed in the fourth column that is labeled “PDZ”. This column provides the gene name for the PDZ portion of the GST-PDZ fusion that interacts with the PDZ-ligand to the left. For PDZ domain-containing proteins with multiple domains the domain number is listed to the right of the PDZ (i.e., in column 5 labeled “PDZ Domain”), and indicates the PDZ domain number when numbered from the amino-terminus to the carboxy-terminus.

The sixth column labeled “Binding Strength” is another measure of the level of binding. In particular, it provides an absorbance value at 450 nm which indicates the amount of PL peptide bound to the PDZ protein. The following numerical values have the following meanings: ‘1’—A450 nm 0-1; ‘2’—A450 nm 1-2; ‘3’—A450 nm 2-3; ‘4’—A450 nm; 3-4; ‘5’—A450 nm of 4. All interactions have been repeated a total of at least 4 times, and all show A450 nm values that are at least two times that of controls.

Further information regarding these PL proteins and PDZ proteins is provided in TABLES 3 and 6. In particular, TABLE 3 provides a partial listing of known G-protein coupled receptors, along with the amino acid sequence of the carboxyl-terminal 20 amino acids. When numbered from left to right, the first column labeled “Gene Name(Synonyms)” provides the most commonly used name for that gene, with synonyms or acronyms listed in parentheses. Genbank reference numbers (Accession number and GI number) are listed in column 2, labeled “Genbank Reference.” Columns 3 and 4, labeled “Last 20 aa” and “Last 4 aa,” respectively, list the-last 20 amino acids, and the last 4 amino acids of each protein. Column 5, labeled “PL?” marks with an “X” those carboxy-terminal sequences that are predicted to display a classic PL amino acid motif. Many of the carboxyl-terminal motifs that are not marked in column 6 may exhibit binding to PDZ proteins, and the designation as a classic PL motif is in no way intended to predictor restrict GPCR binding patterns to PDZ proteins. The sixth column labeled “AVC PL ID” provides the internal designation number used to refer to a particular PL protein and correlates with the designation used in column 1 of TABLE 2. Those PL proteins that have been assigned an internal AVC ID

Many of the genes listed in TABLE 3 express more than one amino acid sequence depending on alternative exon splicing and single amino acid changes. The Genbank reference presented is intended to represent one isoform of the gene listed. Alternatively spliced and point mutated isoforms of the genes listed in TABLE 3 are given separate Genbank references, and have been omitted for the purpose of brevity. In all cases, the carboxyl-terminal sequence is unaffected by these changes. In cases where the carboxyl-terminal sequence is affected by point mutations or alternative splicing, both isoforms have been included. In addition, those GPCR's for which no known receptor ligand has been identified (so-called “orphan GPCR's”) have also been omitted. As indicated supra, all peptides are biotinylated at the amino terminus and the amino acid sequences correspond to the C-terminal sequence of the gene name listed to the left.

TABLE 6 lists the sequences of the PDZ domains cloned into a vector (PGEX-3X vector) for production of GST-PDZ fusion proteins (Pharmacia). More specifically,, the first column (left to right) entitled “PDZ Gene Name” lists the name of the gene containing the PDZ domain. The second column labeled “GI or Acc#” is a unique Genbank identifier for the gene used to design primers for PCR amplification of the listed sequence. The next column labeled “Domain#” indicates the Pfam-predicted PDZ domain number, as numbered from the amino-terminus of the gene to the carboxy-terminus. The last column entitled “Sequence fused to GST construct” provides the actual amino acid sequence inserted into the GST-PDZ expression vector as determined by DNA sequencing of the constructs.

As discussed in detail herein, the PDZ proteins listed in TABLES 2 and 6 are naturally occurring proteins containing a PDZ domain. Only significant interactions are presented in this table. Thus, the present invention is particularly directed to the detection and modulation of interactions between a PDZ protein and PL protein pair listed in TABLE 2. As used herein the phrase “protein pair” or “protein binding pair” when used in reference to a PDZ protein and PL protein refers to a PD protein and PDZ protein listed in TABLE 2 which bind to one another. It should be understood that TABLE 2 is set up to show that certain PL proteins bind to a plurality of PDZ proteins. For example, PL protein AA329 binds to PDZ proteins KIAA0973 and KIAA0807.

Interactions between GPCR proteins containing a PDZ ligand and PDZ proteins are not limited to those listed in TABLE 2. TABLE 4 presents a list of interactions between GPCR proteins and PDZ proteins. When numbered from left to right, the first column, labeled “GPCR gene,” lists the GPCR protein that binds to a PDZ domain-containing protein. The second column labeled “PDZ-containing Gene” lists the specific PDZ-containing gene that binds to the GPCR gene listed to the left. The PDZ domain that binds to the GPCR is listed in column 3, labeled “PDZ domain(s).” These interactions were confirmed using assays other than the “G” or “A” assays described infra, and suggest that changes in PDZ-PL binding patterns may occur with the use of different assays or with the use of assay variations described infra.

The interactions summarized in TABLE 2 can occur in a wide variety of cell types. Examples of such cells include hematopoietic, stem, neuronal, muscle, epidermal, epithelial, endothelial, and cells from essentially any tissue such as liver, lung, placenta, uterus, kidney, ovaries, testes, stomach, colon and intestine. Because the interactions disclosed herein can occur in such a wide variety of cell types, these interactions can also play an important role in a variety of biological functions.

Thus, for example, in certain embodiments of the invention, the PL proteins of the invention bind a PDZ protein expressed in T lymphocytes, B lymphocytes, or both T and B lymphocytes. In an embodiment, the PL protein binds a PDZ protein expressed in endothelial cells. In various embodiments, the PL proteins and/or the PDZ protein to which it binds are not expressed in the nervous system (e.g., neurons). In still other instances the PL protein binds a PDZ protein that is expressed in neuronal cells.

In various embodiments of the invention, the PL protein is expressed or up-regulated upon cell activation(e.g., in activated B lymphocytes, T lymphocytes) or upon entry into mitosis (e.g., up-regulation in rapidly proliferating cell populations).

In certain other various embodiments of the invention, the PL protein is (i) a protein that mediates immune cell (e.g., hematopoietic cell) activation or migration, (ii) a protein that does not mediate apoptosis in a cell type, (iii) a protein that is a G-protein coupled seven transmembrane helix receptor but not a serotonin receptor, (iv) a protein that is G-protein coupled seven transmembrane helix receptor but not a cytokine receptor, or (v) a protein that is a G-protein coupled seven transmembrane helix receptor and is a cytokine receptor.

IV. Classification of Interactions

A. General

The interactions summarized in TABLE 2 can occur in a wide variety of cell types. Examples of such cells include hematopoietic, stem, neuronal, muscle, epidermal, eipthelial, endothelial, and cells from essentially any tissue such as liver, ling, placenta, uterus, kidney, ovaries, testes, stomach, colon and intestine. Because the interactions disclosed herein can occur in such a wide variety of cell types, these interactions can also play an important role in a variety of biological functions. Consequently, modulation of the interactions between PDZ proteins and PL proteins that are described herein can be utilized to regulate biological function in a wide range of cells.

B. Exemplary PDZ Classification

The PDZ proteins identified herein as interacting with particular PL proteins can be grouped into a number of different categories. Thus, as described in greater detail below, the methods and reagents that are provided herein can be utilized to modulate PDZ interactions, and thus biological functions, that are regulated or otherwise involve the following classes of proteins. It should be recognized, however, that modulation of the interactions that are identified herein can be utilized to affect biological functions involving other protein classes.

1. Protein Kinases

A number of protein kinases contain PDZ domains. Protein kinases are widely involved in cellular metabolism and regulation of protein activity through phosphorylation of amino acids on proteins. An example of this is the regulation of signal transduction pathways such as T cell activation through the T cell Receptor where ZAP-70 kinase function is required for transmission of the activation signal to downstream effector molecules. These molecules include, but are not limited to KIAA0303, KIAA0561, KIAA0807, KIAA0973, and CASK.

2. Guaialyte Kinases

A number of guanalyte kinases contain PDZ domains. These molecules include, but are not limited to Atrophin 1, CARD11 CARD14, DLG1, DLG2, DLG5, FLJ12615, MPP1, MPP2, NeDLG, p55T, PSD95, ZO-1, ZO-2, and ZO-3.

3. Guanine Exchange Factors

A number of guanine exchange factors contain PDZ domains. Guanine exchange factors regulate signal transduction pathways and other biological processes through facilitating the exchange of differently phosphorylated guanine residues. These molecules include, but are not limited to GTPase, Guanine Exchange, KIAA0313, KIAA0380, KIAA0382, KIAA1389, KIAA1415, TIAM1, and TAIM2.

4. LIM PDZ's

A number of LIM PDZ's contain PDZ domains. These molecules include, but are not limited to α-Actinin 2, ELFIN1, ENIGMA, HEMBA 1003117. KIAA0613, KIAA0858, KIAA0631, LIM Mystique, LIM protein, LIM-RIL, LIMK1, LIMK2, and LU-1.

5. Proteins Containing only PDZ Domains

A number of proteins contain PDZ domains without any other predicted functional domains. These include, but are not limited to 26s subunit p27, AIPC, Cytohesion Binding Protein, EZRIN Binding Protein, FLJ00011, FLJ20075, FLJ21687, GRIP1, HEMBA1000505, KIAA0545, KIAA0967, KIAA1202, KIAA1284. KIAA1526, KIAA1620, KIAA1719, MAG11, Novel PDZ gene, Outer Membrane, PAR3, PAR6, PAR6γ, PDZ-73, PDZK1, PICK1, PIST, prIL16, Shank1, SIP1, SITAC-18, Syntenin, Syntrophin γ2, TIP1, TIP2, and TIP43.

6. Tyrosine Phosphatases

A number of Tyrosine phosphatases contain PDZ domains. Tyrosine phosphatases regulate biological processes such as signal transduction pathways through removal of phosphate groups required for function of the target protein. Examples of such enzymes include, but are not limited to PTN-3, PTN-4, and PTPL1.

7. Serine Proteases

A number of serine proteases contain PDZ domains. Proteases affect biological molecules by cleaving them to either activate or repress their functional ability. These enzymes have a variety of functions, including roles in digestion, blood coagulation and lysis of blood clots. These include, but are not limited to Novel Serine Protease and Serine Protease.

8. Viral Oncogene Interacting Proteins that Contain PDZ Domains

A number of TAX interacting proteins contain PDZ domains. Many of these also bind to multiple viral on coproteins such as Adenovirus E4, Papillomavirus E6, and HBV protein X. These include, but are not limited to AIPC, Connector Enhancer, DLG1, DLG2, ERBIN, FLJ00011, FLJ11215, HEMBA1003117, INADL, KIAA0147, KIAA0807, KIAA1526, KIAA1634, LIMK1, LIM Mystique, LIM-RIL, MUPP1, NeDLG, Outer Membrane, PSD95, PTN-4, PTPL-1, Syntrophin γ1 Syntrophin TAX2-like protein, TIP2, TIP1, TIP33, and TIP43.

9. Proteins Containing RA and/or RHA and/or DIL and/or IGFBP and/or WW and/or L27 and/or SAM and/or PH and/or DIX and/or DIP and/or Dishevelled and/or LRR and/or Hormone 3 and/or C2 and/or RPH3A and/or zf-TRAF and/or zf-C3HC4 and/or PID and/or NO Synthase and/or Flavodoxin and/or FAD binding and/or NAD binding and/or Kazal and/or Trypsin and/or RBD and/or RGS and/or GoLoco and/or HR1 and/or BR01 That Contain PDZ Domains

A number of proteins containing RA and/or RHA and/or DIL and/or IGFBP and/or WW and/or L27 and/or SAM and/or PH and/or DIX and/or DIP and/or Dishevelled and/or LRR and/or Hormone 3 and/or C2 and/or RPH3A and/or zf-TRAF and/or zf-C3HC4 and/or PID and/or NO_Synthase and/or Flavodoxin and/or FAD binding and/or NAD binding and/or Kazal and/or Trypsin and/or RBD and/or RGS and/or GoLoco and/or HR1 and/or BR01, contain PDZ domains. These include, but are not limited to AF6, APXL-1, MAG11, DVL1, DVL2, DVL3, KIAA0417, KIAA0316, KIAA0340, KIAA0559, KIAA0751, KIAA0902, KIAA1095, KIAA1222, KIAA1634, MINT1, NOS1, RGS12, Rhophilin-like, Shank 3, Syntrophin 1α, Syntrophin β2, and X11β.

C. Exemplary PL Classification

The GPCR-PL proteins involved in the interactions listed in TABLE 2 are from a number of different classes. Consequently, the methods and reagents that are disclosed herein can be utilized to modulate interactions involving the following classes of GPCR-PL proteins to modulate a biological function in cells, but are not intended to be limiting in scope of biological processes or diseases affected. The following classes, however, should not be considered exhaustive of the: types of classes of GPCR proteins whose activity can be modulated using the methods and reagents that are provided herein.

1. Serotonin Receptors

Serotonin receptors are involved in a variety of physiological functions, including nociception, motor control, endocrine secretion thermoregulation, appetite, control of exchanges between the central-nervous system and cerebospinal fluid, prostate cancer, hormone overproduction by endocrine tumors, migrane, irritable bowel syndrome, Alziemer's disease, drug withdrawls, and a number of psychological disorders, including but not limited to depression, obsessive compulsive disorder, schizophrenia, and anxiety. Representative members of this family include, but are not limited to, 5-HT1A, 5-HT1B, 5-HT1D, 5HT1F, 5-HT2A, 5-HT2B, 5-HT2C, 5-HT4, 5-HT5A, 5-HT6, and 5-HT7. Modulation of serotonin receptor interactions with PDZ proteins may provide an effective means for treating a number of diseases, including but not limited to those listed above.

2. Histamine Receptors

Histamine receptors are involved in histamine responses, and affect several systems that result in asthma, allergy and inflammation responses. In addition, histamine receptors have been implicated in anaphylaxis, rhinoconjunctivitis, and Gastroesophageal reflux disease (GERD). Representative GPCRs include, but are not limited to, Histamine H1 receptor, histamine H2 receptor, histamine H3 receptor, and histmane H4 receptor. Modulation of histamine receptor interactions with PDZ proteins may provide effective treatments for these and many other diseases.

3. Acetylcholine Receptors

Acetylcholine receptors are involved in activation of neurons. Inappropriate activation can lead to Parkinson's like symptoms in animal models, increased metabolic activity, increased cardiac activity, epilepsy, and psuchological disorders and responses. Representative members include, but are not limited to, ACM1, ACM2, ACM3, ACM4 and ACM5.

4. Adrenoceptors

Adrenoceptors are involved a number of biological process, including synaptic plasticity, long term potentiation, inflammation, asthma, obesity, rheumatoid arthritis, overactive bladder disorder, and hypertension. In addition, these receptors have been implicated in heroin addiction, chronic heart failure, and other cardiovascular diseases. Representative members of this family include the beta1-, beta2-, beta3- and beta4-adrenergic receptors, and the alpha1- and alpha2-adrenergic receptors. Modulation of adrenergic receptor interactions with PDZ proteins may provide effective treatments for the diseases listed above and other cardiovascular diseases.

5. Dopamine Receptors

Dopamine receptors are known to be essential for normal neurotransmission. Abnormalities in dopamine receptor function or localization can result in a number of neurological diseases, including but not limited to Parkinson's disease, schizophrenia, and Attention Deficit Hyperactivity Disorder (ADHD). Representative members of this group include but are not limited to Dopamine 1 receptor, Dopamine 2 receptor, Dopamine 3 receptor, Dopamine 4 receptor, and Dopamine 5 receptor. Modulation of dopamine receptor interactions with PDZ proteins may provide effective treatments for a variety of neurological disorders, including those listed above.

6. Bradykinin Receptors

Bradykinin receptors are involved in a number of biological functions, including inflammation, tissue injury, asthma, perennial rhinitis, diabetes, and brain edema. Bradykinin receptors have also been implicated in various cardiovascular diseases, including hypertension, myocardial hypertrophy, myocardial infarction, and arrhythmias. Representative members of this group include but are not limited to B1 bradykinin receptor and B2 bradykinin receptor. Modulation of bradykinin receptor interactions with PDZ proteins may provide effective treatments for many diseases, including those listed above.

7. Anaphylatoxin Chemotactic Receptors

Anaphylatoxin chemotactic receptors and their homologues form a group that is highly involved in the inflammatory response, and is involved in other biological functions to a lesser degree. Representative members of this group include C5a anaphylatoxin chemotactic receptor and C3a anaphylatoxin chemotactic receptor. Modulation of anaphylatoxin chemotactic receptor interactions with PDZ proteins may provide potent therapies for inflammation.

8. Interleukin 8 Receptors

Interleukin 8 receptors play a role in lung disease, multiple myeloma, and inflammation. Representative members of this group include IL8RA and IL8RB. Modulation of interleukin receptor interactions with PDZ proteins may provide effective treatments for these and other diseases.

9. Fmet-leu-phe Receptors

Fmet-leu-phe (FMLF or FMLP). receptors are receptors to chemoattractants, and thus are highly involved in inflammation, tissue injury and repair, and phygocytosis of foreign bacteria or microbes. Representative members of this group include but are not limited to FMLP receptor I and FMLP receptor II. Modulation of FMLP receptor interactions with PDZ proteins may provide effective means for regulating chemotaxis and inflammation, and for treating bacterial or viral infections.

10. Angiotensin Receptors

Angiotensin receptors are known to be involved in diabetes, hypertension, cardiovascular disease, renal disease, proteinuria and other diseases. Representative members of this group include but are not limited to type 2 angiotensin II receptor, type 1A angiotensin II receptor, and type 1B angiotensin II receptor. Modulation of angiotensin receptor interactions with PDZ proteins may provide effective treatments for many diseases, including those listed above.

11. Endothelin Receptors

Endothelin receptors play a role in a variety of biological functions, including a major role in the female reproductive cycle. In addition, these receptors have been implicated in a number of diseases, including glaucoma, hypertension, congestive heart failure, and cerebral vasospasm. Representative members of this group include but are not limited to endothelin A receptor and endothelin B receptor. Modulation of endothelin receptor interactions with PDZ proteins may provide effective treatments for a variety of diseases, including those listed above.

12. Melanocortin Receptors

Melanocortin receptors are known to be involved in obesity, anorexia nervosa, nociception, and a variety of other biological processes or disorders. Representative members of this group include but are not limited to adrenocorticotropic hormone receptor, melanocortin receptor 2, melanocortin receptor 3, melanocortin receptor 4, melanocortin receptor 5, and melanocyte stimulating hormone receptor. Modulation of melanocortin receptor interactions with PDZ proteins may provide effective treatment for diseases such as obesity and anorexia nervosa.

13. Neuropeptide Y Receptors

Neuropeptide Y receptors are known to be involved in a number of biological functions and diseases, including stress, cardiovascular disease, feeding disorders, seizures, hypertension, obesity, anxiety, diabetes, and intestinal disorders. Representative members of this group include but are not limited to Neuropeptide Y receptor type 1, Neuropeptide Y receptor type 2, Neuropeptide Y receptor type 4, and Neuropeptide Y receptor type 5. Modulation of neuropeptide receptor interactions with PDZ proteins may provide effective treatments for those diseases listed above and many others.

14. Neurotensin Receptors

Neurotensin receptors are involved in a variety of diseases, including psychological disorders such as Parkinson's disease and schizophrenia. Representative members of this group include but are not limited to neurotensin receptor type 1 and neurotensin receptor type 2. Modulation of neurotensin receptor interactions with PDZ proteins may provide effective treatment for psychological disorders and other diseases.

15. Opioid Receptors

Opioid receptors are involved in a variety of diseases, including but not limited to polycystic ovarian syndrome, irritable bowel syndrome, heroin addiction, and ileus. Representative members of this group include mu-opioid receptor, delta-opioid receptor, kappa-opioid receptor, and nociceptin receptor. Modulation of opioid receptor interactions with PDZ proteins may provide effective treatments for these and many other diseases.

16. Somatostatin Receptors

Somatostatin receptors are involved in the modulation of endocrine and exocrine functions in both nervous and non-nervous tissues, and plays a role in obesity, diabetes mellitus, acromegaly, and many other diseases. Representative members of this family include sst1, sst2A, sst2B, sst3, sst4 and sst5. Modulation of somatostatin receptor interactions with PDZ proteins may provide effective treatments for these diseases and, various cancers, due to somatostatin receptor over expression on many types of tumors.

17. Tachykinin Receptors

Tachykinin receptors are involved in a number of diseases and disorders, such as incontinence, migrane, fibromyalgia, asthma, emesis, psoriasis, central nervous system disorders, and gastrointestinal diseases. Representative members of this group include Substance P receptor, Substance K receptor, Neuromedin K receptor 3, and Neuromedin K receptor 4. Modulation of tachykinin receptor interactions with PDZ proteins may provide. effective treatment for those diseases listed above and many others.

18. Vasopressin-Like Receptors

Vasopressin-like receptors are involved in many biological functions, including reproductive regulation and water metabolism. Oxytocin receptor is highly involved in the reproductive system, regulating parturition, lactation, and other reproductive functions. Representative members of this group include Vasopressin V1A, Vasopressin V1B, Vasopressin V2, and Oxytocin receptor. Modulation of vasopressin-like receptor interactions with PDZ proteins may provide an effective means for regulating, among others, reproductive function and water metabolism.

19. Galanin-Like Receptors

Galanin-like receptors are involved in a variety of diseases and disorders, including obesity, Alzheimer's disease, epilepsy, eating disorders, and depression. Representative members of this group include but are not limited to Galanin receptor type 1, and Galanin receptor type 2. Modulation of galanin-like receptor interactions with PDZ proteins may provide effective treatments for many diseases, including those listed above.

20. Proteinase-Activated Like Receptors

Proteinase-activated like receptors are involved in vascular and cardiovascular disease, cancer, gastrointestinal disease and inflammation. Representative members of this group include but are not limited to Proteinase-activated receptor 2, Proteinase-activated receptor 3, and Thrombin receptor. Modulation of proteinase-activated like receptor interactions with PDZ proteins may provide effective treatments for those diseases listed above, in addition to many others.

21. Orexin & Neuropeptide FF Receptors

Orexin and Neuropeptide FF receptors are involved in many diseases, such as eating disorders and narcolepsy. Representative members of this group include but are not limited to Neuropeptide FF receptor 1, Neuropeptide FF receptor 2, Orexin receptor 1, and Orexin receptor 2. Modulation of orexin and neuropeptide FF receptor interactions with PDZ proteins may provide effective treatments for many diseases, including those listed above.

22. Urotensin II Receptors

23. Adrenomedullin Receptors

24. Endothelin B-Like Receptors

25. Chemokine Receptors

Chemokine receptors and their homologues form a group that is involved in many biological processes, including but not limited to immunosurveillance, inflammation, viral infection, lung disease, graft/transplant rejection, HIV infection, autoimmune disease, angiogenesis, tumorigenesis, wound healing, and metastasis. Modulation of chemokine receptor interactions with PDZ proteins may provide effective treatments for these and other diseases.

26. Neuromedin U Receptors

27. Hormone Receptors

Hormone receptors are involved in a number of endocrine functions and diseases, including but not limited to Graves' disease, autoimmune hypothyroidism, and thyroid cancer. Representative members of this group include follicle-stimulating hormone receptor, lutropin-choriogonadotropic hormone receptor, thyrotropin receptor, luteinizing hormone receptor, and gonadotropin receptor. Modulation of hormone receptor interactions with PDZ proteins may provide effective treatments for these and other endocrine diseases.

28. Rhodopsin Receptors

Rhodopsin receptors are highly involved in the visual system, regulating signal transduction in response to light stimuli. Representative members of this group include but are not limited to blue-sensitiveopsin receptor, red-sensitiveopsin receptor, green-sensitive opsin receptor, and rhodopsin. Modulation of rhodopsin receptor interactions with PDZ proteins may provide treatment for many diseases of the visual system.

29. Olfactory Receptors

Olfactory receptors are involved primarily in the sense of smell. Representative members of this group include OR1A1, OR1C1, OR2A4, OR2B2, OR2W1, and OR2J3, in addition to many others. Modulation of olfactory receptor interactions with PDZ proteins may provide treatments for temporary loss of smell and permanent anosmia.

30. Adenosine Receptors

Adenosine receptors and their homologues form a group that is involved in renal disease, asthma, Parkinson's disease, and many other diseases. Representative members of this group include, but are not limited to Adenosine A1 receptor, Adenosine A2A receptor, Adenosine A2B receptor, and Adenosine A3 receptor. Modulation of adenosine receptor interactions with PDZ proteins may provide effective treatments for the diseases listed above, and others.

31. Cannabis Receptors

Cannabis receptors have been implicated in psychological disorders, hypotension, cardiovascular regulation, pain regulation, movement, memory, and appetite. In addition, they have been investigated as potential therapies for Huntington's Disease, Parkinson's disease, schizophrenia, and tremor. Representative members of this group include but are not limited to Cannabinoid receptor 1 and Cannabinoid receptor 2. Modulation of cannabis receptor interactions with PDZ proteins may provide therapies such as those listed above.

32. Platelet Activating Factor Receptors

33. Gonadotropin-Releasing Hormone Receptors

34. Thyrotropin-Releasing Hormone & Secretagogue Receptors

Thyrotropin-releasing hormone & secretagogue receptors are known to be involved in many thyroid diseases, including hypo- and hyperthyroidism, amyotrophic lateral sclerosis (ALS), obesity, and gastrointestinal disorders such as inflammatory bowel disease and ulcerative colitis. Representative members of this group include but are not limited to Growth Hormone Secretagogue receptor type 1, Motilin receptor, and thyrotropin-releasing hormone receptor. Modulation of thyrotropin-releasing hormone & secretagogue receptor interactions with PDZ proteins may provide relief for the

35. Melatonin Receptors

Melatonin receptors are most commonly recognized for their role in the circadian rhythm, however,these receptors also play a role in the cerebrovascular, reproductive, visual, neuroendocrine, and neuroinmunological systems. In addition, they are associated with cancer, rheumatoid arthritis, and reduction of NSAID-caused lesions. Representative members of this group include but are not limited to melatonin receptor 1A, melatonin receptor 1B, and melatonin-related receptor. Modulation of melatonin receptor interactions with PDZ proteins may provide effective therapies and treatments for a variety of diseases, including those listed above.

36. Lysosphinpolipid & LPA (EDG) Receptors

37. Leukotrine Receptors

38. Calcitonin Receptors

Calcitonin receptors play a role in bone mineral density, osteoporosis, and prostate cancer. In addition, calcitonin receptors have been implicated in renal function, embryonic development, and sperm function and physiology. Modulation of calcitonin receptor interactions with PDZ proteins may provide an effective means for treating diseases such as osteoporosis or prostate cancer.

39. Corticotropin-releasing factor Receptors Corticotropin-releasing factor (CRF) receptors are known to be involved in the stress response, irritable bowel syndrome, obesity, depression, eating disorders, and cardiac and inflammatory diseases. Modulation of CRF receptor interactions with PDZ proteins may provide an effective means for treating stress and diseases associated with stress, including those listed above.

40. Gastric Inhibitory Peptide Receptors

41. Glucagon Receptors

Glucagon peptide and Glucagon-like peptide receptors form a group that is known to play a role in diabetes mellitus, obesity, and gastrointestinal repair and cytoprotection. In addition, glucagon receptors are integral to glucagonoma syndrome, which can be related to endocrine tumors. Modulation of glucagon receptor interactions with PDZ proteins may provide an effective treatment for diabetes, obesity, glucagonoma, and disorders characterized by injury and/or dysfunction of the intestinal mucosal epithelium.

42. Growth Hormone-Releasing Hormone Receptors

43. Parathyroid Hormone Receptors

44. PACAP Receptors

45. Secretin-Like Receptors

Representative members of this group include but are not limited to gastric inhibitory peptide receptor, growth hormone-releasing hormone receptor, parathyroid hormone receptor, brain-specific angiogenesis inhibitor receptors, calcitonin receptors, CD97, cadherin EGF LAG receptor, corticotropin releasing factor receptors, cell surface glycoprotein EMR1, glucagon-like peptide receptors, Latrophilin-1 receptor, PACAP receptor, Lectomedin receptors, and VIP receptors. Modulation of secretin-like receptor interactions with PDZ proteins may provide effective treatment for a variety of diseases and disorders.

46. Vasoactive intestinal polypeptide Receptors

Vasoactive Intestinal Peptide (VIP) receptors play a role in a number of autoimmune diseases, including but not limited to septic shock, rheumatoid arthritis, multiple sclerosis, Crohn's disease, asthma, and autoimmune diabetes. In addition, VIP receptors are known to be involved in the inflammatory response and pulmonary hypertension, and are fundamental to Verner-Morrison syndrome. Modulation of vasoactive intestinal peptide receptor interactions with PDZ proteins may provide an effective-means for treating autoimune diseases, affecting inflammatory responses, or alleviating the symptoms of Verner-Morrison Syndrome.

47. Diuretic Hormone Receptors

48. EMR1 Receptors

49. Latrophilin Receptors

50. Brain-Specific Angiogenesis Inhibitor (BAI) Receptors

51. Methuselah-like Protein (MTH) Receptors

52. Metabotropic Glutamate Receptors

Metabotropic glutamate receptors are involved in inflammatory pain, anxiety, neurodegenerative diseases such as Parkinson's disease and Alzheimer's disease, brain ischemia, amyotrophic lateral sclerosis, and seizure disorders. Modulation of metabotropic glutamate receptor interactions with PDZ proteins may provide effective anticonvulsant and neuroprotective therapies and treatments for inflammatory and other disorders.

53. GABA Receptors

GABA receptors, or gamma-aminobutyricacid receptors, play a critical role in the fine-tuning of central nervous system synaptic transmission and are attractive targets for the treatment of epilepsy, anxiety, depression, cognitive deficits, and nociceptive disorders. This family includes GBR1 and GBR2.

54. Ocular Albinism Protein Receptors

55. Frizzled/Smoothened Receptors

56. Vomeronasal Receptors

57. Thromboxane Receptors

Thromboxane receptors and their homologues form a group that is involved in inflammation, asthma, and cardiovascular disorders such as myocardial ischemia, hypertension, stroke, thrombosis, and restenosis. Modulation of thromboxane receptor interactions with PDZ proteins may provide effective treatments for many diseases, including but not limited to asthma, inflammation, and cardiovascular diseases.

58. Prostaglandin Receptors

Prostaglandin receptors' are involved in arthritis, insomnia, colon cancer, and many other diseases. Prostaglandin receptors also play a large role in vascular contraction and thus are important effectors in, among others, inflammation, myocardial ischemia, hypertension, stroke, and thrombosis. Modulation of prostaglandin receptor interactions with PDZ proteins may provide effective treatments for vascular diseases, arthritis, colon cancer, insomnia, and other diseases.

59. GPCR Receptors Expressed on T Cell Surface

GPCRs are used for a number of function on the surface of T cells, including chemokine sensing, cytokine sensing, and environment sensing. Modulation of interactions between these receptors and PDZ proteins could be used to treat a wide variety of immune and inflammatory disorders.

60. GPCR Receptors Expressed on B Cell Surface

GPCRs are used for a number of function on the surface of B cells, including chemokine sensing, cytokine sensing, and environment sensing. Modulation of interactions between these receptors and PDZ proteins could be used to treat a wide variety of immune and inflammatory disorders.

61. GPCR Receptors Expressed on NK Cell Surface

62. GPCR Receptors Expressed on Monocyte Surface

63. GPCR Receptors Expressed on Granulocyte Surface

64. GPCR Receptors Expressed on Endothelial Cell Surface

65. GPCR Receptors Involved in the Immune Response

66. GPCR Receptors Involved in the Cardiovascular System

67. GPCR Receptors Involved in the Neural System

68. GPCR Receptors Involved in the Inflammatory Response

Many GPCRs are involved in inflammatory responses,whether vascular, histamine related or other inflammatory responses. Modulation of PDZ:GPCRPL interactions could be used to treat these symptoms.

69. GPCR Receptors Involved in Asthma and Allergic Inflammation

A number of GPCR proteins containing a PL motif are involved in asthma and the allergic inflammatory response. These include, but are not limited to adrenergic receptors and leukotriene receptors. Modulation of PDZ:GPCRPL interactions could be used to treat these symptoms.

70. GPCR Receptors Involved in Parkinson's Disease

Glutamate, GABA and NMDA receptors have been implicated as potential drug targets that may slow progression of Parkinson's disease or treat the symptoms such that quality of life improves.

71. Group 71

Members of Group 71 include alphalA-Adrenergic receptor, beta2-Adrenergic receptor, P2Y1 purinergic receptor, GRK6A, betal-Adrenergic receptor, parathyroid hormone 1 receptor, parathyroid hormone 1 receptor, 5HT2B, platelet-derived growth factor receptor, mGLUR1a, mGLUR5, SSTR2, SSTR2, IL8RB, CL1 (a-latrotoxin), 5HT2B, B1AR, rat SSTR2, 5HT2C, SSTR2A, CIRL1, CIRL2, CIRL1 & 2, prolactin-releasing peptide receptor, kappa opioid receptor, mGLUR7,

V. Detection of PDZ Domain-Containing Proteins

As noted supra, the present inventors have identified a number of PDZ protein and PL protein interactions that can play a role in modulation of a number of biological functions in a variety of cell types. A comprehensive list of PDZ domain-containing proteins was retrieved from the Sanger Centre database (Pfam) searching for the keyword, “PDZ”. The corresponding cDNA sequences were retrieved from GenBank using the NCBI “entrez” database (hereinafter, “GenBank PDZ protein cDNA sequences”). The DNA portion encoding PDZ domains was identified by alignment of cDNA and protein sequence using CLUSTALW. Based on the DNA/protein alignment information, primers encompassing the PDZ domains were designed. The expression of certain PDZ-containing proteins in cells was detected by polymerase chain reaction (“PCR”) amplification of cDNAs obtained by reverse transcription (“RT”) of cell-derived RNA (i.e., “RT-PCR”). PCR, RT-PCR and other methods for analysis and manipulation of nucleic acids are well known and are described generally in Sambrook et al., (1989) MOLECULAR CLONING: A LABORATORY MANUAL, 2ND ED., VOLS. 1-3, Cold Spring Harbor Laboratory hereinafter, “Sambrook”); and Ausubel et al., CURRENT PROTOCOLS IN MOLECULAR BIOLOGY, Greene Publishing and Wiley-Interscience, New York (1997), as supplemented through January 1999 (hereinafter “Ausubel”).

Samples of cDNA for those sequences identified through the foregoing search were obtained and then amplified. In general a sample of the cDNA (typically, ⅕ of a 20 μl reaction) was used to conduct PCR. PCR was conducted using primers designed to amplify specifically PDZ domain-containing regions of PDZ proteins of interest. Oligonucleotide primers were designed to amplify one or more PDZ-encoding domains. The DNA sequences encoding the various PDZ domains of interest were identified by inspection (i.e., conceptual translation of the PDZ protein cDNA sequences obtained from GenBank, followed by alignment with the PDZ domain amino acid sequence). TABLE 6 shows the PDZ-encoded domains amplified, and the GenBank accession number of the PDZ-domain containing proteins. To facilitate subsequent cloning of PDZ domains, the PCR primers included endonuclease restriction sequences at their ends to allow ligation with pGEX-3X cloning vector (Pharmacia, GenBank XXI13852) in frame with glutathione-S transferase (GST).

TABLE 6 lists the proteins isolated for use in the aforementioned assays.

VI. Assays for Detection of Interactions Between PDZ-Domain Polypeptides and Candidate PDZ Ligand Proteins (PL Proteins)

Two complementary assays, termed “A′ and “G,″” were developed to detect binding between a PDZ-domain polypeptide and candidate PDZ ligand. In each of the two different assays, binding is detected between a peptide having a sequence corresponding to the C-terminus of a protein anticipated to bind to one or more PDZ domains (i.e. a candidate PL peptide) and a PDZ-domain polypeptide (typically a fusion protein containing a PDZ domain). In the “A” assay, the candidate PL peptide is immobilized and binding of a soluble PDZ-domain polypeptide to the immobilized peptide is detected (the “A′” assay is named for the fact that in one embodiment an avidin surface is used to immobilize the peptide). In the “G” assay, the PDZ-domain polypeptide is immobilized and binding of a soluble PL peptide is detected (The “G” assay is named for the fact that in one embodiment a GST-binding surface is used to immobilize the PDZ-domain polypeptide). Preferred embodiments of these assays are described in detail infra. However, it will be appreciated by ordinarily skilled practitioners that these assays can be modified in numerous ways while remaining useful for the purposes of the present invention.

A. Production of Fusion Proteins Containing PDZ-Domains

GST-PDZ domain fusion proteins were prepared for use in the assays of the invention. PCR products containing PDZ encoding domains (as described supra) were subcloned into an expression vector to permit expression of fusion proteins containing a PDZ domain and a heterologous domain (i.e., a glutathione-S transferase sequence, “GST”). PCR products (i.e., DNA fragments) representing PDZ domain encoding DNA was extracted from agarose gels using the “sephaglas” gel extraction system (Pharmacia) according to the manufacturer's recommendations.

As noted supra, PCR primers were designed to include endonuclease restriction sites to facilitate ligation of PCR fragments into a GST gene fusion vector (pGEX-3X; Pharmacia, GenBank accession no. XXU13852) in-frame with the glutathione-S transferase coding sequence. This vector contains a IPTG inducible lacZ promoter. The pGEX-3X vector was linearized using Bam HI and Eco RI or, in some cases, Eco RI or Sma I, and dephosphorylated. For most cloning approaches, double digestion with Bam HI and Eco RI was performed, so that the ends of the PCR fragments to clone were Bam HI and Eco RI. In some cases, restriction endonuclease combinations used were Bgl II and Eco RI, Bam HI and Mfe I, or Eco RI only, Sma I only, or BamHI only. When more than one PDZ domain was cloned, the DNA portion cloned represents the PDZ domains and the cDNA portion located between individual domains. Precise locations of cloned fragments used in the assays are indicated in TABLE 6. DNA linker sequences between the GST portion and the PDZ domain containing DNA portion vary slightly, dependent on which of the above described cloning sites and approaches were used. As a consequence,the amino acid sequence of the GST-PDZ fusion protein varies in the linker region between GST and PDZ domain. Protein linkers sequences corresponding to different cloning sites/approaches are shown below. Linker sequences (vector DNA encoded) are bold, PDZ domain containing gene derived sequences are in italics.
1) GST-BamHI/BamHI-PDZ domain insert
   Gly-Ile-PDZ domain insert
2) GST-BamHI/BglII-PDZ domain insert
   Gly-Ile-PDZ domain insert
3) GST-EcoRI/EcoI-PDZ domain insert
   Gly-Ile Pro-Gly-Asn-PDZ domain insert
4) GST-SmaI/SmaI-PDZ domain insert
   Gly-Ile Pro-PDZ domain insert

The PDZ-encoding PCR fragment and linearized pGEX-3X vector were ethanol precipitated and resuspended in 10 ul standard ligation buffer. Ligation was performed for 4-10 hours at 7° C. using T4 DNA ligase. It will be understood that some of the resulting constructs include very short linker sequences and that, when multiple PDZ domains were cloned, the constructs included some DNA located between individual PDZ domains.

The ligation products were transformed in DH5α or BL-21 E. coli bacteria strains. Colonies were screened for presence and identity of the cloned PDZ domain containing DNA as well as for correct fusion with the glutathione S-transferase encoding DNA portion by PCR and by sequence analysis. Positive clones were tested in a small scale assay for expression of the GST/PDZ domain fusion protein and, if expressing, these clones were subsequently grown up for large scale preparations of GST/PDZ fusion protein.

GST-PDZ domain fusion protein was over expressed following addition of IPTG to the culture medium and purified. Detailed procedure of small scale and large scale fusion protein expression and purification are described in“GST Gene Fusion System” (second edition, revision 2; published by Pharmacia). In brief, a small culture (3-5 mls) containing a bacterial strain (DH5α, BL21 or JM109) with the fusion protein construct was grown overnight in LB-media at 37° C. with the appropriate antibiotic selection (100 ug/ml ampicillin; a.k.a. LB-amp). The overnight culture was poured into a fresh preparation of LB-amp (typically 250-500 mls) and grown until the optical density (OD) of the culture was between 0.5 and 0.9 (approximately 2.5 hours). IPTG (isopropyl β-D-thiogalactopyranoside) was added to a final concentration of 1.0 mM to induce production of GST fusion protein, and culture was grown an additional 1.5-2.5 hours. Bacteria were collect by centrifugation (4500 g) and resuspended in Buffer A− (50 mM Tris, pH 8.0, 50 mM dextrose, 1 mM EDTA, 200 uM phenylmethylsulfonylfluoride). An equal volume of Buffer A+ (Buffer A−, 4 mg/ml lysozyme) was added and incubated on ice for 3 min to lyse bacteria. An equal volume of Buffer B (10 mM Tris, pH 8.0, 50 mM KCl, 1 mM EDTA. 0.5% Tween-20, 0.5% NP40 (a.k.a. IGEPAL CA-630), 200 uM phenylmethylsulfonylfluoride) was added and incubated for an additional 20 min. The bacterial cell lysate was centrifuged (×20,000 g), and supernatant was added to glutathione Sepharose 4B (Pharmacia, cat no.17-0765-01) previously swelled (rehydrated) in 1× phosphate-buffered saline (PBS). The supernatant-Sepharose slurry was poured into a column and washed with at least 20 bed volumes of 1×PBS. GST fusion protein was eluted off the glutathione sepharose by applying 0.5-1.0 ml aliquots of 5 mM glutathione and collected as separate fractions. Concentrations of fractions were determined using BioRad Protein Assay (cat no.500-0006) according to manufacturer's specifications. Those fractions containing the highest concentration of fusion protein were pooled and dialyzed against 1×PBS/35% glycerol. Fusion proteins were assayed for size and quality by SDS gel electrophoresis (PAGE) as described in “Sambrook.” Fusion protein aliquots were stored at minus 80° C. and at minus 20° C.

B. Identification of Candidate PL Proteins and Synthesis of Peptides

Certain PDZ domains are bound by the C-terminal residues of PDZ-binding proteins. To identify PL proteins the C-terminal residues of sequences were visually inspected for sequences that one might predict would bind to PDZ-domain containing proteins (see, e.g., Doyle et al., 1996, Cell 85, 1067; Songyang et al., 1997, Science 275, 73). TABLE 3 lists these proteins, and provides corresponding C-terminal sequences and GenBank accession numbers.

Synthetic peptides of defined sequence (e.g., corresponding to the carboxyl-termini of the indicated proteins) can be synthesized by any standard resin-based method (see, e.g., U.S. Pat. No. 4,108,846; see also, Caruthers et al., 1980, Nucleic Acids Res. Symp. Ser., 215-223; Horn et al., 1980, Nucleic Acids Res. Symp. Ser., 225-232; Roberge, et al., 1995, Science 269:202). The peptides used in the assays described herein were prepared by the FMOC (see, e.g., Guy and Fields, 1997, Meth. Enz. 289:67-83; Wellings and Atherton, 1997, Meth. Enz.289:44-67). In some cases (e.g., for use in the A and G assays of the invention), peptides were labeled with biotin at the amino-terminus by reaction with a four-fold excess of biotin methyl ester in dimethylsulfoxide with a catalytic amount of base. The peptides were cleaved from the resin using a halide containing acid (e.g. trifluoroacetic acid) in the presence of appropriate antioxidants (e.g. ethanedithiol) and excess solvent lyophilized.

Following lyophilization, peptides can be redissolved and purified by reverse phase high performance liquid chromatography (HPLC). One appropriate HPLC solvent system involves a Vydac C-18 semi-preparative column running at 5 mL per minute with increasing quantities of acetonitrile plus 0.1% trifluoroaceticacid in a base solventof water plus 0.1% trifluoroacetic acid. After HPLC purification, the identities of the peptides are confirmed by MALDI cation-mode mass spectrometry. As noted, exemplary biotinylated peptides are provided in TABLE 3.

C. Detecting PDZ-PL Interactions

The present inventors were able in part to identify the interactions summarized in TABLE 2 by developing new high throughput screening assays which are described in greater detail infra. Various other assay formats known in the art can be used to select ligands that are specifically reactive with a particular protein. For example, solid-phase ELISA immunoassays, immunoprecipitation, Biacore, and Western blot assays can be used to identify peptides that specifically bind PDZ-domain polypeptides. As discussed supra, two different, complementary assays were developed to detect PDZ-PL interactions. In each, one binding partner of a PDZ-PL pair is immobilized, and the ability of the second binding partner to bind is determined. These assays, which are described infra, can be readily used to screen for hundreds to thousand of potential PDZ-ligand interactions in a few hours. Thus these assays can be used to identify yet more novel PDZ-PL interactions in hematopoietic cells. In addition, they can be used to identify antagonists of PDZ-PL interactions (see infra).

In various embodiments, fusion protein are used in the assays and devices of the invention. Methods for constructing and expressing fusion proteins are well known. Fusion proteins generally are described in Ausubel et al., supra, Kroll et al., 1993, DNA Cell. Biol. 12:441, and Imai et al., 1997, Cell 91:521-30. Usually, the fusion protein includes a domain to facilitate immobilization of the protein to a solid substrate (“an immobilization domain”). Often, the immobilization domain includes an epitope tag (i.e., a sequence recognized by a antibody, typically a monoclonal antibody) such as polyhistidine (Bush et al, 1991, J. Biol. Chem 266:13811-14), SEAP (Berger et al, 1988, Gene 66:1-10), or M1 and M2 flag (see, e.g, U.S. Pat. Nos. 5,011,912; 4,851,341; 4,703,004; 4,782,137). In an embodiment, the immobilization domain is a GST coding region. It will be recognized that, in addition to the PDZ-domain and the particular residues bound by an immobilized antibody, protein A, or otherwise contacted with the surface, the protein (e.g., fusion protein), will contain additional residues. In some embodiments these are residues naturally associated with the PDZ-domain (i.e., in a particular PDZ-protein) but they may include residues of synthetic (e.g., poly(alanine)) or heterologous origin (e.g., spacers of, e.g., between 10 and 300 residues).

PDZ domain-containing polypeptide used in the methods of the invention (e.g., PDZ fusion proteins) of the invention are typically made by (1) constructing a vector (e.g., plasmid, phage or phagemid) comprising a polynucleotide sequence encoding the desired polypeptide, (2) introducing the vector into an suitable expression system (e.g., a prokaryotic, insect, mammalian, or cell free expression system), (3) expressing the fusion protein and (4) optionally purifying the fusion protein.

(1) In one embodiment, expression of the protein comprises inserting the coding sequence into an appropriate expression vector (i.e., a vector that contains the necessary elements for the transcription and translation of the inserted coding sequence required for the expression system employed, e.g., control elements including enhancers, promoters, transcription terminators, origins of replication, a suitable initiation codon (e.g., methionine), open reading frame, and translational regulatory signals (e.g., a ribosome binding site, a termination codon and a polyadenylation sequence. Depending on the vector system and host utilized, any number of suitable transcription and translation elements, including constitutive and inducible promoters, can be used.

The coding sequence of the fusion protein includes a PDZ domain and an immobilization domain as described elsewhere herein. Polynucleotides encoding the amino acid sequence for each domain can be obtained in a variety of ways known in the art; typically the polynucleotides are obtained by PCR amplification of cloned plasmids, cDNA libraries, and cDNA generated by reverse transcription of RNA, using primers designed based on sequences determined by the practitioner or, more often, publicly available (e.g., through GenBank). The primers include linker regions (e.g., sequences including restriction sites) to facilitate cloning and manipulation in production of the fusion construct. The polynucleotides corresponding to the PDZ and immobilization regions are joined in-frame to produce the fusion protein-encoding sequence.

The fusion proteins of the invention may be expressed as secreted proteins (e.g., by including the signal sequence encoding DNA in the fusion gene; see, e.g., Lui et al, 1993, PNAS USA, 90:8957-61) or as nonsecreted proteins.

In some embodiments, the PDZ-containing proteins are immobilized on a solid surface. The substrate to which the polypeptide is bound may in any of a variety of forms, e.g., a microtiter dish, a test tube, a dipstick, a microcentrifuge tube, a bead, a spinnable disk, and the like. Suitable materials include glass, plastic (e.g., polyethylene, PVC, polypropylene, polystyrene, and the like), protein, paper, carbohydrate, lipip monolayer or supported lipid bilayer, and other solid supports. Other materials that may be employed include ceramics, metals, metalloids, semiconductive materials, cements and the like.

In some embodiments, the fusion proteins are organized as an array. The term “array,” as used herein, refers to an ordered arrangement of immobilized fusion proteins, in which particular different fusion proteins (i.e., having different PDZ domains) are located at different predetermined sites on the substrate. Because the location of particular fusion proteins on the array is known, binding at that location can be correlated with binding to the PDZ domain situated at that location. Immobilization of fusion proteins on beads (individually or in groups) is another particularly useful approach. In one embodiment, individual fusion proteins are immobilized on beads. In one embodiment, mixtures of distinguishable beads are used. Distinguishable beads are beads that can be separated from each other on the basis of a property such as size, magnetic property, color (e.g., using FACS) or affinity tag (e.g., a bead coated with protein A can be separated from a bead not coated with protein A by using IgG affinity methods). Binding to particular PDZ domain may be determined; similarly, the effect of test compounds (i.e., agonists and antagonists of binding) may be determined.

Methods for immobilizing proteins are known, and include covalent and non-covalent methods. One suitable immobilization method is antibody-mediated immobilization. According to this method, an antibody specific for the sequence of an “immobilization domain” of the PDZ-domain containing protein is itself immobilized on the substrate (e.g., by adsorption). One advantage of this approach is that a single antibody may be adhered to the substrate and used for immobilization of a number of polypeptides (sharing the same immobilization domain). For example, an immobilization domain consisting of poly-histidine (Bush et al, 1991, J. Biol Chem 266:13811-14) can be bound by an anti-histidine monoclonal antibody (R&D Systems, Minneapolis, Minn.); an immobilization domain consisting of secreted alkaline phosphatase (“SEAP”) (Berger et al, 1988, Gene 66:1-10) can be bound by anti-SEAP (Sigma Chemical Company, St. Louis, Mo.); an immobilization domain consisting of a FLAG epitope can be bound by anti-FLAG. Other ligand-antiligand immobilization methods are also suitable (e.g., an immobilization domain consisting of protein A sequences (Harlow and Lane, 1988, Antibodies A Laboratory Manual, Cold Spring Harbor Laboratory; Sigma Chemical Co., St. Louis, Mo.) can be bound by IgG; and an immobilization domain consisting of streptavidin can be bound by biotin (Harlow & Lane, supra; Sigma Chemical Co., St. Louis, Mo.). In a preferred embodiment, the immobilization domain is a GST moiety, as described herein.

When antibody-mediated immobilization methods are used, glass and plastic are especially useful substrates. The substrates may be printed with a hydrophobic (e.g., Teflon) mask to form wells. Preprinted glass slides with 3, 10 and 21 wells per 14.5 cm² slide “working area” are available from, e.g., SPI Supplies, West Chester, Pa.; also see U.S. Pat. No. 4,011,350). In certain applications, a large format (12.4 cm×8.3 cm) glass slide is printed in a 96 well format is used; this format facilitates the use of automated liquid handling equipment and utilization of 96 well format plate readers of various types (fluorescent, colorimetric, scintillation). However, higher densities may be used (e.g., more than 10 or 100 polypeptides per cm²). See, e.g., MacBeath et al, 2000, Science 289:1760-63.

Typically, antibodies are bound to substrates (e.g., glass substrates) by adsorption. Suitable adsorption conditions are well known in the art and include incubation of 0.5-50 μg/ml (e.g., 10 μg/ml) mAb in buffer (e.g., PBS, or 50 to 300 mM Tris, MOPS, HEPES, PIPES, acetate buffers, pHs 6.5 to 8, at 4° C.) to 37° C. and from 1 hr to more than 24 hours.

Proteins may be covalently bound or noncovalently attached through nonspecific bonding. If covalent bonding between a the fusion protein and the surface is desired, the surface will usually be polyfunctional or be capable of being polyfunctionalized. Functional groups which may be present on the surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature.

“A Assay” Detection of PDZ-Ligand Binding using Immobilized PL Peptide.

In one aspect, the invention provides an assay in which biotinylated candidate PL peptides are immobilized on an avidin coated surface. The binding of PDZ-domain fusion protein to this surface is then measured. In a preferred embodiment, the PDZ-domain fusion protein is a GST/PDZ fusion protein and the assay is carried out as follows:

(1) Avidin is bound to a surface, e.g. a protein binding surface. In one embodiment, avidin is bound to a polystyrene 96 well plate (e.g., Nunc Polysorb (cat #475094) by addition of 100 μL per well of 20 μg/mL of avidin (Pierce) in phosphate buffered saline without calcium and magnesium, pH 7.4 (“PBS”, GibcoBRL) at 4° C. for 12 hours. The plate is then treated to block nonspecific interactions by addition of 200 μL per well of PBS containing 2 g per 100 mL protease-free bovine serum albumin (“PBS/BSA”)for 2 hours at 4° C. The plate is then washed 3 times with PBS by repeatedly adding 200 μL per well of PBS to each well of the, plate and then dumping the contents of the plate into a waste container and tapping the plate gently on a dry surface.

(2) Biotinylated PL peptides (or candidate PL peptides, e.g. see TABLE 3) are immobilized on the surface of wells of the plate by addition of 50 μL per well of 0.4 μM peptide in PBS/BSA for 30 minutes at 4° C. Usually, each different peptide is added to at least eight different wells so that multiple measurements (e.g. duplicates and also measurements using different (3ST/PDZ-domain fusion proteins and a GST alone negative control) can be made, and also additional negative control wells are prepared in which no peptide is immobilized. Following immobilization of the PL peptide on the surface, the plate is washed 3 times with PBS.

(3) GST/PDZ-domain fusion protein (prepared as described supra) is allowed to react with the surface by addition of 50 μL per well of a solution containing 5 μg/mL GST/PDZ-domain fusion protein in PBS/BSA for 2 hours at 4° C. As a negative control, GST alone (i.e. not a fusion protein) is added to specified wells, generally at least 2 wells (i.e. duplicate measurements) for each immobilized peptide. After the 2 hour reaction, the plate is washed 3 times with PBS to remove unbound fusion protein.

(4) The binding of the GST/PDZ-domain fusion protein to the avidin-biotinylated peptide surface can be detected using a variety of methods, and detectors known in the art. In one embodiment, 50 μL per well of an anti-GST antibody in PBS/BSA (e.g. 2.5 μg/mL of polyclonal goat-anti-GST antibody, Pierce) is added to the plate and allowed to react for 20 minutes at 4° C. The plate is washed 3 times with PBS and a second, detectably labeled antibody is added. In one embodiment, 50 μL per well of 2.5 μg/L of horseradish peroxidase (HRP)-conjugated polyclonal rabbit anti-goat immunoglobulin antibody is added to the plate and allowed to react for 20 minutes at 4° C. The plate is washed 5 times with 50 mM Tris pH 8.0 containing 0.2% Tween 20, and developed by addition of 100 μL per well of HRP-substrate solution (TMB, Dako) for 20 minutes at room temperature (RT). The reaction of the HRP and its substrate is terminated by the addition of 100 μL per well of 1 M sulfuric acid and the optical density (O.D.) of each well of the plate is read at 450 nm.

(5) Specific binding of a PL peptide and a PDZ-domain polypeptide is detected by comparing the signal from the well(s) in which the PL peptide and PDZ domain polypeptide are combined with the background signal(s). The background signal is the signal found in the negative controls. Typically a specific or selective reaction will be at least twice background signal, more typically more than 5 times background, and most typically 10 or more times the background signal. In addition, a statistically significant reaction will involve multiple measurements of the reaction with the signal and the background differing by at least two standard errors, more typically four standard errors, and most typically six or more standard errors. Correspondingly, a statistical test (e.g. a T-test) comparing repeated measurements of the signal with repeated measurements of the background will result in a p-value<0.05, more typically a p-value<0.01, and most typically a p-value<0.001 or less.

As noted, in an embodiment of the “A” assay, the signal from binding of a GST/PDZ-domain fusion protein to an avidin surface not exposed to (i.e. not covered with) the PL peptide is one suitable negative control (sometimes referred to as “B”). The signal from binding of GST polypeptide alone (i.e. not a fusion protein) to an avidin-coated surface that has been exposed to (i.e. covered with) the PL peptide is a second suitable negative control (sometimes referred to as “B2”). Because all measurements are done in multiples (i.e. at least duplicate) the arithmetic mean (or, equivalently, average) of several measurements is used in determining the binding, and the standard error of the mean is used in determining the probable error in the measurement of the binding. The standard error of the mean of N measurements equals the square root of the following: the sum of the squares of the difference between each measurement and the mean, divided by the product of (N) and (N-1). Thus, in one embodiment, specific binding of the PDZ protein to the plate-bound PL peptide is determined by comparing the mean signal (“mean S”) and standard error of the signal (“SE”) for a particular PL-PDZ combination with the mean B1 and/or mean B2.

“G Assay”—Detection of PDZ-Ligand Binding using Immobilized PDZ-Domain Fusion Polypeptide

In one aspect, the invention provides an assay in which a GST/PDZ fusion protein is immobilized on a surface (“G” assay). The binding of labeled PL peptide (e.g., as listed in TABLE 2) to this surface is then measured. In a preferred embodiment, the assay is carried out as follows:

(1) A PDZ-domain polypeptide is bound to a surface, e.g. a protein binding surface. In a preferred embodiment, a GST/PDZ fusion protein containing one or more PDZ domains is bound to a polystyrene 96-well plate. The GST/PDZ fusion protein can be bound to the plate by any of a variety of standard methods known to one of skill in the art, although some care must be taken that the process of binding the fusion protein to the plate does not alter the ligand-binding properties of the PDZ domain. In one embodiment, the GST/PDZ fusion protein is bound via an anti-GST antibody that is coated onto the 96-well plate. Adequate binding to the plate can be achieved when:
a. 100 μL per well of 5 μg/mL goat anti-6ST polyclonal antibody (Pierce) in PBS is added to a polystyrene 96-well plate (e.g., Nunc Polysorb) at 4° C. for 12 hours.
b. The plate is blocked by addition of 200 μL per well of PBS/BSA for 2 hours at 4° C.
c. The plate is washed 3 times with PBS.
d. 50 μL per well of 5 μg/mL GST/PDZ fusion protein) or, as a negative control, GST polypeptide alone (i.e. not a fusion protein) in PBS/BSA is added to the plate for 2 hours at 4° C.
e. the plate is again washed 3 times with PBS.

(2) Biotinylated PL peptides are allowed to react with the surface by addition of 50 μL per well of 20 μM solution of the biotinylated peptide in PBS/BSA for 10 minutes at 4° C., followed by an additional 20 minute incubation at 25° C. The plate is washed 3 times with ice cold PBS.

(3) The binding of the biotinylated peptide to the GST/PDZ fusion protein surface can be detected using a variety of methods and detectors known to one of skill in the art. In one embodiment, 100 μL per well of 0.5 μg/mL streptavidin-horse radish peroxidase (HRP) conjugate dissolved in BSA/PBS is added and allowed to react for 20 minutes at 4° C. The plate is then washed 5 times with 50 mM Tris pH 8.0 containing 0.2% Tween 20, and developed by addition of 100 μL per well of HRP-substrate solution (TMB, Dako) for 20 minutes at room temperature (RT). The reaction of the HRP and its substrate is terminated by addition of 100 μL per well of 1 M sulfuric acid, and the optical density (O.D.) of each well of the plate is read at 450 um.

(4) Specific binding of a PL peptide and a PDZ domain polypeptide is determined by comparing the signal from the well(s) in which the PL peptide and PDZ domain polypeptide are combined, with the background signal(s). The background signal is the signal found in the negative control(s). Typically a specific or selective reaction will be at least twice background signal, more typically more than 5 times background, and most typically 10 or more times the background signal. In addition, a statistically significant reaction will involve multiple measurements of the reaction with the signal and the background differing by at least two standard errors, more typically four standard errors, and most typically six or more standard errors. Correspondingly, a statistical test (e.g. a T-test) comparing repeated measurements of the signal with -repeated measurements of the background will result in a p-value<0.05, more typically a p-value<0.01, and most typically a p-value<0.001 or less. As noted, in an embodiment of the “G” assay, the signal from binding of a given PL peptide to immobilized (surface bound) GST polypeptide alone is one suitable negative control (sometimes referred to as “B 1”). Because all measurement are done in multiples (i.e. at least duplicate) the arithmetic mean (or, equivalently, average.) of several measurements is used in determining the binding, and the standard error of the mean is used in determining the probable error in the measurement of the binding. The standard error of the mean of N measurements equals the square root of the following: the sum of the squares of the difference between each measurement and the mean, divided by the product of (N) and (N-1). Thus, in one embodiment, specific binding of the PDZ protein to the plate bound peptide is determined by comparing the mean signal (“mean S”) and standard error-of the signal (“SE”) for a particular PL-PDZ combination with the mean B1.

“G′ Assay” and “G″ Assay”

Two specific modifications of the specific conditions described supra for the “G′ assay” are particularly useful. The modified assays use lesser quantities of labeled PL peptide and have slightly different biochemical requirements for detection of PDZ-ligand binding compared to the specific assay conditions described supra.

For convenience, the assay conditions described in this section are referred to as the “G′ assay” and the “G″ assay,” with the specific conditions described in the preceding section on G assays being referred to as the “G⁰ assay.” The “G′ assay” is identical to the “G⁰ assay” except at step (2) the peptide concentration is 10 uM instead of 20 uM. This results in slightly lower sensitivity for detection of interactions with low affinity and/or rapid dissociation rate. Correspondingly, it slightly increases the certainty that detected interactions are of sufficient affinity and half-life to be of biological importance and useful therapeutic targets.

The “G″ assay” is identical to the “G⁰ assay” except that at step (2) the peptide concentration is 1 μM instead of 20 μM and the incubation is performed for 60 minutes at 25° C. (rather than, e.g., 10 minutes at 4° C. followed by 20 minutes at 25° C.). This results in lower sensitivity for interactions of low affinity, rapid dissociation rate, and/or affinity that is less at 25° C. than at 4° C. Interactions will have lower affinity at 25° C. than at 4° C. if (as we have found to be generally true for PDZ-ligand binding) the reaction entropy is negative (i.e. the entropy of the products is less than the entropy of the reactants). In contrast, the PDZ-PL binding signal may be similar in the “G″ assay” and the “G⁰ assay” for interactions of slow association and dissociation rate, as the PDZ-PL complex will accumulate during the longer incubation of the “G″ assay.” Thus comparison of results of the “G″ assay” and the “G⁰ assay” can be used to estimate the relative entropies, enthalpies, and kinetics of different PDZ-PL interactions. (Entropies and enthalpies are related to binding affinity by the equations delta G=RT 1 n (Kd)=delta H-T delta S where delta G, H, and S are the reaction free energy, enthalpy, and entropy respectively, T is the temperature in degrees Kelvin, R is the gas constant, and Kd is the equilibrium dissociation constant). In particular, interactions that are detected only or much more strongly in the “G⁰ assay” generally have a rapid dissociation rate at 25° C. (t1/2<10 minutes) and a negative reaction entropy, while interactions that are detected similarly strongly in the “G″ assay” generally have a slower dissociation rate at 25° C. (t1/2>10 minutes). Rough estimation of the thermodynamics and kinetics of PDZ-PL interactions (as can be achieved via comparison of results of the “G⁰ assay” versus the “G″ assay” as outlined supra) can be used in the design of efficient inhibitors of the interactions. For example, a small molecule inhibitor based on the chemical structure of a PL that dissociates slowly from a given PDZ domain (as evidenced by similar binding in the “G″ assay” as in the “G⁰ assay”) may itself dissociate slowly and thus be of high affinity.

In this manner, variation of the temperature and duration of step (2) of the “G assay” can be used to provide insight into the kinetics and thermodynamics of the PDZ-ligand binding reaction and into design of inhibitors of the reaction.

Assay Variations

As discussed supra, it will be appreciated that many of the steps in the above-described assays can be varied, for example, various substrates can be used for binding the PL and PDZ-containing proteins; different types of PDZ containing fusion proteins can be used; different labels for detecting PDZ/PL interactions can be employed; and different ways of detection can be used.

The PL protein used in the assay is not intended to be limited to a 20 amino acid peptide. Full length or partial protein may be used, either alone or as a fusion protein. For example, a GST-PL protein fusion may be bound to the anti-GST antibody, with PDZ protein added to the bound PL protein or peptide.

The PDZ-PL detection assays can employ a variety of surfaces to bind the PL and PDZ-containing proteins. For example, a surface can be an “assay plate” which is formed from a material (e.g. polystyrene) which optimizes adherence of either the PL protein or PDZ-containing protein thereto. Generally, the individual wells of the assay plate will have a high surface area to volume ratio and therefore a suitable shape is a flat bottom well (where the proteins of the assays are adherent). Other surfaces include, but are not limited to, polystyrene or glass beads, polystyrene or glass slides, and the like.

For example, the assay plate can be a “microtiter” plate. The term “microtiter” plate when used herein refers to a multiwell assay plate, e.g., having between about 30 to 200 individual wells, usually 96 wells. Alternatively, high density arrays can be used. Often, the individual wells of the microtiter plate will hold a maximum volume of about 250 ul. Conveniently, the assay plate is a 96 well polystyrene plate (such as that sold by Becton Dickinson Labware, Lincoln Park, N.J.), which allows for automation and high throughput screening. Other surfaces include polystyrene microtiter ELISA plates such as that sold by Nunc-Maxisorp, Inter Med, Denmark. Often, about 50 ul to 300 ul, more preferably 100 ul to 200 ul, of an aqueous sample comprising buffers suspended therein will be added to each well of the assay plate.

The detectable labels of the invention can be any detectable compound or composition which is conjugated directly or indirectly with a molecule (such as described above). The label can be detectable by itself (e.g., radioisotope labels or fluorescent labels) or, in the case of an enzymatic label, can catalyze a chemical alteration of a substrate compound or composition which is detectable. The preferred label is an enzymatic one which catalyzes a color change of a non-radioactive color reagent.

Sometimes, the label is indirectly conjugated with the antibody. One of skill is aware of various techniques for indirect conjugation. For example, the antibody can be conjugated with biotin and any of the categories of labels mentioned above can be conjugated with avidin, or vice versa (see also “A” and “G” assay above). Biotin binds selectively to avidin and thus, the label can be conjugated with the antibody in this indirect manner. See, Ausubel, supra, for a review of techniques involving biotin-avidin conjugation and similar assays. Alternatively, to achieve indirect conjugation of the label with the antibody, the antibody is conjugated with a small hapten (e.g. digoxin) and one of the different types of labels mentioned above is conjugated with an anti-haptenantibody (e.g. anti-digoxinantibody). Thus, indirect conjugation of the label with the antibody can be achieved.

Assay variations can include different washing steps. By “washing” is meant exposing the solid phase to an aqueous solution (usually a buffer or cell culture media) in such a way that unbound material (e.g., non-adhering cells, non-adhering capture agent, unbound ligand, receptor, receptor construct, cell lysate, or HRP antibody) is removed therefrom. To reduce background noise, it is convenient to include a detergent (e.g., Triton X) in the washing solution. Usually, the aqueous washing solution is decanted from the wells of the assay plate following washing. Conveniently, washing can be achieved using an automated washing device. Sometimes, several washing steps (e.g., between about 1 to 10 washing steps) can be required.

Various buffers can also be used in PDZ-PL detection assays. For example, various blocking buffers can be used to reduce assay background. The term “blocking buffer” refers to an aqueous, pH buffered solution containing at least one blocking compound which is able to bind to exposed surfaces of the substrate which are not coated with a PL or PDZ-containing protein. The blocking compound is normally a protein such as bovine serum albumin (BSA), gelatin, casein or milk powder and does not cross-react with any of the reagents in the assay. The block buffer is generally provided at a pH between about 7 to 7.5 and suitable buffering agents include phosphate and TRIS.

Various enzyme-substrate combinations can also be utilized in detecting PDZ-PL interactions. Examples of enzyme-substrate combinations include, for example:
(i) Horseradish peroxidase (HRPO) with hydrogen peroxidase as a substrate, wherein the hydrogen peroxidaseoxidizes a dye precursor (e.g. orthophenylene diamine [OPD] or 3,3′,5,5′-tetramethyl benzidine hydrochloride [TMB]) (as described above).
(ii) alkaline phosphatase (AP) with para-Nitrophenyl phosphate as chromogenic substrate.
(iii) β-D-galactosidase (β D-Gal) with a chromogenic substrate (e.g. p-nitrophenyl-β-D-galactosidase) or fluorogenic substrate 4-methylumbelliferyl-β-D-galactosidase.

Numerous other enzyme-substrate combinations are available to those skilled in the art. For a general review of these, see U.S. Pat. Nos. 4,275,149 and 4,318,980, both of which are herein incorporated by reference.

Further, it will be appreciated, that, although, for convenience, the present discussion primarily refers antagonists of PDZ-PL interactions, agonists of PDZ-PL interactions can be identified using the methods disclosed herein or readily apparent variations thereof.

VII. Results of PDZ-PL Interaction Assays

TABLE 2, supra, shows the results of assays in which specific binding was detected using the “G′” assay described herein.

VIII. Measurement of PDZ-Ligand Binding Affinity

The “A” and “G” assays of the invention can be used to determine the “apparent affinity” of binding of a PDZ ligand peptide to a PDZ-domain polypeptide. Apparent affinity is determined based on the concentration of one molecule required to saturate the binding of a second molecule (e.g., the binding of a ligand to a receptor). Two particularly useful approaches for quantitation of apparent affinity of PDZ-ligand binding are provided infra.

(1) A GST/PDZ fusion protein, as well as GST alone as a negative control, are bound to a surface (e.g., a 96-well plate) and the surface blocked and washed as described supra for the “G” assay.

(2) 50 μL per well of a solution of biotinylated PL peptide (e.g. as shown in TABLE 3) is added to the surface in increasing concentrations in PBS/BSA (e.g. at 0.1 μM, 0.33 μM, 1 μM, 3.3 μM, 100 μM, 33 μM, and 100 μM). In one embodiment, the PL peptide is allowed to react with the bound GST/PDZ fusion protein (as well as the GST alone negative control) for 10 minutes at 4° C. followed by 20 minutes at 25° C. The plate is washed 3 times with ice cold PBS to remove unbound labeled peptide.

(3) The binding of the PL peptide to the immobilized PDZ-domain polypeptide is detected as described supra for the “G” assay.

(4) For each concentration of peptide, the net binding signal is determined by subtracting the binding of the peptide to GST alone from the binding of the peptide to the GST/PDZ fusion protein. The net binding signal is then plotted as a function of ligand concentration and the plot is fit (e.g. by using the Kaleidagraph software package curve fitting algorithm) to the following equation, where “Signal_{[ligand]}” is the net binding signal at PL peptide concentration “[ligand],” “Kd” is the apparent affinity of the binding event, and “Saturation Binding” is a constant determined by the curve fitting algorithm to optimize the fit to the experimental data:
Signal_{[ligand]}=Saturation Binding×([ligand]/([ligand]+Kd))

For reliable application of the above equation it is necessary that the highest peptide ligand concentration successfully tested experimentally be greater than, or at least similar to, the calculated Kd (equivalently, the maximum observed binding should be similar to the calculated saturation binding). In cases where satisfying the above criteria proves difficult, an alternative approach (infra) can be used.

Approach 2:

(1) A fixed concentration of a PDZ-domain polypeptide and increasing concentrations of a labeled PL peptide (labeled with, for example, biotin or fluorescein, see TABLE 2 for representative peptide amino acid sequences) are mixed together in solution and allowed to react. In one embodiment, preferred peptide concentrations are 0.1 μM, 1 μM, 10 μM, 100 μM, 1 mM. In various embodiments, appropriate reaction times can range from 10 minutes to 2 days at temperatures ranging from 4° C. to 37° C. In some embodiments, the identical reaction can also be carried out using a non-PDZ domain-containing protein as a control (e.g., if the PDZ-domain polypeptide is fusion protein, the fusion partner can be used).

(2) PDZ-ligand complexes can be separated from unbound labeled peptide using a variety of methods known in the art. For example, the complexes can be separated using high performance size-exclusion chromatography (HPSEC, gel filtration) (Rabinowitz et al., 1998, Immunity 9:699), affinity chromatography(e.g. using glutathione Sepharose beads), and affinity absorption (e.g., by binding to an anti-GST-coated plate as described supra).

(3) The PDZ-ligand complex is detected based on presence of the label on the peptide ligand using a variety of methods and detectors known to one of skill in the art. For example, if the label is fluorescein and the separation is achieved using HPSEC, an in-line fluorescence detector can be used. The binding can also be detected as described supra for the G assay.

(4) The PDZ-ligand binding signal is plotted as a function of ligand concentration and the plot is fit. (e.g., by using the Kaleidagraph software package curve fitting algorithm) to the following equation, where “Signal_{[ligand]}” is the binding signal at PL peptide concentration “[ligand],” “Kd” is the apparent affinity of the binding event, and “Saturation Binding” is a constant determined by the curve fitting algorithm to optimize the fit to the experimental data:
Signal_{[Ligand]}=Saturation Binding×([ligand]/([ligand+Kd])

Measurement of the affinity of a labeled peptide ligand binding to a PDZ-domain polypeptide n is useful because knowledge of the affinity (or apparent affinity) of this interaction allows rational design of inhibitors of the interaction with known potency. The potency of inhibitors in inhibition would be similar to (i.e. within one-order of magnitude of) the apparent affinity of the labeled peptide ligand binding to the PDZ-domain.

Thus, in one aspect, the invention provides a method of determining the apparent affinity of binding between a PDZ domain and a ligand by immobilizing a polypeptide comprising the PDZ domain and a non-PDZ domain on a surface, contacting the immobilized polypeptide with a plurality of different concentrations of the ligand, determining the amount of binding of the ligand to the immobilized polypeptide at each of the concentrations of ligand, and calculating the apparent affinity of the binding based on that data. Typically, the polypeptide comprising the PDZ domain and a non-PDZ domain is a fusion protein. In one embodiment, the e.g., fusion protein is GST-PDZ fusion protein, but other polypeptides can also be used (e.g., a fusion protein including a PDZ domain and any of a variety of epitope tags, biotinylation signals and the like) so long as the polypeptide can be immobilized In an orientation that does not abolish the ligand binding properties of the PDZ domain, e.g, by tethering the polypeptide to the surface via the non-PDZ domain via an anti-domain antibody and leaving the PDZ domain as the free end. It was discovered, for example, reacting a PDZ-GST fusion polypeptide directly to a plastic plate provided suboptimal results. The calculation of binding affinity itself can be determined using any suitable equation (e.g., as shown supra; also see Cantor and Schimmel (1980) BIOPHYSICAL CHEMISTRY W H Freeman & Co., San Francisco) or software.

Thus, in a preferred embodiment,the polypeptide is immobilized by binding the polypeptide to an immobilized immunoglobulin that binds the non-PDZ domain (e.g., an anti-GST antibody when a GST-PDZ fusion polypeptide is used). In a preferred embodiment, the step of contacting the ligand and PDZ-domain polypeptide is carried out under the conditions provided supra in the description of the “G” assay. It will be appreciated that binding assays are conveniently carried out in multiwell plates (e.g., 24-well, 96-well plates, or 384 well plates).

The present method has considerable advantages over other methods for measuring binding affinities PDZ-PL affinities, which typically involve contacting varying concentrations of a GST-PDZ fusion protein to a ligand-coated surface. For example, some previously described methods for determining affinity (e.g., using immobilized ligand and GST-PDZ protein in solution) did not account for oligomerization state of the fusion proteins used, resulting in potential errors of more than an order of magnitude.

Although not sufficient for quantitative measurement of PDZ-PL binding affinity, an estimate of the relative strength of binding of different PDZ-PL pairs can be made based on the absolute magnitude of the signals observed in the “G assay.” This estimate will reflect several factors, including biologically relevant aspects of the interaction, including the affinity and the dissociation rate. For comparisons of different ligands binding to a given PDZ domain-containing protein, differences in absolute binding signal likely relate primarily to the affinity and/or dissociation rate of the interactions of interest.

IX. Assays to Identify Novel PDZ Domain Binding Moieties and to Identify Modulators of PDZ Protein-PL Protein Binding

Although described supra primarily in terms of identifying interactions between PDZ-domain polypeptides and PL proteins, the assays described supra and other assays can also be used to identify the binding of other molecules (e.g., peptide mimetics, small molecules, and the like) to PDZ domain sequences. For example, using the assays disclosed herein, combinatorial and other libraries of compounds can be screened, e.g., for molecules that specifically bind to PDZ domains. Screening of libraries can be accomplished by any of a variety of commonly known methods. See, e.g., the following references, which disclose screening of peptide libraries: Parmley and Smith, 1989, Adv. Exp. Med. Biol. 251:215-218; Scott and Smith, 1990, Science 249:386-390; Fowlkes et al., 1992; Bio Techniques 13:422-427; Oldenburg et al., 1992, Proc. Natl. Acad. Sci. USA 89:5393-5397; Yu et al., 1994, Cell 76:933-945; Staudt et al., 1988, Science 241:577-580; Bock et al., 1992, Nature 355:564-566; Tuerk et al., 1992, Proc. Natl. Acad. Sci. USA 89:6988-6992; Ellington et al., 1992, Nature 355:850-852; U.S. Pat. No. 5,096,815, U.S. Pat. No. 5,223,409, and U.S. Pat. No. 5,198,346, all to Ladner et al.; Rebar and Pabo, 1993, Science 263:671-673; and PCT Publication No. WO 94/18318.

In a specific embodiment, screening can be carried out by contacting the library members with a PDZ-domain polypeptide immobilized on a solid support (e.g. as described supra in the “G” assay) and harvesting those library members that bind to the protein. Examples of such screening methods, termed “panning” techniques are described by way of example in Parmley and Smith, 1988, Gene 73:305-318; Fowlkes et al., 1992, BioTechniques 13:422-427; PCT Publication No. WO 94/18318; and in references cited hereinabove.

In another embodiment, the two-hybrid system for selecting interacting proteins in yeast (Fields and Song, 1989, Nature 340:245-246; Chien et al., 1991, Proc. Natl. Acad. Sci. USA 88:9578-9582) can be used to identify molecules that specifically bind to a PDZ domain-containing protein. Furthermore, the identified molecules are further tested for their ability to inhibit transmembrane receptor interactions with a PDZ domain.

In one aspect of the invention, antagonists of an interaction between a PDZ protein and a PL protein are identified. In one embodiment, a modification of the “A” assay described supra is used to identify antagonists. In one embodiment, a modification of the “G” assay described supra is used to identify antagonists.

In one embodiment, screening assays are used to detect molecules that specifically bind to PDZ domains. Such molecules are useful as agonists or antagonists of PDZ-protein-mediated cell function (e.g., cell activation, e.g., T cell activation, vesicle transport, cytokine release, growth factors, transcriptional changes, cytoskeleton rearrangement, cell movement, chemotaxis, and the like). In one embodiment, such assays are performed to screen for leukocyte activation inhibitors for drug development. The invention thus provides assays to detect molecules that specifically bind to PDZ domain-containing proteins. For example, recombinant cells expressing PDZ domain-encoding nucleic acids can be used to produce PDZ domains in these assays and to screen for molecules that bind to the domains. Molecules are contacted with the PDZ domain (or fragment thereof) under conditions conducive to binding, and then molecules that specifically bind to such domains are identified. Methods that can be used to carry out the-foregoing are commonly known in the art.

It will be appreciated by the ordinarily skilled practitioner that, in one embodiment, antagonists are identified by conducting the A or G assays in the presence and absence of a known or candidate antagonist. When decreased binding is observed in the presence of a compound, that compound is identified as an antagonist Increased binding in the presence of a compound signifies that the compound is an agonist.

For example, in one assay, a test compound can be identified as an inhibitor (antagonist) of binding between a PDZ protein and a PL protein by contacting a PDZ domain polypeptide and a PL peptide or protein in the presence and absence of the test compound, under conditions in which they would (but for the presence of the test compound) form a complex, and detecting the formation of the complex in the presence and absence of the test compound. It will be appreciated that less complex formation in the presence of the test compound than in the absence of the compound indicates that the test compound is an inhibitor of a PDZ protein-PL protein binding.

In one embodiment, the “G” assay is used in the presence or absence of an candidate inhibitor. In one embodiment, the “A” assay is used in the presence or absence of a candidate inhibitor.

In one embodiment (in which a G assay is used), one or more PDZ domain-containing GST-fusion proteins are bound to the surface of wells of a 96-well plate as described supra (with appropriate controls including nonfusion GST protein). All fusion proteins are bound in multiple wells so that appropriate controls and statistical analysis can be done. A test. compound in BSA/PBS (typically at multiple different concentrations) is added to wells. Immediately thereafter, 30 uL of a detectably labeled (e.g., biotinylated) PL peptide or protein known to bind to the relevant PDZ domain (see, e.g., TABLE 2) is added in each of the wells at a final concentration of, e.g., between about 2 μM and about 40 μM, typically 5 μM, 15 μM, or 25 μM. This mixture is then allowed to react with the PDZ fusion protein bound to the surface for 10 minutes at 4° C. followed by 20 minutes at 25° C. The surface is washed free of unbound PL polypeptide three times with ice cold PBS and the amount of binding of the polypeptide in the presence and absence of the test compound is determined. Usually, the level of binding is measured for each set of replica wells (e.g. duplicates) by subtracting the mean GST alone background from the mean of the raw measurement of polypeptide binding in these wells.

In an alternative embodiment, the A assay is carried out in the presence or absence of a test candidate to identify inhibitors of PL-PDZ interactions.

In one embodiment, a test compound is determined to be a specific inhibitor of the binding of the PDZ domain (P) and a PL (L) sequence when, at a test compound concentration of less than or equal to 1 mM (e.g., less than or equal to: 500 μM, 100 μM, 10 μM, 1 μM, 100 nM or 1 nM) the binding of P to L in the presence of the test compound less than about 50% of the binding in the absence of the test compound. (in various embodiments, less than about 25%, less than about 10%, or less than about 1%). Preferably, the net signal of binding of P to L in the presence of the test compound plus six (6) times the standard error of the signal in the presence of the test compound is less than the binding signal in the absence of the test compound.

In one embodiment, assays for an inhibitor are carried out using a single PDZ protein-PL protein pair (e.g., a PDZ domain fusion protein and a PL peptide or protein). In a related embodiment, the assays are carried out using a plurality of pairs, such as a plurality of different pairs listed in TABLE 2.

In some embodiments, it is desirable to identify compounds that, at a given concentration, inhibit the binding of one PL-PDZ pair, but do not inhibit (or inhibit to a lesser degree) the binding of a specified second PL-PDZ pair. These antagonists can be identified by carrying out a series of assays using a candidate inhibitor and different PL-PDZ pairs (e.g., as shown in the matrix of TABLE 2) and comparing the results of the assays. All such pairwise combinations are contemplated by the invention (e.g., test compound inhibits binding of PL₁ to PDZ₁ to a greater degree than it inhibits binding of PL₁ to PDZ₂ or PL₂ to PDZ₂). Importantly, it will be appreciated that, based on the data provided in TABLE 2 and disclosed herein (and additional data that can be generated using the methods described herein) inhibitors with different specificities can readily be designed.

For example, according to the invention, the Ki (“potency”) of an inhibitor of a PDZ-PL interaction can be determined. Ki is a measure of the concentration of an inhibitor required to have a biological effect. For example, administration of an inhibitor of a PDZ-PL interaction in an amount sufficient to result in an intracellular inhibitor concentration of at least between about 1 and about 100 Ki is expected to inhibit the biological response mediated by the target PDZ-PL interaction. In one aspect of the invention, the Kd measurement of PDZ-PL binding as determined using the methods supra is used in determining Ki.

Thus, in one aspect, the invention provides a method of determining the potency (Ki) of an inhibitor or suspected inhibitor of binding between a PDZ domain and a ligand by immobilizing a polypeptide comprising the PDZ domain and a non-PDZ domain on a surface, contacting the immobilized polypeptide with a plurality of different mixtures of the ligand and inhibitor, wherein the different mixtures comprise a fixed amount of ligand and different concentrations of the inhibitor, determining the amount of ligand bound at the different concentrations of inhibitor, and calculating the Ki of the binding based on the amount of ligand bound in the presence of different concentrations of the inhibitor. In an embodiment, the polypeptide is immobilized by binding the polypeptide to an immobilized immunoglobulin that binds the non-PDZ domain. This method, which is based on the “G” assay described supra, is particularly suited for high-throughput analysis of the Ki for inhibitors of PDZ-ligand interactions. Further, using this method, the inhibition of the PDZ-ligand interaction itself is measured, without distortion of measurements by avidity effects.

Typically, at least a portion of the ligand is detectably labeled to permit easy quantitation of ligand binding.

It will be appreciated that the concentration of ligand and concentrations of inhibitor are selected to allow meaningful detection of inhibition. Thus, the concentration of the ligand whose binding is to be blocked is close to or less than its binding affinity (e.g., preferably less than the 5×Kd of the interaction, more preferably less than 2×Kd, most preferably less than 1×Kd). Thus, the ligand is typically present at a concentration of less than 2 Kd (e.g., between about 0.01 Kd and about 2 Kd) and the concentrations of the test inhibitor typically range from 1 nM to 100 μM (e.g. a 4-fold dilution series with highest concentration 10 μM or 1 mM). In a preferred embodiment, the Kd is determined using the assay disclosed supra.

The Ki of the binding can be calculated by any of a variety of methods routinely used in the art, based on the amount of ligand bound in the presence of different concentrations of the inhibitor in an illustrative embodiment, for example, a plot of labeled ligand binding versus inhibitor concentration is fit to the equation:
S_{inhibitor}=S₀*Ki/([I]+Ki)
where S_{inhibitor} is the signal of labeled ligand binding to immobilized PDZ domain in the presence of inhibitor at concentration [I] and S₀ is the signal in the absence of inhibitor (i.e., [I]=0). Typically [I] is expressed as a molar concentration.

In another aspect of the invention, an enhancer (sometimes referred to as, augmentor or agonist) of binding between a PDZ domain and a ligand is identified by immobilizing a polypeptide comprising the PDZ domain and a non-PDZ domain on a surface, contacting the immobilized polypeptide with the ligand in the presence of a test agent and determining the amount of ligand bound, and comparing the amount of ligand bound in the presence of the test agent with the amount of ligand bound by the polypeptide in the absence of the test agent. At least two-fold (often at least 5-fold) greater binding in the presence of the test agent compared to the absence of the test agent indicates that the test agent is an agent that enhances the binding of the PDZ domain to the ligand. As noted supra, agents that enhance PDZ-ligand interactions are useful for disruption (dysregulation) of biological events requiring normal PDZ-ligand function (e.g., cancer cell division and metastasis, and activation and migration of immune cells).

The invention also provides methods for determining the “potency” or “K_{enhancer}” of an enhancer of a PDZ-ligand interaction. For example, according to the invention, the K_{enhancer} of an enhancer of a PDZ-PL interaction can be determined, e.g., using the Kd of PDZ-PL binding as determined using the methods described supra. K_{enhancer} is a measure of the concentration of an enhancer expected to have a biological effect. For example, administration of an enhancer of a PDZ-PL interaction in an amount sufficient to result in an intracellular inhibitor concentration of at least between about 0.1 and about 100 K_{enhancer} (e.g., between about 0.5 and about 50 K_{enhancer}) is expected to disrupt the biological response mediated by the target PDZ-PL interaction.

Thus, in one aspect the invention provides a method of determining the potency (K_{enhancer}) of an enhancer or suspected enhancer of binding between a PDZ domain and a ligand by immobilizing a polypeptide comprising the PDZ domain and a non-PDZ domain on a surface, contacting the immobilized polypeptide with a plurality of different mixtures of the ligand and enhancer, wherein the different mixtures comprise a fixed amount of ligand, at least a portion of which is detectably labeled, and different concentrations of the enhancer, determining the amount of ligand bound at the different concentrations of enhancer, and calculating the potency (K_{enhancer}) of the enhancer from the binding based on the amount of ligand bound in the presence of different concentrations of the enhancer. Typically, at least a portion of the ligand is detectably labeled to permit easy quantitation of ligand binding. This method, which is based on the “G” assay described supra, is particularly suited for high-throughput analysis of the K_{enhancer} for enhancers of PDZ-ligand interactions.

It will be appreciated that the concentration of ligand and concentrations of enhancer are selected to allow meaningful detection of enhanced binding. Thus, the ligand is typically present at a concentration of between about 0.01 Kd and about 0.5 Kd and the concentrations of the test agent/enhancer typically range from 1 nM to 1 mM (e.g. a 4-fold dilution series with highest concentration 10 μM or 1 mM). In a preferred embodiment, the Kd is determined using the assay disclosed supra.

The potency of the binding can be determined by a variety of standard methods based on the amount of ligand bound in the presence of different concentrations of the enhancer or augmentor. For example, a plot of labeled ligand binding versus enhancer concentration can be fit to the equation:
S([E])=S(0)+(S(0)*(D_{enhancer}−1)*[E]/([E]+K_{enhancer})
where “K_{enhancer}” is the potency of the augmenting compound, and “D_{enhancer}” is the fold-increase in binding of the labeled ligand obtained with addition of saturating amounts of the enhancing compound, [E] is the concentration of the enhancer. It will be understood that saturating amounts are the amount of enhancer such that further addition does not significantly increase the binding signal. Knowledge of “K_{enhancer}” is useful because it describes a concentration of the augmenting compound in a target cell that will result in a biological effect due to dysregulation of the PDZ-PL interaction. Typical therapeutic concentrations are between about 0.1 and about 100 K_{enhancer}.
X. Global Analysis of PDZ-PL Interactions

As described supra, the present invention provides powerful methods for analysis of PDZ-ligand interactions, including high-throughput methods such as the “G” assay and affinity assays described supra. In one embodiment of the invention, the affinity is determined for a particular ligand and a plurality of PDZ proteins. Typically the plurality is at least 5, and often at least 25, or at least 40 different PDZ proteins. In a preferred embodiment, the plurality of different PDZ proteins are from a particular tissue (e.g., central nervous system, spleen, cardiac muscle, kidney) or a particular class or type of cell, (e.g., a hematopoietic cell, a lymphocyte, a neuron) and the like. In a most preferred embodiment, the plurality of different PDZ proteins represents a substantial fraction (e.g., typically a majority, more often at least 80%) of all of the PDZ proteins known to be, or suspected of being, expressed in the tissue or cell(s), e.g., all of the PDZ proteins known to be present in lymphocytes. In an embodiment, the plurality is at least 50%, usually at least 80%, at least 90% or all of the PDZ proteins disclosed herein as being expressed in hematopoietic cells.

In one embodiment of the invention, the binding of a ligand to the plurality of PDZ proteins is determined. Using this method, it is possible to identify a particular PDZ domain bound with particular specificity by the ligand. The binding may be designated as “specific” if the affinity of the ligand to the particular PDZ domain is at least 2-fold that of the binding to other PDZ domains in the plurality (e.g., present in that cell type). The binding is deemed “very specific” if the affinity is at least 10-fold higher than to any other PDZ in the plurality or, alternatively, at least 10-fold higher than to at least 90%, more often 95% of the other PDZs in a defined plurality. Similarly, the binding is deemed “exceedingly specific” if it is at least 100-fold higher. For example, a ligand could bind to 2 different PDZs with an affinity of 1 uM and to no other PDZs out of a set 40 with an affinity of less than 100 uM. This would constitute specific binding to those 2 PDZs. Similar measures of specificity are used to describe binding of a PDZ to a plurality of PLs.

It will be recognized that high specificity PDZ-PL interactions represent potentially more valuable targets for achieving a desired biological effect. The ability of an inhibitor or enhancer to act with high specificity is often desirable. In particular, the most specific PDZ-ligand interactions are also the best therapeutic targets, allowing specific inhibition of the interaction.

Thus, in one embodiment,the invention provides a method of identifying a high specificity interaction between a particular PDZ domain and a ligand known or suspected of binding at least one PDZ domain, by providing a plurality of different immobilized polypeptides, each of said polypeptides comprising a PDZ domain and a non-PDZ domain; determining the affinity of the ligand for each of said polypeptides, and comparing the affinity of binding of the ligand to each of said polypeptides, wherein an interaction between the ligand and a particular PDZ domain is deemed to have high specificity when the ligand binds an immobilized polypeptide comprising the particular PDZ domain with at least 2-fold higher affinity than to immobilized polypeptides not comprising the particular PDZ domain.

In a related aspect, the affinity of binding of a specific PDZ domain to a plurality of ligands (or suspected ligands) is determined. For example, in one embodiment, the invention provides a method of identifying a high specificity interaction between a PDZ domain and a particular ligand known or suspected of binding at least one PDZ domain, by providing an immobilized polypeptide comprising the PDZ domain and a non-PDZ domain; determining the affinity of each of a plurality of ligands for the polypeptide, and comparing the affinity of binding of each of the ligands to the polypeptide, wherein an interaction between a particular ligand and the PDZ domain is deemed to have high specificity when the ligand binds an immobilized polypeptide comprising the PDZ domain with at least 2-fold higher affinity than other ligands tested. Thus, the binding may be designated as “specific” if the affinity of the PDZ to the particular PL is at least 2-fold that of the binding to other PLs in the plurality (e.g., present in that cell type). The binding is deemed “very specific” if the affinity is at least 10-fold higher than to any other PL in the plurality or, alternatively, at least 10-fold higher than to at least 90%, more often 95% of the other PLs in a defined plurality. Similarly, the binding is deemed “exceedingly specific” if it is at least 100-fold higher. Typically the plurality is at least 5 different ligands, more often at least 10.

A. Use of Array for Global Predictions

One discovery of the present inventors relates to the important and extensive roles played by interactions between PDZ proteins and PL proteins, particularly in the biological function of hematopoietic cells and other cells involved in the immune response. Further, it has been discovered that valuable information can be ascertained by analysis (e.g., simultaneous analysis) of a large number of PDZ-PL interactions. In a preferred embodiment, the analysis encompasses all of the PDZ proteins expressed in a particular tissue (e.g., spleen) or type or class of cell (e.g., hematopoietic cell, neuron, lymphocyte, B cell, T cell and the like). Alternatively, the analysis encompasses at least about 5, or at least about 10, or at least about 12, or at least about 15 and often at least 50 different polypeptides, up to about 60, about 80, about 100, about 150, about 200, or even more different polypeptides; or a substantial fraction (e.g., typically a majority, more often at least 80%) of all of the PDZ proteins known to be, or suspected of being, expressed in the tissue or cell(s), e.g., all of the PDZ proteins known to be present in lymphocytes.

It will be recognized that the arrays and methods of the invention are directed to analyze of PDZ and PL interactions, and involve selection of such proteins for analysis. While the devices and methods of the invention may include or involve a small number of control polypeptides, they typically do not include significant numbers of proteins or fusion proteins that do not include either PDZ or PL domains (e.g., typically, at least about 90% of the arrayed or immobilized polypeptides in a method or device of the invention is a PDZ or PL sequence protein, more often at least about 95%, or at least about 99%).

It will be apparent from this disclosure that analysis of the relatively large number of different interactions preferably takes place simultaneously. In this context, “simultaneously” means that the analysis of several different PDZ-PL interactions (or the effect of a test agent on such interactions) is assessed at the same time. Typically the analysis is carried out in a high throughput (e.g., robotic) fashion. One advantage of this method of simultaneous analysis is that it permits rigorous comparison of multiple different PDZ-PL interactions. For example, as explained in detail elsewhere herein, simultaneous analysis (and use of the arrays described infra) facilitates, for example, the direct comparison of the effect of an agent (e.g., an potential interaction inhibitor) on the interactions between a substantial portion of PDZs and/or PLs in a tissue or cell.

Accordingly, in one aspect, the invention provides an array of immobilized polypeptide comprising the PDZ domain and a non-PDZ domain on a surface. Typically, the array comprises at least about 5, or at least about 10, or at least about 12, or at least about 15 and often at least 50 different polypeptides. In one preferred embodiment, the different PDZ proteins are from a particular tissue (e.g., central nervous system, spleen, cardiac muscle, kidney) or a particular class or type of cell, (e.g., a hematopoietic cell, a lymphocyte, a neuron) and the like. In a most preferred embodiment, the plurality of different PDZ proteins represents a substantial fraction (e.g., typically a majority, more often at least 60%, 70% or 80%) of all of the PDZ proteins known to be, or suspected of being, expressed in the tissue or cell(s), e.g., all of the PDZ proteins known to be present in lymphocytes.

Certain embodiments are arrays which include a plurality, usually at least 5, 10, 25, 50 PDZ proteins present in a particular cell of interest. In this context, “array” refers to an ordered series of immobilized polypeptides in which the identity of each polypeptide is associated with its location. In some embodiments the plurality of polypeptides are arrayed in a “common” area such that they can be simultaneously exposed to a solution (e.g., containing a ligand or test agent). For example, the plurality of polypeptides can be on a slide, plate or similar surface, which may be plastic, glass, metal, silica, beads or other surface to which proteins can be immobilized. In a different embodiment, the different immobilized polypeptides are situated in separate areas, such as different wells of multi-well plate (e.g., a 24-well plate, a 96-well plate, a 384 well plate, and the like). It will be recognized that a similar advantage can be obtained by using multiple arrays in tandem.

B. Analysis of PDZ-PL Inhibition Profile

In one aspect, the invention provides a method for determining if a test compound inhibits any PDZ-ligand interaction in large set of PDZ-ligand interaction (e.g., a plurality of the PDZ-ligands interactions described in Table 2; a majority of the PDZ-ligands identified in a particular cell or tissue as described supra (e.g., lymphocytes) and the like. In one embodiment, the PDZ domains of interest are expressed as GST-PDZ fusion proteins and immobilized as described herein. For each PDZ domain, a labeled ligand that binds to the domain with a known affinity is identified as described herein.

For any known or suspected modulator (e.g., inhibitor) of a PDL-PL interaction(s), it is useful to know which interactions are inhibited (or augmented). For example, an agent that inhibits all PDZ-PL interactions in a cell (e.g., a lymphocyte) will have different uses than an agent that inhibits only one, or a small number, of specific PDZ-PL interactions. The profile of PDZ interactions inhibited by a particular agent is referred to as the “inhibition profile” for the agent, and is described in detail below. The profile of PDZ interactions enhanced by a particular agent is referred to as the “enhancement profile” for the agent. It will be readily apparent to one of skill guided by the description of the inhibition profile how to determine the enhancement profile for an agent. The present invention provides methods for determining the PDZ interaction (inhibition/enhancement) profile of an agent in a single assay.

In one aspect, the invention provides a method for determining the PDZ-PL inhibition profile of a compound by providing (i) a plurality of different immobilized polypeptides, each of said polypeptides comprising a PDZ domain and a non-PDZ domain and (ii) a plurality of corresponding ligands, wherein each ligand binds at least one PDZ domain in (i), then contacting each of said immobilized polypeptides in (i) with a corresponding ligand in (ii) in the presence and absence of a test compound, and determining for each polypeptide-ligand pair whether the test compound inhibits binding between the immobilized polypeptide and the corresponding ligand.

Typically the plurality is at least 5, and often at least 25, or at least 40 different PDZ proteins. In a preferred embodiment, the plurality of different ligands and the plurality of different PDZ proteins are from the same tissue or a particular class or type of cell, e.g., a hematopoietic cell, a lymphocyte, a neuron and the like. In a most preferred embodiment, the plurality of different PDZs represents a substantial fraction (e.g., at least 80%) of all of the PDZs known to be, or suspected of being, expressed in the tissue or cell(s), e.g., all of the PDZs known to be present in lymphocytes (for example, at least 80%, at least 90% or all of the PDZs disclosed herein as being expressed in hematopoietic cells).

In one embodiment, the inhibition profile is determined as follows: A plurality (e.g., all known) PDZ domains expressed in a cell (e.g., lymphocytes) are expressed as GST-fusion proteins and immobilized without altering their ligand binding properties as described supra. For each PDZ domain, a labeled ligand that binds to this domain with a known affinity is identified. If the set of PDZ domains expressed in lymphocytes is denoted by {P1 . . . Pn}, any given PDZ domain Pi binds a (labeled) ligand Li with affinity K_{d}i. To determine the inhibition profile for a test agent “compound X” the “G” assay (supra) can be performed as follows in 96-well plates with rows A-H and columns 1-12. Column 1 is coated with P1 and washed. The corresponding ligand L1 is added to each washed coated well of column 1 at a concentration 0.5 K_{d}1 with (rows B, D, F, H) or without (rows A, C, E, F) between about 1 and about 1000 uM) of test compound X. Column 2 is coated with P2, and L2 (at a concentration 0.5 K^{d}2) is added with or without inhibitor X. Additional PDZ domains and ligands are similarly tested.

Compound X is considered to inhibit the binding of Li to Pi if the average signal in the wells of column i containing X is less than half the signal in the equivalent wells of the column lacking X. Thus, in this single assay one determines the full set of lymphocyte PDZs that are inhibited by compound X.

In some embodiments, the test compound X is a mixture of compounds, such as the product of a combinatorial chemistry synthesis as described supra. In some embodiments, the test compound is known to have a desired biological effect, and the assay is used to determine the mechanism of action (i.e., if the biological effect is due to modulating a PDZ-PL interaction).

It will be apparent that an agent that modulates only one, or a few PDZ-PL interactions, in a panel (e.g., a panel of all known PDZs lymphocytes, a panel of at least 10, at least 20 or at least 50 PDZ domains) is a more specific modulator than an agent that modulate many or most interactions. Typically, an agent that modulates less than 20% of PDZ domains in a panel (e.g., Table 2) is deemed a “specific” inhibitor, less than 6% a “very specific” inhibitor, and a single PDZ domain a “maximally specific” inhibitor.

It will also be appreciated that “compound X” may be a composition containing mixture of compounds (e.g., generated using combinatorial chemistry methods) rather than a single compound.

Several variations of this assay are contemplated:

In some alternative embodiments, the assay above is performed using varying concentrations of the test compound X, rather than fixed concentration. This allows determination of the Ki of the X for each PDZ as described above.

In an alternative embodiment, instead of pairing each PDZ Pi with a specific labeled ligand Li, a mixture of different labeled ligands is created that such that for every PDZ at least one of the ligands in the mixture binds to this PDZ sufficiently to detect the binding in the “G” assay. This mixture is then used for every PDZ domain.

In one embodiment, compound X is known to have a desired biological effect, but the chemical mechanism by which it has that effect is unknown. The assays of the invention can then be used to determine if compound X has its effect by binding to a PDZ domain.

In one embodiment, PDZ-domain containing proteins are classified in to groups based on their biological function, e.g. into those that regulate chemotaxis versus those that regulate transcription. An optimal inhibitor of a particular function (e.g., including but not limited to an anti-chemotactic agent, an anti-T cell activation agent, cell-cycle control, vesicle transport, apoptosis, etc.) will inhibit multiple PDZ-ligand interactions involved in the function (e.g., chemotaxis, activation) but few other interactions. Thus, the assay is used in one embodiment in screening and design of a drug that specifically blocks a particular function. For example, an agent designed to block chemotaxis might be identified because, at a given concentration, the agent inhibits 2 or more PDZs involved in chemotaxis but fewer than 3 other PDZs, or that inhibits PDZs involved in chemotaxis with a Ki>10-fold better than for other PDZs. Thus, the invention provides a method for identifying an agent that inhibits a first selected PDZ-PL interaction or plurality of interactions but does not inhibit a second selected PDZ-PL interaction or plurality of interactions. The two (or more) sets of interactions can be selected on the basis of the known biological function of the PDZ proteins, the tissue specificity of the PDZ proteins, or any other criteria. Moreover, the assay can be used to determine effective doses (i.e., drug concentrations)that result in desired biological effects while avoiding undesirable effects.

C. Side Effects of PDZ-PL Modulator Interactions

In a related embodiment, the invention provides a method for determining likely side effects of a therapeutic that inhibits PDZ-ligand interactions. The method entails identifying those target tissues, organs or cell types that express PDZ proteins and ligands that are disrupted by a specified inhibitor. If, at a therapeutic dosage, a drug intended to have an effect in one organ system (e.g., hematopoietic system) disrupts PDZ-PL interactions in a different system (e.g., CNS) it can be predicted that the drug will have effects (“side effects”) on the second system. It will be apparent that the information obtained from this assay will be useful in the rational design and selection of drugs that do not have the side-effect.

In one embodiment, for example, a comprehensive PDZ protein set is obtained. A “perfectly comprehensive” PDZ protein set is defined as the set of all PDZ proteins expressed in the subject animal (e.g., humans). A comprehensive set may be obtained by analysis of, for example, the human genome sequence. However, a “perfectly comprehensive” set is not required and any reasonably large set of PDZ domain proteins (e.g., the set of all known PDZ proteins; or the set listed in TABLE 6) will provide valuable information.

In one embodiment, the method involves some of all of the following steps:
a) For each PDZ protein, determine the tissues in which it is highly expressed. This can be done experimentally although the information generally will be available in the scientific literature;
b) For each PDZ protein (or as many as possible), identify the cognate PL(s) bound by the PDZ protein;
c) Determine the Ki at which the test agent inhibits each PDZ-PL interaction, using the methods described supra;
d) From this information it is possible to calculate the pattern of PDZ-PL interactions disrupted at various concentrations of the test agent
   By correlating the set of PDZ-PL interactions disrupted with the expression pattern of the members of that set, it will be possible to identify the tissues likely affected by the agent.

Additional steps can also be carried out, including determining whether a specified tissue or cell type is exposed to an agent following a particular route of administration. This can be determined using basis pharmacokinetic methods and principles.

D. Modulation of Activities

The PDZ binding moieties and PDZ protein-PL protein binding antagonists of the invention are used to modulate biological activities or functions of cells (e.g., hematopoietic cells, such as T cells and B cells and the like), endothelial cells, and other immune system cells, as described herein, and for treatment of diseases and conditions in human and nonhuman animals (e.g., experimental models). Exemplary biological activities are listed supra.

When administered to patients, the compounds of the invention (e.g., PL-PDZ interaction inhibitors) are useful for treating (ameliorating symptoms of) a variety of diseases and conditions, including diseases characterized by inflammatory and humoral immune responses, e.g., inflammation, allergy (e.g., systemic anaphylaxis, hypersensitivity responses, drug allergies, insect sting allergies; inflammatory bowel diseases, ulcerative colitis, ileitis and enteritis; psoriasis and inflammatory dermatoses, scleroderma; respiratory allergic diseases such as asthma, allergic rhinitis, hypersensitivity lung diseases, and the like vasculitis, rh incompatibility, transfusion reactions, drug sensitivities, PIH, atopic dermatitis, eczema, rhinnitis; autoimmune diseases, such as arthritis (rheumatoidand psoriatic), multiple sclerosis, systemic lupus erythematosus, insulin-dependent diabetes, glomerulonephritis, scleroderma, MCTD, IDDM, Hashimoto thyroiditis, Goodpasture syndrome, psoriasis and the like, osteoarthritis, polyarthritis, graft rejection (e.g., allograft rejection, e.g., renal allograft rejection, graft-vs-host disease, transplantation rejection (cardiac, kidney, lung, liver, small bowel, cornea, pancreas, cadaver, autologous, bone marrow, xenotransplantation)), atherosclerosis, angiogenesis-dependent disorders, cancers (e.g., melanomas and breast cancer, prostrate cancer, leukemias, lymphomas, metastatic disease), infectious diseases (e.g., viral infection, such as HIV, measles, parainfluenza, virus-mediated cell fusion,), ischemia (e.g., post-myocardial infarction complications, joint injury, kidney, scleroderma).

E. Agonists and Antagonists of PDZ-PL Interactions

As described herein, interactions between PDZ proteins and PL proteins in cells (e.g., hematopoietic cells, e.g., T cells and B cells) may be disrupted or inhibited by the administration of inhibitors or antagonists. Inhibitors can be identified using screening assays described herein. In embodiment, the motifs disclosed herein are used to design inhibitors. In some embodiments, the antagonists of the invention have a structure (e.g., peptide sequence) based on the C-terminal residues of PL-domain proteins listed in TABLE 3. In some embodiments, the antagonists of the invention have a structure (e.g., peptide sequence) based on a PL motif disclosed herein.

The PDZ/PL antagonists and antagonists of the invention may be any of a large variety of compounds, both naturally occurring and synthetic, organic and inorganic, and including polymers (e.g., oligopeptides, polypeptides, oligonucleotides, and polynucleotides), small molecules, antibodies, sugars, fatty acids, nucleotides and nucleotide analogs, analogs of naturally occurring structures (e.g., peptide mimetics, nucleic acid analogs, and the like), and numerous other compounds. Although, for convenience, the present discussion primarily refers antagonists of PDZ-PL interactions, it will be recognized that PDZ-PL interaction agonists can also be use in the methods disclosed herein.

In one aspect, the peptides and peptide mimetics or analogues of the invention contain an amino acid sequence that binds a PDZ domain in a cell of interest. In one embodiment, the antagonists comprise a peptide that has a sequence corresponding to the carboxy-terminal sequence of a PL protein listed in TABLE 3, e.g., a peptide listed TABLE 3. Typically, the peptide comprises at least the C-terminal two (3), three (3) or four (4) residues of the PL protein, and often the inhibitory peptide comprises more than four residues (e.g., at least five, six, seven, eight, nine, ten, twelve or fifteen residues) from the PL protein C-terminus.

In some embodiments, the inhibitor is a peptide, e.g., having a sequence of a PL C-terminal protein sequence.

In some embodiments, the antagonist is a fusion protein comprising such a sequence. Fusion proteins containing a transmembrane transporter amino acid sequence are particularly useful.

In some embodiments, the inhibitor is conserved variant of the PL C-terminal protein sequence having inhibitory activity.

In some embodiments, the antagonist is a peptide mimetic of a PL C-terminal sequence.

In some embodiments, the inhibitor is a small molecule (i.e., having a molecular weight less than 1 kD). See, e.g. Section 6.5.4, infra.

F. Peptide Antagonists

In one embodiment, the antagonists comprise a peptide that has a sequence of a PL protein carboxy-terminus listed in TABLE 3. The peptide comprises at least the C-terminal two (2) residues of the PL protein, and typically, the inhibitory peptide comprises more than two residues (e.g, at least three, four, five, six, seven, eight, nine, ten, twelve or fifteen residues) from the PL protein C-terminus. The peptide may be any of a variety of lengths (e.g., at least 2, at least 3, at least 4, at least 5, at least 6, at least 8, at least 10, or at least 20 residues) and may contain additional residues not from the PL protein. It will be recognized that short PL peptides are sometime used in the rational design of other small molecules with similar properties.

Although most often, the residues shared by the inhibitory peptide with the PL protein are found at the C-terminus of the peptide. However, in some embodiments, the sequence is internal. Similarly, in some cases, the inhibitory peptide comprises residues from a PL sequence that is near, but not at the c-terminus of a PL protein (see, Gee et al., 1998, J Biological Chem. 273:21980-87).

Sometime the PL protein carboxy-terminus sequence is referred to as the “core PDZ motif sequence” referring to the ability of the short sequence to interact with the PDZ domain. For example, in an embodiment, the “core PDZ motif sequence” contains the last four C-terminus amino acids. As described above, the four amino acid core of a PDZ motif sequence may contain additional amino acids at its amino terminus to further increase its binding affinity and/or stability. Thus, in one embodiment, the PDZ motif sequence peptide can be from four amino acids up to 15 amino acids. It is preferred that the length of the sequence to be 6-10 amino acids. More preferably, the PDZ motif sequence contains 8 amino acids. Additional amino acids at the amino terminal end of the core sequence may be derived from the natural sequence in each hematopoietic cell surface receptor or a synthetic linker. The additional amino acids may also be conservatively substituted. When the third residue from the C-terminus is S, T or Y, this residue may be phosphorylated prior to the use of the peptide.

In some embodiments, the peptide and nonpeptide inhibitors of the are small, e.g., fewer than ten amino acid residues in length if a peptide. Further, it is reported that a limited number of ligand amino acids directly contact the PDZ domain (generally less than eight) (Kozlov et al., 2000, Biochemistry 39, 2572;Doyle et al., 1996, Cell 85,1067) and that peptides as short as the C-terminal three amino acids often retain similar binding properties to longer (>15) amino acids peptides (Yanagisawa et al., 1997, J. Biol. Chem. 272, 8539).

G. Peptide Variants

Having identified PDZ binding peptides and PDZ-PL interaction inhibitory sequences, variations of these sequences can be made and the resulting peptide variants can be tested for PDZ domain binding or PDZ-PL inhibitory activity. In embodiments, the variants have the same or a different ability to bind a PDZ domain as the parent peptide. Typically, such amino acid substitutions are conservative, i.e., the amino acid residues are replaced with other amino acid residues having physical and/or chemical properties similar to the residues they are replacing. Preferably, conservative amino acid substitutions are those wherein an amino acid is replaced with another amino acid encompassed within the same designated class.

H. Peptide Mimetics

Having identified PDZ binding peptides and PDZ-PL interaction inhibitory sequences, peptide mimetics can be prepared using routine methods, and the inhibitory activity of the mimetics can be confirmed using the assays of the invention. Thus, in some embodiments, the antagonist is a peptide mimetic of a PL C-terminal sequence. The skilled artisan will recognize that individual synthetic residues and polypeptides incorporating mimetics can be synthesized using a variety of procedures and methodologies, which are well described in the scientific and patent literature, e.g., Organic Syntheses Collective Volumes, Gilman et al. (Eds) John Wiley & Sons, Inc., NY. Polypeptides incorporating mimetics can also be made using solid phase synthetic procedures, as described, e.g., by Di Marchi, et al., U.S. Pat. No. 5,422,426. Mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi (1998) Mol. Biotechnol. 9:205-223; Hruby (1997) Curr. Opin. Chem. Biol. 1:114-119; Ostergaard (1997) Mol. Divers. 3:17-27; Ostresh (1996) Methods Enzymol. 267:220-234.

I. Small Molecules

In some embodiments, the inhibitor is a small molecule (i.e., having a molecular weight less than 1 kD). Methods for screening small molecules are well known in the art and include those described supra.

XII. Preparation of Peptides

A. Chemical Synthesis

The peptides of the invention or analogues thereof, may be prepared using virtually any art-known technique for the preparation of peptides and peptide analogues. For example, the peptides may be prepared in linear form using conventional solution or solid phase peptide syntheses and cleaved from the resin followed by purification procedures (Creighton, 1983, Protein Structures And Molecular Principles, W. H. Freeman and Co., N.Y.). Suitable procedures for synthesizing the peptides described herein are well known in the art. The composition of the synthetic peptides may be confirmed by amino acid analysis or sequencing (e.g., the Edman degradation procedure and mass spectroscopy).

In addition, analogues and derivatives of the peptides can be chemically synthesized. The linkage between each amino acid of the peptides of the invention may be an amide, a substituted amide or an isostere of amide. Nonclassical amino acids or chemical amino acid analogues can be introduced as a substitution or addition into the sequence. Non-classical amino acids include, but are not limited to, the D-isomers of the common amino acids, α-aimino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogues in general. Furthermore, the amino acid can be D (dextrorotary) or L (levorotary).

B. Recombinant Synthesis

If the peptide is composed entirely of gene-encoded amino acids, or a portion of it is so composed, the peptide or the relevant portion may also be synthesized using conventional recombinant genetic engineering techniques. For recombinant production, a polynucleotide sequence encoding a linear form of the peptide is inserted into an appropriate expression vehicle, i. e., a vector which contains the necessary elements for the transcription and translation of the inserted coding sequence, or in the case of an RNA viral vector, the necessary elements for replication and translation. The expression vehicle is then transfected into a suitable target cell which will express the peptide. Depending on the expression system used, the expressed peptide is then isolated by procedures well-established in the art. Methods for recombinant protein and peptide production are well known in the art (see, e.g., Maniatis et al., 1989, Molecular Cloning A Laboratory Manual, Cold Spring Harbor Laboratory, N.Y.; and Ausubel et al., 1989, Current Protocols in Molecular Biology, Greene Publishing Associates and Wiley Interscience, N.Y.).

A variety of host-expression vector systems may be utilized to express the peptides described herein. These include, but are not limited to, microorganisms such as bacteria transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant virus expression vectors (e.g., baculovirus) containing an appropriate coding sequence; plant cell systems infected with recombinant virus expression vectors (e.g., cauliflower mosaic virus or tobacco mosaic virus) or transformed with recombinant plasmid expression vectors (e.g., Ti plasmid) containing an appropriate coding sequence; or animal cell systems.

The expression elements of the expression systems vary in their strength and specificities. Depending on the host/vector system utilized, any of a number of suitable transcription and translation elements, including constitutive and inducible promoters, may be used in the expression vector. For example, when cloning in bacterial systems, inducible promoters such as pL of bacteriophage λ, plac, ptrp, ptac (ptrp-lac hybrid promoter) and the like may be used; when cloning in insect cell systems, promoters such as the baculovirus polyhedron promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter) or from mammalian viruses(e.g., the adenoviruslate promoter; the vaccinia virus 7.5 K promoter) may be used; when generating cell lines that contain multiple copies of expression product, SV40-, BPV- and EBV-based vectors may be used with an appropriate selectable marker.

In cases where plant expression vectors are used, the expression of sequences encoding the peptides of the invention may be driven by any of a number of promoters. For example, viral promoters such as the 35S RNA and 19S RNA promoters of CaMV (Brisson et al., 1984, Nature 310:511-514), or the coat protein promoter of TMV (Takamatsu et al., 1987, EMBO J. 6:307-311) may be used; alternatively, plant promoters such as the small subunit of RUBISCO (Coruzzi et al., 1984, EMBO J. 3:1671-1680;Broglie et al., 1984, Science 224:838-843) or heat shock promoters, e.g., soybean hsp 17.5-E or hsp17.3-B (Gurley et al., 1986, Mol. Cell. Biol. 6:559-565) may be used. These constructs can be introduced into planleukocytes using Ti plasmids, Ri plasmids, plant virus vectors, direct DNA transformation, microinjection, electroporation, etc. For reviews of such techniques see, e.g., Weissbach & Weissbach, 1988, Methods for Plant Molecular Biology, Academic Press, NY, Section VIII, pp. 421-463; and Grierson & Corey, 1988, Plant Molecular Biology,2d Ed., Blackie, London, Ch. 7-9.

In one insect expression system that may be used to produce the peptides of the invention, Autographa californica nuclear polyhidrosis virus (AcNPV) is used as a vector to express the foreign genes. The virus grows in Spodopiera frugiperda cells. A coding sequence may be cloned into non-essential regions (for example the polyhedron gene) of the virus and placed under control of an AcNPV promoter (for example, the polyhedron promoter). Successful insertion of a coding sequence will result in inactivation of the polyhedron gene and production of non-occluded recombinant virus (i. e., virus lacking the proteinaceous coat coded for by the polyhedron gene). These recombinant viruses are then used to infect Spodoptera frugiperda cells in which the inserted gene is expressed. (e.g., see Smith et al., 1983, J. Virol. 46:584; Smith, U.S. Pat. No. 4,215,051). Further examples of this expression system may be found in Current Protocols in Molecular Biology, Vol. 2, Ausubel et al., eds., Greene Publish. Assoc. & Wiley Interscience.

In mammalian host cells, a number of viral based expression systems may be utilized. In cases where an adenovirus is used as an expression vector, a coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing peptide in infected hosts. (e.g., See Logan& Shenk,1984, Proc. Natl. Acad. Sci. USA 81:3655-3659). Alternatively,the vaccinia 7.5 K promoter may be used, (see, e.g., Mackettet al., 1982, Proc. Natl. Acad. Sci. USA 79:7415-7419; Mackett et al., 1984, J. Virol. 49:857-864; Panicali et al., 1982, Proc. Natl. Acad. Sci. USA 79:4927-4931).

Other expression systems for producing linear peptides of the invention will be apparent to those having skill in the art.

Purification of the Peptides and Peptide Analogues

The peptides and peptide analogues of the invention can be purified by art-known techniques such as high performance liquid chromatography, ion exchange chromatography, gel electrophoresis, affinity chromatography and the like. The actual conditions used to purify a particular peptide or analogue will depend, in part, on factors such as net charge, hydrophobicity, hydrophilicity, etc., and will be apparent to those having skill in the art. The purified peptides can be identified by assays based on their physical or functional properties, including radioactive labeling followed by gel electrophoresis, radioimmuno-assays, ELISA, bioassays, and the like.

For affinity chromatography purification, any antibody which specifically binds the peptides or peptide analogues may be used. For the production of antibodies, various host animals, including but not limited to rabbits, mice, rats, etc., may be immunized by injection with a peptide. The peptide may be attached to a suitable carrier, such as BSA or KLH, by means of a side chain functional group or linkers attached to a side chain functional group. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol,and potentially useful human adjuvants such as BCG (bacilli Calmette-Guerin) and Corynebacterium parvum.

Monoclonal antibodies to a peptide may be prepared using any technique which provides for the production of antibody molecules by continuous cell lines in culture. These include but are not limited to the hybridoma technique originally described by Koehler and Milstein, 1975, Nature 256:495-497, the human B-cell hybridoma technique, Kosbor et al., 1983, Immunology Today 4:72; Cote et al., 1983, Proc. Natl. Acad. Sci. U.S.A. 80:2026-2030 and the EBV-hybridoma technique (Cole et al., 1985, Monoclonal Antibodies and Cancer Therapy, Alan R. Liss, Inc., pp. 77-96 (1985)). In addition, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule of appropriate antigen specificity together with genes from a human antibody molecule of appropriate biological activity can be used. Alternatively, techniques described for the production of single chain antibodies (U.S. Pat. No.4,946,778) can be adapted to produce peptide-specific single chain antibodies.

Antibody fragments which contain deletions of specific binding sites may be generated by known techniques. For example, such fragments include but are not limited to F(ab′)₂ fragments, which can be produced by pepsin digestion of the antibody molecule and Fab fragments, which can be generated by reducing the disulfide bridges of the F(ab′)₂ fragments. Alternatively, Fab expression libraries may be constructed (Huse et al., 1989, Science 246:1275-1281) to allow rapid and easy identification of monoclonal Fab fragments with the desired specificity for the peptide of interest.

The antibody or antibody fragment specific for the desired peptide can be attached, for example, to agarose, and the antibody-agarose complex is used in immunochromatography to purify peptides of the invention. See, Scopes, 1984, Protein Purification: Principles and Practice, Springer-Verlag New York, Inc., NY, Livingstone, 1974, Methods Enzymology: Immunoaffinity Chromatography of Proteins 34:723-731.

XIII. Uses of PDZ Domain Binding and Antagonist Compounds

As indicated in the Background section, PDZ domain-containing proteins are involved in a number of biological functions, including, but not limited to, vesicular trafficking, tumor suppression, protein sorting, establishment of membrane polarity, apoptosis, regulation of immune response and organization of synapse formation. In general, this family of proteins has a common function of facilitating the assembly of multi-protein complexes, often serving as a bridge between several proteins, or regulating the function of other proteins. Additionally, as also noted supra, these proteins are found in essentially all cell types.

Consequently, modulation of these interactions can be utilized to control a wide variety of biological conditions and physiological conditions. In particular, modulation of interactions such as those disclosed herein can be utilized to control movement of vesicles within a cell, inhibition of tumor formation, as well as in the treatment of immune disorders, neurological disorders, muscular disorders, and intestinal disorders.

Certain compounds which modulate binding of the PDZ proteins and PL proteins can be used to inhibit leukocyte activation, which is manifested in measurable events including but not limited to, cytokine production, cell adhesion, expansion of cell numbers, apoptosis and cytotoxicity. Thus, some compounds of the invention can be used to treat diverse conditions associated with undesirable leukocyte activation, including but not limited to, acute and chronic inflammation, graft-versus-host disease, transplantation rejection, hypersensitivities and autoimmunity such as multiple sclerosis, rheumatoid arthritis, peridontal disease, systemic lupus erythematosis, juvenile diabetes mellitis, non-insulin-dependent diabetes, and allergies, and other conditions listed herein (see, e.g., Section 6.4, supra).

Thus, the invention also relates to methods of using such compositions in modulating leukocyte activation as measured by, for example, cytotoxicity, cytokine production, cell proliferation, and apoptosis.

XIV. Formulation and Route of Administration

A. Introduction of Agonists or Antagonists (e.g.* Peptides and Fusion Proteins) into Cells

In one aspect, the PDZ-PL antagonists of the invention are introduced into a cell to modulate (i.e., increase or decrease) a biological function or activity of the cell. Many small organic molecules readily cross the cell membranes (or can be modified by one of skill using routine methods to increase the ability of compounds to enter cells, e.g., by reducing or eliminating charge, increasing lipophilicity, conjugating the molecule to a moiety targeting a cell surface receptor such that after interacting with the receptor). Methods for introducing larger molecules, e.g., peptides and fusion proteins are also well known, including, e.g., injection, liposome-mediated fusion, application of a hydrogel, conjugation to a targeting moiety conjugate endocytozed by the cell, electroporation, and the like).

In one embodiment, the antagonist or agent is a fusion polypeptide or derivatized polypeptide. A fusion or derivatized protein may include a targeting moiety that increases the ability of the polypeptide to traverse a cell membrane or causes the polypeptide to be delivered to a specified cell type (e.g., liver cells or tumor cells) preferentially or cell compartment (e.g., nuclear compartment) preferentially. Examples of targeting moieties include lipid tails, amino acid sequences such as antennapoedia peptide or a nuclear localization signal (NLS; e.g., Xenopus nucleoplasmin Robbins et al., 1991, Cell 64:615).

In one embodiment of the invention, a peptide sequence or peptide analog determined to inhibit a PDZ domain-PL protein binding, in an assay of the invention is introduced into a cell by linking the sequence to an amino acid sequence that facilitates its transport through the plasma membrane (a “transmembrane transporter sequence”). The peptides of the invention may be used directly or fused to a transmembrane transporter sequence to facilitate their entry into cells. In the case of such a fusion peptide, each peptide may be fused with a heterologous peptide at its amino terminus directly or by using a flexible polylinker such as the pentamer G-G-G-G-S repeated 1 to 3 times. Such linker has been used in constructing single chain antibodies (scFv) by being inserted between V_{H} and V_{L} (Bird et al., 1988, Science 242:423-426; Huston et al., 1988, Proc. Natl. Acad. Sci. USA. 85:5979-5883). The linker is designed to enable the correct interaction between two beta-sheets forming the variable region of the single chain antibody. Other linkers which may be used include Glu-Gly-Lys-Ser-Ser-Gly-Ser-Gly-Ser-Glu-Ser-Lys-Val-Asp (Chaudhary et al, 1990, Proc. Natl. Acad. Sci. U.S.A. 87:1066-1070) and Lys-Glu-Ser-Gly-Ser-Val-Ser-Ser-Glu-Gln-Leu-Ala-Gln-Phe-Arg-Ser-Leu-Asp (Bird et al., 1988, Science 242:423-426).

A number of peptide sequences have been described in the art as capable of facilitating the entry of a peptide linked to these sequences into a cell through the plasma membrane (Derossi et al., 1998, Trends in Cell Biol. 8:84). For the purpose of this invention, such peptides are collectively referred to as transmembrane transporter peptides. Examples of these peptide include, but are not limited to, tat derived from HIV (Vives et al., 1997, J. Biol. Chem. 272:16010; Nagahara et al., 1998, Nat. Med. 4:1449), antennapedia from Drosophila (Derossi et al., 1994, J. Biol. Chem. 261:10444), VP22 from herpes simplex virus (Elliot and D'Hare, 1997, Cell 88:223-233), complementarity-determining regions (CDR) 2 and 3 of anti-DNA antibodies (Avrameas et al., 1998, Proc. Natl Acad. Sci. U.S.A., 95:5601-5606), 70 KDa heat shock protein (Fujihara, 1999, EMBO J. 18:411-419) and transportan(Poogaet al., 1998,

FASEB J. 12:67-77). In a preferred embodiment of the invention, a truncated HIV tat peptide having the sequence of GYGRKKRRQRRRG is used.

It is preferred that a transmembrane transporter sequence is fused to a hematopoietic cell, surface receptor carboxyl terminal sequence at its amino-terminus with or without a linker. Generally, the C-terminus of a PDZ motif sequence (PL sequence) must be free in order to interact with a PDZ domain. The Arg-Ser-Leu-Asp transmembrane transporter sequence may be used in whole or in part as long as it is capable of facilitating entry of the peptide into a cell.

In an alternate embodiment of the invention, a hematopoietic cell surface receptor C-terminal sequence may be used alone when it is delivered in a manner that allows its entry into cells in the absence of a transmembrane transporter sequence. For example, the peptide may be delivered in a liposome formulation or using a gene therapy approach by delivering a coding sequence for the PDZ motif alone or as a fusion molecule into a target cell.

The compounds of the of the invention may also be administered via liposomes, which serve to target the conjugates to a particular tissue, such as lymphoid tissue, or targeted selectively to infected cells, as well as increase the half-life of the peptide composition. Liposomes include emulsions, foams, micelles, insoluble monolayers, liquid crystals, phospholipid dispersions, lamellar layers and the like. In these preparations the peptide to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired peptide or conjugate of the invention can be directed to the site of lymphoid cells, where the liposomes then deliver the selected inhibitor compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028.

The targeting of liposomes using a variety of targeting agents is well known in the art (see, e.g., U.S. Pat. Nos. 4,957,773 and 4,603,044). For targeting to the immune cells, a ligand to be incorporated into the liposome can include, e.g., antibodies or fragments thereof specific for cell surface determinants of the desired immune system cells. A liposome suspension containing a peptide or conjugate may be administered intravenously, locally, topically, etc. in a dose which varies according to, inter alia, the manner of administration, the conjugate being delivered, and the stage of the disease being treated.

In order to specifically deliver a PDZ motif sequence (PL sequence) peptide into a specific cell type, the peptide may be linked to a cell-specific targeting moiety, which include but are not limited to, ligands for diverse leukocyte surface molecules such as growth factors, hormones and cytokines, as well as antibodies or antigen-binding fragments thereof. Since a large number of cell surface receptors have been identified in leukocytes, ligands or antibodies specific for these receptors may be used as cell-specific targeting moieties. For example, interleukin-2, B7-1 (CD80), B7-2 (CD86) and CD40 or peptide fragments thereof may be used to specifically target activated T cells (The Leucocyte Antigen Facts Book, 1997, Barclay et al. (eds.), Academic Press). CD28, CTLA-4 and CD40L or peptide fragments thereof may be used to specifically target B cells. Furthermore, Fc domains may be used to target certain Fc receptor-expressing cells such as monocytes.

Antibodies are the most versatile cell-specific targeting moieties because they can be generated against any cell surface antigen. Monoclonal antibodies have been generated against leukocyte lineage-specific markers such as certain CD antigens. Antibody variable region genes can be readily isolated from hybridoma cells by methods well known in the art. However, since antibodies are assembled between two heavy chains and two light chains, it is preferred that a scFv be used as a cell-specific targeting moiety in the present invention. Such scFv are comprised of V_{H} and V_{L} domains linked into a single polypeptide chain by a flexible linker peptide.

The PDZ motif sequence (PL sequence) may be linked to a transmembrane transporter sequence and a cell-specific targeting moiety to produce a tri-fusion molecule. This molecule can bind to a leukocyte surface molecule, passes through the membrane and targets PDZ domains. Alternatively, a PDZ motif sequence (PL sequence) may be linked to a cell-specific targeting moiety that binds to a surface molecule that internalizes the fusion peptide.

In an other approach, microspheres of artificial polymers of mixed amino acids (proteinoids) have been used to deliver pharmaceuticals. For example, U.S. Pat. No.4,925,673 describes drug-containing proteinoid microsphere carriers as well as methods for their preparation and use. These proteinoid microspheres are useful for the delivery of a number of active agents. Also see, U.S. Pat. Nos. 5,907,030 and 6,033,884, which are incorporated herein by reference.

B. Introduction of Polynucleotides into Cells

By introducing gene sequences into cells, gene therapy can be used to treat conditions in which leukocytes are activated to result in deleterious consequences. In one embodiment, a polynucleotide that encodes a PL sequence peptide of the invention is introduced into a cell where it is expressed. The expressed peptide then inhibits the interaction of PDZ proteins and PL proteins in the cell.

Thus, in one embodiment, the polypeptides of the invention are expressed in a cell by introducing a nucleic acid (e.g., a DNA expression vector or mRNA) encoding the desired protein or peptide into the cell. Expression may be either constitutive or inducible depending on the vector and choice of promoter. Methods for introduction and expression of nucleic acids into a cell are well known in the art and described herein.

In a specific embodiment, nucleic acids comprising a sequence encoding a peptide disclosed herein, are administered to a human subject. In this embodiment of the invention, the nucleic acid produces its encoded product that mediates a therapeutic effect. Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 1991, Biotherapy 3:87-95; Tolstoshev,1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In a preferred embodiment of the invention, the therapeutic composition comprises a coding sequence that is part of an expression vector. In particular, such a nucleic acid has a promoter operably linked to the coding sequence, said promoter being inducible or constitutive, and, optionally, tissue-specific. In another specific embodiment, a nucleic acid molecule is used in which the coding sequence and any other desired sequences are flanked by regions that promote homologous recombination at a desired site in the genome, thus providing for intrachromosomal expression of the nucleic acid (Koller and Smithies, 1989, Proc. Natl. Acad. Sci. USA 86:8932-8935; Zijlstra et al., 1989, Nature 342:435-438).

Delivery of the nucleic acid into a patient may be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy.

In a specific embodiment,the nucleic acid is directly administered in vivo, where it is expressed to produce the encoded product. This can be accomplished by any methods known in the art, e.g., by constructing it as part of an appropriate nucleic acid expression vector and administering it so that it becomes intracellular, e.g., by infection using a defective or attenuated retroviralor other viral vector (see U.S. Pat. No. 4,980,286), by direct injection of naked DNA, by use of microparticle bombardment (e.g., a gene gun; Biolistic, Dupont), by coating with lipids or cell-surface receptors or transfecting agents, by encapsulation in liposomes, microparticles, or microcapsules, by administering it in linkage to a peptide which is known to enter the nucleus, or by administering it in linkage to a ligand subject to receptor-mediated endocytosis (see e.g., Wu and Wu, 1987, J. Biol. Chem. 262:4429-4432) which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In. yet another embodiment, the nucleic acid can be targeted in vivo for cell specific uptake and expression, by targeting a specific receptor (see, e.g., PCT Publications WO 92/06180 dated Apr. 16, 1992; WO 92/22635 dated Dec. 23, 1992; WO92/20316 dated Nov. 26, 1992; WO93/14188 dated Jul. 22, 1993; WO 93/20221 dated Oct. 14, 1993). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a preferred embodiment of the invention, adenoviruses as viral vectors can be used in gene therapy. Adenoviruses have the advantage of being capable of infecting non-dividing cells (Kozarsky and Wilson, 1993, Current Opinion in Genetics and Development 3:499-503). Other instances of the use of adenoviruses in gene therapy can be found in Rosenfeld et al., 1991, Science 252:431-434; Rosenfeld et al., 1992, Cell 68:143-155; and Mastrangeli et al., 1993, J. Clin. Invest. 91:225-234. Furthermore, adenoviral vectors with modified tropism may be used for cell specific targeting (WO98/40508). Adeno-associated virus (AAV) has also been proposed for use in gene therapy (Walsh et al., 1993, Proc. Soc. Exp. Biol. Med. 204:289-300).

In addition, retroviral vectors (see Miller et al., 1993, Meth. Enzymol. 217:581-599) have been modified to delete retroviral sequences that are not necessary for packaging of the viral genome and integration into host cell DNA. The coding sequence to be used in gene therapy is cloned into the vector, which facilitates delivery of the gene into a patient. More detail about retroviral vectors can be found in Boesen et al., 1994, Biotherapy 6:291-302,which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Another approach to gene therapy involves transferring a gene to cells in tissue culture. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, lipofection, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny. In a preferred embodiment, the cell used for gene therapy is autologous to the patient.

In a specific embodiment, the nucleic acid to be introduced for purposes of gene therapy comprises an inducible promoter operably linked to the coding sequence, such that expression of the nucleic acid is controllable by controlling the presence or absence of the appropriate inducer of transcription.

Oligonucleotides such as anti-sense RNA and DNA molecules, and ribozymes that function to inhibit the translation of a targeted mRNA, especially its C-terminus are also within the scope of the invention. Anti-sense RNA and DNA molecules act to directly block the translation of mRNA by binding to targeted mRNA and preventing protein translation. In regard to antisense DNA, oligodeoxyribonucleotides derived from the translation initiation site, e.g., between −10 and +10 regions of a nucleotide sequence, are preferred.

The antisense oligonucleotide may comprise at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Within the scope of the invention are engineered hammerhead motif ribozyme molecules that specifically and efficiently catalyze endonucleolytic cleavage of target RNA sequences.

Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complementary oligonucleotides, using ribonuclease protection assays.

The anti-sense RNA and DNA molecules and ribozymes of the invention may be prepared by any method known in the art for the synthesis of nucleic acid molecules. These include techniques for chemically synthesizing oligodeoxyrbonucleotides well known in the art such as for example solid phase phosphoramidite chemical synthesis. Alternatively, RNA molecules may be generated by in vitro and in vivo transcription of DNA sequences encoding the RNA molecule. Such DNA sequences may be incorporated into a wide variety of vectors which contain suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various modifications to the DNA molecules may be introduced as a means of increasing intracellular stability and half-life. Possible modifications include, but are not limited to, the addition of flanking sequences of ribo- or deoxy-nucleotides to the 5′ and/or 3′ ends of the molecule or the use of phosphorothioate or 2′ O-methyl rather than phosphodiesterase linkages within the oligodeoxyribonucleotide backbone.

C. Other Pharmaceutical Compositions

The compounds of the invention, may be administered to a subject per se or in the form of a sterile composition or a pharmaceutical composition. Pharmaceutical compositions comprising the compounds of the invention may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries that facilitate processing of the active peptides or peptide analogues into preparations which can be used pharmaceutically. Proper formulation is dependent upon the route of administration chosen.

For topical administration the compounds of the invention may be formulated as solutions, gels, ointments, creams, suspensions, etc. as are well-known in the art.

Systemic formulations include those designed for administration by injection, e.g. subcutaneous, intravenous, intramuscular, intrathecal or intraperitoneal injection, as well as those designed for transdermal, transmucosal, oral or pulmonary administration.

For injection, the compounds of the invention may be formulated in aqueous solutions, preferably in physiologically compatible buffers such as Hanks's solution, Ringer's solution, or physiological saline buffer. The solution may contain formulatory agents such as suspending, stabilizing and/or dispersing agents.

Alternatively, the compounds may be in powder form for constitution with a suitable vehicle, e.g., sterile pyrogen-free water, before use.

For transmucosal administration, penetrants appropriate to the barrier to be permeated are used in the formulation. Such penetrants are generally known in the art. This route of administration may be used to deliver the compounds to the nasal cavity.

For oral administration, the compounds can be readily formulated by combining the active peptides or peptide analogues with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate.

If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like may be added.

For buccal administration, the compounds may take the form of tablets, lozenges, etc. formulated in conventional manner.

For administration by inhalation, the compounds for use according to the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of apressurized aerosol the dosage unit may be determined by providing a valve to deliver a metered amount. Capsules and cartridges of e.g. gelatin for use in an inhaleror insufflator may be formulated containing a powder mix of the compound and a suitable powder base such as lactose or starch.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well known examples of delivery vehicles that may be used to deliver peptides and peptide analogues of the invention. Certain organic solvents such as dimethylsulfoxide also may be employed, although usually at the cost of greater toxicity. Additionally, the compounds may be delivered using a sustained-release system, such as semipermeable matrices of solid polymers containing the therapeutic agent. Various of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

As the compounds of the invention may contain charged side chains or termini, they may be included in any of the above-described formulations as the free acids or bases or as pharmaceutically acceptable salts. Pharmaceutically acceptable salts are those salts which substantially retain the biologic activity of the free bases and which are prepared by reaction with inorganic acids. Pharmaceutical salts tend to be more soluble in aqueous and other protic solvents than are the corresponding free base forms.

D. Effective Dosages

The compounds of the invention will generally be used in an amount effective to achieve the intended purpose. The compounds of the invention or pharmaceutical compositions thereof, are administered or applied in a therapeutically effective amount. By therapeutically effective amount is meant an amount effective ameliorate or prevent the symptoms, or prolong the survival of, the patient being treated. Determination of a therapeutically effective amount is well within the capabilities of those skilled in the art, especially in light of the detailed disclosure provided herein. An “inhibitory amount” or “inhibitory concentration” of a PL-PDZ binding inhibitor is an amount that reduces binding by at least about 40%, preferably at least about 50%, often at least about 70%, and even as much as at least about 90%. Binding can as measured in vitro (e.g., in an A assay or G assay) or in situ.

For systemic administration, a therapeutically effective dose can be estimated initially from in vitro assays. For example, a dose can be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the compounds that are sufficient to maintain therapeutic effect. Usual patient dosages for administration by injection range from about 0.1 to 5 mg/kg/day, preferably from about 0.5 to 1 mg/kg/day. Therapeutically effective serum levels may be achieved by administering multiple doses each day.

In cases of local administration or selective uptake, the effective local concentration of the compounds may not be related to plasma concentration. One having skill in the art will be able to optimize therapeutically effective local dosages without undue experimentation.

The amount of compound administered will, of course, be dependent on the subject being treated, on the subject's weight, the severity of the affliction, the manner of administration and the judgment of the prescribing physician.

The therapy may be repeated intermittently while symptoms detectable or even when they are not detectable. The therapy may be provided alone or in combination with other drugs. In the case of conditions associated with leukocyte activation such as transplantation rejection and autoimmunity, the drugs that may be used in combination with the compounds of the invention include, but are not limited to, steroid and non-steroid anti-inflammatory agents.

E. Toxicity

Preferably, a therapeutically effective dose of the compounds described herein will provide therapeutic benefit without causing substantial toxicity.

Toxicity of the compounds described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. Compounds which exhibit high therapeutic indices are preferred. The data obtained from these cell culture assays and animal studies can be used in formulating a dosage range that is not toxic for use in human. The dosage of the compounds described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1, p. 1).

### EXAMPLE 1

### Generation of Eukaryotic Expression Constructs Bearing DNA Fragments that Encode PDZ Domain Containing Genes or Portions of PDZ Domain Genes

This example describes the cloning of PDZ domain containing genes or portions of PDZ domain containing genes were into eukaryotic expression vectors in fusion with a number of protein tags, including but not limited to Glutathione S-Transferase (GST), Enhanced Green Fluorescent Protein (EGFP), or Hemagglutinin (HA).

A. Strategy

DNA fragments corresponding to PDZ domain containing genes were generated by RT-PCR from RNA from a library of individual cell lines (CLONTECH Cat# K4000-1) derived RNA, using random (oligo-nucleotide) primers (Invitrogen Cat.# 48190011). DNA fragments corresponding to PDZ domain containing genes or portions of PDZ domain containing genes were generated by standard PCR, using above purified cDNA fragments and specific primers (see Table 5). Primers used were designed to create restriction nuclease recognition sites at the PCR fragment's ends, to allow cloning of those fragments into appropriate expression vectors. Subsequent to PCR, DNA samples were submitted to agarose gel electrophoresis. Bands corresponding to the expected size were excised. DNA was extracted by Sephaglas Band Prep Kit (Amersham Pharmacia Cat# 27-9285-01) and digested with appropriate restriction endonuclease. Digested DNA samples were purified once more by gel electrophoresis, according to the same protocol used above. Purified DNA fragments were coprecipitated and ligated with the appropriate linearized vector. After transformation into E. coli, bacterial colonies were screened by colony PCR and restriction digest for the presence and correct orientation of insert. Positive clones were innoculated in liquid culture for large scale DNA purification. The insert and flanking vector sites from the purified plasmid DNA were sequenced to ensure correct sequence of fragments and junctions between the vectors and fusion proteins.

B. Vectors:

All PDZ domain-containing genes were cloned into the vector pGEX-3X (Amersham Pharmacia #27-4803-01, Genemed Acc#U13852, GI#595717), containing a tac promoter, GST, Factor Xa, β-lactamase, and lac repressor.

The amino acid sequence of the pGEX-3X coding region including GST, Factor Xa, and the multiple cloning site is listed below. Note that linker sequences between the cloned inserts and GST-Factor Xa vary depending on the restriction endonuclease used for cloning. Amino acids in the translated region below that may change depending on the insertion used are indicated in small caps, and are included as changed in the construct sequence listed in (C).

aa 1-aa232:

MSPILGYWKIKGLVQPTRLLLEYLEEKYEEHLYERDEGDKWRNKKFELGLEFP
NLPYYIDGDVKLTQSMAIIRYIADKHNMLGGCPKERAEISMLEGAVLDIRYGV
SRIAYSKDFETLKVDFLSKLPEMLKMFEDRLCHKTYLNGDHVTHPDFMLYDA
LDVVLYMDPMCLDAFPKLVCFKKRIEAIPQIDKYLKSSKYIAWPLQGWQATF
GGGDHPPKSDLIEGRgipgnss

In addition, TAX Interacting Protein 1 (TIP1), in whole or part, was cloned into many other expression vectors, including but not limited to CD5γ, PEAK10 (both provided by the laboratory of Dr. Brian Seed at Harvard University and generated by recombinant DNA technology, containing an IgG region), and MIN (a derivative of MSCV, containing IRES and 15 NGFR, generated by recombinant DNA technology).

C. Constructs:

Primers used to generate DNA fragments by PCR are listed in Table 5. PCR primer combinations and restriction sites for insert and vector are listed below, along with amino acid translation for insert and restriction sites. Non-native amino acid sequences are shown in lower case.

TABLE 5

Primers used in cloning of DLG 1 (domain 2 of 3), MAGI 1 (domain
2 of 6), and TIP1 into representative expression vectors.

| | | |
|---|---|---|
| ID # (Primer Name) | Primer Sequence | Description |

| | | |
|---|---|---|
| 1928 (654DL1 2F) | AATGGGGATCCAGCT | Forward (5′ to 3′) primer corresponding to |
| | CATTAAAGG | DLG 1, domain 2 of 3. Generates a Bam H1 |
| | | site upstream (5′) of the PDZ boundary. Used |
| | | for cloning into pGEX-3X. |
| 1929 (655DL1 2R) | ATACATACTTGTGGA | Reverse (3′ to 5′) primer corresponding to |
| | ATTCGCCAC | DLG 1, domain 2 of 3. Generates an EcoR1 |
| | | site downstream (3′) of the PDZ boundary. |
| | | Used for cloning into pGEX-3X. |
| 1453 (435BAF) | CACGGATCCCTTCTG | Forward (5′ to 3′) primer corresponding to |
| | AGTTGAAAGGC | MAGI 1, domain 2 of 6. Generates a BamH1 |
| | | site upstream (5′) of the PDZ boundary. Used |
| | | for cloning into pGEX-3X. |
| 1454 (436BAR) | TATGAATTCCATCTG | Reverse (3′ to 5′) primer corresponding to |
| | GATCAAAAGGCAATG | MAGI 1, domain 2 of 6. Generates an EcoR1 |
| | | site downstream (3′) of the PDZ boundary. |
| | | Used for cloning into pGEX-3X. |
| 399 (86TAF) | CAGGGATCCAAAGAG | Forward (5′ to 3′) primer corresponding to |
| | TTGAAATTCACAAGC | TIP1. Generates a Bam H1 site upstream (5′) |
| | | of the PDZ boundary. Used for cloning into |
| | | pGEX-3X. |
| 400 (87TAR) | ACGGAATTCTGCAGC | Reverse (3′ to 5′) primer corresponding to |
| | GACTGCCGCGTC | TIP1. Generates an EcoR1 site downstream |
| | | (3′) of the PDZ boundary. Used for cloning |
| | | into pGEX-3X. |
| 1319 (TIP G5-1) | AGGATCCAGATGTCC | Forward (5′ to 3′) primer corresponding to |
| | TACATCCC | TIP1. Generates a Bam H1 site upstream (5′) |
| | | of the start codon. Used for cloning into |
| | | pGEX-3X. |
| 1320 (TIP G3-1) | GGAATTCATGGACTG | Reverse (3′ to 5′) primer corresponding to |
| | CTGCACGG | TIP1. Generates an EcoR1 site downstream |
| | | (3′) of the stop codon. Used for cloning into |
| | | pGEX-3X. |
| 2753 (1109T1F) | AGAGAATTCTCGAGA | Forward (5′ to 3′) primer corresponding to |
| | TGTCCTACATCCC | TIP1. Generates an EcoR1 site upstream (5′) |
| | | of the start codon. Used for cloning into MIN. |
| 2762 (1117TIR) | TGGGAATTCCTAGGA | Reverse (3′ to 5′) primer corresponding to |
| | CAGCATGGACTG | TIP1. Generates an EcoR1 site downstream |
| | | (3′) of the stop codon. Used for cloning into |
| | | MIN. |
| 2584 (1080TIF) | CTAGGATCCGGGCCA | Forward (5′ to 3′), primer corresponding to |
| | GCCGGTCACC | TIP1. Generates a Bam H1 site upstream (5′) |
| | | of the PDZ boundary. Used for cloning into |
| | | PEAK10 or CD5γ. |
| 2585 (1082TIR) | GACGGATCCCCCTGC | Reverse (3′ to 5′) primer corresponding to |
| | TGCACGGCCTTCTG | TIP1. Generates a Bam H1 site downstream |
| | | (3′) of the PDZ boundary. Used for cloning |
| | | into PEAK10 or CD5γ. |
| 2586 (1082TIR) | GACGAATTCCCCTGC | Reverse (3′ to 5′) primer corresponding to |
| | TGCACGGCCTTCTG | TIP1. Generates an EcoR1 site downstream |
| | | (3′) of the PDZ boundary. Used for cloning |
| | | into PEAK 10 or CD5γ. |
| 2587 (1083TIF) | CTAGAATTCGGGCCA | Forward (5′ to 3′) primer corresponding to |
| | GCCGGTCACC | TIP1. Generates an Eco R1 site upstream (5′) |
| | | of the PDZ boundary. Used for cloning into |
| | | PEAK10 or CD5γ. |

1. DLG 1, PDZ domain 2 of 3:
Acc#: U13897
GI#: 558437
Construct: DLG 1, PDZ domain 2 of 3-pGEX-3X
   Primers: 1928 & 1929
   Vector Cloning Sites(5′/3′): Bam H1/EcoR1

Insert Cloning Sites(5′/3′): Bam1 /EcoR1

| | |
|---|---|
| aa 1- aa 88 | giqLIKGPKGLGFSIAGGVGNQHIPGDNSIYVTKIIEGGAAHKDGKLQIG |
| | DKLLAVNNVCLEEVTHEEAVTALKNTSDFVYLKVAnss |

2. MAGI 1. PDZ domain 2 of 6:
Acc#: AB010894
GI#: 3370997
Construct: MAGI 1, PDZ domain 2 of 6-pGEX-3X
   Primers: 1453 & 1454
   Vector Cloning Sites(5′/3′): Bam H1/EcoR1

Insert Cloning Sites(5′/3′): BamH1/EcoR1

| | |
|---|---|
| aa 1- aa 108 | giPSELKGKFIHTKLRKSSRGFGFTVVGGDEPDEFLQIKSLVLD |
| | GPAALDGKMETGDVIVSVNDTCVLGHTHAQVVKIFQSIPIG |
| | ASVDLELCRGYPLPFDPDgihrd |

3. TAX Interacting Protein 1 (TIP1):
Acc#: AF028823.2
GI#: 11908159
Construct: TIP1, PDZ domain 1 of 1-pGEX-3X
   Primers: 399& 400
   Vector Cloning Sites(5′/3′): Bam H1/EcoR1

Insert Cloning Sites(5′/3′): BamH1/EcoR1

| | |
|---|---|
| aa 1- aa 107 | giQRVEIHKLRQGENLILGFSIGGGIDQDPSQNPFSEDKTDKGI |
| | YVTRVSEGGPAEIAGLQIGDKIMQVNGWDMTMVTHDQAR |
| | KRLTKRSEEVVRLLVTRQSLQnss |

Construct: TIP1-pGEX-3X
   Primers: 1319& 1320
   Vector Cloning Sites(5′/3′): Bam H1/EcoR1

Insert Cloning Sites(5′/3′): BamH1/EcoR1

| | |
|---|---|
| aa 1- aa 128 | giqMSYIPGQPVTAVVQRVEIHKLRQGENLILGFSIGGGIDQDPSQNPF |
| | SEDKTDKGIYVTRVSEGGPAEIAGLQIGDKIMQVNGWDMTMVTHD |
| | QARKRLTKRSEEVVRLLVTRQSLQKAVQQSMnss |

Construct: TIP1-MIN
   Primers: 2753& 2762
   Vector Cloning Sites(5′/3′): EcoR1/EcoR1

Insert Cloning Sites(5′/3′): EcoR1/EcoR1

| | |
|---|---|
| aa 1-aa 129 | agilEMSYIPGQPVTAVVQRVEIHKLRQGENLILGFSIGGGIDQ |
| | DPSQNPFSEDKTDKGIYVTRVSEGGPAEIAGLQIGDKIMQVN |
| | GWDMTMVTHDQARKRLTKRSEEVVRLLVTRQSLQKAVQQ |
| | SMLS |

Construct: TIP1-CD5γ
   Primers: 2584& 2585
   Vector Cloning Sites(5′/3′): Bam H1/Ban H1

Insert Cloning Sites(5′/3′): BamH1/Bam H1

| | |
|---|---|
| aa 1- aa 122 | adPGQPVTAVVQRVEIHKLRQGENLILGFSIGGGIDQDPSQNP |
| | FSEDKTDKGIYVTRVSEGGPAEIAGLQIGDKIMQVNGWDM |
| | TMVTHDQARKRLTKRSEEVVRLLVTRQSLQKAVQQSdpe |

D. GST Fusion Protein Production and Purification

The constructs using pGEX-3X expression vector were used to make fusion proteins according to the protocol outlined in the GST Fusion System, Second Edition, Revision 2, Pharmacia Biotech. Method II and was optimized for a 1 L LgPP.

Purified DNA was transformed into E. coli and allowed to grow to an OD of 0.4-0.8 (600λ). Protein expression was induced for 1-2 hours by addition of IPTG to cell culture. Cells were harvested and lysed. Lysate was collected and GS4B beads (Pharmacia Cat #17-0756-01) were added to bind GST fusion proteins. Beads were isolated and GST fusion proteins were eluted with GEB II. Purified proteins were stored in GEB II at −80° C.

Purified proteins were used for ELISA-based assays and antibody production.

E. IgG Fusion Protein Production and Purification

The constructs using the CD5gamma or Peak I OIgG expression vectors were used to make fusion protein. Purified DNA vectors were transfected into 293 EBNA T cells under standard growth conditions (DMEM+10% FCS) using standard calcium phosphate precipitation methods (Sambrook, Fritsch and Maniatis, Cold Spring Harbor Press) at a ratio of ˜1 ug vector DNA for 1 million cells. This vector results in a fusion protein that is secreted into the growth medium. Transiently transfected cells are tested for peak expression, and growth media containing fusion protein is collected at that maxima (usually 1-2 days). Fusion proteins are either purified using Protein A chromatography or frozen directly in the growth media without addition.

### EXAMPLE 2

### Identification of Interleukin 8 Receptor A (IL8RA) Interactions with MAGI1 (domain 2), TIP1 (domain 1) and MINT2 (domains 1 & 2) in vitro

This example describes the binding of IL8RA to MAGI1 (domain 2 of 6), TIP1, and Mint2 (domains 1 & 2), assessed using a modified ELISA. Briefly, a GST-PDZ fusion was produced that contained the entire PDZ domain of human MAGI1 or TIP1 (see Example 2). In the case of Mint2, domains 1 and 2, the GST-PDZ fusion contained the entire PDZ domain for both domains 1 and 2. In addition, biotinylated peptide corresponding to the C-terminal 20 amino acids of IL8RA was synthesized and purified by HPLC. Binding between these entities was detected through the “G” Assay, a calorimetric assay using avidin-HRP to bind the biotin and a peroxidase substrate.

A. Peptide Purification

Peptide representing the C-terminal 20 amino acids of IL8RA was synthesized by standard FMOC chemistry and biotinylated if not used as an unlabeled competitor. Peptide was purified by reverse phase high performance liquid chromatography (HPLC) using a Vydac 218TP C18 Reversed Phase column having the dimensions of 10*25 mm, 5 um. Approximately 40 mg of peptide was dissolved in 2.0 ml of aqueous solution of 49.9% acetonitrile and 0.1% Tri-Fluoro acetic acid (TFA). This solution was then injected into the HPLC machine through a 25 micron syringe filter (Millipore). Buffers used to get a good separation are (A) distilled water with 0.1% TFA and (B) 0.1% TFA with Acetonitrile. Gradient Segment setup is listed in Table 7.

TABLE 7

| | | | | Flow rate |
|---|---|---|---|---|
| Time | A | B | C | (ml/min) |
| 0 | 96% | 4% | 0 | 5.00 |
| 30 | 100% | 100% | 0 | 5.00 |
| 35 | 100% | 100% | 0 | 5.00 |
| 40 | 96% | 4% | 0 | 5.00 |

The separation occurs based on the nature of the peptides. A peptide of overall hydrophobic nature will elute off later than a peptide of a hydrophilic nature. Fractions containing the “pure” peptide were collected and checked by Mass Spectrometer (MS). Purified peptides are lyophilized for stability and later use.
B. “G” Assay for Identification of Interactions Between Peptides and Fusion Proteins
Reagents and Materials
Nunc Polysorp 96 well Immuno-plate (Nunc cat #62409-005) (Maxisorp plates have been shown to have higher background signal)
PBS pH 7.4 (Gibco BRL cat #16777-148) or AVC phosphate buffered saline, 8 gm NaCl, 0.29 gm KCl, 1.44 gm Na₂HPO4, 0.24 gm KH₂PO4, add H2O to 1 L and pH 7.4; 0.2_filter
2% BSA/PBS (10 gm of bovine serum albumin, fraction V (ICN Biomedicals cat #IC15142983) into 500 ml PBS
Goat anti-GST mAb stock @ 5 mg/ml, store at 4° C., (Amersham Pharmacia cat #27-4577-01), dilute 1:1000 in PBS, final concentration 5_g/ml
HRP-Streptavidin, 2.5 mg/2 ml stock stored at 4° C. (Zymed cat #43-4323), dilute 1:2000 into 2% BSA, final concentration at 0.5_g/ml
Wash Buffer, 0.2% Tween 20 in 50 mM Tris pH 8.0
TMB ready to use (Dako cat #S1600)
1M H2SO4
12 w multichannel pipettor,
50 ml reagent reservoirs,
15 ml polypropylene conical tubes
   Protocol
1) Coat plate with 100 ul of 5 ug/ml goat anti GST, O/N @ 4° C.
2) Dump coating antibodies out and tap dry
3) Blocking—Add 200 ul per well 2% BSA, 2 hrs at 4° C.
4) Prepare proteins in 2% BSA (2 ml per row or per two columns)
5) 3 washes with cold PBS (must be cold through entire experiment) (at last wash leave PBS in wells until immediately adding next step)
6) Add proteins at 50 ul per well on ice (1 to 2 hrs at 4° C.)
7) Prepare Peptides in 2% BSA (2 ml/row or /columns)
8) 3× wash with cold PBS
9) Add peptides at 50 ul per well on ice (time on/time off) keep on ice after last peptide has been added for 10 minutes exactly place at room temp for 20 minutes exactly
10) Prepare 12 ml/plate of HRP-Streptavidin (1:2000 dilution in 2% BSA)
11) 3× wash with cold PBS
12) Add HRP-Streptavidin at 100 ul per well on ice, 20 minutes at 4° C.
13) Turn on plate reader and prepare files
14) 5× washes, avoid bubbles
15) Using gloves, add TMB substrate at 100 ul per well

incubate in dark at room temp

check plate periodically (5, 10, & 20 minutes)

take early readings, if necessary, at 650 nm (blue)

at 20 minutes, stop reaction with 100 ul of 1M H2SO4

take last reading at 450 nm (yellow)

C. Results of Binding Experiments

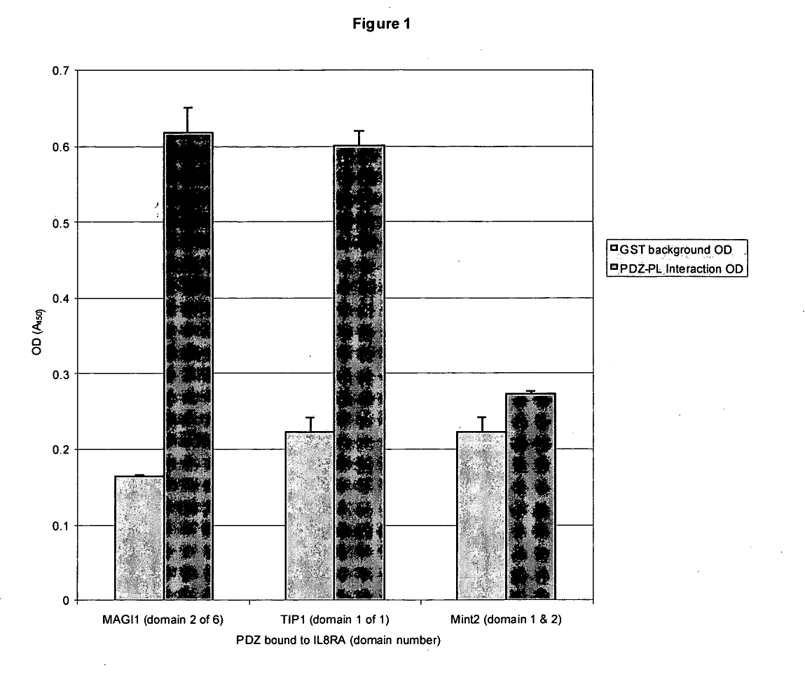

Results of peptides representing the carboxy-terminal 20 amino acids of IL8RA binding to MAGI1, domain 2 of 6, TIP1, and Mint2, domains 1 and 2, are shown in FIG. 1. Clearly, IL8RA binds GST-MAGI 1 domain 2 and GST-TIP1 with much higher affinity than it does to GST-Mint2 domains 1 & 2 at equivalent peptide concentrations and with equivalent amount of GST-PDZ fusion protein. Because the interaction between 118RA and Mint2 is not significantly higher than background, Mint2 PDZ's may not interact with IL8RA PL peptide when tested in this assay.

D. Conclusions and Summary MAGI1 (domain 2) and TIP1 bind to IL8RA better than Mint2 (domains 1 and 2) bind

to the same peptide.

The “G” Assay provides an accurate method for testing the binding of PDZ proteins to PL peptides in vitro, and highlights the specificity of PDZ-PL pairing. The same peptide can interact more or less strongly with different PDZs, and binding strength is not relative for the same PDZ. However, binding affinity and binding patterns of PDZ's and PL's are not predictable, and binding profiles may change with assay variations and data interpretation.

### EXAMPLE 3

### Identification of Alpha Adrenergic Receptor Interactions with PDZ Proteins

This example describes the binding of a subset of alpha adrenergic receptors and PDZ domains using the modified ELISA described in the previous example. Biotinylated peptides corresponding to the C-terminal 20 amino acids of A1A, A1B and A1C synthesized and purified by HPLC. Binding between these entities was detected through the “G” Assay, a calorimetric assay using avidin-HRP to bind the biotin and a peroxidase substrate.

TABLE 8A

| | | | | |
|---|---|---|---|---|
| AVC NAME | SEQUENCE | GENE NAME | DOMAIN | AVERAGE OD |

| | | | | |
|---|---|---|---|---|
| ALPHA-2A AR | HDFRRAFKKILARGDRKRIV | AF6 | 1 | 0.3325 |
| ALPHA-2A AR | HDFRRAFKKILARGDRKRIV | AF6 | 1 | 0.5125 |
| ALPHA-2A AR | HDFRRAFKKILARGDRKRIV | AIPC | 1 | 2.594 |
| ALPHA-2A AR | HDFRRAFKKILARGDRKRIV | AIPC | 1 | 1.93 |
| ALPHA-2A AR | HDFRRAFKKILARGDRKRIV | AIPC | 3 | 0.1465 |
| ALPHA-2A AR | HDFRRAFKKILARGDRKRIV | AIPC | 3 | 0.2165 |
| ALPHA-2A AR | HDFRRAFKKILARGDRKRIV | AIPC | 4 | 0.197 |
| ALPHA-2A AR | HDFRRAFKKILARGDRKRIV | AIPC | 4 | 0.2285 |
| ALPHA-2A AR | HDFRRAFKKILARGDRKRIV | APXL1 | 1 | 1.194 |
| ALPHA-2A AR | HDFRRAFKKILARGDRKRIV | APXL1 | 1 | 0.5545 |
| ALPHA-2A AR | HDFRRAFKKILARGDRKRIV | CARD14 | 1 | 1.06 |
| ALPHA-2A AR | HDFRRAFKKILARGDRKRIV | CARD14 | 1 | 0.6535 |
| ALPHA-2A AR | HDFRRAFKKILARGDRKRIV | CASK | 1 | 0.2825 |
| ALPHA-2A AR | HDFRRAFKKILARGDRKRIV | CASK | 1 | 0.475 |
| ALPHA-2A AR | HDFRRAFKKILARGDRKRIV | CNK1 | 1 | 0.275 |
| ALPHA-2A AR | HDFRRAFKKILARGDRKRIV | CNK1 | 1 | 0.4505 |
| ALPHA-2A AR | HDFRRAFKKILARGDRKRIV | CYTOHESIN BINDING | 1 | 0.2515 |
| | | PROTEIN | | |
| ALPHA-2A AR | HDFRRAFKKILARGDRKRIV | CYTOHESIN BINDING | 1 | 0.38 |
| | | PROTEIN | | |
| ALPHA-2A AR | HDFRRAFKKILARGDRKRIV | DLG1 | 1 | 0.142 |
| ALPHA-2A AR | HDFRRAFKKILARGDRKRIV | DLG1 | 1 | 0.204 |
| ALPHA-2A AR | HDFRRAFKKILARGDRKRIV | DLG1 | 2 | 0.1665 |
| ALPHA-2A AR | HDFRRAFKKILARGDRKRIV | DLG1 | 2 | 0.272 |
| ALPHA-2A AR | HDFRRAFKKILARGDRKRIV | DLG1 | 3 | 0.2415 |
| ALPHA-2A AR | HDFRRAFKKILARGDRKRIV | DLG1 | 3 | 0.5315 |
| ALPHA-2A AR | HDFRRAFKKILARGDRKRIV | DLG1 | 1, 2 | 0.2435 |
| ALPHA-2A AR | HDFRRAFKKILARGDRKRIV | DLG1 | 1, 2 | 0.3955 |
| ALPHA-2A AR | HDFRRAFKKILARGDRKRIV | DLG2 | 1 | 0.1185 |
| ALPHA-2A AR | HDFRRAFKKILARGDRKRIV | DLG2 | 1 | 0.2255 |
| ALPHA-2A AR | HDFRRAFKKILARGDRKRIV | DLG2 | 2 | 0.208 |
| ALPHA-2A AR | HDFRRAFKKILARGDRKRIV | DLG2 | 2 | 0.3005 |
| ALPHA-2A AR | HDFRRAFKKILARGDRKRIV | DLG5 | 1 | 0.1955 |
| ALPHA-2A AR | HDFRRAFKKILARGDRKRIV | DLG5 | 1 | 0.168 |
| ALPHA-2A AR | HDFRRAFKKILARGDRKRIV | DLG5 | 2 | 0.3655 |
| ALPHA-2A AR | HDFRRAFKKILARGDRKRIV | DLG5 | 2 | 0.6325 |
| ALPHA-2A AR | HDFRRAFKKILARGDRKRIV | DLG5 | 2 | 0.648 |
| ALPHA-2A AR | HDFRRAFKKILARGDRKRIV | DLG5 | 2 | 0.474 |
| ALPHA-2A AR | HDFRRAFKKILARGDRKRIV | DVL2 | 1 | 0.294 |
| ALPHA-2A AR | HDFRRAFKKILARGDRKRIV | DVL2 | 1 | 0.456 |
| ALPHA-2A AR | HDFRRAFKKILARGDRKRIV | DVL3 | 1 | 0.4915 |
| ALPHA-2A AR | HDFRRAFKKILARGDRKRIV | DVL3 | 1 | 0.8465 |
| ALPHA-2A AR | HDFRRAFKKILARGDRKRIV | EBP50 | 1 | 0.406 |
| ALPHA-2A AR | HDFRRAFKKILARGDRKRIV | EBP50 | 1 | 0.1385 |
| ALPHA-2A AR | HDFRRAFKKILARGDRKRIV | EBP50 | 2 | 0.2395 |
| ALPHA-2A AR | HDFRRAFKKILARGDRKRIV | EBP50 | 2 | 0.139 |
| ALPHA-2A AR | HDFRRAFKKILARGDRKRIV | EBP50 | 1, 2 | 0.2515 |
| ALPHA-2A AR | HDFRRAFKKILARGDRKRIV | EBP50 | 1, 2 | 0.1295 |
| ALPHA-2A AR | HDFRRAFKKILARGDRKRIV | ENIGMA | 1 | 0.3955 |
| ALPHA-2A AR | HDFRRAFKKILARGDRKRIV | ENIGMA | 1 | 0.144 |
| ALPHA-2A AR | HDFRRAFKKILARGDRKRIV | ERBIN | 1 | 0.2285 |
| ALPHA-2A AR | HDFRRAFKKILARGDRKRIV | ERBIN | 1 | 0.451 |
| ALPHA-2A AR | HDFRRAFKKILARGDRKRIV | FLJ00011 | 1 | 0.2725 |
| ALPHA-2A AR | HDFRRAFKKILARGDRKRIV | FLJ00011 | 1 | 0.402 |
| ALPHA-2A AR | HDFRRAFKKILARGDRKRIV | FLJ11215 | 1 | 0.141 |
| ALPHA-2A AR | HDFRRAFKKILARGDRKRIV | FLJ11215 | 1 | 0.2065 |
| ALPHA-2A AR | HDFRRAFKKILARGDRKRIV | FLJ12615 | 1 | 0.157 |
| ALPHA-2A AR | HDFRRAFKKILARGDRKRIV | FLJ12615 | 1 | 0.26 |
| ALPHA-2A AR | HDFRRAFKKILARGDRKRIV | FLJ21687 | 1 | 0.9965 |
| ALPHA-2A AR | HDFRRAFKKILARGDRKRIV | FLJ21687 | 1 | 0.8225 |
| ALPHA-2A AR | HDFRRAFKKILARGDRKRIV | GRIP 1 | 4 | 0.402 |
| ALPHA-2A AR | HDFRRAFKKILARGDRKRIV | GRIP 1 | 4 | 0.339 |
| ALPHA-2A AR | HDFRRAFKKILARGDRKRIV | GRIP 1 | 5 | 0.405 |
| ALPHA-2A AR | HDFRRAFKKILARGDRKRIV | GRIP 1 | 5 | 0.3185 |
| ALPHA-2A AR | HDFRRAFKKILARGDRKRIV | GRIP 1 | 6 | 0.3795 |
| ALPHA-2A AR | HDFRRAFKKILARGDRKRIV | GRIP 1 | 6 | 0.177 |
| ALPHA-2A AR | HDFRRAFKKILARGDRKRIV | GRIP 1 | 7 | 0.26 |
| ALPHA-2A AR | HDFRRAFKKILARGDRKRIV | GRIP 1 | 7 | 0.187 |
| ALPHA-2A AR | HDFRRAFKKILARGDRKRIV | HEMBA 1003117 | 1 | 0.558 |
| ALPHA-2A AR | HDFRRAFKKILARGDRKRIV | HEMBA 1003117 | 1 | 0.415 |
| ALPHA-2A AR | HDFRRAFKKILARGDRKRIV | HEMBA 1003117 | 1 | 0.5875 |
| ALPHA-2A AR | HDFRRAFKKILARGDRKRIV | HEMBA 1003117 | 1 | 0.8515 |
| ALPHA-2A AR | HDFRRAFKKILARGDRKRIV | INADL | 1 | 0.336 |
| ALPHA-2A AR | HDFRRAFKKILARGDRKRIV | INADL | 1 | 0.5975 |
| ALPHA-2A AR | HDFRRAFKKILARGDRKRIV | INADL | 3 | 1.095 |
| ALPHA-2A AR | HDFRRAFKKILARGDRKRIV | INADL | 3 | 2.1295 |
| ALPHA-2A AR | HDFRRAFKKILARGDRKRIV | INADL | 4 | 0.6395 |
| ALPHA-2A AR | HDFRRAFKKILARGDRKRIV | INADL | 4 | 1.049 |
| ALPHA-2A AR | HDFRRAFKKILARGDRKRIV | INADL | 5 | 0.2175 |
| ALPHA-2A AR | HDFRRAFKKILARGDRKRIV | INADL | 5 | 0.3455 |
| ALPHA-2A AR | HDFRRAFKKILARGDRKRIV | INADL | 7 | 0.372 |
| ALPHA-2A AR | HDFRRAFKKILARGDRKRIV | INADL | 7 | 0.5995 |
| ALPHA-2A AR | HDFRRAFKKILARGDRKRIV | INADL | 8 | 0.2785 |
| ALPHA-2A AR | HDFRRAFKKILARGDRKRIV | INADL | 8 | 0.47 |
| ALPHA-2A AR | HDFRRAFKKILARGDRKRIV | KIAA0316 | 1 | 0.1965 |
| ALPHA-2A AR | HDFRRAFKKILARGDRKRIV | KIAA0316 | 1 | 0.18 |
| ALPHA-2A AR | HDFRRAFKKILARGDRKRIV | KIAA0340 | 1 | 0.855 |
| ALPHA-2A AR | HDFRRAFKKILARGDRKRIV | KIAA0340 | 1 | 1.224 |
| ALPHA-2A AR | HDFRRAFKKILARGDRKRIV | KIAA0380 | 1 | 2.061 |
| ALPHA-2A AR | HDFRRAFKKILARGDRKRIV | KIAA0380 | 1 | 2.5805 |
| ALPHA-2A AR | HDFRRAFKKILARGDRKRIV | KIAA0382 | 1 | 0.2085 |
| ALPHA-2A AR | HDFRRAFKKILARGDRKRIV | KIAA0382 | 1 | 0.3865 |
| ALPHA-2A AR | HDFRRAFKKILARGDRKRIV | KIAA0440 | 1 | 1.176 |
| ALPHA-2A AR | HDFRRAFKKILARGDRKRIV | KIAA0440 | 1 | 0.733 |
| ALPHA-2A AR | HDFRRAFKKILARGDRKRIV | KIAA0559 | 1 | 0.2355 |
| ALPHA-2A AR | HDFRRAFKKILARGDRKRIV | KIAA0559 | 1 | 0.3155 |
| ALPHA-2A AR | HDFRRAFKKILARGDRKRIV | KIAA0751 | 1 | 0.667 |
| ALPHA-2A AR | HDFRRAFKKILARGDRKRIV | KIAA0751 | 1 | 1.1525 |
| ALPHA-2A AR | HDFRRAFKKILARGDRKRIV | KIAA0751 | 1 | 3.4115 |
| ALPHA-2A AR | HDFRRAFKKILARGDRKRIV | KIAA0751 | 1 | 2.67 |
| ALPHA-2A AR | HDFRRAFKKILARGDRKRIV | KIAA0858 | 1 | 0.23 |
| ALPHA-2A AR | HDFRRAFKKILARGDRKRIV | KIAA0858 | 1 | 0.3835 |
| ALPHA-2A AR | HDFRRAFKKILARGDRKRIV | KIAA0967 | 1 | 0.2555 |
| ALPHA-2A AR | HDFRRAFKKILARGDRKRIV | KIAA0967 | 1 | 0.1555 |
| ALPHA-2A AR | HDFRRAFKKILARGDRKRIV | KIAA1095 | 1 | 0.2225 |
| ALPHA-2A AR | HDFRRAFKKILARGDRKRIV | KIAA1095 | 1 | 0.328 |
| ALPHA-2A AR | HDFRRAFKKILARGDRKRIV | KIAA1095 | 2 | 0.2635 |
| ALPHA-2A AR | HDFRRAFKKILARGDRKRIV | KIAA1095 | 2 | 0.3465 |
| ALPHA-2A AR | HDFRRAFKKILARGDRKRIV | KIAA1222 | 1 | 0.3325 |
| ALPHA-2A AR | HDFRRAFKKILARGDRKRIV | KIAA1222 | 1 | 0.2375 |
| ALPHA-2A AR | HDFRRAFKKILARGDRKRIV | KIAA1284 | 1 | 0.8405 |
| ALPHA-2A AR | HDFRRAFKKILARGDRKRIV | KIAA1284 | 1 | 0.845 |
| ALPHA-2A AR | HDFRRAFKKILARGDRKRIV | KIAA1415 | 1 | 0.3215 |
| ALPHA-2A AR | HDFRRAFKKILARGDRKRIV | KIAA1415 | 1 | 0.3045 |
| ALPHA-2A AR | HDFRRAFKKILARGDRKRIV | KIAA1526 | 1 | 0.209 |
| ALPHA-2A AR | HDFRRAFKKILARGDRKRIV | KIAA1526 | 1 | 0.3675 |
| ALPHA-2A AR | HDFRRAFKKILARGDRKRIV | KIAA1526 | 1 | 3.8915 |
| ALPHA-2A AR | HDFRRAFKKILARGDRKRIV | KIAA1526 | 1 | 4 |
| ALPHA-2A AR | HDFRRAFKKILARGDRKRIV | KIAA1526 | 2 | 0.8305 |
| ALPHA-2A AR | HDFRRAFKKILARGDRKRIV | KIAA1526 | 2 | 1.511 |
| ALPHA-2A AR | HDFRRAFKKILARGDRKRIV | KIAA1526 | 2 | 0.2085 |
| ALPHA-2A AR | HDFRRAFKKILARGDRKRIV | KIAA1526 | 2 | 0.4095 |
| ALPHA-2A AR | HDFRRAFKKILARGDRKRIV | KIAA1620 | 1 | 0.231 |
| ALPHA-2A AR | HDFRRAFKKILARGDRKRIV | KIAA1620 | 1 | 0.152 |
| ALPHA-2A AR | HDFRRAFKKILARGDRKRIV | KIAA1719 | 1 | 0.2835 |
| ALPHA-2A AR | HDFRRAFKKILARGDRKRIV | KIAA1719 | 1 | 0.1895 |
| ALPHA-2A AR | HDFRRAFKKILARGDRKRIV | KIAA1719 | 2 | 0.2545 |
| ALPHA-2A AR | HDFRRAFKKILARGDRKRIV | KIAA1719 | 2 | 0.203 |
| ALPHA-2A AR | HDFRRAFKKILARGDRKRIV | KIAA1719 | 3 | 0.338 |
| ALPHA-2A AR | HDFRRAFKKILARGDRKRIV | KIAA1719 | 3 | 0.2555 |
| ALPHA-2A AR | HDFRRAFKKILARGDRKRIV | KIAA1719 | 4 | 2.4485 |
| ALPHA-2A AR | HDFRRAFKKILARGDRKRIV | KIAA1719 | 4 | 2.433 |
| ALPHA-2A AR | HDFRRAFKKILARGDRKRIV | KIAA1719 | 5 | 0.417 |
| ALPHA-2A AR | HDFRRAFKKILARGDRKRIV | KIAA1719 | 5 | 0.356 |
| ALPHA-2A AR | HDFRRAFKKILARGDRKRIV | KIAA1719 | 6 | 0.264 |
| ALPHA-2A AR | HDFRRAFKKILARGDRKRIV | KIAA1719 | 6 | 0.1695 |
| ALPHA-2A AR | HDFRRAFKKILARGDRKRIV | LIM MYSTIQUE | 1 | 0.8755 |
| ALPHA-2A AR | HDFRRAFKKILARGDRKRIV | LIM MYSTIQUE | 1 | 0.8705 |
| ALPHA-2A AR | HDFRRAFKKILARGDRKRIV | LIM PROTEIN | 1 | 0.5305 |
| ALPHA-2A AR | HDFRRAFKKILARGDRKRIV | LIM PROTEIN | 1 | 0.732 |
| ALPHA-2A AR | HDFRRAFKKILARGDRKRIV | LIM-RIL | 1 | 0.407 |
| ALPHA-2A AR | HDFRRAFKKILARGDRKRIV | LIM-RIL | 1 | 0.4955 |
| ALPHA-2A AR | HDFRRAFKKILARGDRKRIV | LIMK1 | 1 | 0.354 |
| ALPHA-2A AR | HDFRRAFKKILARGDRKRIV | LIMK1 | 1 | 0.3655 |
| ALPHA-2A AR | HDFRRAFKKILARGDRKRIV | LIMK2 | 1 | 0.344 |
| ALPHA-2A AR | HDFRRAFKKILARGDRKRIV | LIMK2 | 1 | 0.4015 |
| ALPHA-2A AR | HDFRRAFKKILARGDRKRIV | LU-1 | 1 | 0.2425 |
| ALPHA-2A AR | HDFRRAFKKILARGDRKRIV | LU-1 | 1 | 0.19 |
| ALPHA-2A AR | HDFRRAFKKILARGDRKRIV | MAGI 1 | 1 | 0.247 |
| ALPHA-2A AR | HDFRRAFKKILARGDRKRIV | MAGI 1 | 1 | 0.365 |
| ALPHA-2A AR | HDFRRAFKKILARGDRKRIV | MAGI 1 | 1 | 0.3645 |
| ALPHA-2A AR | HDFRRAFKKILARGDRKRIV | MAGI 1 | 1 | 0.4925 |
| ALPHA-2A AR | HDFRRAFKKILARGDRKRIV | MAGI 1 | 3 | 0.2915 |
| ALPHA-2A AR | HDFRRAFKKILARGDRKRIV | MAGI 1 | 3 | 0.4715 |
| ALPHA-2A AR | HDFRRAFKKILARGDRKRIV | MAGI 1 | 3 | 2.564 |
| ALPHA-2A AR | HDFRRAFKKILARGDRKRIV | MAGI 1 | 3 | 3.664 |
| ALPHA-2A AR | HDFRRAFKKILARGDRKRIV | MAGI 1 | 4 | 0.3085 |
| ALPHA-2A AR | HDFRRAFKKILARGDRKRIV | MAGI 1 | 4 | 0.4115 |
| ALPHA-2A AR | HDFRRAFKKILARGDRKRIV | MAGI 1 | 5 | 0.245 |
| ALPHA-2A AR | HDFRRAFKKILARGDRKRIV | MAGI 1 | 5 | 0.3925 |
| ALPHA-2A AR | HDFRRAFKKILARGDRKRIV | MAGI 2 | 1 | 0.2595 |
| ALPHA-2A AR | HDFRRAFKKILARGDRKRIV | MAGI 2 | 1 | 0.1815 |
| ALPHA-2A AR | HDFRRAFKKILARGDRKRIV | MAGI 2 | 2 | 0.205 |
| ALPHA-2A AR | HDFRRAFKKILARGDRKRIV | MAGI 2 | 2 | 0.136 |
| ALPHA-2A AR | HDFRRAFKKILARGDRKRIV | MAGI 2 | 3 | 0.2925 |
| ALPHA-2A AR | HDFRRAFKKILARGDRKRIV | MAGI 2 | 3 | 0.1885 |
| ALPHA-2A AR | HDFRRAFKKILARGDRKRIV | MAGI 2 | 4 | 0.144 |
| ALPHA-2A AR | HDFRRAFKKILARGDRKRIV | MAGI 2 | 4 | 0.18 |
| ALPHA-2A AR | HDFRRAFKKILARGDRKRIV | MAGI 2 | 5 | 0.7415 |
| ALPHA-2A AR | HDFRRAFKKILARGDRKRIV | MAGI 2 | 5 | 0.8035 |
| ALPHA-2A AR | HDFRRAFKKILARGDRKRIV | MAGI 2 | 6 | 0.763 |
| ALPHA-2A AR | HDFRRAFKKILARGDRKRIV | MAGI 2 | 6 | 0.9055 |
| ALPHA-2A AR | HDFRRAFKKILARGDRKRIV | MAGI 3 | 1 | 0.272 |
| ALPHA-2A AR | HDFRRAFKKILARGDRKRIV | MAGI 3 | 1 | 0.499 |
| ALPHA-2A AR | HDFRRAFKKILARGDRKRIV | MAGI 3 | 2 | 0.701 |
| ALPHA-2A AR | HDFRRAFKKILARGDRKRIV | MAGI 3 | 2 | 1.192 |
| ALPHA-2A AR | HDFRRAFKKILARGDRKRIV | MAGI 3 | 3 | 0.243 |
| ALPHA-2A AR | HDFRRAFKKILARGDRKRIV | MAGI 3 | 3 | 0.566 |
| ALPHA-2A AR | HDFRRAFKKILARGDRKRIV | MAGI 3 | 4 | 0.2545 |
| ALPHA-2A AR | HDFRRAFKKILARGDRKRIV | MAGI 3 | 4 | 0.4775 |
| ALPHA-2A AR | HDFRRAFKKILARGDRKRIV | MAGI 3 | 5 | 0.2745 |
| ALPHA-2A AR | HDFRRAFKKILARGDRKRIV | MAGI 3 | 5 | 0.5265 |
| ALPHA-2A AR | HDFRRAFKKILARGDRKRIV | MAST1 | 1 | 0.4675 |
| ALPHA-2A AR | HDFRRAFKKILARGDRKRIV | MAST1 | 1 | 0.355 |
| ALPHA-2A AR | HDFRRAFKKILARGDRKRIV | MAST2 | 1 | 0.6125 |
| ALPHA-2A AR | HDFRRAFKKILARGDRKRIV | MAST2 | 1 | 0.5255 |
| ALPHA-2A AR | HDFRRAFKKILARGDRKRIV | MAST2 | | 0.98 |
| ALPHA-2A AR | HDFRRAFKKILARGDRKRIV | MAST2 | | 1.7505 |
| ALPHA-2A AR | HDFRRAFKKILARGDRKRIV | MAST4 | 1 | 0.264 |
| ALPHA-2A AR | HDFRRAFKKILARGDRKRIV | MAST4 | 1 | 0.3355 |
| ALPHA-2A AR | HDFRRAFKKILARGDRKRIV | MINT1 | 1 | 1.0045 |
| ALPHA-2A AR | HDFRRAFKKILARGDRKRIV | MINT1 | 1 | 0.781 |
| ALPHA-2A AR | HDFRRAFKKILARGDRKRIV | MINT1 | 2 | 0.299 |
| ALPHA-2A AR | HDFRRAFKKILARGDRKRIV | MINT1 | 2 | 0.1895 |
| ALPHA-2A AR | HDFRRAFKKILARGDRKRIV | MINT1 | 1, 2 | 3.184 |
| ALPHA-2A AR | HDFRRAFKKILARGDRKRIV | MINT1 | 1, 2 | 3.8385 |
| ALPHA-2A AR | HDFRRAFKKILARGDRKRIV | MPP1 | 1 | 0.479 |
| ALPHA-2A AR | HDFRRAFKKILARGDRKRIV | MPP1 | 1 | 0.685 |
| ALPHA-2A AR | HDFRRAFKKILARGDRKRIV | MPP2 | 1 | 0.464 |
| ALPHA-2A AR | HDFRRAFKKILARGDRKRIV | MPP2 | 1 | 0.318 |
| ALPHA-2A AR | HDFRRAFKKILARGDRKRIV | MUPP1 | 1 | 0.4445 |
| ALPHA-2A AR | HDFRRAFKKILARGDRKRIV | MUPP1 | 1 | 0.7405 |
| ALPHA-2A AR | HDFRRAFKKILARGDRKRIV | MUPP1 | 2 | 0.4995 |
| ALPHA-2A AR | HDFRRAFKKILARGDRKRIV | MUPP1 | 2 | 0.5935 |
| ALPHA-2A AR | HDFRRAFKKILARGDRKRIV | MUPP1 | 3 | 0.4815 |
| ALPHA-2A AR | HDFRRAFKKILARGDRKRIV | MUPP1 | 3 | 0.742 |
| ALPHA-2A AR | HDFRRAFKKILARGDRKRIV | MUPP1 | 4 | 1.08 |
| ALPHA-2A AR | HDFRRAFKKILARGDRKRIV | MUPP1 | 4 | 1.923 |
| ALPHA-2A AR | HDFRRAFKKILARGDRKRIV | MUPP1 | 5 | 0.3005 |
| ALPHA-2A AR | HDFRRAFKKILARGDRKRIV | MUPP1 | 5 | 0.706 |
| ALPHA-2A AR | HDFRRAFKKILARGDRKRIV | MUPP1 | 6 | 1.1875 |
| ALPHA-2A AR | HDFRRAFKKILARGDRKRIV | MUPP1 | 6 | 1.909 |
| ALPHA-2A AR | HDFRRAFKKILARGDRKRIV | MUPP1 | 7 | 0.377 |
| ALPHA-2A AR | HDFRRAFKKILARGDRKRIV | MUPP1 | 7 | 0.676 |
| ALPHA-2A AR | HDFRRAFKKILARGDRKRIV | MUPP1 | 8 | 0.835 |
| ALPHA-2A AR | HDFRRAFKKILARGDRKRIV | MUPP1 | 8 | 1.5405 |
| ALPHA-2A AR | HDFRRAFKKILARGDRKRIV | MUPP1 | 9 | 0.2845 |
| ALPHA-2A AR | HDFRRAFKKILARGDRKRIV | MUPP1 | 9 | 0.5165 |
| ALPHA-2A AR | HDFRRAFKKILARGDRKRIV | MUPP1 | 10 | 0.3165 |
| ALPHA-2A AR | HDFRRAFKKILARGDRKRIV | MUPP1 | 10 | 0.514 |
| ALPHA-2A AR | HDFRRAFKKILARGDRKRIV | MUPP1 | 11 | 0.309 |
| ALPHA-2A AR | HDFRRAFKKILARGDRKRIV | MUPP1 | 11 | 0.6785 |
| ALPHA-2A AR | HDFRRAFKKILARGDRKRIV | MUPP1 | 12 | 0.23 |
| ALPHA-2A AR | HDFRRAFKKILARGDRKRIV | MUPP1 | 12 | 0.3145 |
| ALPHA-2A AR | HDFRRAFKKILARGDRKRIV | MUPP1 | 13 | 0.5555 |
| ALPHA-2A AR | HDFRRAFKKILARGDRKRIV | MUPP1 | 13 | 0.842 |
| ALPHA-2A AR | HDFRRAFKKILARGDRKRIV | NEDLG | 1 | 0.2175 |
| ALPHA-2A AR | HDFRRAFKKILARGDRKRIV | NEDLG | 1 | 0.143 |
| ALPHA-2A AR | HDFRRAFKKILARGDRKRIV | NEDLG | 2 | 0.159 |
| ALPHA-2A AR | HDFRRAFKKILARGDRKRIV | NEDLG | 2 | 0.2355 |
| ALPHA-2A AR | HDFRRAFKKILARGDRKRIV | NEDLG | 3 | 0.137 |
| ALPHA-2A AR | HDFRRAFKKILARGDRKRIV | NEDLG | 3 | 0.2555 |
| ALPHA-2A AR | HDFRRAFKKILARGDRKRIV | NEDLG | 1, 2 | 0.3165 |
| ALPHA-2A AR | HDFRRAFKKILARGDRKRIV | NEDLG | 1, 2 | 0.401 |
| ALPHA-2A AR | HDFRRAFKKILARGDRKRIV | NOS1 | 1 | 0.7285 |
| ALPHA-2A AR | HDFRRAFKKILARGDRKRIV | NOS1 | 1 | 0.96 |
| ALPHA-2A AR | HDFRRAFKKILARGDRKRIV | NOVEL PDZ GENE | 1 | 0.8105 |
| ALPHA-2A AR | HDFRRAFKKILARGDRKRIV | NOVEL PDZ GENE | 1 | 2.973 |
| ALPHA-2A AR | HDFRRAFKKILARGDRKRIV | NOVEL PDZ GENE | 2 | 0.363 |
| ALPHA-2A AR | HDFRRAFKKILARGDRKRIV | NOVEL PDZ GENE | 2 | 0.844 |
| ALPHA-2A AR | HDFRRAFKKILARGDRKRIV | OUTER MEMBRANE | 1 | 0.21 |
| ALPHA-2A AR | HDFRRAFKKILARGDRKRIV | OUTER MEMBRANE | 1 | 0.4655 |
| ALPHA-2A AR | HDFRRAFKKILARGDRKRIV | P55T | 1 | 0.236 |
| ALPHA-2A AR | HDFRRAFKKILARGDRKRIV | P55T | 1 | 0.1785 |
| ALPHA-2A AR | HDFRRAFKKILARGDRKRIV | PAR3 | 2 | 0.2675 |
| ALPHA-2A AR | HDFRRAFKKILARGDRKRIV | PAR3 | 2 | 0.2085 |
| ALPHA-2A AR | HDFRRAFKKILARGDRKRIV | PAR3 | 3 | 1.451 |
| ALPHA-2A AR | HDFRRAFKKILARGDRKRIV | PAR3 | 3 | 1.2735 |
| ALPHA-2A AR | HDFRRAFKKILARGDRKRIV | PAR6 | 1 | 0.381 |
| ALPHA-2A AR | HDFRRAFKKILARGDRKRIV | PAR6 | 1 | 0.568 |
| ALPHA-2A AR | HDFRRAFKKILARGDRKRIV | PAR6 GAMMA | 1 | 0.2065 |
| ALPHA-2A AR | HDFRRAFKKILARGDRKRIV | PAR6 GAMMA | 1 | 0.2425 |
| ALPHA-2A AR | HDFRRAFKKILARGDRKRIV | PDZ-73 | 2 | 0.251 |
| ALPHA-2A AR | HDFRRAFKKILARGDRKRIV | PDZ-73 | 2 | 0.4365 |
| ALPHA-2A AR | HDFRRAFKKILARGDRKRIV | PDZ-73 | 3 | 0.2225 |
| ALPHA-2A AR | HDFRRAFKKILARGDRKRIV | PDZ-73 | 3 | 0.369 |
| ALPHA-2A AR | HDFRRAFKKILARGDRKRIV | PDZK1 | 1 | 0.3415 |
| ALPHA-2A AR | HDFRRAFKKILARGDRKRIV | PDZK1 | 1 | 0.608 |
| ALPHA-2A AR | HDFRRAFKKILARGDRKRIV | PDZK1 | 2 | 0.29 |
| ALPHA-2A AR | HDFRRAFKKILARGDRKRIV | PDZK1 | 2 | 0.4915 |
| ALPHA-2A AR | HDFRRAFKKILARGDRKRIV | PDZK1 | 3 | 0.5655 |
| ALPHA-2A AR | HDFRRAFKKILARGDRKRIV | PDZK1 | 3 | 0.5355 |
| ALPHA-2A AR | HDFRRAFKKILARGDRKRIV | PDZK1 | 4 | 0.199 |
| ALPHA-2A AR | HDFRRAFKKILARGDRKRIV | PDZK1 | 4 | 0.2365 |
| ALPHA-2A AR | HDFRRAFKKILARGDRKRIV | PDZK1 | 2, 3, 4 | 0.441 |
| ALPHA-2A AR | HDFRRAFKKILARGDRKRIV | PDZK1 | 2, 3, 4 | 0.5115 |
| ALPHA-2A AR | HDFRRAFKKILARGDRKRIV | PICK1 | 1 | 0.535 |
| ALPHA-2A AR | HDFRRAFKKILARGDRKRIV | PICK1 | 1 | 0.769 |
| ALPHA-2A AR | HDFRRAFKKILARGDRKRIV | PIST | 1 | 0.144 |
| ALPHA-2A AR | HDFRRAFKKILARGDRKRIV | PIST | 1 | 0.35 |
| ALPHA-2A AR | HDFRRAFKKILARGDRKRIV | PRIL16 | 1 | 0.3105 |
| ALPHA-2A AR | HDFRRAFKKILARGDRKRIV | PRIL16 | 1 | 0.292 |
| ALPHA-2A AR | HDFRRAFKKILARGDRKRIV | PRIL16 | 2 | 0.2165 |
| ALPHA-2A AR | HDFRRAFKKILARGDRKRIV | PRIL16 | 2 | 0.173 |
| ALPHA-2A AR | HDFRRAFKKILARGDRKRIV | PRIL16 | 1, 2 | 0.5495 |
| ALPHA-2A AR | HDFRRAFKKILARGDRKRIV | PRIL16 | 1, 2 | 0.6355 |
| ALPHA-2A AR | HDFRRAFKKILARGDRKRIV | PSD95 | 1 | 0.2165 |
| ALPHA-2A AR | HDFRRAFKKILARGDRKRIV | PSD95 | 1 | 0.115 |
| ALPHA-2A AR | HDFRRAFKKILARGDRKRIV | PSD95 | 3 | 0.161 |
| ALPHA-2A AR | HDFRRAFKKILARGDRKRIV | PSD95 | 3 | 0.1085 |
| ALPHA-2A AR | HDFRRAFKKILARGDRKRIV | PSD95 | 1, 2, 3 | 0.341 |
| ALPHA-2A AR | HDFRRAFKKILARGDRKRIV | PSD95 | 1, 2, 3 | 0.4045 |
| ALPHA-2A AR | HDFRRAFKKILARGDRKRIV | PTN-4 | 1 | 0.384 |
| ALPHA-2A AR | HDFRRAFKKILARGDRKRIV | PTN-4 | 1 | 0.425 |
| ALPHA-2A AR | HDFRRAFKKILARGDRKRIV | PTPL1 | 1 | 0.2225 |
| ALPHA-2A AR | HDFRRAFKKILARGDRKRIV | PTPL1 | 1 | 0.163 |
| ALPHA-2A AR | HDFRRAFKKILARGDRKRIV | PTPL1 | 2 | 1.6145 |
| ALPHA-2A AR | HDFRRAFKKILARGDRKRIV | PTPL1 | 2 | 1.452 |
| ALPHA-2A AR | HDFRRAFKKILARGDRKRIV | PTPL1 | 3 | 0.1595 |
| ALPHA-2A AR | HDFRRAFKKILARGDRKRIV | PTPLi | 3 | 0.1905 |
| ALPHA-2A AR | HDFRRAFKKILARGDRKRIV | PTPL1 | 4 | 0.265 |
| ALPHA-2A AR | HDFRRAFKKILARGDRKRIV | PTPL1 | 4 | 0.4135 |
| ALPHA-2A AR | HDFRRAFKKILARGDRKRIV | PTPL1 | 5 | 0.1895 |
| ALPHA-2A AR | HDFRRAFKKILARGDRKRIV | PTPL1 | 5 | 0.3 |
| ALPHA-2A AR | HDFRRAFKKILARGDRKRIV | SHANK 1 | 1 | 0.281 |
| ALPHA-2A AR | HDFRRAFKKILARGDRKRIV | SHANK 1 | 1 | 0.2205 |
| ALPHA-2A AR | HDFRRAFKKILARGDRKRIV | SIP1 | 2 | 0.332 |
| ALPHA-2A AR | HDFRRAFKKILARGDRKRIV | SIP1 | 2 | 0.205 |
| ALPHA-2A AR | HDFRRAFKKILARGDRKRIV | SITAC 18 | 1 | 3.8915 |
| ALPHA-2A AR | HDFRRAFKKILARGDRKRIV | SITAC 18 | 1 | 3.297 |
| ALPHA-2A AR | HDFRRAFKKILARGDRKRIV | SITAC 18 | 2 | 3.8365 |
| ALPHA-2A AR | HDFRRAFKKILARGDRKRIV | SITAC 18 | 2 | 4 |
| ALPHA-2A AR | HDFRRAFKKILARGDRKRIV | SYNTROPHIN 1 ALPHA | 1 | 1.11 |
| ALPHA-2A AR | HDFRRAFKKILARGDRKRIV | SYNTROPHIN 1 ALPHA | 1 | 1.78 |
| ALPHA-2A AR | HDFRRAFKKILARGDRKRIV | SYNTROPHIN GAMMA 1 | 1 | 1.2705 |
| ALPHA-2A AR | HDFRRAFKKILARGDRKRIV | SYNTROPHIN GAMMA 1 | 1 | 1.126 |
| ALPHA-2A AR | HDFRRAFKKILARGDRKRIV | SYNTROPHIN GAMMA 2 | 1 | 0.265 |
| ALPHA-2A AR | HDFRRAFKKILARGDRKRIV | SYNTROPHIN GAMMA 2 | 1 | 0.155 |
| ALPHA-2A AR | HDFRRAFKKILARGDRKRIV | TAX2-LIKE PROTEIN | 1 | 0.2445 |
| ALPHA-2A AR | HDFRRAFKKILARGDRKRIV | TAX2-LIKE PROTEIN | 1 | 0.558 |
| ALPHA-2A AR | HDFRRAFKKILARGDRKRIV | TIAM1 | 1 | 0.3445 |
| ALPHA-2A AR | HDFRRAFKKILARGDRKRIV | TIAM1 | 1 | 0.435 |
| ALPHA-2A AR | HDFRRAFKKILARGDRKRIV | TIAM2 | 1 | 0.2445 |
| ALPHA-2A AR | HDFRRAFKKILARGDRKRIV | TIAM2 | 1 | 0.378 |
| ALPHA-2A AR | HDFRRAFKKILARGDRKRIV | TIAM2 | 1 | 0.378 |
| ALPHA-2A AR | HDFRRAFKKILARGDRKRIV | TIP1 | 1 | 0.802 |
| ALPHA-2A AR | HDFRRAFKKILARGDRKRIV | TIP1 | 1 | 1.309 |
| ALPHA-2A AR | HDFRRAFKKILARGDRKRIV | TIP2 | 1 | 0.4165 |
| ALPHA-2A AR | HDFRRAFKKILARGDRKRIV | TIP2 | 1 | 0.6065 |
| ALPHA-2A AR | HDFRRAFKKILARGDRKRIV | VARTUL | 1 | 0.287 |
| ALPHA-2A AR | HDFRRAFKKILARGDRKRIV | VARTUL | 1 | 0.1525 |
| ALPHA-2A AR | HDFRRAFKKILARGDRKRIV | VARTUL | 2 | 0.3335 |
| ALPHA-2A AR | HDFRRAFKKILARGDRKRIV | VARTUL | 2 | 0.2375 |
| ALPHA-2A AR | HDFRRAFKKILARGDRKRIV | VARTUL | 3 | 0.2985 |
| ALPHA-2A AR | HDFRRAFKKILARGDRKRIV | VARTUL | 3 | 0.1235 |
| ALPHA-2A AR | HDFRRAFKKILARGDRKRIV | VARTUL | 4 | 0.302 |
| ALPHA-2A AR | HDFRRAFKKILARGDRKRIV | VARTUL | 4 | 0.1805 |
| ALPHA-2A AR | HDFRRAFKKILARGDRKRIV | VARTUL | 1, 2 | 0.3665 |
| ALPHA-2A AR | HDFRRAFKKILARGDRKRIV | VARTUL | 1, 2 | 0.555 |
| ALPHA-2A AR | HDFRRAFKKILARGDRKRIV | X-11 BETA | 1 | 1.3435 |
| ALPHA-2A AR | HDFRRAFKKILARGDRKRIV | X-11 BETA | 1 | 1.0755 |
| ALPHA-2A AR | HDFRRAFKKILARGDRKRIV | X-11 BETA | 2 | 0.5205 |
| ALPHA-2A AR | HDFRRAFKKILARGDRKRIV | X-11 BETA | 2 | 0.345 |
| ALPHA-2A AR | HDFRRAFKKILARGDRKRIV | X-11 BETA | 1, 2 | 2.63 |
| ALPHA-2A AR | HDFRRAFKKILARGDRKRIV | X-11 BETA | 1, 2 | 3.6965 |
| ALPHA-2A AR | HDFRRAFKKILARGDRKRIV | ZO-1 | 1 | 3.758 |
| ALPHA-2A AR | HDFRRAFKKILARGDRKRIV | ZO-1 | 2 | 3.0035 |
| ALPHA-2A AR | HDFRRAFKKILARGDRKRIV | ZO-1 | 2 | 3.2305 |
| ALPHA-2A AR | HDFRRAFKKILARGDRKRIV | ZO-1 | 3 | 0.3305 |
| ALPHA-2A AR | HDFRRAFKKILARGDRKRIV | ZO-1 | 3 | 0.7565 |
| ALPHA-2A AR | HDFRRAFKKILARGDRKRIV | ZO-2 | 1 | 0.5655 |
| ALPHA-2A AR | HDFRRAFKKILARGDRKRIV | ZO-2 | 1 | 0.4095 |
| ALPHA-2A AR | HDFRRAFKKILARGDRKRIV | ZO-2 | 2 | 1.3775 |
| ALPHA-2A AR | HDFRRAFKKILARGDRKRIV | ZO-2 | 2 | 1.5355 |
| ALPHA-2A AR | HDFRRAFKKILARGDRKRIV | ZO-2 | 3 | 0.1415 |
| ALPHA-2A AR | HDFRRAFKKILARGDRKRIV | ZO-2 | 3 | 0.2935 |
| ALPHA-2A AR | HDFRRAFKKILARGDRKRIV | ZO-3 | 1 | 0.578 |
| ALPHA-2A AR | HDFRRAFKKILARGDRKRIV | ZO-3 | 1 | 0.746 |
| ALPHA-2A AR | HDFRRAFKKILARGDRKRIV | ZO-3 | 2 | 2.5585 |
| ALPHA-2A AR | HDFRRAFKKILARGDRKRIV | ZO-3 | 2 | 3.245 |
| ALPHA-2A AR | HDFRRAFKKILARGDRKRIV | ZO-3 | 3 | 0.2365 |
| alpha-2A AR | HDFRRAFKKILARGDRKRIV | ZO-3 | 3 | 0.4715 |

TABLE 8B

| | | | | |
|---|---|---|---|---|
| AVC NAME | SEQUENCE | GENE NAME | DOMAIN | AVERAGE OD |

| | | | | |
|---|---|---|---|---|
| alpha-2B AR | QDFRRAFRRILARPWTQTAW | AF6 | 1 | 1.988 |
| alpha-2B AR | ODFRRAFRRILARPWTQTAW | AF6 | 1 | 2.387 |
| alpha-2B AR | QDFRRAFRRILARPWTQTAW | AF6 | 1 | 2.233 |
| alpha-2B AR | QDFRRAFRRILARPWTQTAW | AIPC | 1 | 1.539 |
| alpha-2B AR | QDFRRAFRRILARPWTQTAW | AIPC | 1 | 0.576 |
| alpha-2B AR | QDFRRAFRRILARPWTQTAW | AIPC | 1 | 1.028 |
| alpha-2B AR | QDFRRAFRRILARPWTQTAW | AIPC | 1 | 1.7515 |
| alpha-26 AR | QDFRRAFRRILARPWTQTAW | AIPC | 3 | 0.404 |
| alpha-2B AR | QDFRRAFRRILARPWTQTAW | AIPC | 3 | 0.788 |
| alpha-2B AR | QDFRRAFRRILARPWTQTAW | AIPC | 4 | 1.117 |
| alpha-2B AR | QDFRRAFRRILARPWTQTAW | AIPC | 4 | 0.508 |
| alpha-2B AR | QDFRRAFRRILARPWTQTAW | ALP | 1 | 0.953 |
| alpha-2B AR | QDFRRAFRRILARPWTQTAW | ALP | 1 | 1.3375 |
| alpha-2B AR | QDFRRAFRRILARPWTQTAW | APXL1 | 1 | 2.005 |
| alpha-2B AR | QDFRRAFRRILARPWTQTAW | APXL1 | 1 | 0.979 |
| alpha-2B AR | QDFRRAFRRILARPWTQTAW | CARD14 | 1 | 1.8785 |
| alpha-2B AR | QDFRRAFRRILARPWTQTAW | CARD14 | 1 | 1.144 |
| alpha-2B AR | QDFRRAFRRILARPWTQTAW | CASK | 1 | 2.2245 |
| alpha-2B AR | QDFRRAFRRILARPWTQTAW | CASK | 1 | 1.905 |
| alpha-2B AR | QDFRRAFRRILARPWTQTAW | CASK | 1 | 2.139 |
| alpha-2B AR | QDFRRAFRRILARPWTQTAW | CNK1 | 1 | 1.3535 |
| alpha-2B AR | QDFRRAFRRILARPWTQTAW | CNK1 | 1 | 0.8095 |
| alpha-2B AR | QDFRRAFRRILARPWTQTAW | Cytohesin | 1 | 1.968 |
| | | binding | | |
| | | Protein | | |
| alpha-2B AR | QDFRRAFRRILARPWTQTAW | Cytohesin | 1 | 2.1155 |
| | | binding | | |
| | | Protein | | |
| alpha-2B AR | QDFRRAFRRILARPWTQTAW | Cytohesin | 1 | 1.878 |
| | | binding | | |
| | | Protein | | |
| alpha-2B AR | QDFRRAFRRILARPWTQTAW | DLG1 | 1 | 1.49 |
| alpha-2B AR | QDFRRAFRRILARPWTQTAW | DLG1 | 1 | 0.939 |
| alpha-2B AR | QDFRRAFRRILARPWTQTAW | DLG1 | 2 | 1.597 |
| alpha-2B AR | QDFRRAFRRILARPWTQTAW | DLG1 | 2 | 1.1225 |
| alpha-2B AR | QDFRRAFRRILARPWTQTAW | DLG1 | 3 | 1.14 |
| alpha-2B AR | QDFRRAFRRILARPWTQTAW | DLG1 | 3 | 2.0895 |
| alpha-2B AR | QDFRRAFRRILARPWTQTAW | DLG1 | 1, 2 | 2.083 |
| alpha-2B AR | QDFRRAFRRILARPWTQTAW | DLG1 | 1, 2 | 2.4735 |
| alpha-2B AR | QDFRRAFRRILARPWTQTAW | DLG1 | 1, 2 | 2.1545 |
| alpha-2B AR | QDFRRAFRRILARPWTQTAW | DLG2 | 1 | 0.6645 |
| alpha-2B AR | QDFRRAFRRILARPWTQTAW | DLG2 | 1 | 0.885 |
| alpha-2B AR | QDFRRAFRRILARPWTQTAW | DLG2 | 2 | 0.7655 |
| alpha-2B AR | QDFRRAFRRILARPWTQTAW | DLG2 | 2 | 1.3695 |
| alpha-2B AR | QDFRRAFRRILARPWTQTAW | DLG5 | 1 | 1.0645 |
| alpha-2B AR | QDFRRAFRRILARPWTQTAW | DLG5 | 1 | 0.6255 |
| alpha-2B AR | QDFRRAFRRILARPWTQTAW | DLG5 | 2 | 2.2525 |
| alpha-2B AR | QDFRRAFRRILARPWTQTAW | DLG5 | 2 | 2.822 |
| alpha-2B AR | QDFRRAFRRILARPWTQTAW | DLG5 | 2 | 2.4085 |
| alpha-2B AR | QDFRRAFRRILARPWTQTAW | DLG5 | 2 | 1.1375 |
| alpha-2B AR | QDFRRAFRRILARPWTQTAW | DLG5 | 2 | 0.568 |
| alpha-2B AR | QDFRRAFRRILARPWTQTAW | DVL2 | 1 | 1.1125 |
| alpha-2B AR | QDFRRAFRRILARPWTQTAW | DVL2 | 1 | 1.962 |
| alpha-2B AR | QDFRRAFRRILARPWTQTAW | DVL3 | 1 | 2.5155 |
| alpha-2B AR | QDFRRAFRRILARPWTQTAW | DVL3 | 1 | 2.0525 |
| alpha-2B AR | QDFRRAFRRILARPWTQTAW | EBP50 | 1 | 0.7175 |
| alpha-2B AR | QDFRRAFRRILARPWTQTAW | EBP50 | 1 | 1.3475 |
| alpha-2B AR | QDFRRAFRRILARPWTQTAW | EBP50 | 2 | 0.6575 |
| alpha-2B AR | QDFRRAFRRILARPWTQTAW | EBP50 | 2 | 1.14 |
| alpha-2B AR | QDFRRAFRRILARPWTQTAW | EBP50 | 1, 2 | 1.14 |
| alpha-2B AR | QDFRRAFRRILARPWTQTAW | EBP50 | 1, 2 | 0.6035 |
| alpha-2B AR | QDFRRAFRRILARPWTQTAW | ENIGMA | 1 | 0.8495 |
| alpha-2B AR | QDFRRAFRRILARPWTQTAW | ENIGMA | 1 | 1.5175 |
| alpha-2B AR | QDFRRAFRRILARPWTQTAW | ERBIN | 1 | 0.7835 |
| alpha-2B AR | QDFRRAFRRILARPWTQTAW | ERBIN | 1 | 1.4045 |
| alpha-2B AR | QDFRRAFRRILARPWTQTAW | FLJ00011 | 1 | 0.6075 |
| alpha-2B AR | QDFRRAFRRILARPWTQTAW | FLJ00011 | 1 | 1.2535 |
| alpha-2B AR | QDFRRAFRRILARPWTQTAW | FLJ11215 | 1 | 1.1605 |
| alpha-2B AR | QDFRRAFRRILARPWTQTAW | FLJ11215 | 1 | 0.5095 |
| alpha-2B AR | QDFRRAFRRILARPWTQTAW | FLJ12615 | 1 | 0.5005 |
| alpha-2B AR | QDFRRAFRRILARPWTQTAW | FLJ12615 | 1 | 1.013 |
| alpha-2B AR | QDFRRAFRRILARPWTQTAW | FLJ21687 | 1 | 1.204 |
| alpha-2B AR | QDFRRAFRRILARPWTQTAW | FLJ21687 | 1 | 0.628 |
| alpha-2B AR | QDFRRAFRRILARPWTQTAW | GRIP 1 | 4 | 0.5325 |
| alpha-2B AR | QDFRRAFRRILARPWTQTAW | GRIP 1 | 4 | 2.5575 |
| alpha-2B AR | QDFRRAFRRILARPWTQTAW | GRIP 1 | 5 | 0.6365 |
| alpha-2B AR | QDFRRAFRRILARPWTQTAW | GRIP 1 | 5 | 0.9375 |
| alpha-2B AR | QDFRRAFRRILARPWTQTAW | GRIP 1 | 6 | 1.519 |
| alpha-2B AR | QDFRRAFRRILARPWTQTAW | GRIP 1 | 6 | 0.993 |
| alpha-2B AR | QDFRRAFRRILARPWTQTAW | GRIP 1 | 7 | 0.7745 |
| alpha-2B AR | QDFRRAFRRILARPWTQTAW | GRIP 1 | 7 | 0.88 |
| alpha-2B AR | QDFRRAFRRILARPWTQTAW | Guanine | 1 | 0.58 |
| | | exchange | | |
| | | factor | | |
| alpha-2B AR | QDFRRAFRRILARPWTQTAW | Guanine | 1 | 1.2065 |
| | | exchange | | |
| | | factor | | |
| alpha-2B AR | QDFRRAFRRILARPWTQTAW | HEMBA | 1 | 1.3575 |
| | | 1003117 | | |
| alpha-2B AR | QDFRRAFRRILARPWTQTAW | HEMBA | 1 | 0.546 |
| | | 1003117 | | |
| alpha-2B AR | QDFRRAFRRILARPWTQTAW | HEMBA | 1 | 0.7805 |
| | | 1003117 | | |
| alpha-2B AR | QDFRRAFRRILARPWTQTAW | HEMBA | 1 | 1.432 |
| | | 1003117 | | |
| alpha-2B AR | QDFRRAFRRILARPWTQTAW | INADL | 1 | 1.196 |
| alpha-2B AR | QDFRRAFRRILARPWTQTAW | INADL | 1 | 1.2095 |
| alpha-2B AR | QDFRRAFRRILARPWTQTAW | INADL | 2 | 1.2635 |
| alpha-2B AR | QDFRRAFRRILARPWTQTAW | INADL | 2 | 1.2545 |
| alpha-2B AR | QDFRRAFRRILARPWTQTAW | INADL | 3 | 2.2165 |
| alpha-2B AR | QDFRRAFRRILARPWTQTAW | INADL | 3 | 1.3695 |
| alpha-2B AR | QDFRRAFRRILARPWTQTAW | INADL | 4 | 1.799 |
| alpha-2B AR | QDFRRAFRRILARPWTQTAW | INADL | 4 | 1.582 |
| alpha-2B AR | QDFRRAFRRILARPWTQTAW | INADL | 5 | 2.169 |
| alpha-2B AR | QDFRRAFRRILARPWTQTAW | INADL | 5 | 1.646 |
| alpha-2B AR | QDFRRAFRRILARPWTQTAW | INADL | 7 | 1.925 |
| alpha-2B AR | QDFRRAFRRILARPWTQTAW | INADL | 7 | 1.331 |
| alpha-2B AR | QDFRRAFRRILARPWTQTAW | INADL | 8 | 2.5575 |
| alpha-2B AR | QDFRRAFRRILARPWTQTAW | INADL | 8 | 2.4085 |
| alpha-2B AR | QDFRRAFRRILARPWTQTAW | KIAA0316 | 1 | 1.1905 |
| alpha-2B AR | QDFRRAFRRILARPWTQTAW | KIAA0316 | 1 | 0.6525 |
| alpha-2B AR | QDFRRAFRRILARPWTQTAW | KIAA0340 | 1 | 0.606 |
| alpha-2B AR | QDFRRAFRRILARPWTQTAW | KIAA0340 | 1 | 1.175 |
| alpha-2B AR | QDFRRAFRRILARPWTQTAW | KIAA0380 | 1 | 2.442 |
| alpha-2B AR | QDFRRAFRRILARPWTQTAW | KIAA0380 | 1 | 1.8915 |
| alpha-2B AR | QDFRRAFRRILARPWTQTAW | KIAA0380 | 1 | 2.731 |
| alpha-2B AR | QDFRRAFRRILARPWTQTAW | KIAA0382 | 1 | 0.5745 |
| alpha-2B AR | QDFRRAFRRILARPWTQTAW | KIAA0382 | 1 | 1.1175 |
| alpha-2B AR | QDFRRAFRRILARPWTQTAW | KIAA0440 | 1 | 2.6715 |
| alpha-2B AR | QDFRRAFRRILARPWTQTAW | KIAA0440 | 1 | 1.7615 |
| alpha-2B AR | QDFRRAFRRILARPWTQTAW | KIAA0440 | 1 | 2.9815 |
| alpha-2B AR | QDFRRAFRRILARPWTQTAW | KIAA0559 | 1 | 1.3815 |
| alpha-2B AR | QDFRRAFRRILARPWTQTAW | KIAA0559 | 1 | 1.677 |
| alpha-2B AR | QDFRRAFRRILARPWTQTAW | KIAA0751 | 1 | 1.6935 |
| alpha-2B AR | QDFRRAFRRILARPWTQTAW | KIAA0751 | 1 | 2.1475 |
| alpha-2B AR | QDFRRAFRRILARPWTQTAW | KIAA0751 | 1 | 1.485 |
| alpha-2B AR | QDFRRAFRRILARPWTQTAW | KIAA0858 | 1 | 1.7685 |
| alpha-2B AR | QDFRRAFRRILARPWTQTAW | KIAA0858 | 1 | 1.134 |
| alpha-2B AR | QDFRRAFRRILARPWTQTAW | KIAA0967 | 1 | 0.504 |
| alpha-2B AR | QDFRRAFRRILARPWTQTAW | KIAA0967 | 1 | 0.869 |
| alpha-2B AR | QDFRRAFRRILARPWTQTAW | KIAA1095 | 1 | 1.5 |
| alpha-2B AR | QDFRRAFRRILARPWTQTAW | KIAA1095 | 1 | 0.8115 |
| alpha-2B AR | QDFRRAFRRILARPWTQTAW | KIAA1222 | 1 | 0.9555 |
| alpha-2B AR | QDFRRAFRRILARPWTQTAW | KIAA1222 | 1 | 0.57 |
| alpha-2B AR | QDFRRAFRRILARPWTQTAW | KIAA1284 | 1 | 0.5985 |
| alpha-2B AR | QDFRRAFRRILARPWTQTAW | KIAA1284 | 1 | 1.537 |
| alpha-2B AR | QDFRRAFRRILARPWTQTAW | KIAA1415 | 1 | 0.598 |
| alpha-2B AR | QDFRRAFRRILARPWTQTAW | KIAA1415 | 1 | 2.3885 |
| alpha-2B AR | QDFRRAFRRILARPWTQTAW | KIAA1526 | 1 | 0.6885 |
| alpha-2B AR | QDFRRAFRRILARPWTQTAW | KIAA1526 | 1 | 1.462 |
| alpha-2B AR | QDFRRAFRRILARPWTQTAW | KIAA1526 | 1 | 1.3295 |
| alpha-2B AR | QDFRRAFRRILARPWTQTAW | KIAA1526 | 1 | 0.931 |
| alpha-2B AR | QDFRRAFRRILARPWTQTAW | KIAA1526 | 2 | 0.6855 |
| alpha-2B AR | QDFRRAFRRILARPWTQTAW | KIAA1526 | 2 | 1.6875 |
| alpha-2B AR | QDFRRAFRRILARPWTQTAW | KIAA1526 | 2 | 0.804 |
| alpha-2B AR | QDFRRAFRRILARPWTQTAW | KIAA1526 | 2 | 0.534 |
| alpha-2B AR | QDFRRAFRRILARPWTQTAW | KIAA1620 | 1 | 0.575 |
| alpha-2B AR | QDFRRAFRRILARPWTQTAW | KIAA1620 | 1 | 1.9325 |
| alpha-2B AR | QDFRRAFRRILARPWTQTAW | KIAA1719 | 1 | 0.6145 |
| alpha-2B AR | QDFRRAFRRILARPWTQTAW | KIAA1719 | 1 | 1.5 |
| alpha-2B AR | QDFRRAFRRILARPWTQTAW | KIAA1719 | 2 | 1.448 |
| alpha-2B AR | QDFRRAFRRILARPWTQTAW | KIAA1719 | 2 | 0.5935 |
| alpha-2B AR | QDFRRAFRRILARPWTQTAW | KIAA1719 | 3 | 3.5805 |
| alpha-2B AR | QDFRRAFRRILARPWTQTAW | KIAA1719 | 3 | 2.316 |
| alpha-2B AR | QDFRRAFRRILARPWTQTAW | KIAA1719 | 4 | 0.523 |
| alpha-2B AR | QDFRRAFRRILARPWTQTAW | KIAA1719 | 4 | 1.094 |
| alpha-2B AR | QDFRRAFRRILARPWTQTAW | KIAA1719 | 5 | 0.6855 |
| alpha-2B AR | QDFRRAFRRILARPWTQTAW | KIAA1719 | 5 | 1.6365 |
| alpha-2B AR | QDFRRAFRRILARPWTQTAW | KIAA1719 | 6 | 0.85 |
| alpha-2B AR | QDFRRAFRRILARPWTQTAW | KIAA1719 | 6 | 1.713 |
| alpha-2B AR | QDFRRAFRRILARPWTOTAW | LIM mystique | 1 | 0.6555 |
| alpha-2B AR | QDFRRAFRRILARPWTQTAW | LIM mystique | 1 | 0.706 |
| alpha-2B AR | QDFRRAFRRILARPWTQTAW | LIM protein | 1 | 1.847 |
| alpha-2B AR | QDFRRAFRRILARPWTQTAW | LIM protein | 1 | 2.109 |
| alpha-2B AR | QDFRRAFRRILARPWTQTAW | LIM-RIL | 1 | 1.9115 |
| alpha-2B AR | QDFRRAFRRILARPWTQTAW | LIM-RIL | 1 | 1.1165 |
| alpha-2B AR | QDFRRAFRRILARPWTQTAW | LIMK1 | 1 | 1.6515 |
| alpha-2B AR | QDFRRAFRRILARPWTQTAW | LIMK1 | 1 | 1.7335 |
| alpha-2B AR | QDFRRAFRRILARPWTQTAW | LIMK2 | 1 | 2.963 |
| alpha-2B AR | QDFRRAFRRILARPWTQTAW | LIMK2 | 1 | 2.196 |
| alpha-2B AR | QDFRRAFRRILARPWTQTAW | LU-1 | 1 | 0.718 |
| alpha-2B AR | QDFRRAFRRILARPWTQTAW | LU-1 | 1 | 0.6275 |
| alpha-2B AR | QDFRRAFRRILARPWTQTAW | MAGI 1 | 1 | 1.5685 |
| alpha-2B AR | QDFRRAFRRILARPWTQTAW | MAGI 1 | 1 | 0.9585 |
| alpha-2B AR | QDFRRAFRRILARPWTQTAW | MAGI 1 | 1 | 3.5185 |
| alpha-2B AR | QDFRRAFRRILARPWTQTAW | MAGI 1 | 1 | 3.231 |
| alpha-2B AR | QDFRRAFRRILARPWTQTAW | MAGI 1 | 3 | 1.863 |
| alpha-2B AR | QDFRRAFRRILARPWTQTAW | MAGI 1 | 3 | 1.2295 |
| alpha-2B AR | QDFRRAFRRILARPWTQTAW | MAGI 1 | 3 | 1.4925 |
| alpha-2B AR | QDFRRAFRRILARPWTQTAW | MAGI 1 | 3 | 1.6005 |
| alpha-2B AR | QDFRRAFRRILARPWTQTAW | MAGI 1 | 4 | 4 |
| alpha-2B AR | QDFRRAFRRILARPWTQTAW | MAGI 1 | 4 | 4 |
| alpha-2B AR | QDFRRAFRRILARPWTQTAW | MAGI 1 | 5 | 1.267 |
| alpha-2B AR | QDFRRAFRRILARPWTQTAW | MAGI 1 | 5 | 1.295 |
| alpha-2B AR | QDFRRAFRRILARPWTQTAW | Magi 2 | 1 | 0.755 |
| alpha-2B AR | QDFRRAFRRILARPWTQTAW | Magi 2 | 1 | 1.3725 |
| alpha-2B AR | QDFRRAFRRILARPWTQTAW | Magi 2 | 2 | 0.508 |
| alpha-2B AR | QDFRRAFRRILARPWTQTAW | Magi 2 | 2 | 0.8235 |
| alpha-2B AR | QDFRRAFRRILARPWTQTAW | Magi 2 | 3 | 2.228 |
| alpha-2B AR | QDFRRAFRRILARPWTQTAW | Magi 2 | 3 | 2.93 |
| alpha-2B AR | QDFRRAFRRILARPWTQTAW | Magi 2 | 4 | 0.42 |
| alpha-2B AR | QDFRRAFRRILARPWTQTAW | Magi 2 | 4 | 0.9925 |
| alpha-2B AR | QDFRRAFRRILARPWTQTAW | Magi 2 | 5 | 1.9195 |
| alpha-2B AR | QDFRRAFRRILARPWTQTAW | Magi 2 | 5 | 0.772 |
| alpha-2B AR | QDFRRAFRRILARPWTQTAW | Magi 2 | 6 | 1.487 |
| alpha-2B AR | QDFRRAFRRILARPWTQTAW | Magi 2 | 6 | 0.555 |
| alpha-2B AR | QDFRRAFRRILARPWTQTAW | MAGI 3 | 1 | 1.8545 |
| alpha-2B AR | QDFRRAFRRILARPWTQTAW | MAGI 3 | 1 | 2.576 |
| alpha-2B AR | QDFRRAFRRILARPWTQTAW | MAGI 3 | 2 | 2.0285 |
| alpha-2B AR | QDFRRAFRRILARPWTQTAW | MAGI 3 | 2 | 0.1245 |
| alpha-2B AR | QDFRRAFRRILARPWTQTAW | MAGI 3 | 3 | 2.02 |
| alpha-2B AR | QDFRRAFRRILARPWTQTAW | MAGI 3 | 3 | 1.348 |
| alpha-2B AR | QDFRRAFRRILARPWTQTAW | MAGI 3 | 4 | 1.213 |
| alpha-2B AR | QDFRRAFRRILARPWTQTAW | MAGI 3 | 4 | 1.7545 |
| alpha-2B AR | QDFRRAFRRILARPWTQTAW | MAGI 3 | 5 | 2.174 |
| alpha-2B AR | QDFRRAFRRILARPWTQTAW | MAGI 3 | 5 | 1.447 |
| alpha-2B AR | QDFRRAFRRILARPWTQTAW | MAST1 | 1 | 1.856 |
| alpha-2B AR | QDFRRAFRRILARPWTQTAW | MAST1 | 1 | 1.5595 |
| alpha-2B AR | QDFRRAFRRILARPWTQTAW | MAST2 | 1 | 4.0515 |
| alpha-2B AR | QDFRRAFRRILARPWTQTAW | MAST2 | 1 | 2.4955 |
| alpha-2B AR | QDFRRAFRRILARPWTQTAW | MAST4 | 1 | 3.97 |
| alpha-2B AR | QDFRRAFRRILARPWTQTAW | MAST4 | 1 | 2.581 |
| alpha-2B AR | QDFRRAFRRILARPWTQTAW | MAST4 | 1 | 4 |
| alpha-2B AR | QDFRRAFRRILARPWTQTAW | MINT1 | 1 | 1.5615 |
| alpha-2B AR | QDFRRAFRRILARPWTQTAW | MINT1 | 1 | 0.8725 |
| alpha-2B AR | QDFRRAFRRILARPWTQTAW | MINT1 | 2 | 1.3535 |
| alpha-2B AR | QDFRRAFRRILARPWTQTAW | MINT1 | 2 | 0.8485 |
| alpha-2B AR | QDFRRAFRRILARPWTQTAW | MINT1 | 1, 2 | 2.744 |
| alpha-2B AR | QDFRRAFRRILARPWTQTAW | MINT1 | 1, 2 | 3.084 |
| alpha-2B AR | QDFRRAFRRILARPWTQTAW | MPP1 | 1 | 2.216 |
| alpha-2B AR | QDFRRAFRRILARPWTQTAW | MPP1 | 1 | 2.2205 |
| alpha-2B AR | QDFRRAFRRILARPWTQTAW | MPP2 | 1 | 3.5385 |
| alpha-2B AR | QDFRRAFRRILARPWTQTAW | MPP2 | 1 | 2.4015 |
| alpha-2B AR | QDFRRAFRRILARPWTQTAW | MUPP1 | 1 | 0 |
| alpha-2B AR | QDFRRAFRRILARPWTQTAW | MUPP1 | 1 | 3.9855 |
| alpha-2B AR | QDFRRAFRRILARPWTQTAW | MUPP1 | 2 | 0 |
| alpha-2B AR | QDFRRAFRRILARPWTQTAW | MUPP1 | 2 | 3.774 |
| alpha-2B AR | QDFRRAFRRILARPWTQTAW | MUPP1 | 3 | 3.9815 |
| alpha-2B AR | QDFRRAFRRILARPWTQTAW | MUPP1 | 3 | 0 |
| alpha-2B AR | QDFRRAFRRILARPWTQTAW | MUPP1 | 4 | 3.8085 |
| alpha-2B AR | QDFRRAFRRILARPWTQTAW | MUPP1 | 4 | 0 |
| alpha-2B AR | QDFRRAFRRILARPWTQTAW | MUPP1 | 5 | 3.9975 |
| alpha-2B AR | QDFRRAFRRILARPWTQTAW | MUPP1 | 5 | 0 |
| alpha-2B AR | QDFRRAFRRILARPWTQTAW | MUPP1 | 6 | 0 |
| alpha-2B AR | QDFRRAFRRILARPWTQTAW | MUPP1 | 6 | 3.928 |
| alpha-2B AR | QDFRRAFRRILARPWTQTAW | MUPP1 | 7 | 0 |
| alpha-2B AR | QDFRRAFRRILARPWTQTAW | MUPP1 | 7 | 4 |
| alpha-2B AR | QDFRRAFRRILARPWTQTAW | MUPP1 | 8 | 0 |
| alpha-2B AR | QDFRRAFRRILARPWTQTAW | MUPP1 | 8 | 3.818 |
| alpha-2B AR | QDFRRAFRRILARPWTQTAW | MUPP1 | 9 | 0 |
| alpha-2B AR | QDFRRAFRRILARPWTQTAW | MUPP1 | 9 | 4 |
| alpha-2B AR | QDFRRAFRRILARPWTQTAW | MUPP1 | 10 | 0.967 |
| alpha-2B AR | QDFRRAFRRILARPWTQTAW | MUPP1 | 10 | 2.152 |
| alpha-2B AR | QDFRRAFRRILARPWTQTAW | MUPP1 | 11 | 0.579 |
| alpha-2B AR | QDFRRAFRRILARPWTQTAW | MUPP1 | 11 | 1.192 |
| alpha-2B AR | QDFRRAFRRILARPWTQTAW | MUPP1 | 12 | 0.623 |
| alpha-2B AR | QDFRRAFRRILARPWTQTAW | MUPP1 | 12 | 1.173 |
| alpha-2B AR | QDFRRAFRRILARPWTQTAW | MUPP1 | 13 | 1.0195 |
| alpha-2B AR | QDFRRAFRRILARPWTQTAW | MUPP1 | 13 | 2.4275 |
| alpha-2B AR | QDFRRAFRRILARPWTQTAW | NeDLG | 1 | 1.1145 |
| alpha-2B AR | QDFRRAFRRILARPWTQTAW | NeDLG | 1 | 1.953 |
| alpha-2B AR | QDFRRAFRRILARPWTQTAW | NeDLG | 2 | 1.5645 |
| alpha-2B AR | QDFRRAFRRILARPWTQTAW | NeDLG | 2 | 0.9345 |
| alpha-2B AR | QDFRRAFRRILARPWTQTAW | NeDLG | 3 | 3.534 |
| alpha-2B AR | QDFRRAFRRILARPWTQTAW | NeDLG | 3 | 3.8255 |
| alpha-2B AR | QDFRRAFRRILARPWTQTAW | NeDLG | 1, 2 | 2.9895 |
| alpha-2B AR | QDFRRAFRRILARPWTQTAW | NeDLG | 1, 2 | 2.4485 |
| alpha-2B AR | QDFRRAFRRILARPWTQTAW | NOS1 | 1 | 3.5405 |
| alpha-2B AR | QDFRRAFRRILARPWTQTAW | NOS1 | 1 | 2.515 |
| alpha-2B AR | QDFRRAFRRILARPWTQTAW | novel | 1 | 1.7425 |
| | | PDZ gene | | |
| alpha-2B AR | QDFRRAFRRILARPWTQTAW | novel | 1 | 1.193 |
| | | PDZ gene | | |
| alpha-2B AR | QDFRRAFRRILARPWTQTAW | novel | 2 | 2.1985 |
| | | PDZ gene | | |
| alpha-2B AR | QDFRRAFRRILARPWTQTAW | novel | 2 | 1.4345 |
| | | PDZ gene | | |
| alpha-2B AR | QDFRRAFRRILARPWTQTAW | outer | 1 | 0.68 |
| | | membrane | | |
| alpha-2B AR | QDFRRAFRRILARPWTQTAW | outer | 1 | 1.312 |
| | | membrane | | |
| alpha-2B AR | QDFRRAFRRILARPWTQTAW | p55T | 1 | 0.488 |
| alpha-2B AR | QDFRRAFRRILARPWTQTAW | p55T | 1 | 0.8315 |
| alpha-2B AR | QDFRRAFRRILARPWTQTAW | PAR3 | 3 | 1.396 |
| alpha-2B AR | QDFRRAFRRILARPWTQTAW | PAR3 | 3 | 0.597 |
| alpha-2B AR | QDFRRAFRRILARPWTQTAW | PAR6 | 1 | 1.616 |
| alpha-2B AR | QDFRRAFRRILARPWTQTAW | PAR6 | 1 | 2.278 |
| alpha-2B AR | QDFRRAFRRILARPWTQTAW | PAR6 GAMMA | 1 | 0.3865 |
| alpha-2B AR | QDFRRAFRRILARPWTQTAW | PAR6 GAMMA | 1 | 0.914 |
| alpha-2B AR | QDFRRAFRRILARPWTQTAW | PDZ-73 | 2 | 2.112 |
| alpha-2B AR | QDFRRAFRRILARPWTQTAW | PDZ-73 | 2 | 1.3175 |
| alpha-2B AR | QDFRRAFRRILARPWTQTAW | PDZK1 | 1 | 0.7315 |
| alpha-2B AR | QDFRRAFRRILARPWTQTAW | PDZK1 | 1 | 1.502 |
| alpha-2B AR | QDFRRAFRRILARPWTQTAW | PDZK1 | 2 | 1.5125 |
| alpha-2B AR | QDFRRAFRRILARPWTQTAW | PDZK1 | 2 | 2.7415 |
| alpha-2B AR | QDFRRAFRRILARPWTQTAW | PDZK1 | 3 | 0.726 |
| alpha-2B AR | QDFRRAFRRILARPWTQTAW | PDZK1 | 3 | 1.374 |
| alpha-2B AR | QDFRRAFRRILARPWTQTAW | PDZK1 | 4 | 0.826 |
| alpha-2B AR | QDFRRAFRRILARPWTQTAW | PDZK1 | 4 | 1.361 |
| alpha-2B AR | QDFRRAFRRILARPWTQTAW | PDZK1 | 2, 3, 4 | 2.1345 |
| alpha-2B AR | QDFRRAFRRILARPWTQTAW | PDZK1 | 2, 3, 4 | 2.597 |
| alpha-2B AR | QDFRRAFRRILARPWTQTAW | PICK1 | 1 | 2.458 |
| alpha-2B AR | QDFRRAFRRILARPWTQTAW | PICK1 | 1 | 1.3835 |
| alpha-2B AR | QDFRRAFRRILARPWTQTAW | PICK1 | 1 | 0.6615 |
| alpha-2B AR | QDFRRAFRRILARPWTQTAW | PICK1 | 1 | 1.4495 |
| alpha-2B AR | QDFRRAFRRILARPWTQTAW | PIST | 1 | 1.503 |
| alpha-2B AR | QDFRRAFRRILARPWTQTAW | PIST | 1 | 0.587 |
| alpha-2B AR | QDFRRAFRRILARPWTQTAW | prIL16 | 1 | 0.9865 |
| alpha-2B AR | QDFRRAFRRILARPWTQTAW | prIL16 | 1 | 0.474 |
| alpha-2B AR | QDFRRAFRRILARPWTQTAW | prIL16 | 2 | 0.4355 |
| alpha-2B AR | QDFRRAFRRILARPWTQTAW | prIL16 | 2 | 0.897 |
| alpha-2B AR | QDFRRAFRRILARPWTQTAW | prIL16 | 1, 2 | 2.0705 |
| alpha-2B AR | QDFRRAFRRILARPWTQTAW | prIL16 | 1, 2 | 1.9335 |
| alpha-2B AR | QDFRRAFRRILARPWTQTAW | PSD95 | 1 | 0.868 |
| alpha-2B AR | QDFRRAFRRILARPWTQTAW | PSD95 | 1 | 1.5915 |
| alpha-2B AR | QDFRRAFRRILARPWTQTAW | PSD95 | 3 | 2.976 |
| alpha-2B AR | QDFRRAFRRILARPWTQTAW | PSD95 | 3 | 3.742 |
| alpha-2B AR | QDFRRAFRRILARPWTQTAW | PSD95 | 1, 2, 3 | 4 |
| alpha-2B AR | QDFRRAFRRILARPWTQTAW | PSD95 | 1, 2, 3 | 4 |
| alpha-2B AR | QDFRRAFRRILARPWTQTAW | PTN-4 | 1 | 2.1145 |
| alpha-2B AR | QDFRRAFRRILARPWTQTAW | PTN-4 | 1 | 2.1945 |
| alpha-2B AR | QDFRRAFRRILARPWTQTAW | PTPL1 | 1 | 0.4725 |
| alpha-2B AR | QDFRRAFRRILARPWTQTAW | PTPL1 | 1 | 1.011 |
| alpha-2B AR | QDFRRAFRRILARPWTQTAW | PTPL1 | 2 | 0.688 |
| alpha-2B AR | QDFRRAFRRILARPWTQTAW | PTPL1 | 2 | 2.9835 |
| alpha-2B AR | QDFRRAFRRILARPWTQTAW | PTPL1 | 3 | 0.3955 |
| alpha-2B AR | QDFRRAFRRILARPWTQTAW | PTPL1 | 3 | 1.8235 |
| alpha-2B AR | QDFRRAFRRILARPWTQTAW | PTPL1 | 4 | 0.5795 |
| alpha-2B AR | QDFRRAFRRILARPWTQTAW | PTPL1 | 4 | 2.3485 |
| alpha-2B AR | QDFRRAFRRILARPWTQTAW | PTPL1 | 5 | 1.799 |
| alpha-2B AR | QDFRRAFRRILARPWTQTAW | PTPL1 | 5 | 0.49 |
| alpha-2B AR | QDFRRAFRRILARPWTQTAW | Shank 1 | 1 | 3.155 |
| alpha-2B AR | QDFRRAFRRILARPWTQTAW | Shank 1 | 1 | 1.549 |
| alpha-2B AR | QDFRRAFRRILARPWTQTAW | Shank 3 | 1 | 2.6715 |
| alpha-2B AR | QDFRRAFRRILARPWTQTAW | Shank 3 | 1 | 3.408 |
| alpha-2B AR | QDFRRAFRRILARPWTQTAW | Shank 3 | 1 | 1.3155 |
| alpha-2B AR | QDFRRAFRRILARPWTQTAW | SIP1 | 2 | 0.985 |
| alpha-2B AR | QDFRRAFRRILARPWTQTAW | SIP1 | 2 | 1.271 |
| alpha-2B AR | QDFRRAFRRILARPWTQTAW | SITAC 18 | 1 | 0.5955 |
| alpha-2B AR | QDFRRAFRRILARPWTQTAW | SITAC 18 | 1 | 1.087 |
| alpha-2B AR | QDFRRAFRRILARPWTQTAW | SITAC 18 | 2 | 1.058 |
| alpha-2B AR | QDFRRAFRRILARPWTQTAW | SITAC 18 | 2 | 2.0765 |
| alpha-2B AR | QDFRRAFRRILARPWTQTAW | Syntrophin | 1 | 2.426 |
| | | 1 alpha | | |
| alpha-2B AR | QDFRRAFRRILARPWTQTAW | Syntrophin | 1 | 2.69 |
| | | 1 alpha | | |
| alpha-2B AR | QDFRRAFRRILARPWTQTAW | Syntrophin | 1 | 0.5265 |
| | | gamma 1 | | |
| alpha-2B AR | QDFRRAFRRILARPWTQTAW | Syntrophin | 1 | 1.792 |
| | | gamma 1 | | |
| alpha-2B AR | QDFRRAFRRILARPWTQTAW | Syntrophin | 1 | 0.599 |
| | | gamma 2 | | |
| alpha-2B AR | QDFRRAFRRILARPWTQTAW | Syntrophin | 1 | 2.3375 |
| | | gamma 2 | | |
| alpha-2B AR | QDFRRAFRRILARPWTQTAW | TAX2-like | 1 | 1.314 |
| | | protein | | |
| alpha-2B AR | QDFRRAFRRILARPWTQTAW | TAX2-like | 1 | 1.544 |
| | | protein | | |
| alpha-2B AR | QDFRRAFRRILARPWTQTAW | TIAM1 | 1 | 1.639 |
| alpha-2B AR | QDFRRAFRRILARPWTQTAW | TIAM1 | 1 | 2.469 |
| alpha-2B AR | QDFRRAFRRILARPWTQTAW | TIAM2 | 1 | 0.786 |
| alpha-2B AR | QDFRRAFRRILARPWTQTAW | TIAM2 | 1 | 1.3665 |
| alpha-2B AR | QDFRRAFRRILARPWTQTAW | TIP1 | 1 | 4 |
| alpha-2B AR | QDFRRAFRRILARPWTQTAW | TIP1 | 1 | 4 |
| alpha-2B AR | QDFRRAFRRILARPWTQTAW | TIP2 | 1 | 1.439 |
| alpha-2B AR | QDFRRAFRRILARPWTQTAW | TIP2 | 1 | 1.766 |
| alpha-2B AR | QDFRRAFRRILARPWTQTAW | Vartul | 1 | 2.2825 |
| alpha-2B AR | QDFRRAFRRILARPWTQTAW | Vartul | 1 | 1.233 |
| alpha-2B AR | QDFRRAFRRILARPWTQTAW | Vartul | 2 | 0.6885 |
| alpha-2B AR | QDFRRAFRRILARPWTQTAW | Vartul | 2 | 1.187 |
| alpha-2B AR | QDFRRAFRRILARPWTQTAW | Vartul | 3 | 0.6335 |
| alpha-2B AR | QDFRRAFRRILARPWTQTAW | Vartul | 3 | 1.5135 |
| alpha-2B AR | QDFRRAFRRILARPWTQTAW | Vartul | 4 | 0.4915 |
| alpha-2B AR | QDFRRAFRRILARPWTQTAW | Vartul | 4 | 0.998 |
| alpha-2B AR | QDFRRAFRRILARPWTQTAW | Vartul | 1, 2 | 1.492 |
| alpha-2B AR | QDFRRAFRRILARPWTQTAW | Vartul | 1, 2 | 1.401 |
| alpha-2B AR | QDFRRAFRRILARPWTQTAW | Vartul | 1, 2 | 1.912 |
| alpha-2B AR | QDFRRAFRRILARPWTQTAW | X-11 beta | 1 | 1.3255 |
| alpha-2B AR | QDFRRAFRRILARPWTQTAW | X-11 beta | 1 | 0.7545 |
| alpha-2B AR | QDFRRAFRRILARPWTQTAW | X-11 beta | 2 | 0.4925 |
| alpha-2B AR | QDFRRAFRRILARPWTQTAW | X-11 beta | 2 | 0.9995 |
| alpha-2B AR | QDFRRAFRRILARPWTQTAW | X-11 beta | 1, 2 | 2.024 |
| alpha-2B AR | QDFRRAFRRILARPWTQTAW | X-11 beta | 1, 2 | 1.815 |
| alpha-2B AR | QDFRRAFRRILARPWTQTAW | ZO-1 | 1 | 1.7365 |
| alpha-2B AR | QDFRRAFRRILARPWTQTAW | ZO-1 | 1 | 0.711 |
| alpha-2B AR | QDFRRAFRRILARPWTQTAW | ZO-1 | 2 | 0.7205 |
| alpha-2B AR | QDFRRAFRRILARPWTQTAW | ZO-1 | 2 | 1.2305 |
| alpha-2B AR | QDFRRAFRRILARPWTQTAW | ZO-1 | 3 | 0.97 |
| alpha-2B AR | QDFRRAFRRILARPWTQTAW | ZO-1 | 3 | 0.681 |
| alpha-2B AR | QDFRRAFRRILARPWTQTAW | ZO-2 | 1 | 1.538 |
| alpha-2B AR | QDFRRAFRRILARPWTQTAW | ZO-2 | 1 | 0.744 |
| alpha-2B AR | QDFRRAFRRILARPWTQTAW | ZO-2 | 2 | 1.309 |
| alpha-2B AR | QDFRRAFRRILARPWTQTAW | ZO-2 | 2 | 0.808 |
| alpha-2B AR | QDFRRAFRRILARPWTQTAW | ZO-2 | 3 | 0.641 |
| alpha-2B AR | QDFRRAFRRILARPWTQTAW | ZO-2 | 3 | 1.12 |
| alpha-2B AR | QDFRRAFRRILARPWTQTAW | ZO-3 | 1 | 1.7115 |
| alpha-2B AR | QDFRRAFRRILARPWTQTAW | ZO-3 | 1 | 3.358 |
| alpha-2B AR | QDFRRAFRRILARPWTQTAW | ZO-3 | 1 | 1.33 |
| alpha-2B AR | QDFRRAFRRILARPWTQTAW | ZO-3 | 1 | 0 |
| alpha-2B AR | QDFRRAFRRILARPWTQTAW | ZO-3 | 2 | 3.742 |
| alpha-2B AR | QDFRRAFRRILARPWTQTAW | ZO-3 | 2 | 0 |
| alpha-2B AR | QDFRRAFRRILARPWTQTAW | ZO-3 | 3 | 3.4125 |
| alpha-2B AR | QDFRRAFRRILARPWTQTAW | ZO-3 | 3 | 0 |

TABLE 8C:

| | | | | |
|---|---|---|---|---|
| AVG NAME | SEQUENCE | GENE NAME | DOMAIN | AVERAGE OD |

| alpha-2C AR | DFRPSFKHILFRRARRGFRQ | AF6 | 1 | 1.943 |
|---|---|---|---|---|
| alpha-2C AR | DFRPSFKHILFRRARRGFRQ | AF6 | 1 | 1.7465 |
| alpha-2C AR | DFRPSFKHILFRRARRGFRQ | AIPC | 1 | 1.6195 |
| alpha-2C AR | DFRPSFKHILFRRARRGFRQ | AIPC | 1 | 2.454 |
| alpha-2C AR | DFRPSFKHILFRRARRGFRQ | AIPC | 1 | 3.4005 |
| alpha-2C AR | DFRPSFKHILFRRARRGFRQ | AIPC | 1 | 2.5865 |
| alpha-2C AR | DFRPSFKHILFRRARRGFRQ | AIPC | 3 | 1.714 |
| alpha-2C AR | DFRPSFKHILFRRARRGFRQ | AIPC | 3 | 1.3765 |
| alpha-2C AR | DFRPSFKHILFRRARRGFRQ | AIPC | 4 | 1.8395 |
| alpha-2C AR | DFRPSFKHILFRRARRGFRQ | AIPC | 4 | 1.6645 |
| alpha-2C AR | DFRPSFKHILFRRARRGFRQ | ALP | 1 | 3.093 |
| alpha-2C AR | DFRPSFKHILFRRARRGFRQ | ALP | 1 | 1.8765 |
| alpha-2C AR | DFRPSFKHILFRRARRGFRQ | APXL1 | 1 | 2.002 |
| alpha-2C AR | DFRPSFKHILFRRARRGFRQ | APXL1 | 1 | 3.4065 |
| alpha-2C AR | DFRPSFKHILFRRARRGFRQ | CARD 14 | 1 | 4 |
| alpha-2C AR | DFRPSFKHILFRRARRGFRQ | CARD 14 | 1 | 4.0725 |
| alpha-2C AR | DFRPSFKHILFRRARRGFRQ | CASK | 1 | 2.113 |
| alpha-2C AR | DFRPSFKHILFRRARRGFRQ | CASK | 1 | 1.8105 |
| alpha-2C AR | DFRPSFKHILFRRARRGFRQ | CNK1 | 1 | 1.862 |
| alpha-2C AR | DFRPSFKHILFRRARRGFRQ | CNK1 | 1 | 2.769 |
| alpha-2C AR | DFRPSFKHILFRRARRGFRQ | Cytohesin | 1 | 2.343 |
| | | binding | | |
| | | Protein | | |
| alpha-2C AR | DFRPSFKHILFRRARRGFRQ | Cytohesin | 1 | 2.0315 |
| | | binding | | |
| | | Protein | | |
| alpha-2C AR | DFRPSFKHILFRRARRGFRQ | DLG1 | 1 | 1.0915 |
| alpha-2C AR | DFRPSFKHILFRRARRGFRQ | DLG1 | 1 | 1.677 |
| alpha-2C AR | DFRPSFKHILFRRARRGFRQ | DLG1 | 2 | 1.8005 |
| alpha-2C AR | DFRPSFKHILFRRARRGFRQ | DLG1 | 2 | 1.2895 |
| alpha-2C AR | DFRPSFKHILFRRARRGFRQ | DLG1 | 3 | 1.9495 |
| alpha-2C AR | DFRPSFKHILFRRARRGFRQ | DLG1 | 3 | 3.024 |
| alpha-2C AR | DFRPSFKHILFRRARRGFRQ | DLG1 | 1, 2 | 2.013 |
| alpha-2C AR | DFRPSFKHILFRRARRGFRQ | DLG1 | 1, 2 | 2.2535 |
| alpha-2C AR | DFRPSFKHILFRRARRGFRQ | DLG2 | 1 | 1.462 |
| alpha-2C AR | DFRPSFKHILFRRARRGFRQ | DLG2 | 1 | 1.7675 |
| alpha-2C AR | DFRPSFKHILFRRARRGFRQ | DLG2 | 2 | 1.198 |
| alpha-2C AR | DFRPSFKHILFRRARRGFRQ | DLG2 | 2 | 1.6435 |
| alpha-2C AR | DFRPSFKHILFRRARRGFRQ | DLG5 | 1 | 2.2305 |
| alpha-2C AR | DFRPSFKHILFRRARRGFRQ | DLG5 | 1 | 1.7725 |
| alpha-2C AR | DFRPSFKHILFRRARRGFRQ | DLG5 | 2 | 2.6435 |
| alpha-2C AR | DFRPSFKHILFRRARRGFRQ | DLG5 | 2 | 2.722 |
| alpha-2C AR | DFRPSFKHILFRRARRGFRQ | DLG5 | 2 | 2.6385 |
| alpha-2C AR | DFRPSFKHILFRRARRGFRQ | DLG5 | 2 | 2.0345 |
| alpha-2C AR | DFRPSFKHILFRRARRGFRQ | DVL2 | 1 | 2.339 |
| alpha-2C AR | DFRPSFKHILFRRARRGFRQ | DVL2 | 1 | 3.345 |
| alpha-2C AR | DFRPSFKHILFRRARRGFRQ | DVL3 | 1 | 3.165 |
| alpha-2C AR | DFRPSFKHILFRRARRGFRQ | DVL3 | 1 | 3.4795 |
| alpha-2C AR | DFRPSFKHILFRRARRGFRQ | EBP50 | 1 | 1.364 |
| alpha-2C AR | DFRPSFKHILFRRARRGFRQ | EBP50 | 1 | 1.9775 |
| alpha-2C AR | DFRPSFKHILFRRARRGFRQ | EBP50 | 2 | 1.498 |
| alpha-2C AR | DFRPSFKHILFRRARRGFRQ | EBP50 | 2 | 1.894 |
| alpha-2C AR | DFRPSFKHILFRRARRGFRQ | EBP50 | 1, 2 | 1.351 |
| alpha-2C AR | DFRPSFKHILFRRARRGFRQ | EBP50 | 1, 2 | 1.7255 |
| alpha-2C AR | DFRPSFKHILFRRARRGFRQ | ENIGMA | 1 | 1.3755 |
| alpha-2C AR | DFRPSFKHILFRRARRGFRQ | ENIGMA | 1 | 2.0215 |
| alpha-2C AR | DFRPSFKHILFRRARRGFRQ | ERBIN | 1 | 1.5155 |
| alpha-2C AR | DFRPSFKHILFRRARRGFRQ | ERBIN | 1 | 1.6065 |
| alpha-2C AR | DFRPSFKHILFRRARRGFRQ | FLJ00011 | 1 | 1.4995 |
| alpha-2C AR | DFRPSFKHILFRRARRGFRQ | FLJ00011 | 1 | 1.8335 |
| alpha-2C AR | DFRPSFKHILFRRARRGFRQ | FLJ11215 | 1 | 1.292 |
| alpha-2C AR | DFRPSFKHILFRRARRGFRQ | FLJ11215 | 1 | 1.1735 |
| alpha-2C AR | DFRPSFKHILFRRARRGFRQ | FLJ1261S | 1 | 1.3565 |
| alpha-2C AR | DFRPSFKHILFRRARRGFRQ | FLJ12615 | 1 | 1.1595 |
| alpha-2C AR | DFRPSFKHILFRRARRGFRQ | FLJ21687 | 1 | 1.8625 |
| alpha-2C AR | DFRPSFKHILFRRARRGFRQ | FLJ21687 | 1 | 1.428 |
| alpha-2C AR | DFRPSFKHILFRRARRGFRQ | GRIP 1 | 3 | 1.6445 |
| alpha-2C AR | DFRPSFKHILFRRARRGFRQ | GRIP 1 | 3 | 1.331 |
| alpha-2C AR | DFRPSFKHILFRRARRGFRQ | GRIP 1 | 4 | 3.5815 |
| alpha-2C AR | DFRPSFKHILFRRARRGFRQ | GRIP 1 | 4 | 3.0575 |
| alpha-2C AR | DFRPSFKHILFRRARRGFRQ | GRIP 1 | 5 | 2.0285 |
| alpha-2C AR | DFRPSFKHILFRRARRGFRQ | GRIP 1 | 5 | 1.5895 |
| alpha-2C AR | DFRPSFKHILFRRARRGFRQ | GRIP 1 | 5 | 0 |
| alpha-2C AR | DFRPSFKHILFRRARRGFRQ | GRIP 1 | 5 | 1.223 |
| alpha-2C AR | DFRPSFKHILFRRARRGFRQ | GRIP 1 | 6 | 1.628 |
| alpha-2C AR | DFRPSFKHILFRRARRGFRQ | GRIP 1 | 6 | 1.3525 |
| alpha-2C AR | DFRPSFKHILFRRARRGFRQ | GRIP 1 | 7 | 1.77 |
| alpha-2C AR | DFRPSFKHILFRRARRGFRQ | GRIP 1 | 7 | 1.581 |
| alpha-2C AR | DFRPSFKHILFRRARRGFRQ | HEMBA | 1 | 1.522 |
| | | 1003117 | | |
| alpha-2C AR | DFRPSFKHILFRRARRGFRQ | HEMBA | 1 | 1.8805 |
| | | 1003117 | | |
| alpha-2C AR | DFRPSFKHILFRRARRGFRQ | HEMBA | 1 | 2.0185 |
| | | 1003117 | | |
| alpha-2C AR | DFRPSFKHILFRRARRGFRQ | HEMBA | 1 | 1.7865 |
| | | 1003117 | | |
| alpha-2C AR | DFRPSFKHILFRRARRGFRQ | INADL | 1 | 1.6715 |
| alpha-2C AR | DFRPSFKHILFRRARRGFRQ | INADL | 1 | 2.1475 |
| alpha-2C AR | DFRPSFKHILFRRARRGFRQ | INADL | 2 | 1.826 |
| alpha-2C AR | DFRPSFKHILFRRARRGFRQ | INADL | 2 | 2.7205 |
| alpha-2C AR | DFRPSFKHILFRRARRGFRQ | INADL | 3 | 2.009 |
| alpha-2C AR | DFRPSFKHILFRRARRGFRQ | INADL | 3 | 2.436 |
| alpha-2C AR | DFRPSFKHILFRRARRGFRQ | INADL | 4 | 2.9215 |
| alpha-2C AR | DFRPSFKHILFRRARRGFRQ | INADL | 4 | 3.7865 |
| alpha-2C AR | DFRPSFKHILFRRARRGFRQ | INADL | 5 | 1.7905 |
| alpha-2C AR | DFRPSFKHILFRRARRGFRQ | INADL | 5 | 3.2295 |
| alpha-2C AR | DFRPSFKHILFRRARRGFRQ | INADL | 7 | 1.4955 |
| alpha-2C AR | DFRPSFKHILFRRARRGFRQ | INADL | 7 | 2.885 |
| alpha-2C AR | DFRPSFKHILFRRARRGFRQ | INADL | 8 | 3.8525 |
| alpha-2C AR | DFRPSFKHILFRRARRGFRQ | INADL | 8 | 2.6055 |
| alpha-2C AR | DFRPSFKHILFRRARRGFRQ | KIAA0316 | 1 | 1.9455 |
| alpha-2C AR | DFRPSFKHILFRRARRGFRQ | KIAA0316 | 1 | 1.6115 |
| alpha-2C AR | DFRPSFKHILFRRARRGFRQ | KIAA0340 | 1 | 1.365 |
| alpha-2C AR | DFRPSFKHILFRRARRGFRQ | KIAA0340 | 1 | 2.137 |
| alpha-2C AR | DFRPSFKHILFRRARRGFRQ | KIAA0380 | 1 | 2.455 |
| alpha-2C AR | DFRPSFKHILFRRARRGFRQ | KIAA0380 | 1 | 2.4375 |
| alpha-2C AR | DFRPSFKHILFRRARRGFRQ | KIAA0382 | 1 | 1.6405 |
| alpha-2C AR | DFRPSFKHILFRRARRGFRQ | KIAA0382 | 1 | 1.8285 |
| alpha-2C AR | DFRPSFKHILFRRARRGFRQ | KIAA0440 | 1 | 3.2065 |
| alpha-2C AR | DFRPSFKHILFRRARRGFRQ | KIAA0440 | 1 | 2.5755 |
| alpha-2C AR | DFRPSFKHILFRRARRGFRQ | KIAA0559 | 1 | 1.641 |
| alpha-2C AR | DFRPSFKHILFRRARRGFRQ | KIAA0559 | 1 | 3.0505 |
| alpha-2C AR | DFRPSFKHILFRRARRGFRQ | KIAA0751 | 1 | 2.2225 |
| alpha-2C AR | DFRPSFKHILFRRARRGFRQ | KIAA0751 | 1 | 1.8905 |
| alpha-2C AR | DFRPSFKHILFRRARRGFRQ | KIAA0858 | 1 | 1.759 |
| alpha-2C AR | DFRPSFKHILFRRARRGFRQ | KIAA0858 | 1 | 2.306 |
| alpha-2C AR | DFRPSFKHILFRRARRGFRQ | KIAA0967 | 1 | 1.672 |
| alpha-2C AR | DFRPSFKHILFRRARRGFRQ | KIAA0967 | 1 | 1.677 |
| alpha-2C AR | DFRPSFKHILFRRARRGFRQ | KIAA1095 | 1 | 2.102 |
| alpha-2C AR | DFRPSFKHILFRRARRGFRQ | KIAA1095 | 1 | 2.791 |
| alpha-2C AR | DFRPSFKHILFRRARRGFRQ | KIAA1222 | 1 | 1.725 |
| alpha-2C AR | DFRPSFKHILFRRARRGFRO | KIAA1222 | 1 | 1.898 |
| alpha-2C AR | DFRPSFKHILFRRARRGFRQ | KIAA1284 | 1 | 1.0315 |
| alpha-2C AR | DFRPSFKHILFRRARRGFRQ | KIAA1284 | 1 | 1.546 |
| alpha-2C AR | DFRPSFKHILFRRARRGFRQ | KIAA1415 | 1 | 1.253 |
| alpha-2C AR | DFRPSFKHILFRRARRGFRQ | KIAA1415 | 1 | 1.41 |
| alpha-2C AR | DFRPSFKHILFRRARRGFRQ | KIAA1526 | 1 | 1.3335 |
| alpha-2C AR | DFRPSFKHILFRRARRGFRQ | KIAA1526 | 1 | 1.65 |
| alpha-2C AR | DFRPSFKHILFRRARRGFRQ | KIAA1526 | 1 | 1.847 |
| alpha-2C AR | DFRPSFKHILFRRARRGFRQ | KIAA1526 | 1 | 3.8535 |
| alpha-2C AR | DFRPSFKHILFRRARRGFRQ | KIAA1526 | 2 | 2.1255 |
| alpha-2C AR | DFRPSFKHILFRRARRGFRQ | KIAA1526 | 2 | 1.9005 |
| alpha-2C AR | DFRPSFKHILFRRARRGFRQ | KIAA1526 | 2 | 1.309 |
| alpha-2C AR | DFRPSFKHILFRRARRGFRQ | KIAA1526 | 2 | 1.671 |
| alpha-2C AR | DFRPSFKHILFRRARRGFRQ | KIAA1620 | 1 | 1.0375 |
| alpha-2C AR | DFRPSFKHILFRRARRGFRQ | KIAA1620 | 1 | 1.6985 |
| alpha-2C AR | DFRPSFKHILFRRARRGFRQ | KIAA1719 | 1 | 1.908 |
| alpha-2C AR | DFRPSFKHILFRRARRGFRQ | KIAA1719 | 1 | 1.8755 |
| alpha-2C AR | DFRPSFKHILFRRARRGFRQ | KIAA1719 | 2 | 1.541 |
| alpha-2C AR | DFRPSFKHILFRRARRGFRQ | KIAA1719 | 2 | 1.214 |
| alpha-2C AR | DFRPSFKHILFRRARRGFRQ | KIAA1719 | 3 | 4 |
| alpha-2C AR | DFRPSFKHILFRRARRGFRQ | KIAA17I9 | 3 | 4.096 |
| alpha-2C AR | DFRPSFKHILFRRARRGFRQ | KIAA1719 | 5 | 1.841 |
| alpha-2C AR | DFRPSFKHILFRRARRGFRQ | KIAA17I9 | 5 | 1.4085 |
| alpha-2C AR | DFRPSFKHILFRRARRGFRQ | KIAA1719 | 6 | 2.0975 |
| alpha-2C AR | DFRPSFKHILFRRARRGFRQ | KIAA1719 | 6 | 1.8745 |
| alpha-2C AR | DFRPSFKHILFRRARRGFRQ | LIM mystique | 1 | 1.8425 |
| alpha-2C AR | DFRPSFKHILFRRARRGFRQ | LIM mystique | 1 | 1.317 |
| alpha-2C AR | DFRPSFKHILFRRARRGFRQ | LIM protein | 1 | 1.7205 |
| alpha-2C AR | DFRPSFKHILFRRARRGFRQ | LIM protein | 1 | 2.7195 |
| alpha-2C AR | DFRPSFKHILFRRARRGFRQ | LIM-RIL | 1 | 1.87 |
| alpha-2C AR | DFRPSFKHILFRRARRGFRQ | LIM-RIL | 1 | 3.0615 |
| alpha-2C AR | DFRPSFKHILFRRARRGFRQ | LIMK1 | 1 | 1.673 |
| alpha-2C AR | DFRPSFKHILFRRARRGFRQ | LIMK1 | 1 | 2.5345 |
| alpha-2C AR | DFRPSFKHILFRRARRGFRQ | LIMK2 | 1 | 1.905 |
| alpha-2C AR | DFRPSFKHILFRRARRGFRQ | LIMK2 | 1 | 2.895 |
| alpha-2C AR | DFRPSFKHILFRRARRGFRQ | LU-1 | 1 | 3.889 |
| alpha-2C AR | DFRPSFKHILFRRARRGFRQ | LU-1 | 1 | 3.1685 |
| alpha-2C AR | DFRPSFKHILFRRARRGFRQ | MAGI 1 | 1 | 2.852 |
| alpha-2C AR | DFRPSFKHILFRRARRGFRQ | MAGI 1 | 1 | 1.866 |
| alpha-2C AR | DFRPSFKHILFRRARRGFRQ | MAGI 1 | 1 | 3.3655 |
| alpha-2C AR | DFRPSFKHILFRRARRGFRQ | MAGI 1 | 1 | 2.637 |
| alpha-2C AR | DFRPSFKHILFRRARRGFRQ | MAGI 1 | 3 | 2.2145 |
| alpha-2C AR | DFRPSFKHILFRRARRGFRQ | MAGI 1 | 3 | 2.8475 |
| alpha-2C AR | DFRPSFKHILFRRARRGFRQ | MAGI 1 | 3 | 2.166 |
| alpha-2C AR | DFRPSFKHILFRRARRGFRQ | MAGI 1 | 3 | 3.515 |
| alpha-2C AR | DFRPSFKHILFRRARRGFRQ | MAGI 1 | 4 | 1.997 |
| alpha-2C AR | DFRPSFKHILFRRARRGFRQ | MAGI 1 | 4 | 2.597 |
| alpha-2C AR | DFRPSFKHILFRRARRGFRQ | MAGI 1 | 5 | 1.86 |
| alpha-2C AR | DFRPSFKHILFRRARRGFRQ | MAGI 1 | 5 | 2.221 |
| alpha-2C AR | DFRPSFKHILFRRARRGFRQ | Magi 2 | 1 | 1.727 |
| alpha-2C AR | DFRPSFKHILFRRARRGFRQ | Magi 2 | 1 | 1.9255 |
| alpha-2C AR | DFRPSFKHILFRRARRGFRQ | Magi 2 | 2 | 1.772 |
| alpha-2C AR | DFRPSFKHILFRRARRGFRQ | Magi 2 | 2 | 1.1935 |
| alpha-2C AR | DFRPSFKHILFRRARRGFRQ | Magi 2 | 3 | 1.6635 |
| alpha-2C AR | DFRPSFKHILFRRARRGFRQ | Magi 2 | 3 | 1.336 |
| alpha-2C AR | DFRPSFKHILFRRARRGFRQ | Magi 2 | 4 | 1.624 |
| alpha-2C AR | DFRPSFKHILFRRARRGFRQ | Magi 2 | 4 | 1.339 |
| alpha-2C AR | DFRPSFKHILFRRARRGFRQ | Magi 2 | 5 | 1.927 |
| alpha-2C AR | DFRPSFKHILFRRARRGFRQ | Magi 2 | 5 | 2.0965 |
| alpha-2C AR | DFRPSFKHILFRRARRGFRQ | Magi 2 | 6 | 1.701 |
| alpha-2C AR | DFRPSFKHILFRRARRGFRQ | Magi 2 | 6 | 2.0215 |
| alpha-2C AR | DFRPSFKHILFRRARRGFRQ | MAGi 3 | 1 | 1.994 |
| alpha-2C AR | DFRPSFKHILFRRARRGFRQ | MAGI 3 | 1 | 2.8775 |
| alpha-2C AR | DFRPSFKHILFRRARRGFRQ | MAGI 3 | 2 | 1.987 |
| alpha-2C AR | DFRPSFKHILFRRARRGFRQ | MAGI 3 | 2 | 3.067 |
| alpha-2C AR | DFRPSFKHILFRRARRGFRQ | MAGI 3 | 3 | 1.7405 |
| alpha-2C AR | DFRPSFKHILFRRARRGFRQ | MAGI 3 | 3 | 2.8285 |
| alpha-2C AR | DFRPSFKHILFRRARRGFRQ | MAGI 3 | 4 | 2.5175 |
| alpha-2C AR | DFRPSFKHILFRRARRGFRQ | MAGI 3 | 4 | 1.64 |
| alpha-2C AR | DFRPSFKHILFRRARRGFRQ | MAGI 3 | 5 | 2.869 |
| alpha-2C AR | DFRPSFKHILFRRARRGFRQ | MAGI 3 | 5 | 2.0255 |
| alpha-2C AR | DFRPSFKHILFRRARRGFRQ | MAST1 | 1 | 2.06 |
| alpha-2C AR | DFRPSFKHILFRRARRGFRQ | MAST1 | 1 | 3.687 |
| alpha-2C AR | DFRPSFKHILFRRARRGFRQ | MAST2 | 1 | 1.953 |
| alpha-2C AR | DFRPSFKHILFRRARRGFRQ | MAST2 | 1 | 3.8615 |
| alpha-2C AR | DFRPSFKHILFRRARRGFRQ | MAST4 | 1 | 2.493 |
| alpha-2C AR | DFRPSFKHILFRRARRGFRQ | MAST4 | 1 | 2.4575 |
| alpha-2C AR | DFRPSFKHILFRRARRGFRQ | MINT1 | 1 | 1.31 |
| alpha-2C AR | DFRPSFKHILFRRARRGFRQ | MINT1 | 1 | 2.332 |
| alpha-2C AR | DFRPSFKHILFRRARRGFRQ | MINT1 | 2 | 3.8125 |
| alpha-2C AR | DFRPSFKHILFRRARRGFRQ | MINT1 | 2 | 2.2475 |
| alpha-2C AR | DFRPSFKHILFRRARRGFRQ | MINT1 | 1, 2 | 3.001 |
| alpha-2C AR | DFRPSFKHILFRRARRGFRQ | MINT1 | 1, 2 | 3.6895 |
| alpha-2C AR | DFRPSFKHILFRRARRGFRQ | MPP1 | 1 | 2.1305 |
| alpha-2C AR | DFRPSFKHILFRRARRGFRQ | MPP1 | 1 | 2.433 |
| alpha-2C AR | DFRPSFKHILFRRARRGFRQ | MPP2 | 1 | 2.104 |
| alpha-2C AR | DFRPSFKHILFRRARRGFRQ | MPP2 | 1 | 2.958 |
| alpha-2C AR | DFRPSFKHILFRRARRGFRQ | MUPP1 | 1 | 1.981 |
| alpha-2C AR | DFRPSFKHILFRRARRGFRQ | MUPP1 | 1 | 1.2005 |
| alpha-2C AR | DFRPSFKHILFRRARRGFRQ | MUPP1 | 2 | 1.8855 |
| alpha-2C AR | DFRPSFKHILFRRARRGFRQ | MUPP1 | 2 | 1.4785 |
| alpha-2C AR | DFRPSFKHILFRRARRGFRQ | MUPP1 | 3 | 1.0755 |
| alpha-2C AR | DFRPSFKHILFRRARRGFRQ | MUPP1 | 3 | 2.039 |
| alpha-2C AR | DFRPSFKHILFRRARRGFRQ | MUPP1 | 4 | 3.332 |
| alpha-2C AR | DFRPSFKHILFRRARRGFRQ | MUPP1 | 4 | 2.1975 |
| alpha-2C AR | DFRPSFKHILFRRARRGFRQ | MUPP1 | 4 | 0.36 |
| alpha-2C AR | DFRPSFKHILFRRARRGFRQ | MUPP1 | 5 | 1.8985 |
| alpha-2C AR | DFRPSFKHILFRRARRGFRQ | MUPP1 | 5 | 1.8305 |
| alpha-2C AR | DFRPSFKHILFRRARRGFRQ | MUPP1 | 6 | 2.28 |
| alpha-2C AR | DFRPSFKHILFRRARRGFRQ | MUPP1 | 6 | 2.3995 |
| alpha-2C AR | DFRPSFKHILFRRARRGFRQ | MUPP1 | 7 | 1.871 |
| alpha-2C AR | DFRPSFKHILFRRARRGFRQ | MUPP1 | 7 | 1.8225 |
| alpha-2C AR | DFRPSFKHILFRRARRGFRQ | MUPP1 | 8 | 1.5675 |
| alpha-2C AR | DFRPSFKHILFRRARRGFRQ | MUPP1 | 8 | 1.779 |
| alpha-2C AR | DFRPSFKHILFRRARRGFRQ | MUPP1 | 9 | 1.804 |
| alpha-2C AR | DFRPSFKHILFRRARRGFRQ | MUPP1 | 9 | 1.8075 |
| alpha-2C AR | DFRPSFKHILFRRARRGFRQ | MUPP1 | 10 | 1.8495 |
| alpha-2C AR | DFRPSFKHILFRRARRGFRQ | MUPP1 | 10 | 1.9885 |
| alpha-2C AR | DFRPSFKHILFRRARRGFRQ | MUPP1 | 11 | 1.456 |
| alpha-2C AR | DFRPSFKHILFRRARRGFRQ | MUPP1 | 11 | 1.856 |
| alpha-2C AR | DFRPSFKHILFRRARRGFRQ | MUPP1 | 12 | 1.236 |
| alpha-2C AR | DFRPSFKHILFRRARRGFRQ | MUPP1 | 12 | 1.7415 |
| alpha-2C AR | DFRPSFKHILFRRARRGFRQ | MUPP1 | 13 | 1.377 |
| alpha-2C AR | DFRPSFKHILFRRARRGFRQ | MUPP1 | 13 | 2.7545 |
| alpha-2C AR | DFRPSFKHILFRRARRGFRQ | NeDLG | 1 | 1.0965 |
| alpha-2C AR | DFRPSFKHILFRRARRGFRQ | NeDLG | 1 | 1.819 |
| alpha-2C AR | DFRPSFKHILFRRARRGFRQ | NeDLG | 2 | 1.2775 |
| alpha-2C AR | DFRPSFKHILFRRARRGFRQ | NeDLG | 2 | 1.89 |
| alpha-2C AR | DFRPSFKHILFRRARRGFRQ | NeDLG | 3 | 1.2205 |
| alpha-2C AR | DFRPSFKHILFRRARRGFRQ | NeDLG | 3 | 2.2405 |
| alpha-2C AR | DFRPSFKHILFRRARRGFRQ | NeDLG | 1, 2 | 1.8635 |
| alpha-2C AR | DFRPSFKHILFRRARRGFRQ | NeDLG | 1, 2 | 1.962 |
| alpha-2C AR | DFRPSFKHILFRRARRGFRQ | NOS1 | 1 | 3.0205 |
| alpha-2C AR | DFRPSFKHILFRRARRGFRQ | NOS1 | 1 | 2.0945 |
| alpha-2C AR | DFRPSFKHILFRRARRGFRQ | novel | 1 | 2.3495 |
| | | PDZ gene | | |
| alpha-2C AR | DFRPSFKHILFRRARRGFRQ | novel | 1 | 3.235 |
| | | PDZ gene | | |
| alpha-2C AR | DFRPSFKHILFRRARRGFRQ | novel | 2 | 2.3155 |
| | | PDZ gene | | |
| alpha-2C AR | DFRPSFKHILFRRARRGFRQ | novel | 2 | 2.9335 |
| | | PDZ gene | | |
| alpha-2C AR | DFRPSFKHILFRRARRGFRQ | outer | 1 | 1.2725 |
| | | membrane | | |
| alpha-2C AR | DFRPSFKHILFRRARRGFRQ | outer | 1 | 2.043 |
| | | membrane | | |
| alpha-2C AR | DFRPSFKHILFRRARRGFRQ | p55T | 1 | 1.3015 |
| alpha-2C AR | DFRPSFKHILFRRARRGFRQ | p55T | 1 | 1.561 |
| alpha-2C AR | DFRPSFKHILFRRARRGFRQ | PAR3 | 3 | 1.1185 |
| alpha-2C AR | DFRPSFKHILFRRARRGFRQ | PAR3 | 3 | 1.4575 |
| alpha-2C AR | DFRPSFKHILFRRARRGFRQ | PAR6 | 1 | 2.567 |
| alpha-2C AR | DFRPSFKHILFRRARRGFRQ | PAR6 | 1 | 2.997 |
| alpha-2C AR | DFRPSFKHILFRRARRGFRQ | PAR6 GAMMA | 1 | 1.345 |
| alpha-2C AR | DFRPSFKHILFRRARRGFRQ | PAR6 GAMMA | 1 | 1.0305 |
| alpha-2C AR | DFRPSFKHILFRRARRGFRQ | PDZ-73 | 2 | 1.965 |
| alpha-2C AR | DFRPSFKHILFRRARRGFRQ | PDZ-73 | 2 | 3.1455 |
| alpha-2C AR | DFRPSFKHILFRRARRGFRQ | PDZK1 | 1 | 1.4275 |
| alpha-2C AR | DFRPSFKHILFRRARRGFRQ | PDZK1 | 1 | 1.9785 |
| alpha-2C AR | DFRPSFKHILFRRARRGFRQ | PDZK1 | 2 | 1.049 |
| alpha-2C AR | DFRPSFKHILFRRARRGFRQ | PDZK1 | 2 | 1.876 |
| alpha-2C AR | DFRPSFKHILFRRARRGFRQ | PDZK1 | 3 | 1.4145 |
| alpha-2C AR | DFRPSFKHILFRRARRGFRQ | PDZK1 | 3 | 1.9415 |
| alpha-2C AR | DFRPSFKHILFRRARRGFRQ | PDZK1 | 4 | 1.6115 |
| alpha-2C AR | DFRPSFKHILFRRARRGFRQ | PDZK1 | 4 | 1.913 |
| alpha-2C AR | DFRPSFKHILFRRARRGFRQ | PDZK1 | 2, 3, 4 | 1.8195 |
| alpha-2C AR | DFRPSFKHILFRRARRGFRQ | PDZK1 | 2, 3, 4 | 1.8 |
| alpha-2C AR | DFRPSFKHILFRRARRGFRQ | PICK1 | 1 | 2.2435 |
| alpha-2C AR | DFRPSFKHILFRRARRGFRQ | PICK1 | 1 | 3.094 |
| alpha-2C AR | DFRPSFKHILFRRARRGFRQ | PIST | 1 | 1.005 |
| alpha-2C AR | DFRPSFKHILFRRARRGFRQ | PIST | 1 | 1.2995 |
| alpha-2C AR | DFRPSFKHILFRRARRGFRQ | prIL16 | 1 | 1.413 |
| alpha-2C AR | DFRPSFKHILFRRARRGFRQ | prIL16 | 1 | 1.0525 |
| alpha-2C AR | DFRPSFKHILFRRARRGFRQ | prIL16 | 2 | 1.306 |
| alpha-2C AR | DFRPSFKHILFRRARRGFRQ | prIL16 | 2 | 1.0315 |
| alpha-2C AR | DFRPSFKHILFRRARRGFRQ | prIL16 | 1, 2 | 1.6965 |
| alpha-2C AR | DFRPSFKHILFRRARRGFRQ | prIL16 | 1, 2 | 2.653 |
| alpha-2C AR | DFRPSFKHILFRRARRGFRQ | PSD95 | 1 | 1.2595 |
| alpha-2C AR | DFRPSFKHILFRRARRGFRQ | PSD95 | 1 | 2.0535 |
| alpha-2C AR | DFRPSFKHILFRRARRGFRQ | PSD95 | 3 | 1.191 |
| alpha-2C AR | DFRPSFKHILFRRARRGFRQ | PSD95 | 3 | 1.718 |
| alpha-2C AR | DFRPSFKHILFRRARRGFRQ | PSD95 | 1, 2, 3 | 2.4695 |
| alpha-2C AR | DFRPSFKHILFRRARRGFRQ | PSD95 | 1, 2, 3 | 3.968 |
| alpha-2C AR | DFRPSFKHILFRRARRGFRQ | PTN-4 | 1 | 1.873 |
| alpha-2C AR | DFRPSFKHILFRRARRGFRQ | PTN-4 | 1 | 2.8045 |
| alpha-2C AR | DFRPSFKHILFRRARRGFRQ | PTPL1 | 1 | 0.8135 |
| alpha-2C AR | DFRPSFKHILFRRARRGFRQ | PTPL1 | 1 | 1.1115 |
| alpha-2C AR | DFRPSFKHILFRRARRGFRQ | PTPL1 | 2 | 1.378 |
| alpha-2C AR | DFRPSFKHILFRRARRGFRQ | PTPL1 | 2 | 2.249 |
| alpha-2C AR | DFRPSFKHILFRRARRGFRQ | PTPL1 | 3 | 0.5945 |
| alpha-2C AR | DFRPSFKHILFRRARRGFRQ | PTPL1 | 3 | 2.1675 |
| alpha-2C AR | DFRPSFKHILFRRARRGFRQ | PTPL1 | 4 | 1.0465 |
| alpha-2C AR | DFRPSFKHILFRRARRGFRQ | PTPL1 | 4 | 1.851 |
| alpha-2C AR | DFRPSFKHILFRRARRGFRQ | PTPL1 | 5 | 3.292 |
| alpha-2C AR | DFRPSFKHILFRRARRGFRQ | PTPL1 | 5 | 2.0565 |
| alpha-2C AR | DFRPSFKHILFRRARRGFRQ | Shank 1 | 1 | 1.9 |
| alpha-2C AR | DFRPSFKHILFRRARRGFRQ | Shank 1 | 1 | 1.656 |
| alpha-2C AR | DFRPSFKHILFRRARRGFRQ | SIP1 | 2 | 1.5845 |
| alpha-2C AR | DFRPSFKHILFRRARRGFRQ | SIP1 | 2 | 2.137 |
| alpha-2C AR | DFRPSFKHILFRRARRGFRQ | SITAC 18 | 1 | 1.5095 |
| alpha-2C AR | DFRPSFKHILFRRARRGFRQ | SITAC 18 | 1 | 4.088 |
| alpha-2C AR | DFRPSFKHILFRRARRGFRQ | SITAC 18 | 2 | 2.0615 |
| alpha-2C AR | DFRPSFKHILFRRARRGFRQ | SITAC 18 | 2 | 4 |
| alpha-2C AR | DFRPSFKHILFRRARRGFRQ | SYNTENIN | 1 | 1.3545 |
| alpha-2C AR | DFRPSFKHILFRRARRGFRQ | SYNTENIN | 1 | 2.2475 |
| alpha-2C AR | DFRPSFKHILFRRARRGFRQ | SYNTENIN | 2 | 1.36 |
| alpha-2C AR | DFRPSFKHILFRRARRGFRQ | SYNTENIN | 2 | 2.5975 |
| alpha-2C AR | DFRPSFKHILFRRARRGFRQ | Syntrophin | 1 | 2.5625 |
| | | 1 alpha | | |
| alpha-2C AR | DFRPSFKHILFRRARRGFRQ | Syntrophin | 1 | 3.513 |
| | | 1 alpha | | |
| alpha-2C AR | DFRPSFKHILFRRARRGFRQ | Syntrophin | 1 | 1.5995 |
| | | gamma 1 | | |
| alpha-2C AR | DFRPSFKHILFRRARRGFRQ | Syntrophin | 1 | 1.551 |
| | | gamma 1 | | |
| alpha-2C AR | DFRPSFKHILFRRARRGFRQ | Syntrophin | 1 | 1.021 |
| | | gamma 2 | | |
| alpha-2C AR | DFRPSFKHILFRRARRGFRQ | Syntrophin | 1 | 1.1855 |
| | | gamma 2 | | |
| alpha-2C AR | DFRPSFKHILFRRARRGFRQ | TAX2-like | 1 | 2.065 |
| | | protein | | |
| alpha-2C AR | DFRPSFKHILFRRARRGFRQ | TAX2-like | 1 | 3.268 |
| | | protein | | |
| alpha-2C AR | DFRPSFKHILFRRARRGFRQ | TIAM1 | 1 | 2.519 |
| alpha-2C AR | DFRPSFKHILFRRARRGFRQ | TIAM1 | 1 | 3.0965 |
| alpha-2C AR | DFRPSFKHILFRRARRGFRQ | TIAM2 | 1 | 1.5895 |
| alpha-2C AR | DFRPSFKHILFRRARRGFRQ | TIAM2 | 1 | 1.908 |
| alpha-2C AR | DFRPSFKHILFRRARRGFRQ | TIP1 | 1 | 2.941 |
| alpha-2C AR | DFRPSFKHILFRRARRGFRQ | TIP1 | 1 | 2.6375 |
| alpha-2C AR | DFRPSFKHILFRRARRGFRQ | TIP2 | 1 | 2.421 |
| alpha-2C AR | DFRPSFKHILFRRARRGFRQ | TIP2 | 1 | 2.8285 |
| alpha-2C AR | DFRPSFKHILFRRARRGFRQ | Vartul | 1 | 1.0885 |
| alpha-2C AR | DFRPSFKHILFRRARRGFRQ | Vartul | 1 | 1.454 |
| alpha-2C AR | DFRPSFKHILFRRARRGFRQ | Vartul | 2 | 1.663 |
| alpha-2C AR | DFRPSFKHILFRRARRGFRQ | Vartul | 2 | 1.0935 |
| alpha-2C AR | DFRPSFKHILFRRARRGFRQ | Vartul | 3 | 1.174 |
| alpha-2C AR | DFRPSFKHILFRRARRGFRQ | Vartul | 3 | 2.1 |
| alpha-2C AR | DFRPSFKHILFRRARRGFRQ | Vartul | 4 | 1.24 |
| alpha-2C AR | DFRPSFKHILFRRARRGFRQ | Vartul | 4 | 1.8935 |
| alpha-2C AR | DFRPSFKHILFRRARRGFRQ | Vartul | 1, 2 | 1.695 |
| alpha-2C AR | DFRPSFKHILFRRARRGFRQ | Vartul | 1, 2 | 1.6875 |
| alpha-2C AR | DFRPSFKHILFRRARRGFRQ | X-11 beta | 1 | 2.5855 |
| alpha-2C AR | DFRPSFKHILFRRARRGFRQ | X-11 beta | 1 | 2.125 |
| alpha-2C AR | DFRPSFKHILFRRARRGFRQ | X-11 beta | 2 | 1.4175 |
| alpha-2C AR | DFRPSFKHILFRRARRGFRQ | X-11 beta | 2 | 1.31 |
| alpha-2C AR | DFRPSFKHILFRRARRGFRQ | X-11 beta | 1, 2 | 1.1835 |
| alpha-2C AR | DFRPSFKHILFRRARRGFRQ | X-11 beta | 1, 2 | 1.7135 |
| alpha-2C AR | DFRPSFKHILFRRARRGFRQ | ZO-1 | 1 | 1.9365 |
| alpha-2C AR | DFRPSFKHILFRRARRGFRQ | ZO-1 | 1 | 1.715 |
| alpha-2C AR | DFRPSFKHILFRRARRGFRQ | ZO-1 | 2 | 2.4245 |
| alpha-2C AR | DFRPSFKHILFRRARRGFRQ | ZO-1 | 2 | 2.612 |
| alpha-2C AR | DFRPSFKHILFRRARRGFRQ | ZO-1 | 3 | 1.786 |
| alpha-2C AR | DFRPSFKHILFRRARRGFRQ | ZO-1 | 3 | 1.129 |
| alpha-2C AR | DFRPSFKHILFRRARRGFRQ | ZO-2 | 1 | 1.2275 |
| alpha-2C AR | DFRPSFKHILFRRARRGFRQ | ZO-2 | 1 | 1.4125 |
| alpha-2C AR | DFRPSFKHILFRRARRGFRQ | ZO-2 | 2 | 2.141 |
| alpha-2C AR | DFRPSFKHILFRRARRGFRQ | ZO-2 | 2 | 1.8675 |
| alpha-2C AR | DFRPSFKHILFRRARRGFRQ | ZO-2 | 3 | 2.107 |
| alpha-2C AR | DFRPSFKHILFRRARRGFRQ | ZO-2 | 3 | 1.294 |
| alpha-2C AR | DFRPSFKHILFRRARRGFRQ | ZO-3 | 1 | 1.637 |
| alpha-2C AR | DFRPSFKHILFRRARRGFRQ | ZO-3 | 1 | 2.612 |
| alpha-2C AR | DFRPSFKHILFRRARRGFRQ | ZO-3 | 2 | 1.418 |
| alpha-2C AR | DFRPSFKHILFRRARRGFRQ | ZO-3 | 2 | 2.376 |
| alpha-2C AR | DFRPSFKHILFRRARRGFRQ | ZO-3 | 3 | 1.2515 |
| alpha-2C AR | DFRPSFKHILFRRARRGFRQ | ZO-3 | 3 | 1.6585 |
| Table 8 legend: | | | | |
| Tables 8A, 8B and 8C show the results of G assay testing (described supra) between the three alpha 2 adrenergic subunits and a subset of PDZ domains. All tests are performed at 10 uM concentration of peptide, and the peptide sequence is displayed in column 2. The background binding is somewhat high for these peptides (average OD), and a reduced number of interactions would be seen with lower peptide concentrations. Duplicate rows of PDZ GENE NAME and DOMAIN indicate | | | | |
| # independent sets of duplicates. A ‘0’ in the average OD column indicates failure of the test. | | | | |

TABLE 9
Disorders/biological processes demonstrated to be affected
to alpha adrenergic modulation

| Receptor | Disorder/process |
|---|---|
| A1 | Depression |
| A1 | Lower Urinary Tract Storage |
| A1 | Migraine |
| A1 | Prostate apoptosis |
| A1 | Hypertrophy, proliferation and migration of |
| | vascular smooth muscle after carotid injury |
| A2 | Migraine |
| A2 | Coronary Flow Reserve following stenting |
| A2 | Alzheimer's |
| A2 | Parkinson's |
| A2 | Neuroprotection |
| A2 | Glaucoma |
| A2 | Opioid withdrawl |

Conclusions and Summary

Table 8A,8B and 8C are the first demonstrations that we've discovered of alpha 2 adrenergic receptor (A2R) interactions with PDZ domains. Alpha 1 adrenergic receptors (A1R) also contain PL sequences at their C-termini, but different that A1Rs, implying binding to a different subset of PDZ domains. A single report demonstrated an interaction between alpha 1 receptor A and NOS I (a PDZ protein; Pupo et al. (2002) BMC Pharmacology 2: 17), but the authors demonstrated that this interaction was not dependent on the PL of the A1A adrenergic receptor. Without intending to be limiting, blocking interactions between apha adrenergic receptors and PDZ domains can modulate the effect of signaling through these receptors and provide a new set of therapeutic targets for treatment of diseases or disease stemming from misfunctioning biological processes such as those listed in Table 9.

The present invention is not to be limited in scope by the exemplified embodiments which are intended as illustrations of single aspects of the invention and any sequences which are functionally equivalent are within the scope of the invention. Indeed, various modifications of the invention in addition to those shown and described herein will become apparent to those skilled in the art from the foregoing description and accompanying drawings. Such modifications are intended to fall within the scope of the appended claims.

All publications cited herein are incorporated by reference in their entirety and for all purposes.

TABLE 2

| PL | | | | PDZ | Binding |
|---|---|---|---|---|---|
| AVC ID | PL | PL 20Mer Sequence | PDZ | Domain | Strength |
| AA250 | 5HT3A (serotonin receptor 5- | LAVLAYSITLVMLWSIWQYA | HEMBA 1003117 | 1 | 2 |
| | hydroxytryptamine 3A) | | | | |
| AA250 | 5HT3A (serotonin receptor 5- | LAVLAYSITLVMLWSIWQYA | CARD14 | 1 | 2 |
| | hydroxytryptamine 3A) | | | | |
| AA250 | 5HT3A (serotonin receptor 5- | LAVLAYSITLVMLWSIWQYA | MPP2 | 1 | 2 |
| | hydroxytryptamine 3A) | | | | |
| AA233L | 5HT2B (serotonin receptor 5- | DTLLLTENEGDKTEEQVSYV | MAGI 1 | 3 | 4 |
| | hydroxytryptamine 2B) | | | | |
| AA233L | 5HT2B (serotonin receptor 5- | DTLLLTENEGDKTEEQVSYV | HEMBA 1003117 | 1 | 2 |
| | hydroxytryptamine 2B) | | | | |
| AA233L | 5HT2B (serotonin receptor 5- | DTLLLTENEGDKTEEQVSYV | HEMBA 1003117 | 1 | 1 |
| | hydroxytryptamine 28) | | | | |
| AA233L | 5HT2B (serotonin receptor 5- | DTLLLTENEGDKTEEQVSYV | KIAA0316 | 1 | 1 |
| | hydroxytryptamine 2B) | | | | |
| AA233L | 5HT2B (serotonin receptor 5- | DTLLLTENEGDKTEEQVSYV | KIAA0807 | 1 | 1 |
| | hydroxytryptamine 28) | | | | |
| AA233L | 5HT2B (serotonin receptor 5- | DTLLLTENEGDKTEEQVSYV | KIAA1634 | 2 | 5 |
| | hydroxytryptamine 28) | | | | |
| AA233L | 5HT2B (serotonin receptor 5- | DTLLLTENEGDKTEEQVSYV | KIAA0807 | 1 | 1 |
| | hydroxytryptamine 28) | | | | |
| AA233L | 5HT2B (serotonin receptor 5- | DTLLLTENEGDKTEEQVSYV | Mint 1 | 2 | 1 |
| | hydroxytryptamine 2B) | | | | |
| AA233L | 5HT2B (serotonin receptor 5- | DTLLLTENEGDKTEEQVSYV | MINT1 | 1, 2 | 1 |
| | hydroxytryptamine 28) | | | | |
| AA233L | 5HT2B (serotonin receptor 5- | DTLLLTENEGDKTEEQVSYV | PTPL-1 | 2 | 5 |
| | hydroxytryptamine 28) | | | | |
| AA233L | 5HT2B (serotonin receptor 5- | DTLLLTENEGDKTEEQVSYV | PTPL-1 | 4 | 2 |
| | hydroxytryptamine 28) | | | | |
| AA243 | a2A-AR (modified) (adrenergic | HDFRRAFKKILARGDRKRIV | Magi2 | 6 | 1 |
| | receptor alpha-2A) | | | | |
| AA243 | a2A-AR (modified) (adrenergic | HDFRRAFKKILARGDRKRIV | MAGI 1 | 6 | 3 |
| | receptor alpha-2A) | | | | |
| AA243 | a2A-AR (modified) (adrenergic | HDFRRAFKKILARGDRKRIV | CARD14 | 1 | 1 |
| | receptor alpha-2A) | | | | |
| AA243 | a2A-AR (modified) (adrenergic | HDFRRAFKKILARGDRKRIV | HEMBA 1003117 | 1 | 1 |
| | receptor alpha-2A) | | | | |
| AA243 | a2A-AR (modified) (adrenergic | HDFRRAFKKILARGDRKRIV | FLJ21687 | 1 | 1 |
| | receptor alpha-2A) | | | | |
| AA243 | a2A-AR (modified) (adrenergic | HDFRRAFKKILARGDRKRIV | APXL 1 | 1 | 1 |
| | receptor alpha-2A) | | | | |
| AA243 | a2A-AR (modified) (adrenergic | HDFRRAFKKILARGDRKRIV | HEMBA 1003117 | 1 | 1 |
| | receptor alpha-2A) | | | | |
| AA243 | a2A-AR (modified) (adrenergic | HDFRRAFKKILARGDRKRIV | INADL | 3 | 2 |
| | receptor alpha-2A) | | | | |
| AA243 | a2A-AR (modified) (adrenergic | HDFRRAFKKILARGDRKRIV | INADL | 4 | 1 |
| | receptor alpha-2A) | | | | |
| AA243 | a2A-AR (modified) (adrenergic | HDFRRAFKKILARGDRKRIV | KIAA0340 | 1 | 1 |
| | receptor alpha-2A | | | | |
| AA243 | a2A-AR (modified) (adrenergic | HDFRRAFKKILARGDRKRIV | KIAA0751 | 1 | 3 |
| | receptor alpha-2A) | | | | |
| AA243 | a2A-AR (modified) (adrenergic | HDFRRAFKKILARGDRKRIV | KIAA0897 | 1 | 1 |
| | receptor alpha-2A) | | | | |
| AA243 | a2A-AR (modified) (adrenergic | HDFRRAFKKILARGDRKRIV | KIAA1284 | 1 | 1 |
| | receptor alpha-2A) | | | | |
| AA243 | a2A-AR (modified) (adrenergic | HDFRRAFKKILARGDRKRIV | KIAA1526 | 1 | 4 |
| | receptor alpha-2A) | | | | |
| AA243 | a2A-AR (modified) (adrenergic | HDFRRAFKKILARGDRKRIV | K1719 | 4 | 3 |
| | receptor alpha-2A) | | | | |
| AA243 | a2A-AR (modified) (adrenergic | HDFRRAFKKILARGDRKRIV | LIM-Mystique | 1 | 1 |
| | receptor alpha-2A) | | | | |
| AA243 | a2A-AR (modified) (adrenergic | HDFRRAFKKILARGDRKRIV | Mint 1 | 1 | 1 |
| | receptor alpha-2A) | | | | |
| AA243 | a2A-AR (modified) (adrenergic | HDFRRAFKKILARGDRKRIV | MUPP1 | 6 | 2 |
| | receptor alpha-2A) | | | | |
| AA243 | a2A-AR (modified) (adrenergic | HDFRRAFKKILARGDRKRIV | MUPP1 | 8 | 1 |
| | receptor alpha-2A) | | | | |
| AA243 | a2A-AR (modified) (adrenergic | HDFRRAFKKILARGDRKRIV | MUPP1 | 13 | 1 |
| | receptor atpha-2A) | | | | |
| AA243 | a2A-AR (modified) (adrenergic | HDFRRAFKKILARGDRKRIV | PAR3 | 3 | 2 |
| | receptor alpha-2A) | | | | |
| AA243 | a2A-AR (modified) (adrenergic | HDFRRAFKKILARGDRKRIV | PTPL-1 | 2 | 2 |
| | receptor alpha-2A) | | | | |
| AA243 | a2A-AR (modified) (adrenergic | HDFRRAFKKILARGDRKRIV | SITAC-18 | 1 | 4 |
| | receptor alpha-2A) | | | | |
| AA243 | a2A-AR (modified) (adrenergic | HDFRRAFKKILARGDRKRIV | SITAC-18 | 2 | 4 |
| | receptor alpha-2A) | | | | |
| AA243 | a2A-AR (modified) (adrenergic | HDFRRAFKKILARGDRKRIV | KIAA1526 | 2 | 1 |
| | receptor alpha-2A) | | | | |
| AA243 | a2A-AR (modified) (adrenergic | HDFRRAFKKILARGDRKRIV | X11-beta | 1 | 2 |
| | receptor alpha-2A) | | | | |
| AA243 | a2A-AR (modified) (adrenergic | HDFRRAFKKILARGDRKRIV | X11-beta | 2 | 1 |
| | receptor alpha-2A) | | | | |
| AA243 | a2A-AR (modified) (adrenergic | HDFRRAFKKILARGDRKRIV | ZO-1 | 2 | 4 |
| | receptor alpha-2A) | | | | |
| AA243 | a2A-AR (modified) (adrenergic | HDFRRAFKKILARGDRKRIV | ZO-2 | 2 | 2 |
| | receptor alpha-2A) | | | | |
| AA243 | a2A-AR (modified) (adrenergic | HDFRRAFKKILARGDRKRIV | ZO-3 | 2 | 3 |
| | receptor alpha-2A) | | | | |
| AA243 | a2A-AR (modified) (adrenergic | HDFRRAFKKILARGDRKRIV | DLG5 | 2 | 1 |
| | receptor alpha-2A) | | | | |
| AA243 | a2A-AR (modified) (adrenergic | HDFRRAFKKILARGDRKRIV | AIPC | 1 | 2 |
| | receptor afpha-2A) | | | | |
| AA243 | a2A-AR (modified) (adrenergic | HDFRRAFKKILARGDRKRIV | Syntrophin | 1 | 2 |
| | receptor alpha-2A) | | gamma-1 | | |
| AA243 | a2A-AR (modified) (adrenergic | HDFRRAFKKILARGDRKRIV | Magi2 | 5 | 1 |
| | receptor alpha-2A) | | | | |
| AA244 | a2B-AR (modified) (adrenergic | QDFRRAFRRILARPWTQTAW | PSD95 | 1, 2, 3 | 5 |
| | receptor alpha-2B) | | | | |
| AA244 | a28-AR (modified) (adrenergic | QDFRRAFRRILARPWTQTAW | TIP1 | 1 | 5 |
| | receptor alpha-2B) | | | | |
| AA244 | a2B-AR (modified) (adrenergic | QDFRRAFRRILARPWTQTAW | KIAA0807 | 1 | 4 |
| | receptor alpha-2B) | | | | |
| AA244 | a2B-AR (modified) (adrenergic | QDFRRAFRRILARPWTQTAW | KIAA0303 | 1 | 4 |
| | receptor alpha-2B) | | | | |
| AA244 | a2B-AR (modified) (adrenergic | QDFRRAFRRILARPWTQTAW | MAGI 1 | 2 | 4 |
| | receptor alpha-2B) | | | | |
| AA244 | a28-AR (modified) (adrenergic | QDFRRAFRRILARPWTQTAW | MAGI 1 | 4 | 5 |
| | receptor alpha-2B) | | | | |
| AA245 | a2C-AR (modified) (adrenergic | DFRPSFKHILFRRARRGFRQ | GRIP1 | 5 | 1 |
| | receptor alpha-2C) | | | | |
| AA245 | a2C-AR (modified) (adrenergic | DFRPSFKHILFRRARRGFRQ | LU1 | 1 | 4 |
| | receptor alpha-2C) | | | | |
| AA245 | a2C-AR (modified) (adrenergic | DFRPSFKHILFRRARRGFRQ | PTPL-1 | 5 | 3 |
| | receptor alpha-2C) | | | | |
| AA245 | a2C-AR (modified) (adrenergic | DFRPSFKHILFRRARRGFRQ | APXL1 | 1 | 3 |
| | receptor alpha-2C) | | | | |
| AA245 | a2C-AR (modified) (adrenergic | DFRPSFKHILFRRARRGFRQ | KIAA1719 | 3 | 5 |
| | receptor alpha-2C) | | | | |
| AA245 | a2C-AR (modified) (adrenergic | DFRPSFKHILFRRARRGFRQ | Mint 1 | 2 | 3 |
| | receptor alpha-2C) | | | | |
| AA245 | a2C-AR (modified) (adrenergic | DFRPSFKHILFRRAHRGFRQ | MUPP1 | 4 | 3 |
| | receptor alpha-2C) | | | | |
| AA245 | a20-AR (modified) (adrenergic | DFRPSFKHILFRRARRGFRQ | KIAA0973 | 1 | 3 |
| | receptor alpha-2C) | | | | |
| AA245 | a2C-AR (modified) (adrenergic | DFRPSFKHILFRRARRGFRQ | CARD14 | 1 | 5 |
| | receptor alpha-2C) | | | | |
| AA245 | a2C-AR (modified) (adrenergic | DFRPSFKHILFRRARRGFRQ | DVL2 | 1 | 3 |
| | receptor alpha-2C) | | | | |
| AA252 | ACM3 (muscarinic acetylcholine | QQYQQRQSVIFHKRAPEQAL | APXL1 | 1 | 1 |
| | receptor M3) | | | | |
| AA252 | ACM3 (muscarinic acetylcholine | QQYQQRQSVIFHKRAPEQAL | KIAA0807 | 1 | 1 |
| | receptor M3) | | | | |
| AA252 | ACM3 (muscarinic acetylcholine | QQYQQRQSVIFHKRAPEQAL | KIAA0807 | 1 | 1 |
| | receptor M3) | | | | |
| AA252 | ACM3 (muscarinic acetylcholine | QQYQQRQSVIFHKRAPEQAL | AIPC | 1 | 1 |
| | receptor M3) | | | | |
| AA181 | BAI-1 (brain-specific angiogenesis | RSGATIPLVGQDIIDLQTEV | TIP1 | 1 | 1 |
| | inhibitor 1) | | | | |
| AA181 | BAI-1 (brain-specific angiogenesis | RSGATIPLVGQDIIDLQTEV | KIAA1526 | 1 | 1 |
| | inhibitor 1) | | | | |
| AA181 | BAI-1 (brain-specific angiogenesis | RSGATIPLVGQDIIDLQTEV | PSD95 | 2 | 1 |
| | inhibitor 1) | | | | |
| AA181 | BAI-1 (brain-specific angiogenesis | RSGATIPLVGQDIIDLQTEV | TIP 43 | 1 | 1 |
| | inhibitor 1) | | | | |
| AA181 | BAI-1 (brain-specific angiogenesis | RSGATIPLVGQDIIDLQTEV | NeDLG | 1, 2 | 2 |
| | inhibitor 1) | | | | |
| AA181 | BAI-1 (brain-specific angiogenesis | RSGATIPLVGQDIIDLQTEV | KIAA0973 | 1 | 1 |
| | inhibitor 1) | | | | |
| AA181 | BAI-1 (brain-specific angiogenesis | RSGATIPLVGQDIIDLQTEV | INADL | 3 | 1 |
| | inhibitor 1) | | | | |
| AA181 | BAI-1 (brain-specific angiogenesis | RSGATIPLVGQDIIDLQTEV | DLG2 | 2 | 1 |
| | inhibitor 1) | | | | |
| AA45 | BLR-1 (Burkitt's lymphoma | PSWRRSSLSESENATSLTTF | KIAA0561 | 1 | 1 |
| | receptor 1) | | | | |
| AA45 | BLR-1 (Burkitt's lymphoma | PSWRRSSLSESENATSLTTF | PDZK-1 | 2 | 1 |
| | receptor 1) | | | | |
| AA45 | BLR-1 (Burkitt's lymphoma | PSWRRSSLSESENATSLTTF | KIAA0807 | 1 | 2 |
| | receptor 1) | | | | |
| AA45 | BLR-1 (Burkitt's lymphoma | PSWRRSSLSESENATSLTTF | PDZK1 | 2, 3, 4 | 1 |
| | receptor 1) | | | | |
| AA45 | BLR-1 (Burkitt's lymphoma | PSWRRSSLSESENATSLTTF | SHANK | 1 | 1 |
| | receptor 1) | | | | |
| AA45 | BLR-1 (Burkitt's lymphoma | PSWRRSSLSESENATSLTTF | KIAA0807 | 1 | 2 |
| | receptor 1) | | | | |
| AA269 | C5AR (C5a anaphylatoxin | ESKSFTRSTVDTMAQKTQAV | PTPL-1 | 4 | 1 |
| | chemotactic receptor) | | | | |
| AA29.2 | IL8RB (Interleukin-8 receptor B) | KDSRPSFVGSSSGHTSTTL | KIAA0807 | 1 | 5 |
| AA29.2 | IL8RB (Interleukin-8 receptor B) | KDSRPSFVGSSSGHTSTTL | SHANK | 1 | 3 |
| AA29.2 | IL8RB (Interleukin-8 receptor B) | KDSRPSFVGSSSGHTSTTL | KIAA0382 | 1 | 2 |
| AA29.2 | IL8RB (Interleukin-8 receptor B) | KDSRPSFVGSSSGHTSTTL | KIAA0807 | 1 | 5 |
| AA215 | CKR5 (CC Chemokine receptor | ERASSVYTRSTGEQEISVGL | KIAA1719 | 5 | 1 |
| | type 5) | | | | |
| AA215 | CKR5 (CC Chemokine receptor | ERASSVYTRSTGEQEISVGL | KIAA1719 | 2 | 1 |
| | type 5) | | | | |
| AA215 | CKR5 (CC Chemokine receptor | ERASSVYTRSTGEQEISVGL | TAX IP2 | 1 | 1 |
| | type 5) | | | | |
| AA215 | CKR5 (CC Chemokine receptor | ERASSVYTRSTGEQEISVGL | TIP1 | 1 | 1 |
| | type 5) | | | | |
| AA215 | CKR5 (CC Chemokine receptor | ERASSVYTRSTGEQEISVGL | MINT1 | 1, 2 | 1 |
| | type 5) | | | | |
| AA215 | CKR5 (CC Chemokine receptor | ERASSVYTRSTGEQEISVGL | KIAA1634 | 1 | 1 |
| | type 5) | | | | |
| AA124 | CXCR3 (C-X-C Chemokine receptor | SSSRRDSSWSETSEASYSGL | ELFIN 1 | 1 | 1 |
| | type 3) | | | | |
| AA124 | CXCR3 (C-X-C Chemokine receptor | SSSRRDSSWSETSEASYSGL | KIAA0807 | 1 | 2 |
| | type 3) | | | | |
| AA124 | CXCR3 (C-X-C Chemokine receptor | SSSRRDSSWSETSEASYSGL | KIAA0807 | 1 | 1 |
| | type 3) | | | | |
| AA114 | GLUR7 (metabotropic glutamate | VDPNSPAAKKKYVSYNNLVI | KIAA1634 | 1 | 1 |
| | receptor 7) | | | | |
| AA114 | GLUR7 (metabotropic glutamate | VDPNSPAAKKKYVSYNNLVI | DLG1 | 2 | 1 |
| | receptor 7) | | | | |
| AA114 | GLUR7 (metabotropic glutamate | VDPNSPAAKKKYVSYNNLVI | PAR3 | 3 | 2 |
| | receptor 7) | | | | |
| AA29.3 | IL-8RA (Interleukin-8 receptor A) | ARHRVTSYTSSSVNVSSNL | KIAA0807 | 1 | 1 |
| AA29.3 | IL-8RA (Interleukin-8 receptor A) | ARHRVTSYTSSSVNVSSNL | KIAA0380 | 1 | 1 |
| AA29.3 | IL-8RA (Interleukin-8 receptor A) | ARHRVTSYTSSSVNVSSNL | KIAA1634 | 1 | 1 |
| AA29.3 | IL-8RA (Interleukin-8 receptor A) | ARHRVTSYTSSSVNVSSNL | MAGI 1 | 2 | 1 |
| AA29.3 | IL-8RA (Interleukin-8 receptor A) | ARHRVTSYTSSSVNVSSNL | PSD95 | 1, 2, 3 | 1 |
| AA29.3 | IL-8RA (Interleukin-8 receptor A) | ARHRVTSYTSSSVNVSSNL | MAGI 1 | 6 | 1 |
| AA29.3 | IL-8RA (Interleukin-8 receptor A) | ARHRVTSYTSSSVNVSSNL | Syntrophin | 1 | 1 |
| | | | 1 alpha | | |
| AA29.3 | IL-8RA (Interleukin-8 receptor A) | ARHRVTSYTSSSVNVSSNL | KIAA1634 | 5 | 1 |
| AA29.3 | IL-8RA (Interleuk;n-B receptor A) | ARHRVTSYTSSSVNVSSNL | MUPP1 | 13 | 1 |
| AA29.3 | IL-8RA (Interleukin-B receptor A) | ARHRVTSYTSSSVNVSSNL | novel | 2 | 1 |
| | | | | PDZ gene | |
| AA29.3 | IL-8RA (Interleukin-8 receptor A) | ARHRVTSYTSSSVNVSSNL | PDZK1 | 2, 3, 4 | 1 |
| AA29.3 | IL-8RA (Interleukin-B receptor A) | ARHRVTSYTSSSVNVSSNL | TIP1 | 1 | 1 |
| AA330 | P2Y1 (P2Y Purinoceptor 1) | SEDMTLNILPEFKQNGDTSL | ELFIN 1 | 1 | 1 |
| AA330 | P2Y1 (P2Y Purinoceptor 1) | SEDMTLNILPEFKQNGDTSL | KIAA0807 | 1 | 1 |
| AA330 | P2Y1 (P2Y Purinoceptor 1) | SEDMTLNILPEFKQNGDTSL | Magi2 | 5 | 1 |
| AA330 | P2Y1 (P2Y Purinoceptor 1) | SEDMTLNILPEFKQNGDTSL | KIAA0316 | 1 | 1 |
| AA330 | P2Y1 (P2Y Purinoceptor 1) | SEDMTLNILPEFKQNGDTSL | EBP50 | 1 | 1 |
| AA330 | P2Y1 (P2Y Purinoceptor 1) | SEDMTLNILPEFKQNGDTSL | KIAA0807 | 1 | 1 |
| AA330 | P2Y1 (P2Y Purinoceptor 1) | SEDMTLNILPEFKQNGDTSL | APXL1 | 1 | 1 |
| AA330 | P2Y1 (P2Y Purinoceptor 1) | SEDMTLNILPEFKQNGDTSL | PTN-4 | 1 | 1 |
| AA330 | P2Y1 (P2Y Purinoceptor 1) | SEDMTLNILPEFKQNGDTSL | EBP50 | 2 | 1 |
| AA268 | PTR2 (Parathyroid hormone | RPMESNPDTEGAQGETEDVL | APXL1 | 1 | 1 |
| | receptor) | | | | |
| AA268 | PTR2 (Parathyroid hormone | RPMESNPDTEGAQGETEDVL | PAR3 | 3 | 1 |
| | receptor) | | | | |
| AA205L | 5HT2C (serotonin receptor 5- | ENLELPVNPSSVVSERISSV | MUPP1 | 10 | 1 |
| | hydroxytryptamine 2C) | | | | |
| AA205L | 5HT2C (serotonin receptor 5- | ENLELPVNPSSVVSERISSV | INADL | 8 | 1 |
| | hydroxytryptamine 2C) | | | | |
| AA248 | SSR4 (somatostatin receptor | EALQPEPGRKRIPLTRTTTF | MAGI 1 | 5 | 1 |
| | type 4) | | | | |
| AA248 | SSR4 (somatostatin receptor | EALQPEPGRKRIPLTRTTTF | MAGI 1 | 4 | 1 |
| | type 4) | | | | |
| AA248 | SSR4 (somatostatin receptor | EALQPEPGRKRIPLTRTTTF | DLG1 | 1, 2 | 1 |
| | type 4) | | | | |
| AA248 | SSR4 (somatostatin receptor | EALQPEPGRKRIPLTRTTTF | KIAA0807 | 1 | 1 |
| | type 4) | | | | |
| AA248 | SSR4 (somatostatin receptor | EALQPEPGRKRIPLTRTTTF | MINT1 | 1, 2 | 1 |
| | type 4) | | | | |
| AA248 | SSR4 (somatostatin receptor | EALQPEPGRKRIPLTRTTTF | PDZK1 | 2, 3, 4 | 1 |
| | type 4) | | | | |
| AA113 | SSTR2 (somatostatin receptor | LNETTETQRTLLNGDLQTSI | KIAA0382 | 1 | 1 |
| | type 2) | | | | |
| AA113 | SSTR2 (somatostatin receptor | LNETTETQRTLLNGDLQTSI | KIAA0807 | 1 | 2 |
| | type 2) | | | | |
| AA113 | SSTR2 (somatostatin receptor | LNETTETQRTLLNGDLQTSI | KIAA1526 | 1 | 1 |
| | type 2) | | | | |
| AA113 | SSTR2 (somatostatin receptor | LNETTETQRTLLNGDLQTSI | KIAA1719 | 6 | 1 |
| | type 2) | | | | |
| AA113 | SSTR2 (somatostatin receptor | LNETTETQRTLLNGDLQTSI | Mint 1 | 2 | 1 |
| | type 2) | | | | |
| AA113 | SSTR2 (somatostatin receptor | LNETTETQRTLLNGDLQTSI | SHANK | 1 | 1 |
| | type 2) | | | | |
| AA113 | SSTR2 (somatostatin receptor | LNETTETQRTLLNGDLQTSI | GRIP1 | 7 | 1 |
| | type 2) | | | | |
| AA113 | SSTR2 (somatostatin receptor | LNETTETQRTLLNGDLQTSI | KIAA0807 | 1 | 2 |
| | type 2) | | | | |
| AA113 | SSTR2 (somatostatin receptor | LNETTETQRTLLNGDLQTSI | MINT1 | 1, 2 | 1 |
| | type 2) | | | | |
| AA329 | VIPS (vasoactive intestinal | LQFHRGSRAQSFLQTETSVI | SSTRIP | 1 | 3 |
| | polypeptide receptor 2) | | | | |
| AA329 | VIPS (vasoactive intestinal | LQFHRGSRAQSFLQTETSVI | MAGI 1 | 2 | 1 |
| | polypeptide receptor 2) | | | | |
| AA329 | VIPS (vasoactive intestinal | LQFHRGSRAQSFLQTETSVI | MAGI 1 | 5 | 1 |
| | polypeptide receptor 2) | | | | |
| AA329 | VIPS (vasoactive intestinal | LQFHRGSRAQSFLQTETSVI | EBP50 | 1 | 1 |
| | polypeptide receptor 2) | | | | |
| AA329 | VIPS (vasoactive intestinal | LQFHRGSRAQSFLQTETSVI | FLJ00011 | 1 | 2 |
| | polypeptide receptor 2) | | | | |
| AA329 | VIPS (vasoactive intestinal | LQFHRGSRAQSFLQTETSVI | INADL | 8 | 1 |
| | polypeptide receptor 2) | | | | |
| AA329 | VIPS (vasoactive intestinal | LQFHRGSRAQSFLQTETSVI | KIAA0382 | 1 | 1 |
| | polypeptide receptor 2) | | | | |
| AA329 | VIPS (vasoactive intestinal | LQFHRGSRAQSFLQTETSVI | KIAA0807 | 1 | 3 |
| | polypeptide receptor 2) | | | | |
| AA329 | VIPS (vasoactive intestinal | LQFHRGSRAQSFLQTETSVI | KIAA0807 | 1 | 3 |
| | polypeptide receptor 2) | | | | |
| AA329 | VIPS (vasoactive intestinal | LQFHRGSRAQSFLQTETSVI | INADL | 3 | 1 |
| | polypeptide receptor 2) | | | | |
| AA329 | VIPS (vasoactive intestinal | LQFHRGSRAQSFLQTETSVI | KIAA0973 | 1 | 1 |
| | polypeptide receptor 2) | | | | |
| AA329 | VIPS (vasoactive intestinal | LQFHRGSRAQSFLQTETSVI | KIAA1526 | 2 | 2 |
| | polypeptide receptor 2) | | | | |
| AA329 | VIPS (vasoactive intestinal | LQFHRGSRAQSFLQTETSVI | KIAA1526 | 1 | 1 |
| | polypeptide receptor 2) | | | | |
| AA329 | VIPS (vasoactive intestinal | LQFHRGSRAQSFLQTETSVI | NSP | 1 | 1 |
| | polypeptide receptor 2) | | | | |
| AA329 | VIPS (vasoactive intestinal | LQFHRGSRAQSFLQTETSVI | PIST | 1 | 1 |
| | polypeptide receptor 2) | | | | |
| AA329 | VIPS (vasoactive intestinal | LQFHRGSRAQSFLQTETSVI | Shank 1 | 1 | 2 |
| | polypeptide receptor 2) | | | | |
| AA329 | VIPS (vasoactive intestinal | LQFHRGSRAQSFLQTETSVI | Shank 3 | 1 | 2 |
| | polypeptide receptor 2) | | | | |
| AA329 | VIPS (vasoactive intestinal | LQFHRGSRAQSFLQTETSVI | TIP 43 | 1 | 1 |
| | polypeptide receptor 2) | | | | |

TABLE 3

| | Genbank | | Last | | AVC |
|---|---|---|---|---|---|
| Gene Name (Synonyms) | Reference | Last 20 aa | 4 aa | PL ? | PL ID |
| Adenosine A1 receptor (AdenoA1R) | S45235.1 | FRCQPAPPIDEDLPEERPDD | RPDD | | |
| | GI: 256154 | | | | |
| Adenosine A2a receptor (AdenoA2a, ADORA2A) | X68486.1 | VCPEPPGLDDPLAQDGAGVS | AGVS | | |
| | GI: 400451 | | | | |
| Adenosine A2b receptor (AdenoA2b) | M97759.1 | ADVKSGNGQAGVQPALGVGL | GVGL | X | |
| | GI: 178149 | | | | |
| Adenosine A3 receptor (AdenoA3R) | AAA16365.1 | ACVVCHPSDSLDTSIEKNSE | KNSE | | |
| | GI: 349449 | | | | |
| Adrenergic receptor alpha 2B (a2BadrR, alpha- | M34041.1 | QDFRRAFRRILCRPWTQTAW | QTAW | X | AA244 |
| 28 adrenoceptor, subtype C2) | GI: 178197 | | | | |
| Adrenergic receptor alpha-1A (a1AAdrR, | U02569.1 | HQVPTIKVHTISLSENGEEV | GEEV | X | |
| Alpha 1A-adrenoceptor, Alpha-1C adrenergic | GI: 409028 | | | | |
| receptor). | | | | | |
| Adrenergic receptor Alpha-1A isoform4 | AF013261.1 | REHIKHVNFMMPPWRKGLEC | GLEC | X | |
| (a1AAdri4) | GI: 2978555 | | | | |
| Adrenergic receptor alpha-1B (a1BAdrR, | U03865.1 | DVANGQPGFKSNMPLAPGQF | PGQF | X | |
| Alpha 1B-adrenoceptor). | GI: 494982 | | | | |
| Adrenergic receptor alpha-1C isoform 2 | D32202.1 | FLVETGFHHVGQDDLDLLTS | LLTS | | |
| (a1CAdri2) | GI: 927208 | | | | |
| Adrenergic receptor alpha-1C isoform3 | D32201.1 | ITVSKDQSSCTTARGHTPMT | TPMT | | |
| (a1CAdri3) | GI: 927210 | | | | |
| Adrenergic receptor alpha-1D (a1DAdrR) | U03864.1 | GATCQAYELADYSNLRETDI | ETDI | X | |
| | GI: 494980 | | | | |
| Adrenergic receptor alpha-2A (a2AAdrRec, | M23533.1 | HDFRRAFKKILCRGDRKRIV | KRIV | X | AA243 |
| alpha-2A adrenoceptor, subtype C10) | GI: 178195 | | | | |
| Adrenergic receptor alpha-2C (a2CARC4, | J03853.1 | DFRRSFKHILFRRRRRGFRQ | GFRQ | X | AA245 |
| alpha-2C adrenoceptor, SubtypeC4) | GI: 178193 | | | | |
| Adrenergic receptor Beta-1a (b1AdrRec) | J03019.1 | DSDSSLDEPCRPGFASESKV | ESKV | X | |
| | GI: 178199 | | | | |
| Adrenergic receptor beta-2a (2AdrRec) | Y00106.1 | VPSDNIDSQGRNCSTNDSLL | DSLL | X | |
| | GI: 29370 | | | | |
| Adrenergic receptor Beta-3 (b3AdrRec) | X72861.1 | SSPAQPRLCQRLDGASWGVS | WGVS | | |
| | GI: 298094 | | | | |
| Adrenocorticotropic hormone receptor (ACTH | X65633.1 | FRSPELRDAFKKMIFCSRYW | SRYW | | |
| receptor, ACTH-R, Melanocortin-2 receptor, | GI: 28343 | | | | |
| MC2-R, Adrenocorticotropin receptor) | | | | | |
| Adrenomedullin receptor (AM-R, AdrmedR) | Y13583.1 | AHHLLPNTSPISPTQPLTPS | LTPS | | |
| | GI: 2652933 | | | | |
| Angiotensin II receptor type 1 (AT1, AngIIt1, | M91464.1 | RPSDNVSSSTKKPAPCFEVE | FEVE | | |
| AT1AR) | GI: 179121 | | | | |
| Angiotensin II receptor type-1B (AngII1B, | D13814.1 | RPSDNVSSSTKKPAPCFEVE | FEVE | | |
| AT1B, AT1BR) | GI: 471120 | | | | |
| Angiotensin II receptor type-2 (AngII2R, AT2) | U20860.1 | RESMSCRKSSSLREMETFVS | TFVS | | |
| | GI: 747969 | | | | |
| Apelin receptor (G protein-coupled receptor | U03642.1 | GGEQMHEKSIPYSQETLVVD | LVVD | | |
| APJ, Angiotensin receptor-like 1, HG11) | GI: 425351 | | | | |
| Blue-sensitive opsin (Blue cone photoreceptor | M13299.1 | TCSSQKTEVSTVSSTQVGPN | VGPN | | |
| pigment, Bluopsin) | GI: 1469901 | | | | |
| Bombesin receptor subtype-3 (BRS-3) | L08893.1 | SEISVTSFTGCSVKQAEDRF | EDRF | X | |
| | GI: 291876 | | | | |
| Bradykinin receptor B1 (B1 brady R, BK-1 | U22346.1 | SLAPISSSHRKEIFQLFWRN | FWRN | | |
| receptor, B1R) | GI: 727358 | | | | |
| Bradykinin receptor B2 (B2BK2R, BK-2 | X86165.1 | TSISVERQIHKLQDWAGSRQ | GSRQ | X | |
| receptor, B2R) | GI: 1220160 | | | | |
| Brain-specific angiogenesis inhibitor 1 | AB005297.1 | RSGATIPLVGQDIIDLQTEV | QTEV | X | AA181 |
| precursor (BAI1pre) | GI: 2653431 | | | | |
| Brain-specific angiogenesis inhibitor 2 | A8005298.1 | HRAAAWEPTEPPDGDFQTEV | QTEV | X | |
| precursor (BAI2pre, BAI2) | GI: 3021698 | | | | |
| Brain-specific angiogenesis inhibitor 3 | A8005299.1 | WEKCLNLPLDVQEGDFQTEV | QTEV | X | |
| precursor (BAI3pre, BAI3, KIAA0559) | GI: 3021700 | | | | |
| C3a anaphylatoxin chemotadic receptor | U28488.1 | TRSTHCPSNNVISERNSTTV | STTV | X | |
| (C3achemR, C3AR) | GI: 1199577 | | | | |
| C5a anaphylatoxin chemotadic receptor | X58674.1 | ESKSFTRSTVDTMAQKTQAV | TQAV | X | AA269 |
| (C5a-R, CD88, CD88 antigen) | GI: 29568 | | | | |
| Calcitonin gene-related peptide type 1 | L76380.1 | NGKSIHDIENVLLKPENLYN | NLYN | | |
| receptor precursor (CGRP1Rpre, CGRP type 1 | GI: 1321593 | | | | |
| receptor, CALCRL, CGRPR, CGRR). | | | | | |
| Calcitonin receptor precursor (CalcRpre, | L00587.1 | QGEESAEIIPLNIIEQESSA | ESSA | X | |
| CT-R, CALCR, CALR). | GI: 179879 | | | | |
| Calcium-mobilizing lysophosphatidic acid | AF186380.1 | GSQYIEDSISQGAVCNKSTS | KSTS | | |
| receptor LP-A3/EDG-7 (EDG7, EDG7#2) | GI: 6003655 | | | | |
| Cannabinoid receptor 1 (CB1, CB-R, CANN6) | X54937.1 | TVKIAKVTMSVSTDTSAEAL | AEAL | X | |
| | GI: 29914 | | | | |
| Cannabinoid receptor 2 (CB2, CB-2, CX5) | X74328.1 | EADGKITPWPDSRDLDLSDC | LSDC | X | |
| | GI: 407806 | | | | |
| C—C Chemokine binding protein 2 (Chemokine- | U94888.1 | LGERQSENYPNKEDVGNKSA | NKSA | | |
| binding, protein D6, C—C chemokine receptor | GI: 2213808 | | | | |
| D6, Chemokine receptor CCR-9, CC-Chemokine | | | | | |
| receptor CCR10) | | | | | |
| C—C Chemokine receptor 6 (CCR6) | AAB57794.1 | PRGQSAQGTSREEPDHSTEV | STEV | X | |
| | GI: 2104521 | | | | |
| C—C Chemokine receptor 9A (CCR9A) | AJ132337.1 | EGSLKLSSMLLETTSGALSL | ALSL | X | |
| | GI: 4886431 | | | | |
| C—C Chemokine receptor type 1 (MIP1aR, C—C | L09230.1 | LERVSSTSPSTGEHELSAGF | SAGF | | |
| CKR-1, CCR-1, MIP-1alpha-R, RANTES-R, | GI: 179984 | | | | |
| HM145, LD78 receptor) | | | | | |
| C—C chemokine receptor type 10 (CC-CKR-10, | AF215981.1 | RPRLSSCSAPTETHSLSWDN | SWDN | | |
| CCR-10, G- protein coupled receptor 2) | GI: 7546844 | | | | |
| C—C chemokine receptor type 11 (CC-CKR-11, | AF193507.1 | VEEFPFDSEGPTEPTSTFSI | TFSI | X | |
| CCR-11, Chemokine receptor-like 1, CCRL1, | GI: 7363341 | | | | |
| CCX CKR) | | | | | |
| C—C chemokine receptor type 2(C—C CKR-2, | U03882.1 | GKGKSIGRAPEASLQDKEGA | KEGA | X | |
| CCR-2, Monocyte chemoattractant protein 1 | GI: 472555 | | | | |
| receptor, MCP-1-R) | | | | | |
| C—C chemokine receptor type 3(C—C CKR-3, | U28694.1 | LERTSSVSPSTAEPELSIVF | SIVF | X | AA43 |
| CCR-3, CKR3, Eosinophil eotaxin receptor) | GI: 1199579 | | | | |
| C—C chemokine receptor type 4(C—C CKR-4, | AB023888.1 | DTPSSSYTQSTMDHDLHDAL | HDAL | X | |
| CCR-4, K5-5) | GI: 6467134 | | | | |
| CC Ohemokine receptor type 5(C—C CKR5, | X91492.1 | ERASSVYTRSTGEQEISVGL | SVGL | AA215 | |
| CCR5, HIV-1 fusion co-receptor, CHEMR13, | GI: 1262810 | | | | |
| CD195 antigen) | | | | | |
| C—C chemokine receptor type 6 (CC-CKR-6, | U45984.1 | NISRQTSETADNDNASSFTM | SFTM | | |
| CCR-6, LARC receptor, GPRCY4, Chemokine | GI: 2246432 | | | | |
| receptor-like 3, CKR-L3, DRY6) | | | | | |
| C—C chemokine receptor type 7 (CC-CKR-7, | L08176.1 | RHIRRSSMSVEAETTTTFSP | TFSP | | |
| CCR-7, MIP-3 beta receptor, EBV-induced G | GI: 183484 | | | | |
| protein-coupled receptor 1, EBI1, BLR2) | | | | | |
| C—C chemokine receptor type 8 (CC-CKR-8, | U45983.1 | EKSSSOQQHSSRSSSVDYIL | DYIL | X | |
| CCR-8, GPR-CY6, Chemokine receptor-like 1, | GI: 2231165 | | | | |
| CKR-L1, TER1, CMKBRL2, CC- chemokine | | | | | |
| receptor CHEMR1) | | | | | |
| Cell surface glycoprotein EMR1 precursor | X81479.1 | SQSQTSRILLSSMPSASKTG | SKTG | | |
| (EMR1pre, EMR1 hormone receptor). | GI: 784993 | | | | |
| Chemokine receptor-like 1 (G-protein coupled | U79526.1 | TKMSSMNERTSMNERETGML | TGML | | |
| receptor DEZ, G protein- coupled receptor | GI: 1732342 | | | | |
| ChemR23) | | | | | |
| Chemokine receptor-like 2 (IL8-related | Y08162.1 | LKAVIPDSTEQSDVRFSSAV | SSAV | X | |
| receptor DRY12, Flow-induced endothelial G | GI: 1707499 | | | | |
| protein-coupled receptor, FEG-1, G protein- | | | | | |
| coupled receptor GPR30, GPCR-BR) | | | | | |
| Cholecystokinin type A receptor (CCK-A | L13605.1 | TGASLSRFSYSHMSASVPPQ | VPPQ | | |
| receptor, CCKAR) | GI: 306490 | | | | |
| Corticotropin releasing factor receptor 1 | L23333.1 | SIPTSPTRVSFHSIKQSTAV | STAV | X | |
| precursor (CRFR1pre, CRFR, CRF1, CRHR1, | GI: 40869i | | | | |
| CRHR) | | | | | |
| Corticotropin releasing factor receptor 2 | U34587.1 | SIPTSPTRISFHSIKQTAAV | TAAV | X | |
| precursor (CRFR 2, CRF2, Corticotropin- | GI: 1144507 | | | | |
| releasing hormone receptor 2, CRHR 2) | | | | | |
| CX3C chemokine receptor 1 (CX3CR1, | U20350.1 | SVLSSNFTYHTSDGDALLLL | LLLL | X | |
| Fractalkine receptor, GPR13, V28, Beta | GI: 665580 | | | | |
| chemokine receptor-like 1, CMKBLR1) | | | | | |
| C-X-C Chemokine receptor type 3 (CXCR-3, | X95876.1 | SSSRRDSSWSETSEASYSGL | YSGL | X | AA124 |
| CKR-L2, CD183 antigen). | GI: 1552845 | | | | |
| C-X-C chemokine receptor type 4 (CXC-R4, | L01639.1 | KRGGHSSVSTESESSSFHSS | FHSS | | |
| Stromal cell-derived factor 1 receptor, SDF-1 | GI: 189313 | | | | |
| receptor, Fusin, Leukocyte-derived seven | | | | | |
| transmembrane domain receptor, LESTR, | | | | | |
| LCR1, FB22, NPYRL, HM89, CD184 antigen) | | | | | |
| C-X-C chemokine receptor type 5 (CXCR-5, | X68149.1 | PSWRRSSLSESENATSLTTF | LTTF | X | AA45 |
| Burkitt's lymphoma receptor 1, BLR1, | GI: 29459 | | | | |
| Monocyte-derived receptor 15, MDR15) | | | | | |
| C-X-C chemokine receptor type 6 (CXCR-6, G | AF007545.1 | DNSKTFSASHNVEATSMFQL | MFQL | X | |
| protein-coupled receptor bonzo, G protein- | GI: 2253421 | | | | |
| coupled receptor STRL33) | | | | | |
| Dopamine receptor 1A (DopRec1A, DRD1) | X55758.1 | DTDVSLEKIQPITQNGQHPT | QHPT | | |
| | GI: 288931 | | | | |
| Dopamine receptor D2 (DRD2) | M30625.1 | IIYTTFNIEFRKAFLKILHC | ILHC | X | |
| | GI: 181431 | | | | |
| Dopamine receptor D3 (DopRecD3) | U32499.1 | VIYTTFNIEFRKAFLKILSC | ILSC | X | |
| | GI: 927341 | | | | |
| Dopamine receptor D4 (D(2C) dopamine | AAB59386.1 | YTVFNAEFRNVFRKALRACC | RACC | X | |
| receptor) | GI: 291946 | | | | |
| Dopamine receptor D5 (DopRec1B-D, DRD5, | X58454.1 | DCEGEISLDKITPFTPNGFH | NGFH | | |
| D-1B dopamine receptor, D5 dopamine | GI: 32048 | | | | |
| receptor, D1beta dopamine receptor) | | | | | |
| EGF-like module EMR2 (EMR2egf, EMR2) | AF114491.1 | EMHTLSSSAKADTSKPSTVN | STVN | | |
| | GI: 6650688 | | | | |
| EGF-like module-containing mucin-like | AF239764.1 | GPDSKPSEGDVFPGQVKRKY | KRKY | | |
| receptor EMR3 (EMR3) | GI: 13183148 | | | | |
| Endothelial differentiation protein 1 (EDG-1, | M31210.1 | KDEGDNPETIMSSGNVNSSS | NSSS | | |
| G protein-coupled sphingolipid receptor) | GI: 181948 | | | | |
| Endothelial differentiation protein 4 | AF233092.1 | GASTRIMLPENGHPLMDSTL | DSTL | X | |
| (Lysophosphatidic acid G protein-coupled | GI: 7243675 | | | | |
| receptor 4, Endothelial differentiation | | | | | |
| lysophosphatidic acid G-protein-coupled | | | | | |
| receptor 4, EDG4) | | | | | |
| Endothelial differentiation protein 5 | AF034780.1 | LERGMHMPTSPTFLEGNTVV | NTVV | X | |
| (Lysosphingolipid receptor EDG5) | GI: 4090955 | | | | |
| Endothelin B receptor precursor (ET-B, | M74921.1 | FKANDHGYDNFRSSNKYSSS | YSSS | | |
| Endothelin receptor Non-selective type) | GI: 182275 | | | | |
| Endothelin B receptor-like protein-2 | Y16280.1 | SIYFHKPRESPPLLPLGTPC | GTPC | X | |
| precursor (EndoBRp2, ETBR-LP-2, ETBRLP2) | GI: 3059117 | | | | |
| Endothelin-1 receptor precursor (ET-A, | D90348.1 | KNHDQNNHNTDRSSHKDSMN | DSMN | | |
| EndiRpre). | GI: 219649 | | | | |
| ETL protein (EGF-TM7-latrophilin-related | AF192403.1 | IQEEYYRLFKNVPCCFGCLR | GCLR | | |
| protein, ETL) | GI: 11225482 | | | | |
| Extracellular calcium-sensing receptor | X81086.1 | SQSFVISGGGSTVTENVVNS | VVNS | | |
| precursor (CalRec, CASR, Parathyroid Cell | GI: 599819 | | | | |
| calcium-sensing receptor, GPRC2A, PCAR1). | | | | | |
| fMet-Leu-Phe receptor (fMLP receptor, N- | M37128.1 | TSDTATNSTLPSAEVELQAK | LQAK | | |
| formyl peptide receptor, FPR, N-formylpeptide | GI: 189183 | | | | |
| chemoattractant receptor) | | | | | |
| FMLP-related receptor I (FMLP-R-I, Lipoxin A4 | M76672.1 | TNDTAANSASPPAETELQAM | LQAM | | |
| receptor, LXA4 receptor, REP, HM63) | GI: 182666 | | | | |
| FMLP-related receptor II (FMLP-R-II, | M76673.1 | TSNTHTTSASPPEETELQAM | LQAM | | |
| FMPLrelR) | GI: 182668 | | | | |
| Follicle stimulating hormone receptor | M65085.1 | PRVTNGSTYILVPLSHLAQN | LAQN | | |
| precursor (FSH-R, Follitropin receptor) | GI: 182770 | | | | |
| Frizzled 1 precursor (Fzd1pre, Frizzled-1, | AF072872.1 | NSWRKFYTRLTNSKQGETTV | ETTV | X | |
| Fz-1, hFz1, FzE1, FZD1). | GI: 5305406 | | | | |
| Frizzled 10 precursor (Fzd10pre, Frizzled-10, | AB027464.1 | HPQKTHHGKYEIPAQSPTCV | PTCV | X | |
| Fz-10, hFz10, FzE7, FZD10) | GI: 5834487 | | | | |
| Frizzled 2 precursor (Fzd2pre, Frizzled-2, | L37882.1 | HSWRKFYTRLTNSRHGETTV | ETTV | X | |
| Fz-2, hFz2, FzE2, FZD2). | GI: 736678 | | | | |
| Frizzled 3 precursor (Fzd3pre, Frizzled-3, | AB039723.1 | THITHGTSMNRVIEEDGTSA | GTSA | X | |
| Fz-3, hFz3, FZD3). | GI: 7670051 | | | | |
| Frizzled 4 precursor (Fzd4pre, Frizzled-4, | AB032417.1 | KREKRGNGWVKPGKGSETVV | ETVV | X | |
| Fz-4, hFz4, FzE4, FZD4). | GI: 6277265 | | | | |
| Frizzled 5 precursor (Fzd5pre, Frizzled-5, | U43318.1 | RTGPPGPAATYHKQVSLSHV | LSHV | X | |
| Fz-5, hFz5, FzE5, FZD5, HFZ5). | GI: 1151251 | | | | |
| Frizzled 6 precursor (Fzd6pre, Frizzled-6, | A8012911.1 | LVHPVSGVRKEQGGGCHSDT | HSDT | | |
| Fz-6, hFz6, FZD6)). | GI: 3062802 | | | | |
| Frizzled 8 precursor (Fzd8pre, Frizzled-8, | A8043703.1 | WRSGTASSVSYPKQMPLSQV | LSQV | X | |
| Fz-8, hFz8, FZD8). | GI: 13623798 | | | | |
| Frizzled 9 precursor (Fzd9pre, Frizzled-9, | U82169.1 | PTVVLHMTKTDPSLENPTHL | PTHL | X | |
| Fz-9, hFz9, FzE6, FZD9). | GI: 1906597 | | | | |
| Galanin receptor type 1 (GAL1-R, GALR1) | L34339.1 | DTKENKSRIDTPPSTNCTHV | CTHV | X | |
| | GI: 559047 | | | | |
| Galanin receptor type 2 (GAL2-R, GALR2) | AF040630.1 | PGPSWQGPKAGDSILTVDVA | VDVA | X | |
| | GI: 2921759 | | | | |
| Galanin receptor type 3 (GAL3-R, GALR3). | AF073799.1 | QGPEPREGPVHGGEAARGPE | RGPE | | |
| | GI: 3608409 | | | | |
| Gamma-aminobutyric acid type B receptor, | AJ225028.1 | PPEPPDRLSCDGSRVHLLYK | LLYK | | |
| subunit 1 precursor (GABAB1pre, GABA-B-R1, | GI: 3892593 | | | | |
| Gb1, GABBR1, GABA-B receptor 1, GBR1) | | | | | |
| Gamma-aminobutyric acid type B receptor, | AJ012188.1 | TASPRHRHVPPSFRVMVSGL | VSGL | X | |
| subunit 2 precursor (GABABR2p, GABA-B | GI: 3776097 | | | | |
| receptor 2, GBR2, GABBR2, GABA-B-R2, Gb2, | | | | | |
| G protein-coupled receptor 51, GPR51, HG20). | | | | | |
| Gastric inhibitory polypeptide receptor | U39231.1 | SSGTLPGPGNEASRELESYC | ESYC | X | |
| precursor (GIPRpre, GIPR, Glucose-dependent | GI: 1066050 | | | | |
| insulinotropic polypeptide receptor). | | | | | |
| Gastrin/cholecystokinin type B receptor | L08112.1 | PSIASLSRLSYTTISTLGPG | LGPG | | |
| (CCK-B receptor, CCK-BR) | GI: 306488 | | | | |
| Gastrin-releasing peptide receptor (GRP-R, | M73481.1 | NPSVATFSLINGNICHERYV | ERYV | X | |
| GRP-preferring bombesin receptor) | GI: 183649 | | | | |
| GHRH receptor splice variant 1 (GHRHRsp1) | AF282259.1 | TRAKWTTPSRSAAKVLTSMC | TSMC | X | |
| | GI: 10242291 | | | | |
| glucagon receptor precursor (GlucagRp, GL-R, | U03469.1 | DSSAETPLAGGLPRLAESPF | ESPF | X | |
| GCGR) | GI: 439689 | | | | |
| Glucagon-like peptide 1 receptor precursor | U01104.1 | SSGATAGSSMYTATCQASCS | ASCS | | |
| (GLP1Rpre, GLP-1 receptor, GLP-1-R) | GI: 405081 | | | | |
| Glucagon-like peptide 2 receptor precursor | AF105367.1 | SEGDVTMANTMEEILEESEI | ESEI | X | |
| (GLP2Rpre, GLP-2 receptor, GLP-2-R, | GI: 4324490 | | | | |
| GLP2R). | | | | | |
| Gonadotropin-releasing hormone receptor | L03380.1 | FLFAFLNPCFDPLIYGYFSL | YFSL | X | |
| (GNRH-R) | GI: 183421 | | | | |
| G-protein coupled receptor 91 (GPCR91) | AF348078.1 | KSLTSFSRWAHELLLSFREK | FREK | | |
| | GI: 13517982 | | | | |
| G-protein coupled receptor EDG-7 (EDG7) | AF236117.1 | GSQYKEDSSSQGTVCNKNSS | KNSS | | |
| | GI: 9651838 | | | | |
| G-protein-coupled receptor 74 (GPCR74) | AF236083.1 | QNPHGETLLYRKSAENPNRN | PNRN | | |
| | GI: 14279164 | | | | |
| Green-sensitive opsin (Green cone | M13306.1 | SELSSASKTEVSSVSSVSPA | VSPA | X | |
| photoreceptor pigment) | GI: 180688 | | | | |
| Growth hormone secretagogue receptor type 1 | U60179.1 | KLSTLKDESSRAWTESSINT | SINT | | |
| (GHS-R, GH-releasing peptide receptor, | GI: 1504140 | | | | |
| GHRP, Ghrelin receptor) | | | | | |
| Growth hormone-releasing hormone receptor | L01406.1 | TRAKWTTPSRSAAKVLTSMC | TSMC | X | |
| precursor (GHRHRpre, GRFreceptor, GRFR). | GI: 183172 | | | | |
| Histamine H1 receptor (HistH1R) | Z34897.1 | YPLCNENFKKTFKRILHIRS | HIRS | | |
| | GI: 510295 | | | | |
| Histamine H2 receptor (HistH2R, H2R, Gastric | M64799.1 | LKLQVWSGTEVTAPQGATDR | ATDR | | |
| receptor I) | GI: 184087 | | | | |
| Histamine H3 receptor (HH3R, G protein- | AF140538.1 | LLCPQKLKIQPHSSLEHCWK | HCWK | | |
| coupled receptor 97) | GI: 5031290 | | | | |
| Histamine H4 receptor (HH4R, GPRv53, G | AB044934.1 | KIFCIKKQPLPSQHSRSVSS | SVSS | | |
| protein-coupled receptor 105, GPCR105, | GI: 10241846 | | | | |
| SP9 144, AXOR35) | | | | | |
| HOR5′beta13 (HOR5′b13) | AAG41677.1 | HKFMSLCTSNALPNYLFHQD | FHOD | | |
| | GI: 11908212 | | | | |
| HOR5′beta5 (HOR5′b5) | AAG41683.1 | KTKQIQNAILHLFTTHRIGT | RIGT | | |
| | GI: 11908218 | | | | |
| HOR5′beta6 (HOR5′b6) | AAG41682.1 | KTKQIQSGILRLFSLPHSRA | HSRA | X | |
| | GI: 11908217 | | | | |
| HOR5′beta7 (HOR5′b7) | AAG41681.1 | KTKEIHRAIIKLLGLKKASK | KASK | | |
| | GI: 11908216 | | | | |
| HOR5′beta8 (HOR5′b8) | AAG41680.1 | KTKEIHGAIVRMLLEKRRRV | RRRV | X | |
| | GI: 11908215 | | | | |
| human TA2R, beta isoform (TA2Rbiso, | AAC24302.1 | AGVQLLPFEPPTGKALSRKD | SRKD | | |
| TBXA2R) | GI: 3253117 | | | | |
| Interleukin-8 receptor A (IL8RA, high | AAB59436.1 | LARHRVTSYTSSSVNVSSNL | SSNL | X | |
| affinity IL-8 receptor A, IL-8 receptor type | GI: 559050 | | | | |
| 1, CXCR-1, CDw128a) | | | | | |
| Interleukin-8 receptor B (IL8RB, high | M73969.1 | PKDSRPSFVGSSSGHTSTTL | STTL | X | AA29.2 |
| affinity IL-8 receptor B, CXCR-2, GRO/MGSA | GI: 186516 | | | | |
| receptor, CDw128b) | | | | | |
| KIAA0821 protein. | AB020628.1 | PGLEGPGPDGDGQMQLVTSL | VTSL | X | |
| | GI: 4240127 | | | | |
| Latrophilin-2 (LPHH1, LEC1, LATROPH2) | AJ131581.1 | EGCIPEGDVREGQMQLVTSL | VTSL | X | |
| | GI: 4034485 | | | | |
| Lectomedin-1 alpha (LEC1alph, LEC1) | AF104266.1 | GLRAHLQDLYHLELLLGQIA | GQIA | X | |
| | GI: 5880489 | | | | |
| Lectomedin-1 beta (LEC1beta, LEC1) | AF104938.1 | VKASTTRTSARYSSGTQDIH | QDIH | | |
| | GI: 5880491 | | | | |
| Lectomedin-2 (LEC2) | AF307079.1 | PGLEGPGPDGDGQMQLVTSL | VTSL | X | |
| | GI: 11037013 | | | | |
| Lectomedin-3 (Lecmed3, LEC3) | AF307080.1 | IGASEQCQGYKCHGYSTTEW | TTEW | X | |
| | GI: 11037015 | | | | |
| Leucocyte antigen CD97 precursor (CD97pre, | X84700.1 | TTSGTGHNQTRALRASESGI | ESGI | X | |
| CD97) | GI: 840770 | | | | |
| Leukotriene B4 receptor 2 (BLTR2, Seven | AJ278605.1 | GRGNGDPGGGMEKDGPEWDL | EWDL | | |
| transmembrane receptor BLTR2) | GI: 8919627 | | | | |
| Luteinising hormone-choriogonadotropin | X84753.1 | LSTLHCQGTALLDKTRYTEC | YTEC | X | |
| receptor (Luteinizing hormone receptor) | GI: 1225983 | | | | |
| Lysophosphatidic acid receptor (EDG-2). | U80811.1 | ASSLNHTILAGVHSNDHSVV | HSVV | X | |
| | GI: 1857424 | | | | |
| Lysosphingolipid receptor (EDG-3). | X83864.1 | DPSSCIMDKNAALQNGIFCN | IFCN | | |
| | GI: 1770395 | | | | |
| Melanocortin-3 receptor (MC3-R) | L06155.1 | LELRNTFREILCGCNGMNLG | MNLG | | |
| | GI: 188673 | | | | |
| Melanocortin-4 receptor (MC4-R) | L08603.1 | FKEIICCYPLGGLCDLSSRY | SSRY | X | |
| | GI: 291977 | | | | |
| Melanocortin-5 receptor (MC5-R, MC-2) | Z25470.1 | FKEIICCRGFRIACSFPRRD | PRRD | | |
| | GI: 939924 | | | | |
| Melanocyte stimulating hormone receptor | X65634.1 | YAFHSQELRRTLKEVLTCSW | TCSW | | |
| (MSH-R, Melanotropin receptor, Melanocortin-1 | GI: 34790 | | | | |
| receptor, MC1-R) | | | | | |
| Melatonin receptor type 1A (Mel-1A-R) | U14108.1 | VKWKPSPLMTNNNWKVDSV | VDSV | X | |
| | GI: 602129 | | | | |
| Melatonin receptor type 1B (Mel-1B-R) | U25341.1 | EGLQSPAPPIIGVQHQADAL | ADAL | X | |
| | GI: 971193 | | | | |
| Melatonin-related receptor (H9, GPR50) | U52219.1 | NDYHDVVVVDVEDDPDEMAV | EMAV | X | |
| | GI: 1326154 | | | | |
| Metabotropic glutamate receptor 1 precursor | U31215.1 | PNVSYASVILRDYKQSSSTL | SSTL | X | |
| (GluR1pre, GRM1, GPRC1A, mGluR1) | GI: 945096 | | | | |
| Metabotropic glutamate receptor 2 precursor | L35318.1 | QFVPTVCNGREVVDSTTSSL | TSSL | X | |
| (GluR2pre, MGR2, mGluR2, GRM2, GPRC1B) | GI: 999415 | | | | |
| Metabotropic glutamate receptor 3 precursor | X77748.1 | TYVPTVCNGREVLDSTTSSL | TSSL | X | |
| (GluR3pre, GRM3, GPRC1C, mGluR3) | GI: 1171563 | | | | |
| Metabotropic glutamate receptor 4 precursor | X80818.1 | LEAPALATKQTYVTYTNHAI | NHAI | | |
| (GluR4pre, mGluR4, GRM4, GPRC1D, MGR4) | GI: 1160182 | | | | |
| Metabotropic glutamate receptor 5 precursor | D28538.1 | SSPKYDTLIIRDYTQSSSSL | SSSL | X | |
| (GluR5pre, mGluR5, GRM5, GPRC1E, MGR5) | GI: 1408051 | | | | |
| Metabotropic glutamate receptor 6 precursor | U82083.1 | LKATS1VAAPPKGEDAEAHK | EAHK | | |
| (GluR6pre, GRM6, GPRC1F, mGluR6) | GI: 2231437 | | | | |
| Metabotropic glutamate receptor 7 precursor | X94552.1 | VDPNSPAAKKKYVSYNNLVI | NLVI | X | AA114 |
| (GluR7pre, GRM7, GPRC1G, mGluR7) | GI: 1370110 | | | | |
| Metabotropic glutamate receptor 8 precursor | U92459.1 | LETNTSSTKTTYISYSNHSI | NHSI | | |
| (GluR8pre, GRM8, GPRC1H, mGluR8) | GI: 1935042 | | | | |
| Motilin receptor (G protein-coupled receptor | AF034632.1 | DTGGDTVGYTETSANVKTMG | KTMG | | |
| GPR38) | GI: 2654158 | | | | |
| Muscarinic acetylcholine receptor M1 | X52068.1 | RWRKIPKRPGSVHRTPSRQC | SRQC | X | |
| (AchRm1) | GI: 34450 | | | | |
| Muscarinic acetytcholine receptor M2 | M16404.1 | FKKTFKHLLMCHYKNIGATR | GATR | | |
| | GI: 177989 | | | | |
| Muscarinic acetylcholine receptor M3 | X15266.1 | QQYQQRQSVIFHKRAPEQAL | EQAL | X | AA252 |
| (AchRM3) | GI: 32323 | | | | |
| Muscarinic acetylcholine receptor M4 | M16405.1 | FKKTFRHLLLCQYRNIGTAR | GTAR | | |
| (AchRM4) | GI: 177991 | | | | |
| Muscarinic acetylcholine receptor M5 | M80333.1 | RWKKKKVEEKLYWQGNSKLP | SKLP | | |
| | GI: 177987 | | | | |
| Neuromedin K receptor (NKR, Neurokinin B | S86392.1 | SASATSSFISSPYTSVDEYS | DEYS | | |
| receptor, NK-3 receptor, NK-3R) | GI: 246908 | | | | |
| Neuromedin K receptor (NKR, Neurokinin B | M84605.1 | STSTTASFVSSSHMSVEEGS | EEGS | | |
| receptor, NK-4 receptor, NK-4R, K1R, | GI: 189391 | | | | |
| Neurokinin 4 receptor, NK4) | | | | | |
| Neuromedin U receptor 1 (NMUR1) | AF272362.1 | WVHPLAGNDGPEAQQETDPS | TDPS | | |
| | GI: 10946200 | | | | |
| Neuromedin U receptor 2 (NeUR2, Neuromedin | AF272363.1 | ALSSEQMSRTNYQSFHFNKT | FNKT | | |
| U receptor-type 2, G protein-coupled receptor | GI: 10946202 | | | | |
| TGR-1) | | | | | |
| Neuromedin-B receptor (NMB-R, Neuromedin- | M73482.1 | NMVTNSVLLNGHSMKQEMAM | EMAM | | |
| B-preferring bombesin receptor | GI: 189241 | | | | |
| Neuropeptide FF receptor 1 (NepepFF1, RF | AB040104.1 | LPREGPGCSHLPLTIPAWDI | AWDI | X | |
| amide-related peptide receptor OT7T022) | GI: 11125701 | | | | |
| Neuropeptide FE receptor 2 (Neuropeptide G | AF119815.1 | KPQQELVMEELKETTNSSEI | SSEI | X | |
| protein-coupled receptor, G-protein-coupled | GI: 4530468 | | | | |
| receptor HLWAR77) | | | | | |
| Neuropeptide Y receptor type 1 (NepepYR1, | M88461.1 | KQASPVAFKKINNNDDNEKI | NEKI | X | |
| NPY1-R) | GI: 189155 | | | | |
| Neuropeptide Y receptor type 2 (NPY2-R, NPY- | U36269.1 | NLEVRKNSGPNDSFTEATNV | ATNV | X | |
| Y2 receptor, NepepYR2) | GI: 1063633 | | | | |
| Neuropeptkle Y receptor type 4 (NPY4-R, | U35232.1 | TVHTEVSKGSLRLSGRSNPI | SNPI | | |
| Pancreatic polypeptide receptor 1, PP1) | GI: 1063629 | | | | |
| Neuropeptide Y receptor type 5 (NPY5-R, NPY- | U56079.1 | GFLNNGIKADLVSLIHCLHM | CLHM | | |
| Y5 receptor, Y5 receptor, NPYY5) | GI: 1438903 | | | | |
| Neurotensin receptor type 1 (NT-R-1, High- | X70070.1 | ADSVSSNHTLSSNATRETLY | ETLY | X | |
| affinity levocabastine- insensitive | GI: 35020 | | | | |
| neurotensin receptor, NTRH) | | | | | |
| Neurotensin receptor type 2 (NT-R-2, | Y10148.1 | QSPTLMDTASGFGDPPETRT | ETRT | | |
| Levocabastine-sensitive neurotensin receptor, | GI: 3901027 | | | | |
| NTR2 receptor) | | | | | |
| Ocular albinism type 1 protein (OcuAlb1, QA1) | Z48804.1 | ASESCNKNEGDPALPTHGDL | HGDL | X | |
| | GI: 886873 | | | | |
| Odorant receptor HOR3′beta1 (HOR3′b1) | AAG42364.1 | SVKTQQIHTRMLRLFSLKRY | LKRY | X | |
| | GI: 11991863 | | | | |
| Odorant receptor HOR3′beta3 (HOR3′b3) | AAG42366.1 | KIKEIRNSWLTLSRKRGEF | RGEF | X | |
| | GI: 11991865 | | | | |
| Odorant receptor HOR3′beta5 (HOR3′b5) | AAG42368.1 | VKTKQIRDHIVKVFFFKKVT | KKVT | | |
| | GI: 11991867 | | | | |
| Olfactory receptor 10A4 (OlfR10A4, HP2, | AF209506.1 | KEVKAALKRLIHRTLGSQKL | SQKL | | |
| olfactory-like receptor protein JCG5) | GI: 17016309 | | | | |
| Olfactory receptor 10A5 (OlfR10A5, HP3, | AAG45206.1 | VKNALSRTFHKVLALRNCIP | NCIP | | |
| Putative taste receptor JCG6) | GI: 12007436 | | | | |
| Olfactory receptor 10H1 (OlfR10H1) | AAC08454.1 | KVAMKKTFFSKLYPEKNVMM | NVMM | | |
| | GI: 2996652 | | | | |
| Olfactory receptor 10H2 (OlfR10H2) | AA014388.1 | KELKVAMKRTFLSTLYSSGT | SSGT | | |
| | GI: 3068559 | | | | |
| Olfactory receptor 10J1 (OlfR10J1, Olfactory | X64995.1 | TLRNKEVKOALCRAVGGKFS | GKFS | | |
| receptor-like protein HGMP07J) | GI: 32092 | | | | |
| Olfactory receptor 11A1 (OlfR11A1, Hs6M1-18) | AJ302614.1 | KEVHQALRKILCIKOTETLD | ETLD | | |
| | GI: 12054452 | | | | |
| Olfactory receptor 1203 (OlfR12D3, Hs6M1-27) | CAB65796.1 | MMALKKIFGRKLFKDWQQHH | QQHH | | |
| | GI: 6691936 | | | | |
| Olfactory receptor 1A1 (OlfR1A1, Olfactory | AF087918.1 | LRNRDMKAALRKLFNKRISS | RISS | | |
| receptor 17-7, OR17-7). | GI: 7144622 | | | | |
| Olfactory receptor 1A2 (OlfR1A2, Olfactory | AF155225.1 | LRNWDMKAALQKLFSKRISS | RISS | | |
| receptor 17-6, OR17-6) | GI: 5081803 | | | | |
| Olfactory receptor 1D2 (Olfactory receptor- | X65857.1 | NKDMHGALGRLLDKHFKRLT | KRLT | | |
| like protein HGMP07E, Olfactory receptor | GI: 425220 | | | | |
| 17-4, OR17-4) | | | | | |
| Olfactory receptor 1D4 (OlfR1D4, Olfactory | AF087922.1 | NKDMHGAPGRVLWRPFQRPK | QRPK | | |
| receptor 17-30, OR17-30). | GI: 7144627 | | | | |
| Olfactory receptor 1E1 (OlfR1E1, Olfactory | X64994.1 | RDMKGALSRVIHQKKTFFSL | FFSL | X | |
| receptor-like protein HGMP07I) | GI: 32085 | | | | |
| Olfactory receptor 1E2 (OlfR1E2, Olfactory | AF087925.1 | RDMKGALERVICKRKNPFLL | PFLL | X | |
| receptor 17-93/17-135, OR17-93) | GI: 7144633 | | | | |
| Olfactory receptor 1F1 (OlfR1F1, Olfactory | Y14442.1 | RNRYLKGALKKVVGRVVFSV | VFSV | X | |
| receptor 16-35, OR16-35). | GI: 2370144 | | | | |
| Olfactory receptor 1G1 (OlfR1G1, Olfactory | AF087928.1 | NQEIKSSLRKLIVNRKIHSP | IHSP | | |
| receptor 17-209, OR17-209) | GI: 7144638 | | | | |
| Olfactory receptor 1I1 (OlfR1I1, Olfactory | AAC18915.1 | MHPIPYPGGVQSLAGNRDME | RDME | | |
| receptor 19-20, OR19-20) | GI: 3184262 | | | | |
| Olfactory receptor 2A4 (OlfR2A4) | AAD05193.1 | LRNSEVKNTLKRVLGVERAL | ERAL | X | |
| | GI: 4159884 | | | | |
| Olfactory receptor 2AG1 (OlfR2AG1, HT3) | Q9H205 | VMRALRRVLGKYMLPAHSTL | HSTL | X | |
| | GI: 14423804 | | | | |
| Olfactory receptor 2B2 (OlfR2B2, Olfactory | AJ302584.1 | CPIFVITIENYCNLPQRKFP | RKFP | | |
| receptor 6-1, OR6-1, Hs6M1-10) | GI: 12054392 | | | | |
| Olfactory receptor 2B3 (OlfR2B3, Olfactory | CAA18782.1 | NKDMKEAFKRLMPRIFFCKK | FCKK | | |
| receptor 6-4, OR6-4, Hs6M1-1) | GI: 3757726 | | | | |
| Olfactory receptor 2B6 (OlfR2B6, Hs6M1-32, | CAC14158.1 | NKEVKEGFKRLVARVFLIKK | LIKK | | |
| Olfactory receptor 6-31, OR6-31). | GI: 10944516 | | | | |
| Olfactory receptor 2C1 (OlfR2C1, OLFmf3). | AF098664.1 | RNMEVKGALRRLLGKGREVG | REVG | | |
| | GI: 3982606 | | | | |
| Olfactory receptor 2D2 (OlfR2D2, Olfactory | AAG45204.1 | SLRNKDVKAALRKVATRNFP | RNFP | | |
| receptor 11-610, OR11-610, HB2) | GI: 12007434 | | | | |
| Olfactory receptor 2F1 (OlfR2F1, Olfactory | U56421.1 | KGAWQKLLWKFSGLTSKLAT | KLAT | | |
| receptor-like protein OLF3). | GI: 1336042 | | | | |
| Olfactory receptor 2F2 (OlfR2F2, Olfactory | AAC64378.1 | KGAWHKLLEKFSGLTSKLGT | KLGT | | |
| receptor 7-1, OR7-1) | GI: 3766133 | | | | |
| Olfactory receptor 2H1 (OR2H1, OlfR2H1, | AJ302604.1 | RALRRLLGKERDSRESWRAA | WRAA | X | |
| Hs6M1-16, Olfactory receptor 6-2, OR6-2) | GI: 12054432 | | | | |
| Olfactory receptor 2H3 (OlfR2H3, Olfactory | L35475.1 | RAFRRLLGKERDSRESWRAA | WRAA | X | |
| receptor-like protein FAT11) | GI: 1041044 | | | | |
| Olfactory receptor 2J2 (Olfactory receptor | AJ302571.1 | LRNKHVKGAAKRLLGWEWGK | EWGK | | |
| 6-8, OR6-8, Hs6M1-6) | GI: 12054366 | | | | |
| Olfactory receptor 2J3 (OlfR2J3, Olfactory | CAA18783.1 | IYTLRNKVVRGAVKRLMGWE | MGWE | | |
| receptor 6-6, OR6-6, Hs6M1-3). | GI: 3757727 | | | | |
| Olfactory receptor 2T1 (OlfR2T1, OR2T1, | XM_060316.1 | ALKRALGRFKGPQRVSGGVF | GGVF | X | |
| Olfactory receptor 1-25, OR1-25) | GI: 17437062 | | | | |
| Olfactory receptor 2W1 (OlfR2W1, Hs6M1-15). | CAB42853.1 | LKKLMRFHHKSTKIKRNCKS | NCKS | | |
| | GI: 4826521 | | | | |
| Olfactory receptor 3A1 (OlfR3A1, Olfactory | X80391.1 | RNPDVQSAIWRMLTGRRSLA | RSLA | X | |
| receptor 17-40, OR17-40) | GI: 516319 | | | | |
| Olfactory receptor 3A2 (OlfR3A2, Olfactory | AF087930.1 | RNPDVQGALWQIFLGRRSLT | RSLT | | |
| receptor 17-228, OR17-228, OR3A2, OLFRA04) | GI: 7144641 | | | | |
| Olfactory receptor 3A3 (OlfR3A3, Olfactory | AF087926.1 | RNTDVQGALCQLLVGERSLT | RSLT | | |
| receptor 17-201, OR17-201) | GI: 7144635 | | | | |
| Olfactory receptor 4F3 (OlfR4F3) | AAD05195.1 | EMKAAIKRVCKQLVIYKRIS | KRIS | | |
| | GI: 4159886 | | | | |
| Olfactory receptor 51B2 (HOR5′b3, | AAD29425.2 | KTKQIQYGIIRLLSKHRFSR | RFSR | | |
| HOR5′beta3, OR51B2) | GI: 11908208 | | | | |
| Olfactory receptor 51B4 (HOR5′b1, | AAD29426.2 | IKTKQIQRSIIRLFSGQSRA | QSRA | X | |
| HOR5′beta1, OXB4, OR51B4) | GI: 11908209 | | | | |
| Olfactory receptor 51E2 (OlfR51E2, Prostate | AF311306.1 | RVLAMFKISCDKDLQAVGGK | VGGK | | |
| specific G-protein coupled receptor, OXE2, | GI: 11875777 | | | | |
| HPRAJ, OR51E2, PSGR) | | | | | |
| Olfactory receptor 51I1 (HOR5′b11, | AAG41679.1 | SVKTKEIRKGILKFFHKSQA | KSQA | X | |
| HOR5′beta11, OR51I1). | GI: 11908214 | | | | |
| Olfactory receptor 5112 (HOR5′b12, | AAG41678.1 | SAKTKEIRRAIFRMFHHIKI | HIKI | X | |
| HOR5′beta12, OR51I2) | GI: 11908213 | | | | |
| Olfactory receptor 52A1 (HOR3′b4, HPFH1OR, | AAG42367.1 | LVYGAKTTQIRIHVVKMFCS | MFCS | | |
| HOR3′beta4, OR52A1) | GI: 11991866 | | | | |
| Olfactory receptor 52D1 (HOR5′b14, | AAG41676.1 | RTKEIRSRLLKLLHLGKTSI | KTSI | X | |
| HOR5′beta14, OR52D1) | GI: 11908211 | | | | |
| Olfactory receptor 5F1 (OlfR5F1, Olfactory | O95221 | KEVKKALANVISRKRTSSFL | SSFL | X | |
| receptor 11-10, OR11-10) | GI: 14423782 | | | | |
| Olfactory receptor 5I1 (OlfR5I1, Olfactory | U56420.1 | RNKDVKDAAEKVLRSKVDSS | VDSS | | |
| receptor-like protein OLF1) | GI: 1336040 | | | | |
| Olfactory receptor 5U1 (OlfR5U1, Hs6M1-28). | XM_167134.2 | MLSKEELPQRKMCLKAMFKL | MFKL | X | |
| | GI: 22059864 | | | | |
| Olfactory receptor 5V1 (OlfR5V1, Hs6M1-21). | CAB65797.1 | KTIGSKWQPPISSLDSKLTY | KLTY | X | |
| | GI: 6691937 | | | | |
| Olfactory receptor 6A1 (OlfR6A1, Olfactory | AF065870.1 | CILHLYQHQDPDPKKGSRNV | SRNV | X | |
| receptor 11-55, OR11-55) | GI: 3831610 | | | | |
| Olfactory receptor 6B1 (OlfR6B1, Olfactory | AAC64377.1 | NREVKEALKKLAYCQASRSD | SRSD | | |
| receptor 7-3, OR7-3, OR6B1) | GI: 3766132 | | | | |
| Olfactory receptor 7A10 (OlfR7A10, OST027) | AAC25627.1 | YSLRNKHIKGAMKTFFRGKQ | RGKQ | | |
| | GI: 3290001 | | | | |
| Olfactory receptor 7A17 (OlfR7A17) | AAB82060.1 | YSLRNKDIKRALKMSFRGKQ | RGKQ | | |
| | GI: 2447219 | | | | |
| Olfactory receptor 7A5 (OlfR7A5, Olfactory | Y10530.1 | ALGIHLLWGTMKGQFFKKCP | KKCP | | |
| receptor TPCR92). | GI: 2792017 | | | | |
| Olfactory receptor 7C1 (OlfR7C1, Olfactory | AAC25625.1 | LGRLLSRATFFNGDITAGLS | AGLS | | |
| receptor TPCR86). | GI: 3289999 | | | | |
| Olfactory receptor 7C2 (OlfR7C2, Olfactory | AAC15755.1 | LGRLLLRATSLKEGTIAKLS | AKLS | | |
| receptor 19-18, OR19-18) | GI: 3108023 | | | | |
| Olfactory receptor 89 (OlfR89) | AJ132194.1 | NVKGALRNLVRSISALSDSG | SDSG | X | |
| | GI: 4160227 | | | | |
| Olfactory receptor 8B8 (OlfR8B8, Olfactory | AF238488.1 | LRNKDVKVALKKILNKNAFS | NAFS | | |
| receptor TPCR85, olfactory-like receptor | GI: 17016318 | | | | |
| JCG8) | | | | | |
| Olfactory receptor 8D2 (OlfR8D2, Olfactory | AF162668.1 | LRNKDVKNALKKMTRGRQSS | RQSS | | |
| receptor-like protein JCG2) | GI: 12002781 | | | | |
| Olfactory receptor H17 (OlfRH17) | AAG45208.1 | CTLHLYQHQDPDPKKASRNV | SRNV | X | |
| | GI: 12007438 | | | | |
| Opioid receptor mu 1 (m1OpioiR) | CAC15482.1 | RDHPSTANTVDRTNHQVRSL | VRSL | X | |
| | GI: 11128469 | | | | |
| Opioid receptor type delta (d1OpioiR, DOR-1) | U10504.1 | ARERVTACTPSDGPGGGAAA | GAAA | | |
| | GI: 501144 | | | | |
| Opioid receptor type kappa (k1OpioiR, KOR-1) | U11053.1 | RNTVQDPAYLRDIDGMNKPV | NKPV | | |
| | GI: 532059 | | | | |
| Opioid receptor type kappa 3 (k3OpioiR, | X77130.1 | SIAKDVALACKTSETVPRPA | PRPA | X | |
| Nociceptin receptor, (Orphanin FQ receptor, | GI: 471316 | | | | |
| kappa-type 3 opiold receptor, KOR-3) | | | | | |
| Opioid receptor type mu (mOpioiR, MOR-1) | AAA20580.1 | TVDRTNHQLENLEAETAPLP | APLP | | |
| | GI: 452073 | | | | |
| Opsin 3 (Encephalopsin, Panopsin) | AF140242.1 | VDDSDKTNGSKVDVIQVRPL | VRPL | X | |
| | GI: 4894951 | | | | |
| Opsin 4 (Melanopsin) | AF147788.1 | HEAETPGKTKGLIPSQDPRM | DPRM | | |
| | GI: 6693700 | | | | |
| Orexin receptor type 1 (Ox1r, Hypocretin | AF041243.1 | CSISKISEHVVLTSVTTVLP | TVLP | | |
| receptor type 1) | GI: 2897123 | | | | |
| Orexin receptor type 2 (Ox2r, OX2R, | AF041245.1 | VLTSISTLPAANGAGPLQNW | LQNW | | |
| Hypocretin receptor type 2) | GI: 2897127 | | | | |
| Oxytocin receptor (OT-R, OxytocR) | X64878.1 | SFVLSHRSSSQRSCSQPSTA | PSTA | X | |
| | GI: 34764 | | | | |
| P2Y purinoceptor 1 (P2Y1R, ATPreceptor, | Z49205.1 | SEDMTLNILPEFKQNGDTSL | DTSL | X | AA330 |
| P2Y1, Purinergic receptor) | GI: 798835 | | | | |
| P2Y purinoceptor 10 (P2Y10, P2Y-like | AF000545.1 | GSSVTRSRLMSKESGSSMIG | SMIG | | |
| receptor) | GI: 2104786 | | | | |
| P2Y purinoceptor 11 (P2Y11) | AJ298334.1 | PLNATAAPKPSEPQSRELSQ | ELSQ | X | |
| | GI: 12964589 | | | | |
| P2Y purinoceptor 2 (P2Y2, P2U purinoceptor 1, | U07225.1 | DFRRTESTPAGSENTKDIRL | DIRL | X | |
| P2U1, ATP receptor, Purinergic receptor) | GI: 984506 | | | | |
| P2Y purinoceptor 4 (P2Y4, Uridine nucleotide | X91852.1 | CRWAATPQDSSCSTPRADRL | ADRL | X | |
| receptor, UNR, P2P) | GI: 1124904 | | | | |
| P2Y purinoceptor 5 (P2Y5, Purinergic receptor | AF000546.1 | FIQHNLQTLKSKIFDNESAA | ESAA | | |
| 5, RB intron encoded G-protein coupled | GI: 2232068 | | | | |
| receptor) | | | | | |
| P2Y purinoceptor 7 (P2Y7, Leukotriene B4 | U41070.1 | EPGPSESLTASSPLKLNELN | NELN | | |
| receptor, Chemoattractant receptor-like 1) | GI: 1469913 | | | | |
| P2Y purinoceptor 9 (P2Y9R, Purinergic | U66578.1 | EEVSDQTTNNGGELMLESTF | ESTF | | |
| receptor 9, GPCR GPR23, P2Y5-like receptor) | GI: 1753100 | | | | |
| Parathyroid hormone receptor precursor | U25128.1 | RPMESNPDTEGCQGETEDVL | EDVL | X | AA268 |
| (PTH2Rpre, PTH2 receptor, PTHR2) | GI: 887966 | | | | |
| Parathyroid hormone/parathyroid hormone- | L04308.1 | EASGPERPPALLQEEWETVM | ETVM | X | |
| related peptide receptor precursor (PTHRpre, | GI: 190721 | | | | |
| PTH/PTHR receptor, PTHR1, PTHR, PTRR) | | | | | |
| Peropsin (Visual pigment-like receptor | AF012270.1 | PVTSILPMDVSONPLASGRI | SGRI | X | |
| peropsin) | GI: 2307009 | | | | |
| Pituitary adenylate cyclase activating | NP_001109.1 | LSKSSSQIRMSGLPADNLAT | NLAT | | |
| polypeptide type I receptor precursor | GI: 4501923 | | | | |
| (PACAPR21p, ADCYAP1R1, PACR, PACAP | | | | | |
| type I receptor) | | | | | |
| Platelet activating factor receptor (PAF-R) | M80436.1 | DTVTEVVVPFNQIPGNSLKN | SLKN | | |
| | GI: 189537 | | | | |
| Probable G protein-coupled receptor GPR32 | AF045764.1 | RAFGEEEFLSSCPRGNAPRE | APRE | | |
| | GI: 3282838 | | | | |
| Probable G protein-coupled receptor GPR35 | AF027957.1 | AVAPRAKAHKSQDSLCVTLA | VTLA | X | |
| (GPCR35) | GI: 2739108 | | | | |
| Probable G protein-coupled receptor GPR72 | AF236081.1 | SQLQSGKTDLSSVEPIVTMS | VTMS | | |
| precursor (GPR72pre, GPR72, KIAA1540) | GI: 7248881 | | | | |
| Prostacyclin receptor (Prostanoid IP | 129016.1 | SGSAVGTSSKAEASVACSLC | CSLC | X | |
| receptor, PGI receptor, PTGIR, PRIPR) | GI: 495042 | | | | |
| Prostaglandin D2 receptor (ProstD2R, | Q13258 | IRPLRYRSRCSNSTNMESSL | ESSL | X | |
| Prostanoid D Preceptor, PGD receptor) | GI: 2495009 | | | | |
| Prostaglandin E2 receptor, EP1 subtype | L22647.1 | PSAWEASSLRSSRHSGLSHF | LSHF | X | |
| (PE2Rep1, Prostanoid EP1 receptor, PGE | GI: 410208 | | | | |
| receptor, EP1 subtype, PE21, PTGER1). | | | | | |
| Prostaglandin E2 receptor, EP2 subtype | U19487.1 | QDATQTSCSTQSDASKQADL | QADL | X | |
| (PE2Rep2, PTGER2, Prostanoid EP2 receptor, | GI: 639719 | | | | |
| PGE receptor, Ep2subtype). | | | | | |
| Prostaglandin E2 receptor, EP3 subtype | U13218.1 | STSLPCQCSSTLMWSDHLER | HLER | | |
| (PE2Rep3, Prostanoid EP3 receptor, PGE | GI: 532745 | | | | |
| receptor, EP3 subtype) | | | | | |
| Prostaglandin E2 receptor, EP4 subtype | AAA36434.1 | GSSLQVTFPSETLNLSEKCI | EKCI | X | |
| (PE2ep4, Prostanoid EP4 receptor, | GI: 452496 | | | | |
| PGEreceptor, EP4subtype). | | | | | |
| Prostaglandin EP3 receptor (ProsEP3R) | BAA19952.1 | LPLTLASFKLLREPCSVQLS | VQLS | | |
| | GI: 2114191 | | | | |
| Prostaglandin EP3 receptor subtype isoform | D86097.1 | QVPRTWCSSHDREPCSVQLS | VQLS | | |
| (PEP3isof) | GI: 2102644 | | | | |
| Prostaglandin F2-alpha receptor (PF2aR, | AF004021 .1 | NSLKVAAISESPVAEKSAST | SAST | | |
| Prostanoid FP receptor, PGF receptor, PGF2 | GI: 2257849 | | | | |
| alpha receptor, PTGFR) | | | | | |
| Proteinase activated receptor 1 precursor | M62424.1 | SKMDTCSSNLNNSIYKKLLT | KLLT | | |
| (PAR-1, Thrombin receptor, Coagulation factor | GI: 339676 | | | | |
| II receptor) | | | | | |
| Proteinase activated receptor 2 precursor | Z49993.1 | KHSRKSSSYSSSSTTVKTSY | KTSY | X | |
| (PAR-2, Thrombin receptor- like 1, | GI: 1008084 | | | | |
| Coagulation factor II receptor-like 1) | | | | | |
| Proteinase activated receptor 3 precursor | U92971 .1 | PFLYFLMSKTRNHSTAYLTK | YLTK | | |
| (PAR-3, Thrombin receptor- like 2, | GI: 1938374 | | | | |
| Coagulation factor II receptor-like 2) | | | | | |
| Proteinase activated receptor 4 precursor | AF080214.1 | SKASAEGGSRGMGTHSSLLQ | SLLQ | | |
| (PAR-4, Thrombin receptor- like 3, | GI: 3396080 | | | | |
| Coagulation factor II receptor-like 3) | | | | | |
| Putative G protein-coupled receptor 54 | AB051065.1 | GSSGLAARGLCVLGEDNAPL | NAPL | X | |
| (GPCR54, GPR54) | GI: 14041797 | | | | |
| Putative G protein-coupled receptor 92 | AJ272207.1 | RPSDSHSLSSFTQCPQDSAL | DSAL | X | |
| (GPCR92) | GI: 9843745 | | | | |
| Putative G protein-coupled receptor GPR44 | AB008535.1 | SCAASPQTGPLNRALSSTSS | STSS | | |
| (Chemoattractant receptor- homologous | GI: 4204215 | | | | |
| molecule expressed on Th2 cells) | | | | | |
| Putative G-Protein coupled receptor, EDG6 | AJ000479.1 | RSLSFRMREPLSSISSVRSI | VRSI | X | |
| precursor (EDG6pre, Hypothetical protein). | GI: 3805931 | | | | |
| Red-sensitive opsin (Red cone photoreceptor | M13300.1 | SELSSASKTEVSSVSSVSPA | VSPA | X | |
| pigment) | GI: 180696 | | | | |
| Retinal G protein coupled receptor | BC011349.1 | VCRGIWQCLSPQKREKDRTK | DRTK | | |
| | GI: 15030185 | | | | |
| Rhodopsin (Opsin2) | AAC31763.1 | GDDEASA1VSKTETSQVAPA | VAPA | X | |
| | GI: 1236137 | | | | |
| Secretin receptor precursor (SecrRpre, | U20178.1 | NSTKASHLEQSQGTCRTSII | TSII | X | |
| SCT-R). | GI: 662795 | | | | |
| Serotonin receptor 5-hydroxytryptamine 1A | M28269.1 | FNKDFQNAFKKIIKCKFCRQ | FCRQ | | |
| (5HT1A, G-21, ser-5-hydroxytryptamine 1A | GI: 189927 | | | | |
| receptor) | | | | | |
| Serotonin receptor 5-hydroxytryptamine 1B | D10995.1 | MSNEDFKQAFHKLIRFKCTS | KCTS | | |
| (5HT1B, ser-5-hydroxytryptamine 1B rec, 5- | GI: 219678 | | | | |
| HT-1D-beta, serotonin 10 beta receptor, S12) | | | | | |
| serotonin receptor 5-hydroxytryptamine 1D | M89955.1 | VFNEEFRQAFQKIVPFRKAS | RKAS | | |
| (5HT1D, serotonin receptor 5-HT-1D-alpha, | GI: 177771 | | | | |
| HTR1D) | | | | | |
| Serotonin receptor 5-hydroxytryptamine 1E | M91467.1 | SFNEDFKLAFKKLIRCREHT | REHT | | |
| (5HT1E, serotonin receptor 5-HT1E, S31) | GI: 177773 | | | | |
| Serotonin receptor 5-hydroxytryptamine 1F (5- | L05597.1 | YTIFNEDFKKAFQKLVRCRC | RCRC | X | |
| HT-1F, serotonin receptor 5HT1F) | GI: 307419 | | | | |
| Serotonin receptor 5-hydroxytryptamine 2A (5- | S42168.1 | HSEEASKDNSDGVNEKVSCV | VSCV | X | |
| HT-2A, serotonin receptor 5HT2A) | GI: 252946 | | | | |
| Serotonin receptor 5-hydroxytryptamine 2B (5- | X77307.1 | DTLLLTENEGDKTEEQVSYV | VSYV | X | AA233L |
| HT-2B, serotonin receptor 5HT2B) | GI: 475197 | | | | |
| Serotonin receptor 5-hydroxytryptamine 2C (5- | M81778.1 | ENLELPVNPSSVVSERISSV | ISSV | X | AA205L |
| HT-2C, serotonin receptor 5HT2C) | GI: 338027 | | | | |
| Serotonin receptor 5-hydroxytryptamine 4(5- | V12505.1 | ESQCHPPATSPLVAAQPSDT | PSDT | | |
| HT-4, serotonin receptor 5HT4) | GI: 2661756 | | | | |
| Serotonin receptor 5-hydroxytryptamine 5A (5- | X81411.1 | YTAFNKNYNSAFKNFFSRQH | SRQH | | |
| HT-5A, serotonin receptor 5HT5A) | GI: 541776 | | | | |
| Serotonin receptor 5-hydroxytryptamine 6 (5- | L41147.1 | FNIDPAEPELRPHPLGIPTN | IPTN | | |
| HT-6, serotonin receptor 5HT6) | GI: 1162923 | | | | |
| Serotonin receptor 5-hydroxytryptamine 7 (5- | U68488.1 | HNWLADKMLTTVEKKVMIHD | MIHD | | |
| HT-7, serotonin receptor 5HT7, 5HTX) | GI: 1857144 | | | | |
| Smoothened homolog precursor (SMOpre, | U84401.1 | PIHSRTNLMDTELMDADSDF | DSDF | X | |
| SMO, Gx protein). | GI: 1813875 | | | | |
| Somatostatin receptor type 1 (SS1R, SSR1, | M81829.1 | NLESGGVFRNGTCTSRITTL | ITTL | X | |
| SSTR1, SRIF-2) | GI: 307433 | | | | |
| Somatostatin receptor type 2 (SSR2, SS2R, | M81830.1 | LNETTETQRTLLNGDLQTSI | QTSI | X | AA113 |
| SSTR2, SRIF-1). | GI: 307435 | | | | |
| Somatostatin receptor type 3 (55R3, SS3R, | M96738.1 | LLPQEASTGEKSSTMRISYL | ISYL | X | |
| SSR-2B) | GI: 338498 | | | | |
| Somatostatin receptor type 4 (SS4R, SSTR4) | D16826.1 | EALQPEPGRKRIPLTRTTTF | TTTF | X | A4248 |
| | GI: 693907 | | | | |
| Somatostatin receptor type 5 (SS5R, SSTR5) | AAK61266.1 | EATPPAHRAAANGLMQTSKL | TSKL | X | |
| | GI: 14336736 | | | | |
| Sphingosine1-phosphate receptor Edg-8 (SPPR) | AF317676.1 | TGSPGAPTAARTLVSEPAAD | PAAD | | |
| | GI: 11559845 | | | | |
| Substance-K receptor (NKinin2R, SKR, | M57414.1 | GSGLWFGYGLLAPTKTHVEI | HVEI | X | |
| Neurokinin A receptor, NK-2R) | GI: 189134 | | | | |
| Substance-P receptor (SPR, NK-1 receptor, | S62045.1 | SRSDSKTMTESFSFSSNVLS | NVLS | | |
| NK-1R) | GI: 237994 | | | | |
| Thromboxane A2 receptor (TBXA2R, TXA2-R, | U11271.1 | ASRVQAILVPQPPEQLGLQA | GLQA | | |
| Prostanoid TP receptor) | GI: 511793 | | | | |
| Thyrotropin receptor precursor (TSH-R, | M73747.1 | SHLTPKKQGQISEEYMQ1VL | QTVL | X | |
| Thyroid stimulating hormone receptor) | GI: 903759 | | | | |
| Thyrotropin-releasing hormone receptor (TRH- | D16845.1 | ATKVSFDDTCLASEVSFSQS | FSQS | | |
| R, Thyroliberin receptor) | GI: 577631 | | | | |
| Trace amine receptor 1 (AmineR1) | AF380185.1 | FGKIFQKDSSRCKLFLELSS | ELSS | | |
| | GI: 14600073 | | | | |
| Trace amine receptor 3 (AmineR3) | AF380189.1 | KVLRTDSSTTNLFSEEVETD | VETD | | |
| | GI: 14600081 | | | | |
| Trace amine receptor 4 (AmineR4) | AF380192.1 | VTGQVLKNSSATMNLFSEHI | SEHI | X | |
| | GI: 14600087 | | | | |
| Trace amine receptors (TA5, GPR1o2) | AF411116.1 | LILSGDVLKASSSTISLFLE | LFLE | | |
| | GI: 16566343 | | | | |
| Urotensin II receptor (UR-II-R) | AF140631.1 | LVLAPAAPARPAPEGPRAPA | RAPA | X | |
| | GI: 5902615 | | | | |
| Vasoactive intestinal polypeptide receptor 1 | U11087.1 | TRVSPGARRSSSFQAEVSLV | VSLV | | |
| precursor (VIPR1, Pituitary adenylate cyclase | GI: 508258 | | | | |
| activating polypeptide type II receptor, | | | | | |
| PACAP type II receptor, PACAPR2) | | | | | |
| Vasoactive intestinal polypeptide receptor 2 | L40764.1 | LQFHRGSRAQSFLQTETSVI | TSVI | AA329 | |
| precursor (VIPR2pre, VIP-R-2, Pituitary | GI: 712836 | | | | |
| adenylate cydase activating polypeptide type | | | | | |
| III receptor, PACAP type III receptor, PACAP- | | | | | |
| R-3, Helodermin-preferring VIP receptor) | | | | | |
| Vasopressin receptor type 2 (VasoprR2) | AF032388.1 | VQLWAAWDPEAPLEGGCSRG | CSRG | | |
| | GI: 2654030 | | | | |
| Vasopressin V1a receptor (V1aR, | AAA62271.1 | GMWKDSPKSSKSIKFIPVST | PVST | | |
| Vascular/hepatic-type arginine vasopressin | GI: 667068 | | | | |
| receptor, Antidiuretic hormone receptor 1a, | | | | | |
| AVPR V1a | | | | | |
| Vasopressin V1b receptor (V1bR, AVPR V1b, | D31833.1 | ESPRDLELADGEGTAETIIF | TIIF | X | |
| Vasopressin V3 receptor, AVPR V3, | GI: 563981 | | | | |
| Antidiuretic hormone receptor 1b) | | | | | |
| Vasopressin V2 receptor (Renal-type arginine | U04357.1 | GPQDESCTTASSSLAKDTSS | DTSS | | |
| vasopressin receptor, Antidiuretic hormone | GI: 3004498 | | | | |
| receptor, AVPR V2) | | | | | |
| Vomeronasal receptor 1 (VomNasR1, Putative | AF302903.1 | QFCFACRTRKTLFPNLVVMP | VVMP | | |
| pheromone receptor V1RL1 long form, | GI: 10732801 | | | | |
| VNR19I1, V1RL1). | | | | | |
| Y6 encoding protein (Y6 protein) | D86519.1 | GACWLPRISSMSSLTGIMRC | IMRC | X | |
| | GI: 1731789 | | | | |

TABLE 4

| | PDZ-containing | PDZ |
|---|---|---|
| GPCR gene | gene | Domain(s) |
| alpha1A-Adrenergic receptor | nNOS | |
| beta2-Adrenergic receptor (DSLL) | EBP 50 | 1 |
| beta2-Adrenergic receptor (DSLL) | SIP1 | 1 |
| P2Y1 purinergic receptor (DTSL) | EBP50 | 1 |
| GRK6A (TRL) | EBP50 | 1 |
| beta1-Adrenergic receptor (DSLL) | rat PSD95 | 3 |
| parathyroid hormone 1 receptor | SIP1 | 2 |
| parathyroid hormone 1 receptor | EBP50 | na |
| 5HT2B | cNOS | |
| platelet-derived growth factor receptor | EBP50 | |
| mGLUR1a | shank | |
| mGLUR5 | shank | |
| SSTR2 | shank 1 | |
| SSTR2 | shank2 | |
| IL8RB | RGS12 | |
| CL1 (a-latrotoxin) | shank | |
| 5HT2B | Inadl | 6 |
| B1AR | MAGI2 | 1 |
| rat SSTR2 | CBP1 | |
| 5HT2C | MUPP1 | 10 |
| SSTR2A | CBP1 | |
| CIRL1 | shank2 | |
| CIRL2 | shank2 | |
| CIRL1 & 2 | shank family | |
| prolactin-releasing peptide receptor | GRIP | |
| prolactin-releasing peptide receptor | ABP | |
| prolactin-releasing peptide receptor | PICK1 | |
| kappa opioid receptor | EBP50 | 1 |
| mGLUR7 | PICK1 | |

TABLE 6

Domain

| | | | |
|---|---|---|---|
| Gene Name | GI or Acc # | # | Sequence fused to GST Construct |

| | | | |
|---|---|---|---|
| 26s subunit | 9184389 | 1 | RDMAEAHKEAMSRKLGQSESQGPPRAFAKVNSISPGSPSIAGLQVDDEIVEFGS |
| p27 | | | VNTQNFQSLHNIGSVVQHSEGALAPTILLSVSM |
| AF6 | 430993 | 1 | LRKEPEIITVTLKKQNGMGLSIVAAKGAGQDKLGIYVKSVVKGGAADVDGRLAA |
| | | | GDQLLSVDGRSLVGLSQERAAELMTRTSSVVTLEVAKQG |
| AIPC | 12751451 | 1 | LIRPSVISIIGLYKEKGKGLGFSIAGGRDCIRGQMGIFVKTIFPNGSMEDGRLKEGDE |
| | | | ILDVNGIPIKGLTFQEAIHTFKQIRSGLFVLTVRTKLVSPSLTNSS |
| AIPC | 12751451 | 2 | GISSLGRKTPGPKDRIVMEVTLNKEPRVGLGIGACCLALENSPPGIYIHSLAPGSV |
| | | | AKMESNLSRGDQILEVNSVNVRHAALSKVHAILSKCPPGPVRLVIGRHPNPKV |
| | | | SEQEMDEVIARSTYQESKEANSS |
| AIPC | 12751451 | 3 | QSENEEDVCFIVLNRKEGSGLGFSVAGGTDVEPKSITVHRVFSQGAASQEGTMN |
| | | | RGDFLLSVNGASLAGLAHGNVLKVLHQAQLHKDALVVIKKGMDQPRPSNSS |
| AIPC | 12751451 | 4 | LGRSVAVHDALCVEVLKTSAGLGLSLDGGKSSVTGDGPLVIKRVYKGGAAEQA |
| | | | GIIEAGDEILAINGKPLVGLMHFDAWNIMKSVPEGPVQLLIRKHRNSS |
| alpha actinin-2 | 2773059 | 1 | QTVILPGPAAWGFRLSGGIDFNQPLVITRITPGSKAAAANLCPGDVILAIDGFGTE |
| associated LIM | | | SMTHADGQDRIKAAEFIV |
| protein | | | |
| APXL-1 | 13651263 | 1 | ILVEVQLSGGAPWGFTLKGGREHGEPLVITKIEEGSKAAAVDKLLAGDEIVGINDI |
| | | | GLSGFRQEAICLVKGSHKTLKLVVKRNSS |
| Atrophin-1 | 2947231 | 1 | REKPLFTRDASQLKGTFLSTTLKKSNMGFGFTIIGGDEPDEFLQVKSVIPDGPAAQ |
| Interacting | | | DGKMETGDVIVYINEVCVLGHTHADVVKLFQSVPIGQSVNLVLCRGYP |
| Protein | | | |
| Atrophin-1 | 2947231 | 2 | LSGATQAELMTLTIVKGAQGFGFTIADSPTGQRVKQILDIQGCPGLCEGDLIVEIN |
| Interacting | | | QQNVQNLSHTEVVDILKDCPIGSETSLIIHRGGFF |
| Protein | | | |
| Atrophin-1 | 2947231 | 3 | HYKELDVHLRRMESGFGFRILGGDEPGQPILIGAVIAMGSADRDGRLHPGDELVY |
| Interacting | | | VDGIPVAGKTHRYVIDLMHHAARNGQVNLTVRRKVLCG |
| Protein | | | |
| Atrophin-1 | 2947231 | 4 | EGRGISSHSLQTSDAVIHRKENEGFGFVIISSLNRPESGSTITVPHKIGRIIDGSPAD |
| Interacting | | | RCAKLKVGDRILAVNGQSIINMPHADIVKLIKDAGLSVTLRIIPQEEL |
| Protein | | | |
| Atrophin-1 | 2947231 | 5 | LSDYRQPQDFDYFTVDMEKGAKGFGFSIRGGREYKMDLYVLRLAEDGPAIRNGR |
| Interacting | | | MRVGDQIIEINGESTRDMTHARAIELIKSGGRRVRLLLKRGTGQ |
| Protein | | | |
| Atrophin-1 | 2947231 | 6 | HESVIGRNPEGQLGFELKGGAENGQFPYLGEVKPGKVAYESGSKLVSEELLLEV |
| Interacting | | | NETPVAGLTIRDVLAVIKHCKDPLRLKCVKQGGIHR |
| Protein | | | |
| CARD11 | 12382772 | 1 | NLMFRKFSLERPFRPSVTSVGHVRGPGPSVQHTTLNGDSLTSQLTLLGGNARG |
| | | | SFVHSVKPGSLAEKAGLREGHQLLLLEGCIRGERQSVPLDTCTKEEAHWTIQRC |
| | | | SGPVTLHYKVNHEGYRKLV |
| CARD14 | 13129123 | 1 | ILSQVTMLAFQGDALLEQISVIGGNLTGIFIHRVTPGSAADQMALRPGTQIVMVD |
| | | | YEASEPLFKAVLEDTTLEEAVGLLRRVDGFCCLSVKVNTDGYKRL |
| CASK | 3087815 | 1 | TRVRLVQFQKNTDEPMGITLKMNELNHCIVARIMHGGMIHRQGTLHVGDEIREIN |
| | | | GISVANQTVEQLQKMLREMRGSITFKIVPSYRTQS |
| Connector | 3930780 | 1 | LEQKAVLEQVQLDSPLGLEIHTTSNCQHFVSQVDTQVPTDSRLQIQPGDEVV |
| Enhancer | | | QINEQVVVGWPRKNMVRELLREPAGLSLVLKKIPIP |
| Cytohesin | 3192908 | 1 | QRKLVTVEKQDNETFGFEIQSYRPQNQNACSSEMFTLICKIQEDSPAHCAGLQA |
| Binding | | | GDVLANINGVSTEGFTYKQVVDLIRSSGNLLTIETLNG |
| Protein | | | |
| Densin 180 | 16755892 | 1 | RCLIQTKGQRSMDGYPEQFCVRIEKNPGLGFSISGGISGQGNPFKPSDKGIFVTRVQ |
| | | | PDGPASNLLQPGDKILQANGHSFVHMEHEKAVLLLKSFQNTVDLVIQRELTV |
| DLG1 | 475816 | 1 | IQVNGTDADYEYEEITLERGNSGLGFSIAGGTDNPHIGDDSSIFITKIITGGAAAQD |
| | | | GRLRVNDCILQVNEVDVRDVTHSKAVEALKEAGSIVRLYVKRRN |
| DLG1 | 475816 | 2 | IQLIKGPKGLGFSIAGGVGNQHIPGDNSIYVTKIIEGGAAHKDGKLQIGDKLLAVNN |
| | | | VCLEEVTHEEAVTALKNTSDFVYLKVAKPTSMYMNDGN |
| DLG1 | 475816 | 3 | ILHRGSTGLGFNIVGGEDGEGIFISFILAGGPADLSGELRKGDRIISVNSVDLRMS |
| | | | HEQAAAALKNAGQAVTIVAQYRPEEYSR |
| DLG2 | 12736552 | 1 | ISYVNGTEIEYEFEEITLERGNSGLGFSIAGGTDNPHIGDDPGIFITKIIPGGAAAEDG |
| | | | IRLRVNDCILRVNEVDVSEVSHSKAVEALKEAGSIVRLYVRRR |
| DLG2 | 12736552 | 2 | ISVVEIKLFKGPKGLGFSIAGGVGNQHIPGDNSIYVTKIIDGGAAQKDGRLQVGDRL |
| | | | LMVNNYSLEEVTHEEAVAILKNTSEVVYLKVGNPTTI |
| DLG2 | 12736552 | 3 | IWAVSLEGEPRKVVLHKGSTGLGFNIVGGEDGEGIFVSFILAGGPADLSGELQRG |
| | | | DQILSVNGIDLRGASHEQAAAALKGAGQTVTIIAQYQPED |
| DLG5 | 3650451 | 1 | GIPYVEEPRHVKVQKGSEPLGISIVSGEKGGIYVSKVTVGSIAHQAGLEYGDQLLE |
| | | | FNGINLRSATEQQARLIIGQQCDTITILAQYNPHVHQLRNSSZLTD |
| DLG5 | 3650451 | 2 | GILAGDANKKTLEPRVVFIKKSQLELGVHLCGGNLHGVFVAEVEDDSPAKGPD |
| | | | GLVPGDLILEYGSLDVRNKTVEEVYVEMLKPRDGVRLKVQYRPEEFIVTD |
| DLG6, splice | 14647140 | 1 | PTSPEIQELRQMLQAPHFKALLSAHDTIAQKDFEPLLPPLPDNIPESEEAMRIVC |
| variant 1 | | | LVKNQQPLGATIKRHEMTGDILVARIIHGGLAERSGLLYAGDKLVEVNGVSVEG |
| | | | LDPEQVIHLAMSRGTIMFKVVPVSDPPVNSS |
| DLG6, splice | AB053303 | 1 | PTSPEIQELRQMLQAPHFKGATIKRHEMTGDILVARIIHGGLAERSGLLYAGDKL |
| variant 2 | | | VEVNGVSVEGLDPEQVIHILAMSRGTIMFKVVPVSDPPVNSS |
| DVL1 | 2291005 | 1 | LNIVTVTLNMERHHFLGISIVGQSNDRGDGGIYIGSIMKGGAVAADGRIEPGDMLL |
| | | | QVNDVNFENMSNDDAVRVLREIVSQTGPISLTVAKCW |
| DVL2 | 2291007 | 1 | LNIITVTLNMEKYNFLGISIVGQSNERGDGGIYIGSIMKGGAVAADGRIEPGDMLLQ |
| | | | VNDMNFENMSNDDAVRVLRDIVHKPGPIVLTVAKCWDPSPQNS |
| DVL3 | 6806886 | 1 | IITVTLNMEKYNFLGISIVGQSNERGDGGIYIGSIMKGGAVAADGRIEPGDMLLQVN |
| | | | EINFENMSNDDAVRVLREIVHKPGPITLTVAKCWDPSP |
| ELFIN 1 | 2957144 | 1 | TTQQIDLQGPGPWGFRLVGRKDFEQPLAISRVTPGSKAALANLCIGDVITAIDGE |
| | | | NTSNMTHLEAQNRIKGCTDNLTLTVARSEHKVWSPLV |
| ENIGMA | 561636 | 1 | IFMDSFKVVLEGPAPWGFRLQGGKDFNVPLSISRLTPGGKMQAGVAVGDWVL |
| | | | SIDGENAGSLTHIEAQNKIRACGERLSLGLSRAQPV |
| ERBIN | 8923908 | 1 | QGHELAKQEIRVRVEKDPELGFSISGGVGGRGNPFRPDDDGIFVTRVQPEGPASK |
| | | | LLQPGDKIIQANGYSFINIEHGQAVSLLKTFQNTVELIIVREVSS |
| EZRIN Binding | 3220018 | 1 | ILCCLEKGPNGYGFHLHGEKGKLGQYIRLVEPGSPAEKAGLLAGDRLVEVNGEN |
| Protein 50 | | | VEKETHQQVVSRIRAALNAVRLLVVDPEFIVTD |
| EZRIN Binding | 3220018 | 2 | IRLCTMKKGPSGYGFNLHSDKSKPGQFIRSVDPDSPAEASGLRAQDRIVFVNGV |
| Protein 50 | | | CMEGKQHGDVVSAIRAGGDETKLLVVDRETDEFFMNSS |
| FLJ00011 | 110440352 | 1 | KNPSGELKTVTLSKMKQSLGISISGGIESKVQPMVKIEKIFPGGAAFLSGALQAGFE |
| | | | LVAVDGENLEQVTHQRAVDTIRRAYRNKAREPMELVVRVPGPSPRPSPSD |
| FLJ11215 | 11436365 | 1 | EGHSHPRVVELPKTEEGLGFNIMGGKEQNSPIYISRIIPGGLADRHGGLKRGDQLLS |
| | | | VNGVSVEGEHHEKAVELLKAAQGKVKLVVRYTPKVLEEME |
| FLJ12428 | BC012040 | 1 | PGAPYARKTFTIVGDAVGWGFVVRGSKPCHIQAVDPSGPAAAAGMKVCQFVV |
| | | | SVNGLNVLHVDYRTVSNLILTGPRTIVMEVMEELEC |
| FLJ12615 | 10434209 | 1 | GQYGGETVKIVRIEKARDIPLGATVRNEMDSVIISRIVKGGAAEKSGLLHEGDEVL |
| | | | EINGIEIRGKDVNEVFDLLSDMHGTLTFVLIPSQQIKPPPA |
| FLJ20075 | 7019938 | 1 | ILAHVKGIEKEVNVYKSEDSLGLTITDNGVGYAFIKRIKDGGVIDSVKTICVGDHIE |
| | | | SINGENIVGWRHYDVAKKLKELKKEELFTMKLIEPKKAFEI |
| FLJ21687 | 10437836 | 1 | KPSQASGHFSVELVRGYAGFGLTLGGGRDVAGDTPLAVRGLLKDGPAQRCGRL |
| | | | EVGDLVLHINGESTQGLTHAQAVERIRAGGPQLHLVIRRPLETHPGKPRGV |
| FLJ31349 | AK055911 | 1 | PVMSQCACLEEVHLPNIKPGEGLGMYIKSTYDGLHVITGTTENSPADRSQKIHAG |
| | | | DEVIQVNQQTVVGWQLKNLVKKLRENPTGVVLLLKKRPTGSFNFTPEFIVTD |
| FLJ32798 | AK057360 | 1 | LDDEEDSVKIIRLVKNREPLGATIKKDEQTGAIIVARIMRGGMDRSGLIHVGDEL |
| | | | REVNGIPVEDKRPEEIIQILAQSQGAITFKIIPGSKEETPSNSS |
| GRIP 1 | 4539083 | 1 | VVELMKKEGTTLGLTVSGGIDKDGKPRVSNLRQGGIMRSDQLDVGDYIKAVNG |
| | | | INLAKFRHDEIISLLKNVGERVVLEVEYE |
| GRIP 1 | 4539083 | 2 | RSSVIFRTVEVTLHKEGNTFGFVIRGGAHDDRNKSRPVVITCVRPGGPADREGTI |
| | | | KPGDRLLSVDGIRLLGTTHAEAMSILKQCGQEAALLIEYDVSVMDSVATASGN |
| | | | SS |
| GRIP 1 | 4539083 | 3 | HVATASGPLLVEVAKTPGASLGVALTTSMCCNKQVIVIDKIKSASIADRCGALH |
| | | | VGDHILSIDGTSMEYCTLAEATQFLANTTDQVKLEILPHHQTRLALKGPNSS |
| GRIP 1 | 4539083 | 4 | TETTEVVLTADPVTGFGIQLQGSVFATETHSSPPLISYIEADSPAERCGVLQIGD |
| | | | RVMAINGIPTEDSTFEEASQLLRDSSITSKVTLEIEFDVAES |
| GRIP 1 | 4539083 | 1 | AESVIPSSGTFHVKLPKKHNVELGITISSPSSRKPGDPLVISDIKKGSVAHRTGTL |
| | | | ELGDKLLAIDNIRLDNCSMEDAVQILQQCEDLVKLKIRKDEDNSD |
| GRIP 1 | 4539083 | 6 | IYTVELKRYGGPLGITISGTEEPFDPIIISSLTKGGLAERTGAIHIGDRILAINSSSLKG |
| | | | KPLSEAIHLLQMAGETVTLKIKKQTDAQSA |
| GRIP 1 | 4539083 | 7 | IMSPTPVELHKVTLYKDSDMEDFGFSVADGLLEKGVYVKNIRPAGPGDLGGLKP |
| | | | YDRLLQVNHVRTRDFDCCLVVPLIAESGNKLDLVISRNPLA |
| GTPase | 2389003 | 1 | SRGCETRELALPRDGQGRLGFEVDAEGFVTHVERFTFAETAGLRPGARLLRVCG |
| Activating | | | QTLPSLRPEAAAQLLRSAPKVCVTVLPPDESGRP |
| Enzyme | | | |
| Guanine | 6650765 | 1 | AKAKWRQVVLQKASRESPLQFSLNGGSEKGFGIFVEGVEPGSKAADSGLKRGD |
| Exchange | | | QIMEVNGQNFENITFMKAVEILRNNTHLALTVKTNIFVFKEL |
| Factor | | | |
| HEMBA | 10436367 | 1 | LENVIAKSLLIKSNEGSYGFGLEDKNKVPIIKLVEKGSNAEMAGMEVGKKIFAING |
| 1000505 | | | DLVFMRPFNEVDCFLKSCLNSRKPLRVLVSTKP |
| HEMBA | 10436367 | 2 | PRE1VKIPDSADGLGFQIRGFGPSVVHAVGRGTVAAAAGLHPGQCIIKVNGINVS |
| 1000505 | | | KETHASVIAHVTACRKYRRPTKQDSIQ |
| HEMBA | 7022001 | 1 | EDFCYVFTVELERGPSGLGMGLIDGMHTHLGAPGLYIQTLLPGSPAAADGRLSL |
| 1003117 | | | GDRILEVNGSSLLGLGYLRAVDLIRHGGKKMRFLVAKSDVETAKKI |
| HTRA3 | AY040094 | 1 | LTEFQDKQIKDWKKRFIGIRMRTITPSLVDELKASNPDFPEVSSGIYVQEVAPNS |
| | | | PSQRGGIQDGDIIVKVNGRPLVDSSELQEAVLTESPLLLEVRRGNDDLLFSNSS |
| HTRA4 | AL576444 | 1 | HKKYLGLQMLSLTVPLSEELKMHYPDFPDVSSGVYVCKVVEGTMQSSGLRD |
| | | | HDVIVNINGKPITTTTDVVKALDSDSLSMAVLRGKDNLLLTVNSS |
| INADL | 2370148 | 1 | IWQIEYIDIERPSTGGLGFSVVALRSQNLGKVDIFVKDVQPGSVADRDQRLKEN |
| | | | DQILAINHTPLDQNISHQQAIALLQQTTGSLRLIVAREPVHTKSSTSSSE |
| INADL | 2370148 | 2 | PGHVEEVELINDGSGLGFGIVGGKTSGVVVRTIVPGGLADRDGRLQTGDHILKIGG |
| | | | TNVQGMTSEQVAQVLRNCGNSS |
| INADL | 2370148 | 3 | PGSDSSLFETYNVELVRKDGQSLGIRIVGYVGTSHTGEASGIYVKSIIPGSMYHN |
| | | | GHIQVNDKIVAVDGVNIQGFANHDVVEVLRNAGQVVHLTLVRRKTSSSTSRIH |
| | | | RD |
| INADL | 2370148 | 4 | NSDDAELQKYSKLLPIHTLRLGVEVDSFDGHHYISSIVSGGPVDTLGLLQPEDE |
| | | | LLEVNGMQLYGKSRREAVSFLKEVPPPFTLVCCRRLFDDEAS |
| INADL | 2370148 | 5 | LSSPEVKIVELVKDGKGLGFSILDYQDPLDPTRSVIVIRSLVADGVAERSGGLLP |
| | | | GDRLVSVNEYCLDNTSLAEAVEILKAVPPGLVHLGICKPLVEFIVTD |
| INADL | 2370148 | 6 | PNFSHWGPPRIVEIFREPNVSLGISIVVGQTVIKRLKNGEELKGIFIKQVLEDSPAG |
| | | | KTNALKTGDKILEVSGVDLQNASHSEAVEAIKNAGNPVVFIVQSLSSTPRVIPN |
| | | | VHNKANSS |
| INADL | 2370148 | 7 | PGELHIIELEKDKNGLGLSLAGNKDRSRMSIFVVGINPEGPAMDGRMRIGDELLE |
| | | | INNQILYGRSHQNASAIIKTAPSKVKLVFIRNEDAVNQMANSS |
| INADL | 2370148 | 8 | PATCPIVPGQEMIIEISKGRSGLGLSIVGGKDTPLNAIVIHEVYEEGAAARDGRLW |
| | | | AGDQILEVNGVDLRNSSHEEAITALRQTPQKVRLVVY |
| KIAA0147 | 1469875 | 1 | ILTLILRQTGGLGISIAGGKGSTPYKGDDEGIFISRVSEEGPAARAGVRVGDKLLE |
| | | | VNGVALQGAEHHEAVEALRGAGTAVQMRVWRERMVEPENAEFIVTD |
| KIAA0147 | 1469875 | 2 | PLRQRHVACLARSERGLGFSIAGGKGSTPYRAGDAGIFVSRIAEGGAAHRAGTLQ |
| | | | VGDRVLSINGVDVTEARHDHAVSLLTAASPTIALLLEREAGG |
| KIAA0147 | 1469875 | 3 | ILEGPYPVEEIRLPRAGGPLGLSIVGGSDHSSHPFGVQEPGVFISKVLPRGLMRS |
| | | | GLRVGDRILAVNGQDVRDATHQEAVSALLRPCLELSLLVRRDPAEFIVTD |
| KIAA0147 | 1469875 | 4 | RELCIQKAPGERLGISIRGGARGHAGNPRDPTDEGIFISKVSPTGAAGRDGRLRVG |
| | | | LRLLEVNQQSLLGLTHGEAVQLLRSVGDTLTVLVCDGFEASTDAALEVS |
| KIAA0303 | 2224546 | 1 | PHQPIVIHSSGKNYGFTIRAIRVYVGDSDIYTVHHIVWNVEEGSPACQAGLKAGD |
| | | | LITHINGEPVHGLVHTEVIELLLKSGNKVSITTTPF |
| KIAA0313 | 7657260 | 1 | ILACAAKAKRRLMTLTKPSREAPLPFILLGGSEKGFGIFVDSVDSGSKATEAGLKR |
| | | | GDQILEVNGQNFENIQLSKAMEILRNNTHLSITVKTNLFVFKELLTNSS |
| KIAA0316 | 6683123 | 1 | IPPAPRKVEMRRDPVLGFGFVAGSEKPVVVRSVTPGGPSEGKLIPGDQIVMINDE |
| | | | PVSAAPRERVIDLVRSCKESILLTVIQPYPSPK |
| KIAA0340 | 2224620 | 1 | LNKRTTMPKDSGALLGLKVVGGKMTDLGRLGAFITKVKKGSLADVVGHLRAGD |
| | | | EVLEWNGKPLPGATNEEVYNIILESKSEPQVEIIVSRPIGDIPRIHRD |
| KIAA0380 | 2224700 | 1 | QRCVIIQKDQHGFGFTVSGDRIVLVQSVRPGGAAMKAGVKEGDRIIKVNGTMVT |
| | | | NSSHLEVVKLIKSGAYVALTLLGSS |
| KIAA0382 | 7662087 | 1 | ILVQRCVIIQKDDNGFGLTVSGDNPVFVQSVKEDGAAMRAGVQTGDRIIKVNGT |
| | | | LVTHISNHLEVVKLIKSGSYVALTVQGRPPGNSS |
| KIAA0440 | 2662160 | 1 | SVEMTLRRNGLGQLGFHVNYEGIVADVEPYGYAWQAGLRQGSRLVEICKVAV |
| | | | ATLSHEQMIDLLRTSVTVKVVIIPPHD |
| KIAA0545 | 14762850 | 1 | LKVMTSGWETVDMTLRRNGLGQLGFHVKYDGTVAEVEDYGFAWQAGLRQGS |
| | | | RLVEICKVAVVTLTHDQMIDLLRTSVTVKVVIIPPFEDGTPRRGW |
| KIAA0559 | 3043641 | 1 | HYIFPHARIKITRDSKDHTVSGNGLGIRIVGGKEIPGHSGEIGAYIAXILPGGSAEQT |
| | | | GKLMEGMQVLEWNGIPLTSKTYEEVQSIISQQSGEAEICVRLDLNML |
| KIAA0561 | 3043645 | 1 | LCGSLRPPIVIHSSGKKYGFSLRAIRVYMGDSDVYTVHHVVWSVEDGSPAQEA |
| | | | GLRAGDLITHINGESVLGLVHMDVVELLLKSGNKISLRTTALENTSIKVG |
| KIAA0613 | 3327039 | 1 | SYSVTLTGPGPWGFRLQGGKDFNMPLTISRITPGSKMQSQLSQGDLVVAIDGV |
| | | | NTDTMTHLEAQNKIKSASYNLSLTLQKSKNSS |
| KIAA0751 | 12734165 | 1 | ISRDSGAMLGLKVVGGKMTESGRLCAFITKVKKGSLADTVGHLRPGDEVLEWN |
| | | | GRLLQGATFEEVYNIILESKPEPQVELVVSRPIAIHRD |
| KIAA0807 | 3882334 | 1 | ISALGSMRPPIIIHRAGKKYGFTLRAIRVYMGDSDVYTVHHMVWHVEDGGPASE |
| | | | AGLRQGDLITHVNGEPVHGLVHTEVVELILKSGNKVAISTTPLENSS |
| KIAA0858 | 4240204 | 1 | FSDMRISINQTPGKSLDFGFTIKWDIPGIFVASVEAGSPAEFSQLQVDDEIIAINNT |
| | | | KFSYNDSKEWEEAMAKAQETGHLVMDVRRYGKAGSPE |
| KIAA0902 | 4240292 | 1 | QSAHLEVIQLANIKPSEGLGMYIKSTYDGLHVITGTTENSPADRCKKIHAGDEVI |
| | | | QVNHQTVVGWQLKNLVNALREDPSGVILTLKKRPQSMLTSAPA |
| KIAA0967 | 4589577 | 1 | ILTQTLIPVRHTVKIDKDTLLQDYGFHISESLPLTVVAVTAGGSAHGKLFPGDQI |
| | | | LQMNNEPAEDLSWERAVDILREAEDSLSITVVRCTSGVPKSSNSS |
| KIAA0973 | 4589589 | 1 | GLRSPITIQRSGKKYGFTLRAIRVYMGDTDVYSVHHIVWHVEEGGPAQEAGLCA |
| | | | GDLITHVNGEPVHGMVHPEVVELILKSGNKVAVTTTPFE |
| KIAA1095 | 5889526 | 1 | QGEETKSLTLVLHRDSGSLGFNIIGGRPSVDNHDGSSSEGIFVSKIVDSGPAAKE |
| | | | GGLQIHDRIIEVNGRDLSRATHDQAVEAFKTAKEPIVVQVLRRTPRTKMFTP |
| KIAA1095 | 5889526 | 2 | QEMDREELELEEVDLYRMNSQDKLGLTVGYRTDDEDDIGIYISEIDPNSIAAKDG |
| | | | RIREGDRIIQINGIEVQNREEAVALLTSEENKNFSLLIARPELQLD |
| KIAA1202 | 6330421 | 1 | RSFQYVPVQLQGGAPWGFTLKGGLEHCEPLTVSKIEDGGKAALSQKMRTGDEL |
| | | | VNINGTPLYGSRQEALILIKGSFRILKLIVRRRNAPVS |
| KIAA1222 | 6330610 | 1 | ILEKLELFPVELEKDEDGLGISIIGMGVGADAGLEKLGIFVKTVTEGGAAQRDGRIQ |
| | | | VNDQIVEVDGISLVGVTQNFAATVLRNTKGNVRFVIGREKPGQVS |
| KIAA1284 | 6331369 | 1 | KDVNVYVNPKKLTVIKAKEQLKLLEVLVGIIHQTKWSWRRTGKQGDGERLVVH |
| | | | GLLPGGSAMKSGQVLIGDVLVAVNDVDVTTENIERVLSCIPGPMQVKLTFENA |
| | | | YDVKRET |
| KIAA1389 | 7243158 | 1 | TRGCETVEMTLRRNGLGQLGFHVNFEGIVADVEPFGFAWKAGLRQGSRLVEICK |
| | | | VAVATLTHEQMIDLLRTSVTVKVVIIQPHDDGSPRR |
| KIAA1415 | 7243210 | 1 | VENILAKRLLILPQEEDYGFDIEEKNKAVVVKSVQRGSLAEVAGLQVGRKIYSIN |
| | | | EDLVFLRPFSEVESILNQSFCSRRPLRLLVATKAKEIIKIP |
| KIAA1526 | 5817166 | 1 | PDSAGPGEVRLVSLRRAKAHEGLGFSIRGGSEHGVGIYVSLVEPGSLAEKEGLR |
| | | | VGDQILRVNDKSLARVTHAEAVKALKGSKKLVLSVYSAGRIPGGYVTNH |
| KIAA1526 | 5817166 | 2 | LQGGDEKKVNLVLGDGRSLGLTIRGGAEYGLGIYITGVDPGSEAEGSGLKVGDQI |
| | | | LEVNWRSFLNILHDEAVRLLKSSRHLILTVKDVGRLPHARTTVDE |
| KIAA1526 | 5817166 | 3 | WTSGAHVHSGPCEEKGGHPGHRQPLPRIVTIQRGGSAHNCGQLKVGHVILEVN |
| | | | GLTLRGKEHREAARIIAEAFKTKDRDYIDFLDSL |
| KIAA1620 | 10047316 | 1 | ELRRAELVEIIVETEAQTGVSGINVAGGGKEGIFVRELREDSPMRSLSLQEGDQ |
| | | | LLSARVFFENFKYEDALRLLQCAEPYKVSFCLKRTVPTGDLALRP |
| KIAA1634 | 10047344 | 1 | PSQLKGVLVRASLKKSTMGFGFTDGGDRPDEFLQVKNVLKDGPMQDGKIAPG |
| | | | DVIVDINGNCVLGHTHADVVQMFQLVPVNQYVNLTLCRGYPLPDDSED |
| KIAA1634 | 10047344 | 2 | ASSGSSQPELVTIPLIKGPKGFGFAIADSPTGQKVKMILDSQWCQGLQKGDIIKEIY |
| | | | HQNVQNLTHLQVVEVLKQFPVGADVPLLILRGGPPSPTKTAKM |
| KIAA1634 | 10047344 | 3 | LYEDKPPLTNTFLISNPRTTADPRILYEDKPPNTKDLDVFLRKQESGFGFRVLGG |
| | | | DGPDQSIYIGAIIPLGAAEKDGRLRAADELMCIDGIPVKGKSHKQVLDLMTTAAR |
| | | | NGHVLLTVRRKIFYGEKQPEDDSGSPGIHRELT |
| KIAA1634 | 10047344 | 4 | PAPQEPYDVVLQRKENEGFGFVILTSKNKPPPGVIPHKIGRVIEGSPADRCGKLK |
| | | | VGDHISAVNGQSIVELSHDNIVQLIKDAGVTVTLTVIAEEEHHGPPS |
| KIAA1634 | 10047344 | 5 | QNLGCYPVELERGPRGFGFSLRGGKEYNMGLFILRLAEDGPAIKDGRIHVGDQIVE |
| | | | INGEPTQGITHTRAIELIQAGGNKVLLLLRPGTGLIPDHGLA |
| KIAA1719 | 1267982 | 0 | ITVVELIKKEGSTLGLTISGGTDKDGKPRVSNLRPGGLAARSDLLNIGDYIRSVNGI |
| | | | HLTRLRHDEIITLLKNVGERVVLEVEY |
| KIAA1719 | 1267982 | 1 | ILDVSLYKEGNSFGFVLRGGAHEDGHKSRPLVLTYVRPGGPADREGSLKVGDRL |
| | | | LSVDGIPLHGASHATALATLRQCSHEALFQVEYDVATP |
| KIAA1719 | 1267982 | 2 | IHTVANASGPLMVEIVKTPGSALGISLTTTSLRNKSVITIDRIKPASVVDRSGALH |
| | | | PGDHILSIDGTSMEHCSLLEATKLLASISEKVRLEILPVPQSQRPL |
| KIAA1719 | 1267982 | 3 | IQIVHTETTEVVLCGDPLSGFGLQLQGGIFATETLSSPPLVCFIEPDSPAERCGLL |
| | | | QVGDRVLSINGIATEDGTMEEANQLLRDAALAHKVVLEVEFDVAESV |
| KIAA1719 | 1267982 | 4 | IQFDVAESVIPSSGTFHVKLPKKRSVELGITISSASRKRGEPLIISDIKKGSVAHRTG |
| | | | TLEPGDKLLAIDNIRLDNCPMEDAVQILRQCEDLVKLKIRKDEDN |
| KIAA1719 | 1267982 | 5 | IQTTGAVSYTVELKRYGGPLGITISGTEEPFDPIVISGLTKRGLAERTGAIHVGDRIL |
| | | | AINNVSLKGRPLSEAIHLLQVAGETVTLKIKKQLDR |
| KIAA1719 | 1267982 | 6 | ILEMEELLLPTPLEMHKVThHKDPMRHDFGFSVSDGLLEKGVYVHTVRPDGPA |
| | | | HRGGLQPFDRVLQVNHVRTRDFDCCLAVPLLAEAGDVLELIISRKPHTAHSS |
| LIM Mystique | 12734250 | 1 | MALTVDVAGPAPWGFRITGGRDFHTPIMVTKVAERGKAKDADLRPGDIIVAING |
| | | | ESAEGMLHAEAQSKIRQSPSPLRLQLDRSQATSPGQT |
| LIM Protein | 3108092 | 1 | SNYSVSLVGPAPWGFRLQGGKDFNMPLTISSLKDGGKAAQANVRIGDVVLSID |
| | | | GINAQGMTTHLEAQNKIKGCTGSLNMTLQRAS |
| LIMK1 | 4587498 | 1 | TLVEHSKLYCGHCYYQTVVTPVIEQILPDSPGSHLPHTVTLVSIPASSHGKRGL |
| | | | SVSIDPPHGPPGCGTEHSHTVRVQGVDPGCMSPDVKNSIHVGDRILEINGTPIRN |
| | | | VPLDEIDLLIQETSRLLQLTLEHD |
| LIMK2 | 1805593 | 1 | PYSVTLISMPATTEGRRGFSVSVESACSNYATTVQVKEVNRMHISPNNRNAIHP |
| | | | GDRILEINGTPVRTLRVEEVEDAISQTSQTLQLLIEHD |
| LIM-RIL | 1085021 | 1 | IHSVTLRGPSPWGFRLVGRDFSAPLTISRVHAGSKASLAALCPGDLIQAINGEST |
| | | | ELMTLLEAQNRIKGCHDHLTLSVSRPE |
| LU-1 | U52111 | 1 | VCYRTDDEEDLGIYVGEVNPNSIMKDGRIREGDRIIQINGVDVQNREEAVAILS |
| | | | QEENTNISLLVARPESQLA |
| MAGI1 | 3370997 | 1 | IQKKNHWTSRVHECTVKRGPQGELGVTVLGGAEHGEFPYVGAVAAVEAAGLP |
| | | | GGGEGPRLGEGELLLEVQGVRVSGLPRYDVLGVIDSCKEAVTFKAVRQGGR |
| MAGI1 | 3370997 | 2 | PSELKGKFIHTKLRKSSRGFGFTVVGGDEPDEFLQIKSLVLDGPAALDGKMETGD |
| | | | VIVSVNDTCVLGHTHAQVVKIFQSIPIGASVDLELCRGYPLPFDPDDPN |
| MAGI1 | 3370997 | 3 | PATQPELITVHIVKGPMGFGFTIADSPGGGGQRVKQIVDSPRCRGLKEGDLIVEVN |
| | | | KKNVQALTHNQVVDMLVECPKGSEVTLLVQRGGNLS |
| MAGI1 | 3370997 | 4 | PDYQEQDIFLWRKETGFGFRILGGNEPGEPIYIGHIVPLGAADTDGRLRSGDELICV |
| | | | DGTPVIGKSHQLVVQLMQQAAKQGHVNLTVRRKVVFAVPKTENSS |
| MAGI1 | 3370997 | 5 | GVVSTVVQPYDVEIRRGENEGFGFVIVSSVSRPEAGTTFAGNACVAMPHKIGRII |
| | | | EGSPADRCGKLKVGDRILAVNGCSITNKSHSDIVNLIKEAGNTVTLRIIPGDESSN |
| | | | A |
| MAGI1 | 3370997 | 6 | QATQEQDFYTVELERGAKGFGFSLRGGREYNMDLYVLRLAEDGPAERCGKMRIG |
| | | | DEILEINGETTKNMKHSRAIELIKNGGRRVRLFLKRG |
| MGC5395 | BC012477 | 1 | PAKMEKEETTRELLLPNWQGSGSHGLTIAQRDDGVFVQEVTQNSPAARTGVV |
| | | | KEGDQIVGATIYFDNLQSGEVTQLLNTMGHHTVGLKLHRKGDRSPNSS |
| MINT1 | 2625024 | 1 | SENCKdVFIEKQKGEILGVVIVESGWGSILPTVIIANMMHGGPAEKSGKLNIGDQIM |
| | | | SINGTSLVGLPLSTCQSIIKGLKNQSRVKLNIVRCPPVNSS |
| MINT1 | 2625024 | 2 | LRCPPVTTVLIRRPDLRYQLGFSVQNGIICSLMRGGIAERGGVRVGHRIIEINGQSV |
| | | | VATPHEKIVHILSNAVGEIHMKTMPAAMYRLLNSS |
| MINT3 | 3169808 | 1 | LSNSDNCREVHLEKRRGEGLGVALVESGWGSLLPTAVIANLLHGGPAERSGAL |
| | | | SIGDRLTAINGTSLVGLPLAACQAAVRETKSQTSVTLSIVHCPPVTTAIM |
| MINT3 | 3169808 | 2 | LVHCPPVTTAIIHRPHAREQLGFCVEDGIICSLLRGGIAERGGIRVGHRIIEINGQSV |
| | | | VATPHARIIELLTEAYGEVHIKTMPAATYRLLTG |
| MPP1 | 189785 | 1 | RKVRLIQFEKVTEEPMGITLKLNEKQSCTVARILHGGMIHRQGSLHVGDEILEING |
| | | | TNVTNHSVDQLQKAMKETKGMISLKVIPNQ |
| MPP2 | 939884 | 1 | PVPPDAVRMVGIRKTAGEHLGVTFRVEGGELVIARILHGGMVAQQGLLHVGDIIK |
| | | | EVNGQPVGSDPRALQELLRNASGSVILKILPNYQ |
| MUPP1 | 2104784 | 1 | GRHVEVFELLKPPSGGLGFSVVGLRSENRGELGIFVQEIQEGSVAHRDGRLKET |
| | | | DQILAINGQALDQTITHQQAISILQKAKDTVQLVIARGSLPQLV |
| MUPP1 | 2104784 | 2 | PVHWQHMETIELVNDGSGLGFGIIGGKATGVIVKTILPGGVADQHGRLCSGDHIL |
| | | | KIGDTDLAGMSSEQVAQVLRQCGNRVKLMIARGAIEERTAPT |
| MUPP1 | 2104784 | 3 | QESETFDVELTKNVQGLGITIAGYIGDKKLEPSGIFVKSITKSSAVEHDGRIQIGDQI |
| | | | IAVDGTNLQGFTNQQAVEVLRHTGQTVLLTLMRRGMKQEA |
| MUPP1 | 2104784 | 4 | LNYEIVVAHVSKFSENSGLGISLEATVGHHFIRSVLPEGPVGHSGKLFSGDELLE |
| | | | VNGITLLGENHQDVVNILKELPIEVTMVCCRRTVPPT |
| MUPP1 | 2104784 | 5 | WEAGIQHIELEKGSKGLGFSILDYQDPIDPASTVIIIRSLVPGGIAEKDGRLLPGDR |
| | | | LMFVNDVNLENSSLEEAVEALKGAPSGTVRIGVAKPLPLSPEE |
| MUPP1 | 2104784 | 6 | RNVSKESFERTIMAKGNSSLGMIVSANKDGLGMIVRSIIHGGAISRDGRIAIGDCIL |
| | | | SINEESTISVTNAQARAMLRRHSLIGPDIKITYVPAEHLEE |
| MUPP1 | 2104784 | 7 | LNWNQPRRVELWREPSKSLGISIVGGRGMGSRLSNGEVMRGIFIKHVLEDSPAG |
| | | | KNGTLKPGDRIVEVDGMDLRDASHEQAVEAIRKAGNPVVFMVQSIINRPRKSPL |
| | | | PSLL |
| MUPP1 | 2104784 | 8 | LTGELHMIELEKGHSGLGLSLAGNKDRSRMSVFIVGIDPNGAAGKDGRLQIADEL |
| | | | LEINGQILYGRSHQNASSIIKCAPSKVKIIFIRNKDAVNQ |
| MUPP1 | 2104784 | 9 | LSSFKNVQHLELPKDQGGLGIAISEEDTLSGVIIKSLTEHGVAATDGRLKVGDQI |
| | | | LAVDDEIVVGYPIEKFISLLKTAKMTVKLTIHAENPDSQ |
| MUPP1 | 2104784 | 10 | LPGCETTIEISKGRTGLGLSIVGGSDTLLGAIIIHEVYEEGAACKDGRLWAGDQILE |
| | | | VNGIDLRKATHDEAINVLRQTPQRVRLTLYRDEAPYKE |
| MUPP1 | 2104784 | 11 | KEEEVCDTLTIELQKKPGKGLGLSIVGKRNDTGVFVSDIVKGGIADADGRLMQGD |
| | | | QILMVNGEDVRNATQEAVAALLKCSLGTVTLEVGRIKAGPFHS |
| MUPP1 | 2104784 | 12 | LQGLRTVEMKKGPTDSLGISIAGGVGSPLGDVPIFIAMMHPTGVMQTQKLRVG |
| | | | DRIVTICGTSTEGMTHTQAVNLLKNASGSIEMQVVAGGDVSV |
| MUPP1 | 2104784 | 13 | LGPPQCKSITLERGPDGLGFSIVGGYGSPHGDLPIYVKTVFAKGMSEDGRLKRG |
| | | | DQIIAVNGQSLEGVTHEEAVAILKRTKGTVTLMVLS |
| NeDLG | 10863920 | 1 | IQYEEIVLERGNSGLGFSIAGGIDNPHVPDDPGIFITKIIPGGAAAMDGRLGVNDCV |
| | | | LRVNEVEVSEVVHSRAVEALKEAGPVVRLVVRRRQN |
| NeDLG | 10863920 | 2 | ITLLKGPKGLGFSIAGGIGNQHIPGDNSIYITKIIEGGAAQKDGRLQIGDRLLAVNNT |
| | | | NLQDVRHEEAVASLKNTSDMVYLKVAKPGSLE |
| NeDLG | 10863920 | 3 | ILLHKGSTGLGFNIVGGEDGEGIFVSFILAGGPADLSGELRRGDRILSVNGVNLRN |
| | | | ATHEQAAAALKRAGQSVTIVAQYRPEEYSRFESKIHDLREQMMNSSMSSGSGS |
| | | | LRTSEKRSLE |
| Neurabin II | AJ401189 | 1 | CVERLELFPVELEKDSEGLGISIIGMGAGADMGLEKLGIFVKIVTFGGAAHRDGRI |
| | | | QVNDLLVEVDGTSLVGVTQSFAASVLRNTKGRVRFMIGRERPGEQSEVAQRIH |
| | | | RD |
| NOS1 | 642525 | 1 | IQPNVISVRLFKRKVGGLGFLVKERVSKPPVIISDLIRGGAAEQSGLIQAGDIILAV |
| | | | NGRPLVDLSYDSALEVLRGIASETHVVLILRGP |
| novel PDZ | 7228177 | 1 | QANSDESDIIHSVRVEKSPAGRLGFSVRGGSEHGLGIFVSKVEEGSSAERAGLCV |
| gene | | | GDKJTEVNGLSLESTTMGSAVKVLTSSSRLHMMVRRMGRVPGIKFSKEKNSS |
| novel PDZ | 7228177 | 2 | PSDTSSEDGVRRIVHLYTTSDDFCLGFNIRGGKEFGLGIYVSKVDHGGLAEENGIK |
| gene | | | VGDQVLAANGVRFDDISHSQAVEVLKGQTHIMLTIKETGRYPAYKEMNSS |
| Novel Serine | 1621243 | 1 | KIKKFLTESHDRQAKGKAITKKKYIGIRMMSLTSSKAKELKDRHRDFPDVISGAYII |
| Protease | | | EVIPDTPAEAGGLKENDVIISINGQSVVSANDVSDVIKRESTLNMVVRRGNEDI |
| | | | MITV |
| Numb Binding | AK056823 | 1 | PDGEITSIKINRVDPSESLSIRLVGGSETPLVHIIIQHIYRDGVIARDGRLLPGDIILK |
| Protein | | | VNGMDISNVPHNYAVRLLRQPCQVLWLTVMREQKFRSRNSS |
| Numb Binding | AK056823 | 2 | HRPRDDSFHVILNKSSPEEQLGIKLVRKVDEPGVFIFNVLDGGVAYRHGQLEEN |
| Protein | | | DRVLAINGHDLRYGSPESAAHLIQASERRVHLVVSRQVRQRSPENSS |
| Numb Binding | AK056823 | 3 | PTITCHEKVVNIQKDPGESLGMTVAGGASHREWDLPIYVISVEPGGVISRDGRIK |
| Protein | | | TGDILLNVDGVELTEVSRSEAVALLKRTSSSIVLKALEVKEYEPQEFIV |
| Numb Binding | AK056823 | 4 | PRCLYNCKDIVLRRNTAGSLGFCIVGGYEEYNGNKPFFIKSIVEGTPAYNDGRIRCG |
| Protein | | | DILLAVNGRSTSGMIHACLARLLKELKGRITLTIVSWPGTFL |
| Outer | 7023825 | 1 | LLTEEEINLTRGPSGLGFNIVGGTDQQYVSNDSGIYVSRIKENGAAALDGRLQEG |
| Membrane | | | DKILSVNGQDLKNLLHQDAVDLFRNAGYAVSLRVQHRLQVQNGIHS |
| p55T | 12733367 | 1 | PVDAIRILGIHKRAGEPLGVTFRVENNDLVIARILHGGMIDRQGLLHVGDIIKEVN |
| | | | GHEVGNNPKELQELLKNISGSVTLKILPSYRDTITPQQ |
| PAR3 | 8037914 | 1 | DDMVKLVEVPNDGGPLGIHVVPFSARGGRTLGLLVKRLEKGGKAEHENLFREN |
| | | | DCIVRINDGDLRNRRFEQAQHMFRQAMRTPIIWFHVVPAA |
| PAR3 | 8037914 | 2 | GKRLNIQLKKGTEGLGFSITSRDVTIGGSAPIYVKNILPRGAAIQDGRLKAGDRLIE |
| | | | VNGVDLVGKSQEEVVSLLRSTKMEGTVSLLVFRQEDA |
| PAR3 | 8037914 | 3 | TPDGTREFLTFEVPLNDSGSAGLGVSVKGNRSKENHADLGIFVKSIINGGMSKD |
| | | | GRLRVNDQLIAVNGESLLGKTNQDAMETLRRSMSTEGNKRGMIQLIVA |
| PAR6 | 2613011 | 1 | LPETHRRVRLHKHGSDRPLGFYIRDGMSVRVAPQGLERVPGIFISRLVRGGLAES |
| | | | TGLLAVSDEILEVNGIEVAGKTLDQVTDMMVANSHNLIVTVKPANQR |
| PAR6 GAMMA | 13537118 | 1 | IDVDLVPETHRRVRLHRHGCEKPLGFYIRDGASVRVTPHGLEKVPGIFISRMVPG |
| | | | GLAESTGLLAVNDEVLEVNGIEVAGKTLDQVTDMMIANSHNLIVTVKPANQR |
| | | | NNVV |
| PDZ-73 | 5031978 | 1 | RSRKLKEVRLDRLHPEGLGLSVRGGLEFGCGLFISHLIKGGQADSVGLQVGDEIV |
| | | | RINGYSISSCTHEEVINLIRTKKTVSIKVRHIGLIPVKSSPDEFH |
| PDZ-73 | 5031978 | 2 | IPGNRENKEKKVFISLVGSRGLGCSISSGPIQKPGIFISHVKPGSLSAEVGLEIGDQI |
| | | | VEVNGVDFSNLDHKEAVNVLKSSRSLTISIVAAAGRELFMTDEF |
| PDZ-73 | 5031978 | 3 | PEQIMGKDVRLLRIKKEGSLDLALEGGVDSPIGKVVVSAVYERGAAERHGGIVKG |
| | | | DEIMAINGKIVTDYTLAEADAALQKAWNQGGWIDLVVAVCPPKEYDD |
| PDZK1 | 2944188 | 1 | LTSTFNPRECKLSKQEGQNYGFFLRIEKDTEGHLVRVVEKCSPAEKAGLQDGDR |
| | | | VLRINGVFVDKEEHMQVVDLVRKSGNSVTLLVLDGDSYEKAGSPGIHRD |
| PDZK1 | 2944188 | 2 | RLCYLVKEGGSYGFSLKIVQGKKGVYMTDITPQGVAMRAGVLADDHLIEVNGE |
| | | | NVEDASHEEVVEKVKKSGSRVMFLLVDKETDKREFIVTD |
| PDZK1 | 294418 | 3 | QFKRETASLKLLPHQPRIVEMKKGSNGYGFYLRAGSEQKGQIIKDIDSGSPAEEAG |
| | | | LKNNDLVVAVNGESVETLDHDSVVEMIRKGGDQTSLLVVDKETDNMYRLAEF |
| | | | IVTD |
| PDZK1 | 294418 | 4 | PDTTEEVDHKPKLCRLAKGENGYGFHLNAIRGLPGSFIKEVQKGGPADLAGLED |
| | | | EDVIIEVNGVNVLDEPYEKVVDRIQSSGKNVTLLVZGKNSS |
| PICK1 | 4678411 | 1 | PTVPGKVTLQKDAQNLIGISIGGGAQYCPCLYIVQVFDNTPAALDGTVAAGDEIT |
| | | | GVNGRSIKGKTKVEVAKMIQEVKGEVTIHYNKLQ |
| PIST | 98374330 | 1 | SQGVGPIRKVLLLKEDHEGLGISITGGKEHGVPILISEIHPGQPADRCGGLHVGDAI |
| | | | LAVNGVNLRDTKHKEAVTILSQQRGEIEFEVVYVAPEVDSD |
| prIL16 | 147849 | 1 | IHVTILHKEEGAGLGFSLAGGADLENKVITVHRVFPNGLASQEGTIQKGNEVLSI |
| | | | NGKSLKGTTHHDALAILRQAREPRQAVIVTRKLTPEEFIVTD |
| prIL16 | 1478492 | 2 | TAEATVCTVTLEKMSAGLGFSLEGGKGSLHGDKPLTINRIFKGAASEQSETVQP |
| | | | GDEILQLGGTAMQGLTRFEAWNIIKALPDGPVTIVIRRKSLQSK |
| PSD95 | 3318652 | 1 | LEYEeITLERGNSGLGFSIAGGTDNPHIGDDPSIFITKIIPGGAAAQDGRLRVNDSIL |
| | | | FVNEVDVREVTHSAAVEALKEAGSIVRLYVAARRKPPAENSS |
| PSD95 | 3318652 | 2 | HVMRRKPPAEKVMEIKLIKGPKGLGFSIAGGVGNQHIPGDNSIYVTKIIEGGAAHK |
| | | | DGRLQIGDKILAVNSVGLEDVMHEDAVAALKNTYDVVYLKVAKPSNAYL |
| PSD95 | 3318652 | 3 | REDIPREPRRIVIHRGSTGLGFNIVGGEDGEGIFISFILAGGPADLSGELRKGDQILSV |
| | | | NGVDLRNASHEQAAIALKNAGQTVTIIAQYKPEFIVTD |
| PTN-3 | 179912 | 1 | LIRITPDEDGKFGFNLKGGVDQKMPLVVSRINPESPADTCIPKLNEGDQIVLINGR |
| | | | DISEHTHDQVVMFIKASRESHSRELALVIRRR |
| PTN-4 | 190747 | 1 | IRMKPDENGRFGFNVKGGYDQKMPVIVSRVAPGTPADLCVPRLNEGDQVVLING |
| | | | RDIAEHTHDQVVLFIKASCERHSGELMLLVRPNA |
| PTPL1 | 515030 | 1 | PEREITLVNLKKDAKYGLGFQIIGGEKMGRLDLGIFISSVAPGGPADFHGCLKPGDR |
| | | | LISVNSVSLEGVSHHAAIEILQNAPEDVTLVISQPKEKISKVPSTPVHL |
| PTPL1 | 515030 | 2 | GDIFEVELAKNDNSLGISVTGGVNTSVRHGGIYVKAVIPQGAAESDGRIHKGDRV |
| | | | LAVNGVSLEGATHKQAVETLRNTGQVVHLLLEKGQSPTSK |
| PTPL1 | 515030 | 3 | TEENTFEVKLFKNSSGLGFSFSREDNLIPEQINASIVRVKKLFAGQPMESGKIDV |
| | | | GDVILKVNGASLKGLSQQEVISALRGTAPEVFLLLCRPPPGVLPEIDT |
| PTPL1 | 515030 | 4 | ELEVELLITLIKSEKASLGFTVTKGNQRIGCYVHDVIQDPAKSDGRLKPGDRLIKV |
| | | | NDTDVTNMTHTDAVNLLRAASKTVRLVIGRVLELPRIPMLPH |
| PTPL1 | 515030 | 5 | MLPHLLPDITLTCNKEELGFSLCGGHDSLYQVVYISDINPRSVMIEGNLQLLDV |
| | | | IHYVNGVSTQGMTLEEVNRALDMSLPSLVLKATRNDLPV |
| RGS12 | 3290015 | 1 | RPSPPRVRSVEVARGRAGYGFTLSGQAPCVLSCVMRGSPADFVGLRAGDQILA |
| | | | VNEINVKKASHEDVVKLIGKCSGVLHMVIAEGVGRFESCS |
| RGS3 | 18644735 | 1 | LCSERRYRQITIPRGKDGFGFTICCDSPVRVQAVDSGGPAERAGLQQLDTVLQL |
| | | | NERPVEHWKCVELAHEIRSCPSEIILLVWRMVPQVKPGIHRD |
| Rhophilin-like | 14279408 | 1 | ISFSANKRWTPPRSIRFTAEEGDLGFTLRGNAPVQVHFLDPYCSASVAGAREGD |
| | | | YIVSIQLVDCKWLTLSEVMKLLKSFGEDEIEMKVVSLLDSTSSMHNKSAT |
| Serine | 2738914 | 1 | RGEKKNSSSGISGSQRRYIGVMMLTLSPSILAELQLREPSFPDVQHGVLIHKVILG |
| Protease | | | SPAHRAGLRPGDVILAIGEQMVQNAEDVYEAVRTQSQLAVQIRRGRETLTLYV |
| Shank 1 | 6049185 | 1 | EEKIVVLQKKDNEGFGFVLRGAKADTPIEEFTPTPAFPALQYLESVDEGGVAW |
| | | | QAGLRTGDFLIEVNNENVVKVGHRQVVNMIRQGGNHLVLKVVTVTRNLDPDD |
| | | | TARKKA |
| Shank 3 | | 1 | SDYVIDDKVAVLQKRDHEGFGFVLRGAKAETPIEEFTPTPAFPALQYLESVDVE |
| | | | GVAWRAGLRTGDFLIEVNGVNVVKVGHKQVVALIRQGGNRLVMKVVSVTRKP |
| | | | EEDG |
| Shroom | 18652858 | 1 | IYLEAFLEGGAPWGFTLKGGLEHGEPLIISKVEEGGKADTLSSKLQAGDEVVHIN |
| | | | EVTLSSSRKEAVSLVKGSYKTLRLVVRRDVCTDPGH |
| SIP1 | 2047327 | 1 | IRLCRLVRGEQGYGFHLHGEKGRRGQFIRRVEPGSPAEAAALRAGDRLVEVNGV |
| | | | NVEGETHHQVVQRIKAVEGQTRLLVVDQN |
| SIP1 | 2047327 | 2 | IRHLRKGPQGYGFNLHSDKSRPGQYIRSVDPGSPAARSGLRAQDRLIEVNGQNV |
| | | | EGLRHAEVVASIKAREDEARLLVVDPETDE |
| SITAC-18 | 8886071 | 1 | PGVREIHLCKDERGKTGLRLRKVDQGLFVQLVQANTPASLVGLRFGDQLLQIDG |
| | | | RDCAGWSSHKAHQVVKKASGDKIVVVVRDRPFQRTVTM |
| SITAC-18 | 8886071 | 2 | PFQRTVTMHKDSMGHVGFVIKKGKIVSLVKGSSAARNGLLTNHYVCEVDGQNV |
| | | | IGLKDKKIMEILATAGNVVTLTIIPSVIYEHIVEFIV |
| SSTRIP | 7025450 | 1 | LKEKTVLLQKKDSEGFGFVLRGAKAQTPIEEFTPTPAFPALQYLESVDEGGVAW |
| | | | RAGLRMGDFLIEVNGQNVVKVGHRQVVNMIRQGGNTLMVKVVMVTRHPDMD |
| | | | EAVQ |
| SYNTENIN | 2795862 | 1 | LEIKQGIREVILCKDQDGKIGLRLKSIDNGIFVQLVQANSPASLVGLRFGDQVLQI |
| | | | NGENCAGWSSDKAHKVLKQAFGEKITMRIHRD |
| SYNTENIN | 2795862 | 2 | RDRPFERTTTMHKDSTGHVGFIFKNGKITSIVKDSSAARNGLLTEHNICEINGQNV1 |
| | | | GLKDSQIADILSTSGNSS |
| Syntrophin 1 | 1145727 | 1 | QRRRVTVRKADAGGLGISIKGGRENKMPILISKIFKGLAADQTEALFVGDAILSVN |
| alpha | | | GEDLSSATLDEAVQVLKKTGKEVVLEVKYMKDVSPYFK |
| Syntrophin | 476700 | 1 | IRVVKQEAGGLGISIKGGRENRMPILISKIFPGLAADQSRALRLGDAILSVNGTDLR |
| beta 2 | | | QATHDQAVQALKRAGKEVLLEVKFIREFIVTD |
| Syntrophin | 9507162 | 1 | EPFYSGERTVTIRRQTVGGFGLSIKGGAEHNIPVVVSKISKEQRAELSGLLFIGDAI |
| gamma 1 | | | LQINGINVRKCRHEEVVQVLRNAGEEVTLTVSFLKRAPAFLKLP |
| Syntrophin | 9507164 | 1 | SHQGRNRRTVTLRRQPVGGLGLSIKGGSEHNVPVVISKIFEDQAADQTGMLFVG |
| gamma 2 | | | DAVLQVNGIHVENATHEEVVHLLRNAGDEVTITVEYLREAPAFLK |
| TAX2-like | 3253116 | 1 | RGETKEVEVTKTEDALGLTITDNGAGYAFIKRIKEGSIINRIEAVCVGDSIEAINDH |
| protein | | | SIVGCRHYEVAKMLRELPKSQPFTLRLVQPKRAF |
| TIAM 1 | 4507500 | 1 | HSIHIEKSDTAADTYGFSLSSVEEDGIRRLYVNSVKETGLASKKGLKAGDEILEIN |
| | | | NRAADALNSSMLKDFLSQPSLGLLVRTYPELE |
| TIAM 2 | 6912703 | 1 | PLNVYDVQLTKTGSVCDFGFAVTAQVDERQHLSRIFISDVLPDGLAYGEGLRKG |
| | | | NEIMTLNGEAVSDLDLKQMEALFSEKSVGLTLIARPPDTKATL |
| TIP1 | 2613001 | 1 | QRVEIHKLRQGENLILGFSIGGGIDQDPSQNPFSEDKTDKGIYVTRVSEGGPAEIA |
| | | | GLQIGDKIMQVNGWDMTMVTHDQARKRLTKRSEEVVRLLVTRQSLQK |
| TIP2 | 2613003 | 1 | RKEVEVFKSEDALGLTITDNGAGYAFIKRIKEGSVIDHIHLISVGDMIEAINGQSLL |
| | | | GCRHYEVARLLKELPRGRTFTLKLTEPRK |
| TIP33 | 2613007 | 1 | HSHPRVVELPKTDEGLGFNVMGGKEQNSPIYISRIIPGGVAERHGGLKRGDQLLS |
| | | | VNGVSVEGEHHEKAVELLKAAKDSVKLVVRYTPKVL |
| TIP43 | 2613011 | 1 | ISNQKRGVKVLKQELGGLGISIKGGKENKMPILISKIFKGLAADQTQALYVGDAILS |
| | | | VNGADLRDATHDEAVQALKRAGKEVLLEVKYMREATPYV |
| X-11 beta | 3005559 | 1 | IHFSNSENCKELQLEKHKGEILGVVVVESGWGSILPTVILANMMNGGPAARSGK |
| | | | LSIGDQIMSINGTSLVGLPLATCQGIIKGLKNQTQVKLNIVSCPPVTTVLIKRNSS |
| X-11 beta | 3005559 | 2 | IPPVTTVLIKRPDLKYQLGFSVQNGIICSLMRGGIAERGGVRVGHRIIEINGQSVVA |
| | | | TAHEKIVQALSNSVGEIHMKTMPAAMFRLLTGQENSS |
| ZO-1 | 292937 | 1 | IWEQHTVTLHRAPGFGFGIAISGGRDNPHFQSGETSIVISDVLKGGPAEGQLQEN |
| | | | DRVAMVNGVSMDNVEHAFAVQQLRKSGKNAKITIRRKKKVQIPNSS |
| ZO-1 | 292937 | 2 | ISSQPAKPTKVTLVKSRKNEEYGLRLASHIFVKEISQDSLAARDGNIQEGDVVLK |
| | | | INGTVTENMSLTDAKTLIERSKGKLKMVVQRDRATLLNSS |
| ZO-1 | 292937 | 3 | IRMKLVKFRKGDSVGLRLAGGNDVGIFVAGVLEDSPMKEGLEEGDQILRVNNV |
| | | | DFTNIIREEAVLFLLDLPKGEEVTILAQKKKDVFSN |
| ZO-2 | 12734763 | 1 | LIWEQYTVTLQKDSKRGFGIAVSGGRDNPHFENGETSIVISDVLPGGPADGLLQE |
| | | | NDRVVMVNGTPMEDVLHSFAVQQLRKSGKVAAIVVKRPRKV |
| ZO-2 | 12734763 | 2 | RVLLMKSRANEEYGLRLGSQIFVKEMTRTGLATKDGNLHEGDIILKINGTVTENM |
| | | | SLTDARKLIEKSRGKLQLVVLRDS |
| ZO-2 | 12734763 | 3 | HAPNTKMVRFKKGDSVGLRLAGGNDVGIFVAGIQEGTSAEQEGLQEGDQILKVN |
| | | | TQDFRGLVREDAVLYLLEIPKGEMVTILAQSRADVY |
| ZO-3 | 10092690 | 1 | IPGNSTIWEQHTATLSKDPRRGFGIAISGGRDRPGGSMVVSDVVPGGPAEGRLQT |
| | | | GDHIVMVNGVSMENATSAFAIQILKTCTKMANITVKRPRRIHLPAEFIVTD |
| ZO-3 | 10092690 | 2 | QDVQMKPVKSVLVKRRDSEEFGVKLGSQIFIKHITDSGLAARHRGLQEGDLILQI |
| | | | NGVSSQNLSLNDTRRLIEKSEGKLSLLVLRDRGQFLVNIPNSS |
| ZO-3 | 10092690 | 3 | RGYSPDTRVVRFLKGKSIGLRLAGGNDVGIFVSGVQAGSPADGQGIQEGDQILQV |
| | | | NDVPFQNLTREEAVQFLLGLPPGEEMELVTQRKQDIFWKMVQSEFIVTD |

*No GI number for this PDZ domain containing protein - it was computer cloned by J.S. using rat Shank3 seq against human genomic clone AC000036. In silico spliced together nt6400-6496, 6985-7109, 7211-7400 to create hypothetical human Shank3.

1. A method of detecting PDZ polypeptide binding to an alpha adrenergic receptor, comprising:
a) combining a labeled polypeptide containing an alpha adrenergic receptor C-terminal PL sequence with a PDZ polypeptide in vitro, and
b) detecting binding between the PDZ polypeptide and the alpha adrenergic receptor polypeptide

2. The method of claim 1 wherein the PL polypeptide is a biotinylated peptide.

3. The method of claim 1 wherein the PL polypeptide is a fluorescence labeled peptide.

4. The method of claim 1 wherein the PL polypeptide is an epitope tagged protein expressed in a host cell.

5. A method of determining whether a test compound is a modulator of binding between a PDZ polypeptide and an alpha adrenergic PL polypeptide, comprising:
(a) contacting under suitable binding conditions (i) a PDZ polypeptide, and (ii) a PL peptide, wherein
the PL peptide comprises a C-terminal sequence of the PL polypeptide,
the PDZ polypeptide and the PL peptide are a binding pair as specified in Table 8; and
contacting is performed in the presence of the test compound; and
(b) detecting formation of a complex between the PDZ-domain polypeptide and the PL peptide, wherein
(i) presence of the complex at a level that is statistically significantly higher in the presence of the test compound than in the absence of test compound is an indication that the test compound is an agonist, and
(ii) presence of the complex at a level that is statistically significantly lower in the presence of the test compound than in the absence of test compound is an indication that the test compound is an antagonist.

6. The method of claim 5, wherein the modulator is a peptide.

7. A modulator of binding between a specific PDZ polypeptide and an alpha adrenergic receptor PL polypeptide, wherein the modulator is
(a) a peptide comprising at least 3 residues of a C-terminal sequence demonstrated to bind the target PDZ polypeptide; or
(b) a peptide mimetic of the peptide of section (a); or
(c) a small molecule having similar functional activity as the peptide of section (a) with respect to the PDZ polypeptide and PL polypeptide binding pair.

8. The modulator of claim 7 that modulates a specific interaction listed in Table 8.

9. The modulator of claim 7 that is an agonist.

10. The modulator of claim 7 that is an antagonist.

11. A pharmaceutical composition comprising a modulator of claim 7.

12. A method of treating a disorder from Table 9, comprising administering a therapeutically effective amount of a modulator of claim 7, wherein the PDZ polypeptide and the alpha adrenergic receptor PL polypeptide are a binding pair as specified in Table 8.
